SEQUENCE LISTING (589 sequences)

SEQ ID NO: 1 - protein, 20 aa, synthetic construct
  REGION 1..20: note = Synthetic
VPLHGTILTRPLLESELVIG

SEQ ID NO: 2 - protein, 16 aa, synthetic construct
  REGION 1..16: note = Synthetic
VILRGHLRIAGHHLGR

SEQ ID NO: 3 - protein, 18 aa, synthetic construct
  REGION 1..18: note = Synthetic
VILRGHLRIAGHHLGRCD

SEQ ID NO: 4 - protein, 15 aa, synthetic construct
  REGION 1..15: note = Synthetic
ILRGHLRIAGHHLGR

SEQ ID NO: 5 - protein, 16 aa, synthetic construct
  REGION 1..16: note = Synthetic
ILRGHLRIAGHHLGRC

SEQ ID NO: 6 - protein, 17 aa, synthetic construct
  REGION 1..17: note = Synthetic
ILRGHLRIAGHHLGRCD

SEQ ID NO: 7 - protein, 20 aa, synthetic construct
  REGION 1..20: note = Synthetic
ILRGHLRIAGHHLGRCDIKD

SEQ ID NO: 8 - protein, 14 aa, synthetic construct
  REGION 1..14: note = Synthetic
LRGHLRIAGHHLGR

SEQ ID NO: 9 - protein, 15 aa, synthetic construct
  REGION 1..15: note = Synthetic
LRGHLRIAGHHLGRC

SEQ ID NO: 10 - protein, 16 aa, synthetic construct
  REGION 1..16: note = Synthetic
LRGHLRIAGHHLGRCD

SEQ ID NO: 11 - protein, 13 aa, synthetic construct
  REGION 1..13: note = Synthetic
RGHLRIAGHHLGR

SEQ ID NO: 12 - protein, 15 aa, synthetic construct
  REGION 1..15: note = Synthetic
RGHLRIAGHHLGRCD

SEQ ID NO: 13 - protein, 18 aa, synthetic construct
  REGION 1..18: note = Synthetic
RGHLRIAGHHLGRCDIKD

SEQ ID NO: 14 - protein, 22 aa, synthetic construct
  REGION 1..22: note = Synthetic
RGHLRIAGHHLGRCDIKDLPKE

SEQ ID NO: 15 - protein, 12 aa, synthetic construct
  REGION 1..12: note = Synthetic
GHLRIAGHHLGR

SEQ ID NO: 16 - protein, 14 aa, synthetic construct
  REGION 1..14: note = Synthetic
RCDIKDLPKEITVA

SEQ ID NO: 17 - protein, 19 aa, synthetic construct
  REGION 1..19: note = Synthetic
RTLSYYKLGASQRVAGDSG

SEQ ID NO: 18 - protein, 15 aa, synthetic construct
  REGION 1..15: note = Synthetic
TLSYYKLGASQRVAG

SEQ ID NO: 19 - protein, 17 aa, synthetic construct
  REGION 1..17: note = Synthetic
TLSYYKLGASQRVAGDS

SEQ ID NO: 20 - protein, 14 aa, synthetic construct
  REGION 1..14: note = Synthetic
LSYYKLGASQRVAG

SEQ ID NO: 21 - protein, 16 aa, synthetic construct
  REGION 1..16: note = Synthetic
LSYYKLGASQRVAGDS

SEQ ID NO: 22 - protein, 15 aa, synthetic construct
  REGION 1..15: note = Synthetic
TDHSSSSDNIALLVQ

SEQ ID NO: 23 - protein, 13 aa, synthetic construct
  REGION 1..13: note = Synthetic
HSSSSDNIALLVQ

SEQ ID NO: 24 - protein, 16 aa, synthetic construct
  REGION 1..16: note = Synthetic
SDNGPQNQRNAPRITF

SEQ ID NO: 25 - protein, 17 aa, synthetic construct
  REGION 1..17: note = Synthetic
NTASWFTALTQHGKEDL

SEQ ID NO: 26 - protein, 18 aa, synthetic construct
  REGION 1..18: note = Synthetic
NTASWFTALTQHGKEDLK

SEQ ID NO: 27 - protein, 15 aa, synthetic construct
  REGION 1..15: note = Synthetic
TASWFTALTQHGKED

SEQ ID NO: 28 - protein, 16 aa, synthetic construct
  REGION 1..16: note = Synthetic
TASWFTALTQHGKEDL

SEQ ID NO: 29 - protein, 17 aa, synthetic construct
  REGION 1..17: note = Synthetic
TASWFTALTQHGKEDLK

SEQ ID NO: 30 - protein, 14 aa, synthetic construct
  REGION 1..14: note = Synthetic
ASWFTALTQHGKED

SEQ ID NO: 31 - protein, 15 aa, synthetic construct
  REGION 1..15: note = Synthetic
ASWFTALTQHGKEDL

SEQ ID NO: 32 - protein, 15 aa, synthetic construct
  REGION 1..15: note = Synthetic
TGPEAGLPYGANKDG

SEQ ID NO: 33 - protein, 34 aa, synthetic construct
  REGION 1..34: note = Synthetic
MAGNGGDAALALLLLDRLNQLESKMSGKGQQQQG

SEQ ID NO: 34 - protein, 38 aa, synthetic construct
  REGION 1..38: note = Synthetic
MAGNGGDAALALLLLDRLNQLESKMSGKGQQQQGQTVT

SEQ ID NO: 35 - protein, 22 aa, synthetic construct
  REGION 1..22: note = Synthetic
GNGGDAALALLLLDRLNQLESK

SEQ ID NO: 36 - protein, 36 aa, synthetic construct
  REGION 1..36: note = Synthetic
GNGGDAALALLLLDRLNQLESKMSGKGQQQQGQTVT

SEQ ID NO: 37 - protein, 21 aa, synthetic construct
  REGION 1..21: note = Synthetic
NGGDAALALLLLDRLNQLESK

SEQ ID NO: 38 - protein, 20 aa, synthetic construct
  REGION 1..20: note = Synthetic
GGDAALALLLLDRLNQLESK

SEQ ID NO: 39 - protein, 26 aa, synthetic construct
  REGION 1..26: note = Synthetic
GGDAALALLLLDRLNQLESKMSGKGQ

SEQ ID NO: 40 - protein, 30 aa, synthetic construct
  REGION 1..30: note = Synthetic
GGDAALALLLLDRLNQLESKMSGKGQQQQG

SEQ ID NO: 41 - protein, 32 aa, synthetic construct
  REGION 1..32: note = Synthetic
GGDAALALLLLDRLNQLESKMSGKGQQQQGQT

SEQ ID NO: 42 - protein, 34 aa, synthetic construct
  REGION 1..34: note = Synthetic
GGDAALALLLLDRLNQLESKMSGKGQQQQGQTVT

SEQ ID NO: 43 - protein, 19 aa, synthetic construct
  REGION 1..19: note = Synthetic
GDAALALLLLDRLNQLESK

SEQ ID NO: 44 - protein, 18 aa, synthetic construct
  REGION 1..18: note = Synthetic
DAALALLLLDRLNQLESK

SEQ ID NO: 45 - protein, 13 aa, synthetic construct
  REGION 1..13: note = Synthetic
AALALLLLDRLNQ

SEQ ID NO: 46 - protein, 17 aa, synthetic construct
  REGION 1..17: note = Synthetic
AALALLLLDRLNQLESK

SEQ ID NO: 47 - protein, 12 aa, synthetic construct
  REGION 1..12: note = Synthetic
ALALLLLDRLNQ

SEQ ID NO: 48 - protein, 14 aa, synthetic construct
  REGION 1..14: note = Synthetic
ALALLLLDRLNQLE

SEQ ID NO: 49 - protein, 15 aa, synthetic construct
  REGION 1..15: note = Synthetic
ALALLLLDRLNQLES

SEQ ID NO: 50 - protein, 16 aa, synthetic construct
  REGION 1..16: note = Synthetic
ALALLLLDRLNQLESK

SEQ ID NO: 51 - protein, 15 aa, synthetic construct
  REGION 1..15: note = Synthetic
LALLLLDRLNQLESK

SEQ ID NO: 52 - protein, 14 aa, synthetic construct
  REGION 1..14: note = Synthetic
ALLLLDRLNQLESK

SEQ ID NO: 53 - protein, 12 aa, synthetic construct
  REGION 1..12: note = Synthetic
LLLDRLNQLESK

SEQ ID NO: 54 - protein, 19 aa, synthetic construct
  REGION 1..19: note = Synthetic
TATKAYNVTQAFGRRGPEQ

SEQ ID NO: 55 - protein, 13 aa, synthetic construct
  REGION 1..13: note = Synthetic
IAQFAPSASAFFG

SEQ ID NO: 56 - protein, 30 aa, synthetic construct
  REGION 1..30: note = Synthetic
IGMEVTPSGTWLTYTGAIKLDDKDPNFKDQ

SEQ ID NO: 57 - protein, 35 aa, synthetic construct
  REGION 1..35: note = Synthetic
IGMEVTPSGTWLTYTGAIKLDDKDPNFKDQVILLN

SEQ ID NO: 58 - protein, 23 aa, synthetic construct
  REGION 1..23: note = Synthetic
EVTPSGTWLTYTGAIKLDDKDPN

SEQ ID NO: 59 - protein, 22 aa, synthetic construct
  REGION 1..22: note = Synthetic
VTPSGTWLTYTGAIKLDDKDPN

SEQ ID NO: 60 - protein, 23 aa, synthetic construct
  REGION 1..23: note = Synthetic
VTPSGTWLTYTGAIKLDDKDPNF

SEQ ID NO: 61 - protein, 24 aa, synthetic construct
  REGION 1..24: note = Synthetic
VTPSGTWLTYTGAIKLDDKDPNFK

SEQ ID NO: 62 - protein, 26 aa, synthetic construct
  REGION 1..26: note = Synthetic
VTPSGTWLTYTGAIKLDDKDPNFKDQ

SEQ ID NO: 63 - protein, 18 aa, synthetic construct
  REGION 1..18: note = Synthetic
TPSGTWLTYTGAIKLDDK

SEQ ID NO: 64 - protein, 20 aa, synthetic construct
  REGION 1..20: note = Synthetic
TPSGTWLTYTGAIKLDDKDP

SEQ ID NO: 65 - protein, 21 aa, synthetic construct
  REGION 1..21: note = Synthetic
TPSGTWLTYTGAIKLDDKDPN

SEQ ID NO: 66 - protein, 15 aa, synthetic construct
  REGION 1..15: note = Synthetic
GTWLTYTGAIKLDDK

SEQ ID NO: 67 - protein, 13 aa, synthetic construct
  REGION 1..13: note = Synthetic
WLTYTGAIKLDDK

SEQ ID NO: 68 - protein, 12 aa, synthetic construct
  REGION 1..12: note = Synthetic
LTYTGAIKLDDK

SEQ ID NO: 69 - protein, 15 aa, synthetic construct
  REGION 1..15: note = Synthetic
LTYTGAIKLDDKDPN

SEQ ID NO: 70 - protein, 11 aa, synthetic construct
  REGION 1..11: note = Synthetic
TYTGAIKLDDK

SEQ ID NO: 71 - protein, 11 aa, synthetic construct
  REGION 1..11: note = Synthetic
YTGAIKLDDKD

SEQ ID NO: 72 - protein, 27 aa, synthetic construct
  REGION 1..27: note = Synthetic
LDDKDPNFKDQVILLNKHIDAYKTFPP

SEQ ID NO: 73 - protein, 21 aa, synthetic construct
  REGION 1..21: note = Synthetic
DKDPNFKDQVILLNKHIDAYK

SEQ ID NO: 74 - protein, 22 aa, synthetic construct
  REGION 1..22: note = Synthetic
DKDPNFKDQVILLNKHIDAYKT

SEQ ID NO: 75 - protein, 25 aa, synthetic construct
  REGION 1..25: note = Synthetic
DKDPNFKDQVILLNKHIDAYKTFPP

SEQ ID NO: 76 - protein, 19 aa, synthetic construct
  REGION 1..19: note = Synthetic
DPNFKDQVILLNKHIDAYK

SEQ ID NO: 77 - protein, 20 aa, synthetic construct
  REGION 1..20: note = Synthetic
DPNFKDQVILLNKHIDAYKT

SEQ ID NO: 78 - protein, 23 aa, synthetic construct
  REGION 1..23: note = Synthetic
DPNFKDQVILLNKHIDAYKTFPP

SEQ ID NO: 79 - protein, 16 aa, synthetic construct
  REGION 1..16: note = Synthetic
FKDQVILLNKHIDAYK

SEQ ID NO: 80 - protein, 17 aa, synthetic construct
  REGION 1..17: note = Synthetic
FKDQVILLNKHIDAYKT

SEQ ID NO: 81 - protein, 20 aa, synthetic construct
  REGION 1..20: note = Synthetic
FKDQVILLNKHIDAYKTFPP

SEQ ID NO: 82 - protein, 13 aa, synthetic construct
  REGION 1..13: note = Synthetic
KDQVILLNKHIDA

SEQ ID NO: 83 - protein, 14 aa, synthetic construct
  REGION 1..14: note = Synthetic
KDQVILLNKHIDAY

SEQ ID NO: 84 - protein, 15 aa, synthetic construct
  REGION 1..15: note = Synthetic
KDQVILLNKHIDAYK

SEQ ID NO: 85 - protein, 16 aa, synthetic construct
  REGION 1..16: note = Synthetic
KDQVILLNKHIDAYKT

SEQ ID NO: 86 - protein, 19 aa, synthetic construct
  REGION 1..19: note = Synthetic
KDQVILLNKHIDAYKTFPP

SEQ ID NO: 87 - protein, 12 aa, synthetic construct
  REGION 1..12: note = Synthetic
DQVILLNKHIDA

SEQ ID NO: 88 - protein, 14 aa, synthetic construct
  REGION 1..14: note = Synthetic
DQVILLNKHIDAYK

SEQ ID NO: 89 - protein, 15 aa, synthetic construct
  REGION 1..15: note = Synthetic
DQVILLNKHIDAYKT

SEQ ID NO: 90 - protein, 11 aa, synthetic construct
  REGION 1..11: note = Synthetic
ILLNKHIDAYK

SEQ ID NO: 91 - protein, 10 aa, synthetic construct
  REGION 1..10: note = Synthetic
LLNKHIDAYK

SEQ ID NO: 92 - protein, 19 aa, synthetic construct
  REGION 1..19: note = Synthetic
QRQKKQQTVTLLPAADLDD

SEQ ID NO: 93 - protein, 21 aa, synthetic construct
  REGION 1..21: note = Synthetic
QRQKKQQTVTLLPAADLDDFS

SEQ ID NO: 94 - protein, 25 aa, synthetic construct
  REGION 1..25: note = Synthetic
QRQKKQQTVTLLPAADLDDFSKQLQ

SEQ ID NO: 95 - protein, 35 aa, synthetic construct
  REGION 1..35: note = Synthetic
QRQKKQQTVTLLPAADLDDFSKQLQQSMSSADSTQ

SEQ ID NO: 96 - protein, 36 aa, synthetic construct
  REGION 1..36: note = Synthetic
QRQKKQQTVTLLPAADLDDFSKQLQQSMSSADSTQA

SEQ ID NO: 97 - protein, 13 aa, synthetic construct
  REGION 1..13: note = Synthetic
KELITNIGRKLHN

SEQ ID NO: 98 - protein, 15 aa, synthetic construct
  REGION 1..15: note = Synthetic
EVFVKITGFDSLKSN

SEQ ID NO: 99 - protein, 28 aa, synthetic construct
  REGION 1..28: note = Synthetic
MDLFMRIFTIGTVTLKQGEIKDATPSDF

SEQ ID NO: 100 - protein, 13 aa, synthetic construct
  REGION 1..13: note = Synthetic
YIINLIIKNLSKS

SEQ ID NO: 101 - protein, 32 aa, synthetic construct
  REGION 1..32: note = Synthetic
TQLPPAYTNSFTRGVYYPDKVFRSSVLHSTQD

SEQ ID NO: 102 - protein, 17 aa, synthetic construct
  REGION 1..17: note = Synthetic
LPPAYTNSFTRGVYYPD

SEQ ID NO: 103 - protein, 18 aa, synthetic construct
  REGION 1..18: note = Synthetic
SFTRGVYYPDKVFRSSVL

SEQ ID NO: 104 - protein, 16 aa, synthetic construct
  REGION 1..16: note = Synthetic
FTRGVYYPDKVFRSSV

SEQ ID NO: 105 - protein, 17 aa, synthetic construct
  REGION 1..17: note = Synthetic
FTRGVYYPDKVFRSSVL

SEQ ID NO: 106 - protein, 15 aa, synthetic construct
  REGION 1..15: note = Synthetic
TRGVYYPDKVFRSSV

SEQ ID NO: 107 - protein, 14 aa, synthetic construct
  REGION 1..14: note = Synthetic
RGVYYPDKVFRSSV

SEQ ID NO: 108 - protein, 14 aa, synthetic construct
  REGION 1..14: note = Synthetic
GVYYPDKVFRSSVL

SEQ ID NO: 109 - protein, 16 aa, synthetic construct
  REGION 1..16: note = Synthetic
YPDKVFRSSVLHSTQD

SEQ ID NO: 110 - protein, 17 aa, synthetic construct
  REGION 1..17: note = Synthetic
YPDKVFRSSVLHSTQDL

SEQ ID NO: 111 - protein, 23 aa, synthetic construct
  REGION 1..23: note = Synthetic
YPDKVFRSSVLHSTQDLFLPFFS

SEQ ID NO: 112 - protein, 16 aa, synthetic construct
  REGION 1..16: note = Synthetic
RSSVLHSTQDLFLPFF

SEQ ID NO: 113 - protein, 17 aa, synthetic construct
  REGION 1..17: note = Synthetic
RSSVLHSTQDLFLPFFS

SEQ ID NO: 114 - protein, 22 aa, synthetic construct
  REGION 1..22: note = Synthetic
KNIDGYFKIYSKHTPINLVRDL

SEQ ID NO: 115 - protein, 18 aa, synthetic construct
  REGION 1..18: note = Synthetic
TVEKGIYQTSNFRVQPTE

SEQ ID NO: 116 - protein, 16 aa, synthetic construct
  REGION 1..16: note = Synthetic
VEKGIYQTSNFRVQPT

SEQ ID NO: 117 - protein, 18 aa, synthetic construct
  REGION 1..18: note = Synthetic
VEKGIYQTSNFRVQPTES

SEQ ID NO: 118 - protein, 17 aa, synthetic construct
  REGION 1..17: note = Synthetic
TPTWRVYSTGSNVFQTR

SEQ ID NO: 119 - protein, 15 aa, synthetic construct
  REGION 1..15: note = Synthetic
EPQIITTDNTFVSGN

SEQ ID NO: 120 - protein, 14 aa, synthetic construct
  REGION 1..14: note = Synthetic
NVPLHGTILTRPLL

SEQ ID NO: 121 - protein, 14 aa, synthetic construct
  REGION 1..14: note = Synthetic
ESELVIGAVILRGH

SEQ ID NO: 122 - protein, 15 aa, synthetic construct
  REGION 1..15: note = Synthetic
ESELVIGAVILRGHL

SEQ ID NO: 123 - protein, 16 aa, synthetic construct
  REGION 1..16: note = Synthetic
ESELVIGAVILRGHLR

SEQ ID NO: 124 - protein, 11 aa, synthetic construct
  REGION 1..11: note = Synthetic
SELVIGAVILR

SEQ ID NO: 125 - protein, 13 aa, synthetic construct
  REGION 1..13: note = Synthetic
SELVIGAVILRGH

SEQ ID NO: 126 - protein, 14 aa, synthetic construct
  REGION 1..14: note = Synthetic
SELVIGAVILRGHL

SEQ ID NO: 127 - protein, 15 aa, synthetic construct
  REGION 1..15: note = Synthetic
SELVIGAVILRGHLR

SEQ ID NO: 128 - protein, 12 aa, synthetic construct
  REGION 1..12: note = Synthetic
ELVIGAVILRGH

SEQ ID NO: 129 - protein, 14 aa, synthetic construct
  REGION 1..14: note = Synthetic
ELVIGAVILRGHLR

SEQ ID NO: 130 - protein, 11 aa, synthetic construct
  REGION 1..11: note = Synthetic
LVIGAVILRGH

SEQ ID NO: 131 - protein, 12 aa, synthetic construct
  REGION 1..12: note = Synthetic
LVIGAVILRGHL

SEQ ID NO: 132 - protein, 13 aa, synthetic construct
  REGION 1..13: note = Synthetic
LVIGAVILRGHLR

SEQ ID NO: 133 - protein, 10 aa, synthetic construct
  REGION 1..10: note = Synthetic
VIGAVILRGH

SEQ ID NO: 134 - protein, 9 aa, synthetic construct
  REGION 1..9: note = Synthetic
IGAVILRGH

SEQ ID NO: 135 - protein, 10 aa, synthetic construct
  REGION 1..10: note = Synthetic
IGAVILRGHL

SEQ ID NO: 136 - protein, 11 aa, synthetic construct
  REGION 1..11: note = Synthetic
IGAVILRGHLR

SEQ ID NO: 137 - protein, 10 aa, synthetic construct
  REGION 1..10: note = Synthetic
GAVILRGHLR

SEQ ID NO: 138 - protein, 18 aa, synthetic construct
  REGION 1..18: note = Synthetic
TSRTLSYYKLGASQRVAG

SEQ ID NO: 139 - protein, 21 aa, synthetic construct
  REGION 1..21: note = Synthetic
TSRTLSYYKLGASQRVAGDSG

SEQ ID NO: 140 - protein, 16 aa, synthetic construct
  REGION 1..16: note = Synthetic
SRTLSYYKLGASQRVA

SEQ ID NO: 141 - protein, 17 aa, synthetic construct
  REGION 1..17: note = Synthetic
SRTLSYYKLGASQRVAG

SEQ ID NO: 142 - protein, 18 aa, synthetic construct
  REGION 1..18: note = Synthetic
SRTLSYYKLGASQRVAGD

SEQ ID NO: 143 - protein, 20 aa, synthetic construct
  REGION 1..20: note = Synthetic
SRTLSYYKLGASQRVAGDSG

SEQ ID NO: 144 - protein, 15 aa, synthetic construct
  REGION 1..15: note = Synthetic
RTLSYYKLGASQRVA

SEQ ID NO: 145 - protein, 16 aa, synthetic construct
  REGION 1..16: note = Synthetic
RTLSYYKLGASQRVAG

SEQ ID NO: 146 - protein, 17 aa, synthetic construct
  REGION 1..17: note = Synthetic
RTLSYYKLGASQRVAGD

SEQ ID NO: 147 - protein, 19 aa, synthetic construct
  REGION 1..19: note = Synthetic
RTLSYYKLGASQRVAGDSG

SEQ ID NO: 148 - protein, 14 aa, synthetic construct
  REGION 1..14: note = Synthetic
TLSYYKLGASQRVA

SEQ ID NO: 149 - protein, 15 aa, synthetic construct
  REGION 1..15: note = Synthetic
TLSYYKLGASQRVAG

SEQ ID NO: 150 - protein, 16 aa, synthetic construct
  REGION 1..16: note = Synthetic
TLSYYKLGASQRVAGD

SEQ ID NO: 151 - protein, 15 aa, synthetic construct
  REGION 1..15: note = Synthetic
TDHSSSSDNIALLVQ

SEQ ID NO: 152 - protein, 13 aa, synthetic construct
  REGION 1..13: note = Synthetic
HSSSSDNIALLVQ

SEQ ID NO: 153 - protein, 22 aa, synthetic construct
  REGION 1..22: note = Synthetic
GLPNNTASWFTALTQHGKEDLK

SEQ ID NO: 154 - protein, 25 aa, synthetic construct
  REGION 1..25: note = Synthetic
GLPNNTASWFTALTQHGKEDLKFPR

SEQ ID NO: 155 - protein, 30 aa, synthetic construct
  REGION 1..30: note = Synthetic
GLPNNTASWFTALTQHGKEDLKFPRGQGVP

SEQ ID NO: 156 - protein, 34 aa, synthetic construct
  REGION 1..34: note = Synthetic
GLPNNTASWFTALTQHGKEDLKFPRGQGVPINTN

SEQ ID NO: 157 - protein, 28 aa, synthetic construct
  REGION 1..28: note = Synthetic
NTASWFTALTQHGKEDLKFPRGQGVPIN

SEQ ID NO: 158 - protein, 17 aa, synthetic construct
  REGION 1..17: note = Synthetic
TASWFTALTQHGKEDLK

SEQ ID NO: 159 - protein, 21 aa, synthetic construct
  REGION 1..21: note = Synthetic
TASWFTALTQHGKEDLKFPRG

SEQ ID NO: 160 - protein, 29 aa, synthetic construct
  REGION 1..29: note = Synthetic
TASWFTALTQHGKEDLKFPRGQGVPINTN

SEQ ID NO: 161 - protein, 14 aa, synthetic construct
  REGION 1..14: note = Synthetic
ASWFTALTQHGKED

SEQ ID NO: 162 - protein, 16 aa, synthetic construct
  REGION 1..16: note = Synthetic
ASWFTALTQHGKEDLK

SEQ ID NO: 163 - protein, 27 aa, synthetic construct
  REGION 1..27: note = Synthetic
GQGVPINTNSSPDDQIGYYRRATRRIR

SEQ ID NO: 164 - protein, 38 aa, synthetic construct
  REGION 1..38: note = Synthetic
VATEGALNTPKDHIGTRNPANNAAIVLQLPQGTTLPKG

SEQ ID NO: 165 - protein, 37 aa, synthetic construct
  REGION 1..37: note = Synthetic
ATEGALNTPKDHIGTRNPANNAAIVLQLPQGTTLPKG

SEQ ID NO: 166 - protein, 35 aa, synthetic construct
  REGION 1..35: note = Synthetic
TEGALNTPKDHIGTRNPANNAAIVLQLPQGTTLPK

SEQ ID NO: 167 - protein, 36 aa, synthetic construct
  REGION 1..36: note = Synthetic
TEGALNTPKDHIGTRNPANNAAIVLQLPQGTTLPKG

SEQ ID NO: 168 - protein, 32 aa, synthetic construct
  REGION 1..32: note = Synthetic
LNTPKDHIGTRNPANNAAIVLQLPQGTTLPKG

SEQ ID NO: 169 - protein, 27 aa, synthetic construct
  REGION 1..27: note = Synthetic
NTPKDHIGTRNPANNAAIVLQLPQGTT

SEQ ID NO: 170 - protein, 31 aa, synthetic construct
  REGION 1..31: note = Synthetic
NTPKDHIGTRNPANNAAIVLQLPQGTTLPKG

SEQ ID NO: 171 - protein, 24 aa, synthetic construct
  REGION 1..24: note = Synthetic
TPKDHIGTRNPANNAAIVLQLPQG

SEQ ID NO: 172 - protein, 26 aa, synthetic construct
  REGION 1..26: note = Synthetic
TPKDHIGTRNPANNAAIVLQLPQGTT

SEQ ID NO: 173 - protein, 28 aa, synthetic construct
  REGION 1..28: note = Synthetic
TPKDHIGTRNPANNAAIVLQLPQGTTLP

SEQ ID NO: 174 - protein, 29 aa, synthetic construct
  REGION 1..29: note = Synthetic
TPKDHIGTRNPANNAAIVLQLPQGTTLPK

SEQ ID NO: 175 - protein, 30 aa, synthetic construct
  REGION 1..30: note = Synthetic
TPKDHIGTRNPANNAAIVLQLPQGTTLPKG

SEQ ID NO: 176 - protein, 20 aa, synthetic construct
  REGION 1..20: note = Synthetic
HIGTRNPANNAAIVLQLPQG

SEQ ID NO: 177 - protein, 22 aa, synthetic construct
  REGION 1..22: note = Synthetic
HIGTRNPANNAAIVLQLPQGTT

SEQ ID NO: 178 - protein, 19 aa, synthetic construct
  REGION 1..19: note = Synthetic
TRNPANNAAIVLQLPQGTT

SEQ ID NO: 179 - protein, 22 aa, synthetic construct
  REGION 1..22: note = Synthetic
TRNPANNAAIVLQLPQGTTLPK

SEQ ID NO: 180 - protein, 23 aa, synthetic construct
  REGION 1..23: note = Synthetic
TRNPANNAAIVLQLPQGTTLPKG

SEQ ID NO: 181 - protein, 15 aa, synthetic construct
  REGION 1..15: note = Synthetic
RNPANNAAIVLQLPQ

SEQ ID NO: 182 - protein, 16 aa, synthetic construct
  REGION 1..16: note = Synthetic
RNPANNAAIVLQLPQG

SEQ ID NO: 183 - protein, 18 aa, synthetic construct
  REGION 1..18: note = Synthetic
RNPANNAAIVLQLPQGTT

SEQ ID NO: 184 - protein, 21 aa, synthetic construct
  REGION 1..21: note = Synthetic
RNPANNAAIVLQLPQGTTLPK

SEQ ID NO: 185 - protein, 22 aa, synthetic construct
  REGION 1..22: note = Synthetic
RNPANNAAIVLQLPQGTTLPKG

SEQ ID NO: 186 - protein, 11 aa, synthetic construct
  REGION 1..11: note = Synthetic
NPANNAAIVLQ

SEQ ID NO: 187 - protein, 14 aa, synthetic construct
  REGION 1..14: note = Synthetic
NPANNAAIVLQLPQ

SEQ ID NO: 188 - protein, 15 aa, synthetic construct
  REGION 1..15: note = Synthetic
NPANNAAIVLQLPQG

SEQ ID NO: 189 - protein, 17 aa, synthetic construct
  REGION 1..17: note = Synthetic
NPANNAAIVLQLPQGTT

SEQ ID NO: 190 - protein, 20 aa, synthetic construct
  REGION 1..20: note = Synthetic
NPANNAAIVLQLPQGTTLPK

SEQ ID NO: 191 - protein, 21 aa, synthetic construct
  REGION 1..21: note = Synthetic
NPANNAAIVLQLPQGTTLPKG

SEQ ID NO: 192 - protein, 13 aa, synthetic construct
  REGION 1..13: note = Synthetic
PANNAAIVLQLPQ

SEQ ID NO: 193 - protein, 14 aa, synthetic construct
  REGION 1..14: note = Synthetic
PANNAAIVLQLPQG

SEQ ID NO: 194 - protein, 31 aa, synthetic construct
  REGION 1..31: note = Synthetic
AAEASKKPRQKRTATKAYNVTQAFGRRGPEQ

SEQ ID NO: 195 - protein, 29 aa, synthetic construct
  REGION 1..29: note = Synthetic
EASKKPRQKRTATKAYNVTQAFGRRGPEQ

SEQ ID NO: 196 - protein, 20 aa, synthetic construct
  REGION 1..20: note = Synthetic
QKRTATKAYNVTQAFGRRGP

SEQ ID NO: 197 - protein, 21 aa, synthetic construct
  REGION 1..21: note = Synthetic
QKRTATKAYNVTQAFGRRGPE

SEQ ID NO: 198 - protein, 22 aa, synthetic construct
  REGION 1..22: note = Synthetic
QKRTATKAYNVTQAFGRRGPEQ

SEQ ID NO: 199 - protein, 23 aa, synthetic construct
  REGION 1..23: note = Synthetic
QKRTATKAYNVTQAFGRRGPEQT

SEQ ID NO: 200 - protein, 26 aa, synthetic construct
  REGION 1..26: note = Synthetic
QKRTATKAYNVTQAFGRRGPEQTQGN

SEQ ID NO: 201 - protein, 17 aa, synthetic construct
  REGION 1..17: note = Synthetic
KRTATKAYNVTQAFGRR

SEQ ID NO: 202 - protein, 19 aa, synthetic construct
  REGION 1..19: note = Synthetic
KRTATKAYNVTQAFGRRGP

SEQ ID NO: 203 - protein, 20 aa, synthetic construct
  REGION 1..20: note = Synthetic
KRTATKAYNVTQAFGRRGPE

SEQ ID NO: 204 - protein, 21 aa, synthetic construct
  REGION 1..21: note = Synthetic
KRTATKAYNVTQAFGRRGPEQ

SEQ ID NO: 205 - protein, 25 aa, synthetic construct
  REGION 1..25: note = Synthetic
KRTATKAYNVTQAFGRRGPEQTQGN

SEQ ID NO: 206 - protein, 16 aa, synthetic construct
  REGION 1..16: note = Synthetic
RTATKAYNVTQAFGRR

SEQ ID NO: 207 - protein, 17 aa, synthetic construct
  REGION 1..17: note = Synthetic
RTATKAYNVTQAFGRRG

SEQ ID NO: 208 - protein, 18 aa, synthetic construct
  REGION 1..18: note = Synthetic
RTATKAYNVTQAFGRRGP

SEQ ID NO: 209 - protein, 19 aa, synthetic construct
  REGION 1..19: note = Synthetic
RTATKAYNVTQAFGRRGPE

SEQ ID NO: 210 - protein, 20 aa, synthetic construct
  REGION 1..20: note = Synthetic
RTATKAYNVTQAFGRRGPEQ

SEQ ID NO: 211 - protein, 21 aa, synthetic construct
  REGION 1..21: note = Synthetic
RTATKAYNVTQAFGRRGPEQT

SEQ ID NO: 212 - protein, 22 aa, synthetic construct
  REGION 1..22: note = Synthetic
RTATKAYNVTQAFGRRGPEQTQ

SEQ ID NO: 213 - protein, 23 aa, synthetic construct
  REGION 1..23: note = Synthetic
RTATKAYNVTQAFGRRGPEQTQG

SEQ ID NO: 214 - protein, 24 aa, synthetic construct
  REGION 1..24: note = Synthetic
RTATKAYNVTQAFGRRGPEQTQGN

SEQ ID NO: 215 - protein, 25 aa, synthetic construct
  REGION 1..25: note = Synthetic
RTATKAYNVTQAFGRRGPEQTQGNF

SEQ ID NO: 216 - protein, 26 aa, synthetic construct
  REGION 1..26: note = Synthetic
RTATKAYNVTQAFGRRGPEQTQGNFG

SEQ ID NO: 217 - protein, 32 aa, synthetic construct
  REGION 1..32: note = Synthetic
RTATKAYNVTQAFGRRGPEQTQGNFGDQELIR

SEQ ID NO: 218 - protein, 15 aa, synthetic construct
  REGION 1..15: note = Synthetic
TATKAYNVTQAFGRR

SEQ ID NO: 219 - protein, 16 aa, synthetic construct
  REGION 1..16: note = Synthetic
TATKAYNVTQAFGRRG

SEQ ID NO: 220 - protein, 17 aa, synthetic construct
  REGION 1..17: note = Synthetic
TATKAYNVTQAFGRRGP

SEQ ID NO: 221 - protein, 18 aa, synthetic construct
  REGION 1..18: note = Synthetic
TATKAYNVTQAFGRRGPE

SEQ ID NO: 222 - protein, 19 aa, synthetic construct
  REGION 1..19: note = Synthetic
TATKAYNVTQAFGRRGPEQ

SEQ ID NO: 223 - protein, 20 aa, synthetic construct
  REGION 1..20: note = Synthetic
TATKAYNVTQAFGRRGPEQT

SEQ ID NO: 224 - protein, 21 aa, synthetic construct
  REGION 1..21: note = Synthetic
TATKAYNVTQAFGRRGPEQTQ

SEQ ID NO: 225 - protein, 22 aa, synthetic construct
  REGION 1..22: note = Synthetic
TATKAYNVTQAFGRRGPEQTQG

SEQ ID NO: 226 - protein, 23 aa, synthetic construct
  REGION 1..23: note = Synthetic
TATKAYNVTQAFGRRGPEQTQGN

SEQ ID NO: 227 - protein, 24 aa, synthetic construct
  REGION 1..24: note = Synthetic
TATKAYNVTQAFGRRGPEQTQGNF

SEQ ID NO: 228 - protein, 25 aa, synthetic construct
  REGION 1..25: note = Synthetic
TATKAYNVTQAFGRRGPEQTQGNFG

SEQ ID NO: 229 - protein, 26 aa, synthetic construct
  REGION 1..26: note = Synthetic
TATKAYNVTQAFGRRGPEQTQGNFGD

SEQ ID NO: 230 - protein, 27 aa, synthetic construct
  REGION 1..27: note = Synthetic
TATKAYNVTQAFGRRGPEQTQGNFGDQ

SEQ ID NO: 231 - protein, 28 aa, synthetic construct
  REGION 1..28: note = Synthetic
TATKAYNVTQAFGRRGPEQTQGNFGDQE

SEQ ID NO: 232 - protein, 29 aa, synthetic construct
  REGION 1..29: note = Synthetic
TATKAYNVTQAFGRRGPEQTQGNFGDQEL

SEQ ID NO: 233 - protein, 30 aa, synthetic construct
  REGION 1..30: note = Synthetic
TATKAYNVTQAFGRRGPEQTQGNFGDQELI

SEQ ID NO: 234 - protein, 31 aa, synthetic construct
  REGION 1..31: note = Synthetic
TATKAYNVTQAFGRRGPEQTQGNFGDQELIR

SEQ ID NO: 235 - protein, 32 aa, synthetic construct
  REGION 1..32: note = Synthetic
TATKAYNVTQAFGRRGPEQTQGNFGDQELIRQ

SEQ ID NO: 236 - protein, 33 aa, synthetic construct
  REGION 1..33: note = Synthetic
TATKAYNVTQAFGRRGPEQTQGNFGDQELIRQG

SEQ ID NO: 237 - protein, 34 aa, synthetic construct
  REGION 1..34: note = Synthetic
TATKAYNVTQAFGRRGPEQTQGNFGDQELIRQGT

SEQ ID NO: 238 - protein, 35 aa, synthetic construct
  REGION 1..35: note = Synthetic
TATKAYNVTQAFGRRGPEQTQGNFGDQELIRQGTD

SEQ ID NO: 239 - protein, 14 aa, synthetic construct
  REGION 1..14: note = Synthetic
ATKAYNVTQAFGRR

SEQ ID NO: 240 - protein, 15 aa, synthetic construct
  REGION 1..15: note = Synthetic
ATKAYNVTQAFGRRG

SEQ ID NO: 241 - protein, 16 aa, synthetic construct
  REGION 1..16: note = Synthetic
ATKAYNVTQAFGRRGP

SEQ ID NO: 242 - protein, 17 aa, synthetic construct
  REGION 1..17: note = Synthetic
ATKAYNVTQAFGRRGPE

SEQ ID NO: 243 - protein, 18 aa, synthetic construct
  REGION 1..18: note = Synthetic
ATKAYNVTQAFGRRGPEQ

SEQ ID NO: 244 - protein, 19 aa, synthetic construct
  REGION 1..19: note = Synthetic
ATKAYNVTQAFGRRGPEQT

SEQ ID NO: 245 - protein, 20 aa, synthetic construct
  REGION 1..20: note = Synthetic
ATKAYNVTQAFGRRGPEQTQ

SEQ ID NO: 246 - protein, 22 aa, synthetic construct
  REGION 1..22: note = Synthetic
ATKAYNVTQAFGRRGPEQTQGN

SEQ ID NO: 247 - protein, 24 aa, synthetic construct
  REGION 1..24: note = Synthetic
ATKAYNVTQAFGRRGPEQTQGNFG

SEQ ID NO: 248 - protein, 13 aa, synthetic construct
  REGION 1..13: note = Synthetic
TKAYNVTQAFGRR

SEQ ID NO: 249 - protein, 14 aa, synthetic construct
  REGION 1..14: note = Synthetic
TKAYNVTQAFGRRG

SEQ ID NO: 250 - protein, 15 aa, synthetic construct
  REGION 1..15: note = Synthetic
TKAYNVTQAFGRRGP

SEQ ID NO: 251 - protein, 16 aa, synthetic construct
  REGION 1..16: note = Synthetic
TKAYNVTQAFGRRGPE

SEQ ID NO: 252 - protein, 17 aa, synthetic construct
  REGION 1..17: note = Synthetic
TKAYNVTQAFGRRGPEQ

SEQ ID NO: 253 - protein, 21 aa, synthetic construct
  REGION 1..21: note = Synthetic
TKAYNVTQAFGRRGPEQTQGN

SEQ ID NO: 254 - protein, 12 aa, synthetic construct
  REGION 1..12: note = Synthetic
KAYNVTQAFGRR

SEQ ID NO: 255 - protein, 13 aa, synthetic construct
  REGION 1..13: note = Synthetic
KAYNVTQAFGRRG

SEQ ID NO: 256 - protein, 14 aa, synthetic construct
  REGION 1..14: note = Synthetic
KAYNVTQAFGRRGP

SEQ ID NO: 257 - protein, 15 aa, synthetic construct
  REGION 1..15: note = Synthetic
KAYNVTQAFGRRGPE

SEQ ID NO: 258 - protein, 16 aa, synthetic construct
  REGION 1..16: note = Synthetic
KAYNVTQAFGRRGPEQ

SEQ ID NO: 259 - protein, 17 aa, synthetic construct
  REGION 1..17: note = Synthetic
KAYNVTQAFGRRGPEQT

SEQ ID NO: 260 - protein, 20 aa, synthetic construct
  REGION 1..20: note = Synthetic
KAYNVTQAFGRRGPEQTQGN

SEQ ID NO: 261 - protein, 13 aa, synthetic construct
  REGION 1..13: note = Synthetic
AYNVTQAFGRRGP

SEQ ID NO: 262 - protein, 15 aa, synthetic construct
  REGION 1..15: note = Synthetic
AYNVTQAFGRRGPEQ

SEQ ID NO: 263 - protein, 19 aa, synthetic construct
  REGION 1..19: note = Synthetic
AYNVTQAFGRRGPEQTQGN

SEQ ID NO: 264 - protein, 12 aa, synthetic construct
  REGION 1..12: note = Synthetic
NVTQAFGRRGPE

SEQ ID NO: 265 - protein, 13 aa, synthetic construct
  REGION 1..13: note = Synthetic
NVTQAFGRRGPEQ

SEQ ID NO: 266 - protein, 10 aa, synthetic construct
  REGION 1..10: note = Synthetic
VTQAFGRRGP

SEQ ID NO: 267 - protein, 11 aa, synthetic construct
  REGION 1..11: note = Synthetic
VTQAFGRRGPE

SEQ ID NO: 268 - protein, 12 aa, synthetic construct
  REGION 1..12: note = Synthetic
VTQAFGRRGPEQ

SEQ ID NO: 269 - protein, 9 aa, synthetic construct
  REGION 1..9: note = Synthetic
TQAFGRRGP

SEQ ID NO: 270 - protein, 10 aa, synthetic construct
  REGION 1..10: note = Synthetic
TQAFGRRGPE

SEQ ID NO: 271 - protein, 11 aa, synthetic construct
  REGION 1..11: note = Synthetic
TQAFGRRGPEQ

SEQ ID NO: 272 - protein, 15 aa, synthetic construct
  REGION 1..15: note = Synthetic
TQAFGRRGPEQTQGN

SEQ ID NO: 273 - protein, 9 aa, synthetic construct
  REGION 1..9: note = Synthetic
QAFGRRGPE

SEQ ID NO: 274 - protein, 10 aa, synthetic construct
  REGION 1..10: note = Synthetic
QAFGRRGPEQ

SEQ ID NO: 275 - protein, 31 aa, synthetic construct
  REGION 1..31: note = Synthetic
RIGMEVTPSGTWLTYTGAIKLDDKDPNFKDQ

SEQ ID NO: 276 - protein, 30 aa, synthetic construct; identical to SEQ ID NO: 56
  REGION 1..30: note = Synthetic

SEQ ID NO: 277 - protein, 35 aa, synthetic construct; identical to SEQ ID NO: 57
  REGION 1..35: note = Synthetic

SEQ ID NO: 278 - protein, 25 aa, synthetic construct
  REGION 1..25: note = Synthetic
MEVTPSGTWLTYTGAIKLDDKDPNF

SEQ ID NO: 279 - protein, 24 aa, synthetic construct
  REGION 1..24: note = Synthetic
EVTPSGTWLTYTGAIKLDDKDPNF

SEQ ID NO: 280 - protein, 27 aa, synthetic construct
  REGION 1..27: note = Synthetic
EVTPSGTWLTYTGAIKLDDKDPNFKDQ

SEQ ID NO: 281 - protein, 30 aa, synthetic construct
  REGION 1..30: note = Synthetic
EVTPSGTWLTYTGAIKLDDKDPNFKDQVIL

SEQ ID NO: 282 - protein, 32 aa, synthetic construct
  REGION 1..32: note = Synthetic
EVTPSGTWLTYTGAIKLDDKDPNFKDQVILLN

SEQ ID NO: 283 - protein, 22 aa, synthetic construct
  REGION 1..22: note = Synthetic
VTPSGTWLTYTGAIKLDDKDPN

SEQ ID NO: 284 - protein, 23 aa, synthetic construct
  REGION 1..23: note = Synthetic
VTPSGTWLTYTGAIKLDDKDPNF

SEQ ID NO: 285 - protein, 24 aa, synthetic construct
  REGION 1..24: note = Synthetic
VTPSGTWLTYTGAIKLDDKDPNFK

SEQ ID NO: 286 - protein, 26 aa, synthetic construct
  REGION 1..26: note = Synthetic
VTPSGTWLTYTGAIKLDDKDPNFKDQ

SEQ ID NO: 287 - protein, 27 aa, synthetic construct
  REGION 1..27: note = Synthetic
VTPSGTWLTYTGAIKLDDKDPNFKDQV

SEQ ID NO: 288 - protein, 18 aa, synthetic construct
  REGION 1..18: note = Synthetic
TPSGTWLTYTGAIKLDDK

SEQ ID NO: 289 - protein, 20 aa, synthetic construct
  REGION 1..20: note = Synthetic
TPSGTWLTYTGAIKLDDKDP

SEQ ID NO: 290 - protein, 21 aa, synthetic construct
  REGION 1..21: note = Synthetic
TPSGTWLTYTGAIKLDDKDPN

SEQ ID NO: 291 - protein, 22 aa, synthetic construct
  REGION 1..22: note = Synthetic
TPSGTWLTYTGAIKLDDKDPNF

SEQ ID NO: 292 - protein, 32 aa, synthetic construct
  REGION 1..32: note = Synthetic
KKKADETQALPQRQKKQQTVTLLPAADLDDFS

SEQ ID NO: 293 - protein, 29 aa, synthetic construct
  REGION 1..29: note = Synthetic
KKADETQALPQRQKKQQTVTLLPAADLDD

SEQ ID NO: 294 - protein, 27 aa, synthetic construct
  REGION 1..27: note = Synthetic
KADETQALPQRQKKQQTVTLLPAADLD

SEQ ID NO: 295 - protein, 28 aa, synthetic construct
  REGION 1..28: note = Synthetic
KADETQALPQRQKKQQTVTLLPAADLDD

SEQ ID NO: 296 - protein, 30 aa, synthetic construct
  REGION 1..30: note = Synthetic
KADETQALPQRQKKQQTVTLLPAADLDDFS

SEQ ID NO: 297 - protein, 34 aa, synthetic construct
  REGION 1..34: note = Synthetic
KADETQALPQRQKKQQTVTLLPAADLDDFSKQLQ

SEQ ID NO: 298 - protein, 26 aa, synthetic construct
  REGION 1..26: note = Synthetic
ADETQALPQRQKKQQTVTLLPAADLD

SEQ ID NO: 299 - protein, 27 aa, synthetic construct
  REGION 1..27: note = Synthetic
ADETQALPQRQKKQQTVTLLPAADLDD

SEQ ID NO: 300 - protein, 29 aa, synthetic construct
  REGION 1..29: note = Synthetic
ADETQALPQRQKKQQTVTLLPAADLDDFS

SEQ ID NO: 301 - protein, 31 aa, synthetic construct
  REGION 1..31: note = Synthetic
ADETQALPQRQKKQQTVTLLPAADLDDFSKQ

SEQ ID NO: 302 - protein, 32 aa, synthetic construct
  REGION 1..32: note = Synthetic
ADETQALPQRQKKQQTVTLLPAADLDDFSKQL

SEQ ID NO: 303 - protein, 33 aa, synthetic construct
  REGION 1..33: note = Synthetic
ADETQALPQRQKKQQTVTLLPAADLDDFSKQLQ

SEQ ID NO: 304 - protein, 35 aa, synthetic construct
  REGION 1..35: note = Synthetic
ADETQALPQRQKKQQTVTLLPAADLDDFSKQLQQS

SEQ ID NO: 305 - protein, 25 aa, synthetic construct
  REGION 1..25: note = Synthetic
DETQALPQRQKKQQTVTLLPAADLD

SEQ ID NO: 306 - protein, 26 aa, synthetic construct
  REGION 1..26: note = Synthetic
DETQALPQRQKKQQTVTLLPAADLDD

SEQ ID NO: 307 - protein, 30 aa, synthetic construct
  REGION 1..30: note = Synthetic
DETQALPQRQKKQQTVTLLPAADLDDFSKQ

SEQ ID NO: 308 - protein, 32 aa, synthetic construct
  REGION 1..32: note = Synthetic
DETQALPQRQKKQQTVTLLPAADLDDFSKQLQ

SEQ ID NO: 309 - protein, 34 aa, synthetic construct
  REGION 1..34: note = Synthetic
DETQALPQRQKKQQTVTLLPAADLDDFSKQLQQS

SEQ ID NO: 310 - protein, 25 aa, synthetic construct
  REGION 1..25: note = Synthetic
QALPQRQKKQQTVTLLPAADLDDFS

SEQ ID NO: 311 - protein, 21 aa, synthetic construct
  REGION 1..21: note = Synthetic
ALPQRQKKQQTVTLLPAADLD

SEQ ID NO: 312 - protein, 22 aa, synthetic construct
  REGION 1..22: note = Synthetic
ALPQRQKKQQTVTLLPAADLDD

SEQ ID NO: 313 - protein, 24 aa, synthetic construct
  REGION 1..24: note = Synthetic
ALPQRQKKQQTVTLLPAADLDDFS

SEQ ID NO: 314 - protein, 25 aa, synthetic construct
  REGION 1..25: note = Synthetic
ALPQRQKKQQTVTLLPAADLDDFSK

SEQ ID NO: 315 - protein, 26 aa, synthetic construct
  REGION 1..26: note = Synthetic
ALPQRQKKQQTVTLLPAADLDDFSKQ

SEQ ID NO: 316 - protein, 27 aa, synthetic construct
  REGION 1..27: note = Synthetic
ALPQRQKKQQTVTLLPAADLDDFSKQL

SEQ ID NO: 317 - protein, 28 aa, synthetic construct
  REGION 1..28: note = Synthetic
ALPQRQKKQQTVTLLPAADLDDFSKQLQ

SEQ ID NO: 318 - protein, 29 aa, synthetic construct
  REGION 1..29: note = Synthetic
ALPQRQKKQQTVTLLPAADLDDFSKQLQQ

SEQ ID NO: 319 - protein, 30 aa, synthetic construct
  REGION 1..30: note = Synthetic
ALPQRQKKQQTVTLLPAADLDDFSKQLQQS

SEQ ID NO: 320 - protein, 31 aa, synthetic construct
  REGION 1..31: note = Synthetic
ALPQRQKKQQTVTLLPAADLDDFSKQLQQSM

SEQ ID NO: 321 - protein, 32 aa, synthetic construct
  REGION 1..32: note = Synthetic
ALPQRQKKQQTVTLLPAADLDDFSKQLQQSMS

SEQ ID NO: 322 - protein, 33 aa, synthetic construct
  REGION 1..33: note = Synthetic
ALPQRQKKQQTVTLLPAADLDDFSKQLQQSMSS

SEQ ID NO: 323 - protein, 34 aa, synthetic construct
  REGION 1..34: note = Synthetic
ALPQRQKKQQTVTLLPAADLDDFSKQLQQSMSSA

SEQ ID NO: 324 - protein, 35 aa, synthetic construct
  REGION 1..35: note = Synthetic
ALPQRQKKQQTVTLLPAADLDDFSKQLQQSMSSAD

SEQ ID NO: 325 - protein, 20 aa, synthetic construct
  REGION 1..20: note = Synthetic
LPQRQKKQQTVTLLPAADLD

SEQ ID NO: 326 - protein, 21 aa, synthetic construct
  REGION 1..21: note = Synthetic
LPQRQKKQQTVTLLPAADLDD

SEQ ID NO: 327 - protein, 16 aa, synthetic construct
  REGION 1..16: note = Synthetic
QRQKKQQTVTLLPAAD

SEQ ID NO: 328 - protein, 17 aa, synthetic construct
  REGION 1..17: note = Synthetic
QRQKKQQTVTLLPAADL

SEQ ID NO: 329 - protein, 18 aa, synthetic construct
  REGION 1..18: note = Synthetic
QRQKKQQTVTLLPAADLD

SEQ ID NO: 330 - protein, 19 aa, synthetic construct
  REGION 1..19: note = Synthetic
QRQKKQQTVTLLPAADLDD

SEQ ID NO: 331 - protein, 20 aa, synthetic construct
  REGION 1..20: note = Synthetic
QRQKKQQTVTLLPAADLDDF

SEQ ID NO: 332 - protein, 21 aa, synthetic construct
  REGION 1..21: note = Synthetic
QRQKKQQTVTLLPAADLDDFS

SEQ ID NO: 333 - protein, 22 aa, synthetic construct
  REGION 1..22: note = Synthetic
QRQKKQQTVTLLPAADLDDFSK

SEQ ID NO: 334 - protein, 23 aa, synthetic construct
  REGION 1..23: note = Synthetic
QRQKKQQTVTLLPAADLDDFSKQ

SEQ ID NO: 335 - protein, 24 aa, synthetic construct
  REGION 1..24: note = Synthetic
QRQKKQQTVTLLPAADLDDFSKQL

SEQ ID NO: 336 - protein, 25 aa, synthetic construct
  REGION 1..25: note = Synthetic
QRQKKQQTVTLLPAADLDDFSKQLQ

SEQ ID NO: 337 - protein, 26 aa, synthetic construct
  REGION 1..26: note = Synthetic
QRQKKQQTVTLLPAADLDDFSKQLQQ

SEQ ID NO: 338 - protein, 27 aa, synthetic construct
  REGION 1..27: note = Synthetic
QRQKKQQTVTLLPAADLDDFSKQLQQS

SEQ ID NO: 339 - protein, 28 aa, synthetic construct
  REGION 1..28: note = Synthetic
QRQKKQQTVTLLPAADLDDFSKQLQQSM

SEQ ID NO: 340 - protein, 29 aa, synthetic construct
  REGION 1..29: note = Synthetic
QRQKKQQTVTLLPAADLDDFSKQLQQSMS

SEQ ID NO: 341 - protein, 30 aa, synthetic construct
  REGION 1..30: note = Synthetic
QRQKKQQTVTLLPAADLDDFSKQLQQSMSS

SEQ ID NO: 342 - protein, 31 aa, synthetic construct
  REGION 1..31: note = Synthetic
QRQKKQQTVTLLPAADLDDFSKQLQQSMSSA

SEQ ID NO: 343 - protein, 32 aa, synthetic construct
  REGION 1..32: note = Synthetic
QRQKKQQTVTLLPAADLDDFSKQLQQSMSSAD

SEQ ID NO: 344 - protein, 33 aa, synthetic construct
  REGION 1..33: note = Synthetic
QRQKKQQTVTLLPAADLDDFSKQLQQSMSSADS

SEQ ID NO: 345 - protein, 34 aa, synthetic construct
  REGION 1..34: note = Synthetic
QRQKKQQTVTLLPAADLDDFSKQLQQSMSSADST

SEQ ID NO: 346 - protein, 35 aa, synthetic construct; identical to SEQ ID NO: 95
  REGION 1..35: note = Synthetic

SEQ ID NO: 347 - protein, 36 aa, synthetic construct; identical to SEQ ID NO: 96
  REGION 1..36: note = Synthetic

SEQ ID NO: 348 - protein, 15 aa, synthetic construct
  REGION 1..15: note = Synthetic
RQKKQQTVTLLPAAD

SEQ ID NO: 349 - protein, 16 aa, synthetic construct
  REGION 1..16: note = Synthetic
RQKKQQTVTLLPAADL

SEQ ID NO: 350 - protein, 17 aa, synthetic construct
  REGION 1..17: note = Synthetic
RQKKQQTVTLLPAADLD

SEQ ID NO: 351 - protein, 18 aa, synthetic construct
  REGION 1..18: note = Synthetic
RQKKQQTVTLLPAADLDD

SEQ ID NO: 352 - protein, 19 aa, synthetic construct
  REGION 1..19: note = Synthetic
RQKKQQTVTLLPAADLDDF

SEQ ID NO: 353 - protein, 20 aa, synthetic construct
  REGION 1..20: note = Synthetic
RQKKQQTVTLLPAADLDDFS

SEQ ID NO: 354 - protein, 22 aa, synthetic construct
  REGION 1..22: note = Synthetic
RQKKQQTVTLLPAADLDDFSKQ

SEQ ID NO: 355 - protein, 24 aa, synthetic construct
  REGION 1..24: note = Synthetic
RQKKQQTVTLLPAADLDDFSKQLQ

SEQ ID NO: 356 - protein, 14 aa, synthetic construct
  REGION 1..14: note = Synthetic
QKKQQTVTLLPAAD

SEQ ID NO: 357 - protein, 15 aa, synthetic construct
  REGION 1..15: note = Synthetic
QKKQQTVTLLPAADL

SEQ ID NO: 358 - protein, 16 aa, synthetic construct
  REGION 1..16: note = Synthetic
QKKQQTVTLLPAADLD

SEQ ID NO: 359 - protein, 18 aa, synthetic construct
  REGION 1..18: note = Synthetic
TVTLLPAADLDDFSKQLQ

SEQ ID NO: 360 - protein, 15 aa, synthetic construct
  REGION 1..15: note = Synthetic
LLPAADLDDFSKQLQ

SEQ ID NO: 361 - protein, 19 aa, synthetic construct
  REGION 1..19: note = Synthetic
THNSIKGADFLAPTLIYHL

SEQ ID NO: 362 - protein, 24 aa, synthetic construct
  REGION 1..24: note = Synthetic
QSPYCLMKPASASVTLLKSRSSIN

SEQ ID NO: 363 - protein, 16 aa, synthetic construct
  REGION 1..16: note = Synthetic
DGSEDNQTTTIQTIVE

SEQ ID NO: 364 - protein, 17 aa, synthetic construct
  REGION 1..17: note = Synthetic
SPDAVTAYNGYLTSSSK

SEQ ID NO: 365 - protein, 18 aa, synthetic construct
  REGION 1..18: note = Synthetic
MDGSIIQFPNTYLEGSVR

SEQ ID NO: 366 - protein, 15 aa, synthetic construct
  REGION 1..15: note = Synthetic
DGSIIQFPNTYLEGS

SEQ ID NO: 367 - protein, 17 aa, synthetic construct
  REGION 1..17: note = Synthetic
DGSIIQFPNTYLEGSVR

SEQ ID NO: 368 - protein, 14 aa, synthetic construct
  REGION 1..14: note = Synthetic
GSIIQFPNTYLEGS

SEQ ID NO: 369 - protein, 16 aa, synthetic construct
  REGION 1..16: note = Synthetic
GSIIQFPNTYLEGSVR

SEQ ID NO: 370 - protein, 28 aa, synthetic construct
  REGION 1..28: note = Synthetic
MDLFMRIFTIGTVTLKQGEIKDATPSDF

SEQ ID NO: 371 - protein, 24 aa, synthetic construct
  REGION 1..24: note = Synthetic
MRIFTIGTVTLKQGEIKDATPSDF

SEQ ID NO: 372 - protein, 22 aa, synthetic construct
  REGION 1..22: note = Synthetic
IFTIGTVTLKQGEIKDATPSDF

SEQ ID NO: 373 - protein, 19 aa, synthetic construct
  REGION 1..19: note = Synthetic
IGTVTLKQGEIKDATPSDF

SEQ ID NO: 374 - protein, 14 aa, synthetic construct
  REGION 1..14: note = Synthetic
ALHFLLFFRALPKS

SEQ ID NO: 375 - protein, 16 aa, synthetic construct
  REGION 1..16: note = Synthetic
MDPKISEMHPALRLVD

SEQ ID NO: 376 - protein, 17 aa, synthetic construct
  REGION 1..17: note = Synthetic
MDPKISEMHPALRLVDP

SEQ ID NO: 377 - protein, 18 aa, synthetic construct
  REGION 1..18: note = Synthetic
MDPKISEMHPALRLVDPQ

SEQ ID NO: 378 - protein, 20 aa, synthetic construct
  REGION 1..20: note = Synthetic
MDPKISEMHPALRLVDPQIQ

SEQ ID NO: 379 - protein, 21 aa, synthetic construct
  REGION 1..21: note = Synthetic
MDPKISEMHPALRLVDPQIQL

SEQ ID NO: 380 - protein, 22 aa, synthetic construct
  REGION 1..22: note = Synthetic
MDPKISEMHPALRLVDPQIQLA

SEQ ID NO: 381 - protein, 28 aa, synthetic construct
  REGION 1..28: note = Synthetic
MDPKISEMHPALRLVDPQIQLAVTRMEN

SEQ ID NO: 382 - protein, 29 aa, synthetic construct
  REGION 1..29: note = Synthetic
MDPKISEMHPALRLVDPQIQLAVTRMENA

SEQ ID NO: 383 - protein, 16 aa, synthetic construct
  REGION 1..16: note = Synthetic
VGPKVYPIILRLGSPL

SEQ ID NO: 384 - protein, 17 aa, synthetic construct
  REGION 1..17: note = Synthetic
VGPKVYPIILRLGSPLS

SEQ ID NO: 385 - protein, 26 aa, synthetic construct
  REGION 1..26: note = Synthetic
FKNLREFVFKNIDGYFKIYSKHTPIN

SEQ ID NO: 386 - protein, 15 aa, synthetic construct
  REGION 1..15: note = Synthetic
NLREFVFKNIDGYFK

SEQ ID NO: 387 - protein, 17 aa, synthetic construct
  REGION 1..17: note = Synthetic
NLREFVFKNIDGYFKIY

SEQ ID NO: 388 - protein, 18 aa, synthetic construct
  REGION 1..18: note = Synthetic
NLREFVFKNIDGYFKIYS

SEQ ID NO: 389 - protein, 19 aa, synthetic construct
  REGION 1..19: note = Synthetic
NLREFVFKNIDGYFKIYSK

SEQ ID NO: 390 - protein, 20 aa, synthetic construct
  REGION 1..20: note = Synthetic
NLREFVFKNIDGYFKIYSKH

SEQ ID NO: 391 - protein, 21 aa, synthetic construct
  REGION 1..21: note = Synthetic
NLREFVFKNIDGYFKIYSKHT

SEQ ID NO: 392 - protein, 22 aa, synthetic construct
  REGION 1..22: note = Synthetic
NLREFVFKNIDGYFKIYSKHTP

SEQ ID NO: 393 - protein, 23 aa, synthetic construct
  REGION 1..23: note = Synthetic
NLREFVFKNIDGYFKIYSKHTPI

SEQ ID NO: 394 - protein, 24 aa, synthetic construct
  REGION 1..24: note = Synthetic
NLREFVFKNIDGYFKIYSKHTPIN

SEQ ID NO: 395 - protein, 14 aa, synthetic construct
  REGION 1..14: note = Synthetic
LREFVFKNIDGYFK

SEQ ID NO: 396 - protein, 16 aa, synthetic construct
  REGION 1..16: note = Synthetic
LREFVFKNIDGYFKIY

SEQ ID NO: 397 - protein, 17 aa, synthetic construct
  REGION 1..17: note = Synthetic
LREFVFKNIDGYFKIYS

SEQ ID NO: 398 - protein, 18 aa, synthetic construct
  REGION 1..18: note = Synthetic
LREFVFKNIDGYFKIYSK

SEQ ID NO: 399 - protein, 19 aa, synthetic construct
  REGION 1..19: note = Synthetic
LREFVFKNIDGYFKIYSKH

SEQ ID NO: 400 - protein, 21 aa, synthetic construct
  REGION 1..21: note = Synthetic
LREFVFKNIDGYFKIYSKHTP

SEQ ID NO: 401 - protein, 23 aa, synthetic construct
  REGION 1..23: note = Synthetic
LREFVFKNIDGYFKIYSKHTPIN

SEQ ID NO: 402 - protein, 13 aa, synthetic construct
  REGION 1..13: note = Synthetic
REFVFKNIDGYFK

SEQ ID NO: 403 - protein, 14 aa, synthetic construct
  REGION 1..14: note = Synthetic
REFVFKNIDGYFKI

SEQ ID NO: 404 - protein, 15 aa, synthetic construct
  REGION 1..15: note = Synthetic
REFVFKNIDGYFKIY

SEQ ID NO: 405 - protein, 16 aa, synthetic construct
  REGION 1..16: note = Synthetic
REFVFKNIDGYFKIYS

SEQ ID NO: 406 - protein, 17 aa, synthetic construct
  REGION 1..17: note = Synthetic
REFVFKNIDGYFKIYSK

SEQ ID NO: 407 - protein, 18 aa, synthetic construct
  REGION 1..18: note = Synthetic
REFVFKNIDGYFKIYSKH

SEQ ID NO: 408 - protein, 20 aa, synthetic construct
  REGION 1..20: note = Synthetic
REFVFKNIDGYFKIYSKHTP

SEQ ID NO: 409 - protein, 22 aa, synthetic construct
  REGION 1..22: note = Synthetic
REFVFKNIDGYFKIYSKHTPIN

SEQ ID NO: 410 - protein, 12 aa, synthetic construct
  REGION 1..12: note = Synthetic
EFVFKNIDGYFK

SEQ ID NO: 411 - protein, 14 aa, synthetic construct
  REGION 1..14: note = Synthetic
EFVFKNIDGYFKIY

SEQ ID NO: 412 - protein, 15 aa, synthetic construct
  REGION 1..15: note = Synthetic
EFVFKNIDGYFKIYS

SEQ ID NO: 413 - protein, 16 aa, synthetic construct
  REGION 1..16: note = Synthetic
EFVFKNIDGYFKIYSK

SEQ ID NO: 414 - protein, 17 aa, synthetic construct
  REGION 1..17: note = Synthetic
EFVFKNIDGYFKIYSKH

SEQ ID NO: 415 - protein, 21 aa, synthetic construct
  REGION 1..21: note = Synthetic
EFVFKNIDGYFKIYSKHTPIN

SEQ ID NO: 416 - protein, 16 aa, synthetic construct
  REGION 1..16: note = Synthetic
FVFKNIDGYFKIYSKH

SEQ ID NO: 417 - protein, 20 aa, synthetic construct
  REGION 1..20: note = Synthetic
FVFKNIDGYFKIYSKHTPIN

SEQ ID NO: 418 - protein, 17 aa, synthetic construct
  REGION 1..17: note = Synthetic
INITRFQTLLALHRSYL

SEQ ID NO: 419 - protein, 16 aa, synthetic construct
  REGION 1..16: note = Synthetic
VEKGIYQTSNFRVQPT

SEQ ID NO: 420 - protein, 17 aa, synthetic construct
  REGION 1..17: note = Synthetic
VEKGIYQTSNFRVQPTE

SEQ ID NO: 421 - protein, 18 aa, synthetic construct
  REGION 1..18: note = Synthetic
VEKGIYQTSNFRVQPTES

SEQ ID NO: 422 - protein, 14 aa, synthetic construct
  REGION 1..14: note = Synthetic
ATRFASVYAWNRKR

SEQ ID NO: 423 - protein, 16 aa, synthetic construct
  REGION 1..16: note = Synthetic
ATRFASVYAWNRKRIS

SEQ ID NO: 424 - protein, 17 aa, synthetic construct
  REGION 1..17: note = Synthetic
ATRFASVYAWNRKRISN

SEQ ID NO: 425 - protein, 16 aa, synthetic construct
  REGION 1..16: note = Synthetic
DSFVIRGDEVRQIAPG

SEQ ID NO: 426 - protein, 20 aa, synthetic construct
  REGION 1..20: note = Synthetic
NYKLPDDFTGCVIAWNSNNL

SEQ ID NO: 427 - protein, 24 aa, synthetic construct
  REGION 1..24: note = Synthetic
NYKLPDDFTGCVIAWNSNNLDSKV

SEQ ID NO: 428 - protein, 22 aa, synthetic construct
  REGION 1..22: note = Synthetic
YKLPDDFTGCVIAWNSNNLDSK

SEQ ID NO: 429 - protein, 24 aa, synthetic construct
  REGION 1..24: note = Synthetic
YKLPDDFTGCVIAWNSNNLDSKVG

SEQ ID NO: 430 - protein, 21 aa, synthetic construct
  REGION 1..21: note = Synthetic
KLPDDFTGCVIAWNSNNLDSK

SEQ ID NO: 431 - protein, 23 aa, synthetic construct
  REGION 1..23: note = Synthetic
KLPDDFTGCVIAWNSNNLDSKVG

SEQ ID NO: 432 - protein, 18 aa, synthetic construct
  REGION 1..18: note = Synthetic
LPDDFTGCVIAWNSNNLD

SEQ ID NO: 433 - protein, 20 aa, synthetic construct
  REGION 1..20: note = Synthetic
LPDDFTGCVIAWNSNNLDSK

SEQ ID NO: 434 - protein, 22 aa, synthetic construct
  REGION 1..22: note = Synthetic
LPDDFTGCVIAWNSNNLDSKVG

SEQ ID NO: 435 - protein, 17 aa, synthetic construct
  REGION 1..17: note = Synthetic
DFTGCVIAWNSNNLDSK

SEQ ID NO: 436 - protein, 16 aa, synthetic construct
  REGION 1..16: note = Synthetic
FTGCVIAWNSNNLDSK

SEQ ID NO: 437 - protein, 15 aa, synthetic construct
  REGION 1..15: note = Synthetic
TGCVIAWNSNNLDSK

SEQ ID NO: 438 - protein, 14 aa, synthetic construct
  REGION 1..14: note = Synthetic
GCVIAWNSNNLDSK

SEQ ID NO: 439 - protein, 16 aa, synthetic construct
  REGION 1..16: note = Synthetic
GCVIAWNSNNLDSKVG

SEQ ID NO: 440 - protein, 16 aa, synthetic construct
  REGION 1..16: note = Synthetic
TEIYQAGSTPCNGVEG

SEQ ID NO: 441 - protein, 15 aa, synthetic construct
  REGION 1..15: note = Synthetic
EIYQAGSTPCNGVEG

SEQ ID NO: 442 - protein, 28 aa, synthetic construct
  REGION 1..28: note = Synthetic
ESNKKFLPFQQFGRDIADTTDAVRDPQT

SEQ ID NO: 443 - protein, 15 aa, synthetic construct
  REGION 1..15: note = Synthetic
WRVYSTGSNVFQTRA

SEQ ID NO: 444 - protein, 16 aa, synthetic construct
  REGION 1..16: note = Synthetic
WRVYSTGSNVFQTRAG

SEQ ID NO: 445 - protein, 15 aa, synthetic construct
  REGION 1..15: note = Synthetic
ICASYQTQTNSPRRA

SEQ ID NO: 446 - protein, 18 aa, synthetic construct
  REGION 1..18: note = Synthetic
SVASQSIIAYTMSLGAEN

SEQ ID NO: 447 - protein, 13 aa, synthetic construct
  REGION 1..13: note = Synthetic
ASQSIIAYTMSLG

SEQ ID NO: 448 - protein, 16 aa, synthetic construct
  REGION 1..16: note = Synthetic
ASQSIIAYTMSLGAEN

SEQ ID NO: 449 - protein, 14 aa, synthetic construct
  REGION 1..14: note = Synthetic
CSNLLLQYGSFCTQ

SEQ ID NO: 450 - protein, 13 aa, synthetic construct
  REGION 1..13: note = Synthetic
SNLLLQYGSFCTQ

SEQ ID NO: 451 - protein, 14 aa, synthetic construct
  REGION 1..14: note = Synthetic
SNLLLQYGSFCTQL

SEQ ID NO: 452 - protein, 15 aa, synthetic construct
  REGION 1..15: note = Synthetic
SNLLLQYGSFCTQLN

SEQ ID NO: 453 - protein, 16 aa, synthetic construct
  REGION 1..16: note = Synthetic
SNLLLQYGSFCTQLNR

SEQ ID NO: 454 - protein, 18 aa, synthetic construct
  REGION 1..18: note = Synthetic
LNRALTGIAVEQDKNTQE

SEQ ID NO: 455 - protein, 14 aa, synthetic construct
  REGION 1..14: note = Synthetic
NRALTGIAVEQDKN

SEQ ID NO: 456 - protein, 15 aa, synthetic construct
  REGION 1..15: note = Synthetic
NRALTGIAVEQDKNT

SEQ ID NO: 457 - protein, 16 aa, synthetic construct
  REGION 1..16: note = Synthetic
NRALTGIAVEQDKNTQ

SEQ ID NO: 458 - protein, 14 aa, synthetic construct
  REGION 1..14: note = Synthetic
RALTGIAVEQDKNT

SEQ ID NO: 459 - protein, 20 aa, synthetic construct
  REGION 1..20: note = Synthetic
VLPPLLTDEMIAQYTSALLA

SEQ ID NO: 460 - protein, 24 aa, synthetic construct
  REGION 1..24: note = Synthetic
VLPPLLTDEMIAQYTSALLAGTIT

SEQ ID NO: 461 - protein, 21 aa, synthetic construct
  REGION 1..21: note = Synthetic
LPPLLTDEMIAQYTSALLAGT

SEQ ID NO: 462 - protein, 23 aa, synthetic construct
  REGION 1..23: note = Synthetic
LPPLLTDEMIAQYTSALLAGTIT

SEQ ID NO: 463 - protein, 20 aa, synthetic construct
  REGION 1..20: note = Synthetic
PPLLTDEMIAQYTSALLAGT

SEQ ID NO: 464 - protein, 15 aa, synthetic construct
  REGION 1..15: note = Synthetic
LTDEMIAQYTSALLA

SEQ ID NO: 465 - protein, 16 aa, synthetic construct
  REGION 1..16: note = Synthetic
LTDEMIAQYTSALLAG

SEQ ID NO: 466 - protein, 17 aa, synthetic construct
  REGION 1..17: note = Synthetic
LTDEMIAQYTSALLAGT

SEQ ID NO: 467 - protein, 19 aa, synthetic construct
  REGION 1..19: note = Synthetic
LTDEMIAQYTSALLAGTIT

SEQ ID NO: 468 - protein, 13 aa, synthetic construct
  REGION 1..13: note = Synthetic
TDEMIAQYTSALL

SEQ ID NO: 469 - protein, 14 aa, synthetic construct
  REGION 1..14: note = Synthetic
TDEMIAQYTSALLA

SEQ ID NO: 470 - protein, 15 aa, synthetic construct
  REGION 1..15: note = Synthetic
TDEMIAQYTSALLAG

SEQ ID NO: 471 - protein, 16 aa, synthetic construct
  REGION 1..16: note = Synthetic
TDEMIAQYTSALLAGT

SEQ ID NO: 472 - protein, 17 aa, synthetic construct
  REGION 1..17: note = Synthetic
TDEMIAQYTSALLAGTI

SEQ ID NO: 473 - protein, 18 aa, synthetic construct
  REGION 1..18: note = Synthetic
TDEMIAQYTSALLAGTIT

SEQ ID NO: 474 - protein, 12 aa, synthetic construct
  REGION 1..12: note = Synthetic
DEMIAQYTSALL

SEQ ID NO: 475 - protein, 13 aa, synthetic construct
  REGION 1..13: note = Synthetic
DEMIAQYTSALLA

SEQ ID NO: 476 - protein, 14 aa, synthetic construct
  REGION 1..14: note = Synthetic
DEMIAQYTSALLAG

SEQ ID NO: 477 - protein, 15 aa, synthetic construct
  REGION 1..15: note = Synthetic
DEMIAQYTSALLAGT

SEQ ID NO: 478 - protein, 16 aa, synthetic construct
  REGION 1..16: note = Synthetic
DEMIAQYTSALLAGTI

SEQ ID NO: 479 - protein, 17 aa, synthetic construct
  REGION 1..17: note = Synthetic
DEMIAQYTSALLAGTIT

SEQ ID NO: 480 - protein, 12 aa, synthetic construct
  REGION 1..12: note = Synthetic
EMIAQYTSALLA

SEQ ID NO: 481 - protein, 13 aa, synthetic construct
  REGION 1..13: note = Synthetic
EMIAQYTSALLAG

SEQ ID NO: 482 - protein, 14 aa, synthetic construct
  REGION 1..14: note = Synthetic
EMIAQYTSALLAGT

SEQ ID NO: 483 - protein, 16 aa, synthetic construct
  REGION 1..16: note = Synthetic
EMIAQYTSALLAGTIT

SEQ ID NO: 484 - protein, 14 aa, synthetic construct
  REGION 1..14: note = Synthetic
AALQIPFAMQMAYR

SEQ ID NO: 485 - protein, 16 aa, synthetic construct
  REGION 1..16: note = Synthetic
AALQIPFAMQMAYRFN

SEQ ID NO: 486 - protein, 14 aa, synthetic construct
  REGION 1..14: note = Synthetic
LQIPFAMQMAYRFN

SEQ ID NO: 487 - protein, 15 aa, synthetic construct
  REGION 1..15: note = Synthetic
LQIPFAMQMAYRFNG

SEQ ID NO: 488 - protein, 17 aa, synthetic construct
  REGION 1..17: note = Synthetic
LQIPFAMQMAYRFNGIG

SEQ ID NO: 489 - protein, 13 aa, synthetic construct
  REGION 1..13: note = Synthetic
QIPFAMQMAYRFN

SEQ ID NO: 490 - protein, 14 aa, synthetic construct
  REGION 1..14: note = Synthetic
QIPFAMQMAYRFNG

SEQ ID NO: 491 - protein, 16 aa, synthetic construct
  REGION 1..16: note = Synthetic
QIPFAMQMAYRFNGIG

SEQ ID NO: 492 - protein, 12 aa, synthetic construct
  REGION 1..12: note = Synthetic
IPFAMQMAYRFN

SEQ ID NO: 493 - protein, 13 aa, synthetic construct
  REGION 1..13: note = Synthetic
IPFAMQMAYRFNG

SEQ ID NO: 494 - protein, 15 aa, synthetic construct
  REGION 1..15: note = Synthetic
IPFAMQMAYRFNGIG

SEQ ID NO: 495 - protein, 13 aa, synthetic construct
  REGION 1..13: note = Synthetic
TQQLIRAAEIRAS

SEQ ID NO: 496 - protein, 15 aa, synthetic construct
  REGION 1..15: note = Synthetic
TQQLIRAAEIRASAN

SEQ ID NO: 497 - protein, 16 aa, synthetic construct
  REGION 1..16: note = Synthetic
TQQLIRAAEIRASANL

SEQ ID NO: 498 - protein, 17 aa, synthetic construct
  REGION 1..17: note = Synthetic
TQQLIRAAEIRASANLA

SEQ ID NO: 499 - protein, 12 aa, synthetic construct
  REGION 1..12: note = Synthetic
QQLIRAAEIRAS

SEQ ID NO: 500 - protein, 13 aa, synthetic construct
  REGION 1..13: note = Synthetic
QQLIRAAEIRASA

SEQ ID NO: 501 - protein, 14 aa, synthetic construct
  REGION 1..14: note = Synthetic
QQLIRAAEIRASAN

SEQ ID NO: 502 - protein, 15 aa, synthetic construct
  REGION 1..15: note = Synthetic
QQLIRAAEIRASANL

SEQ ID NO: 503 - protein, 16 aa, synthetic construct
  REGION 1..16: note = Synthetic
QQLIRAAEIRASANLA

SEQ ID NO: 504 - protein, 11 aa, synthetic construct
  REGION 1..11: note = Synthetic
QLIRAAEIRAS

SEQ ID NO: 505 - protein, 10 aa, synthetic construct
  REGION 1..10: note = Synthetic
LIRAAEIRAS

SEQ ID NO: 506 - protein, 11 aa, synthetic construct
  REGION 1..11: note = Synthetic
LIRAAEIRASA

SEQ ID NO: 507 - protein, 12 aa, synthetic construct
  REGION 1..12: note = Synthetic
LIRAAEIRASAN

SEQ ID NO: 508 - protein, 13 aa, synthetic construct
  REGION 1..13: note = Synthetic
LIRAAEIRASANL

SEQ ID NO: 509 - protein, 51 aa, synthetic construct
  REGION 1..51: note = Synthetic
NVPLHGTILTRPLLESELVIGAVILRGHLRIAGHHLGRCDIKDLPKEITVA

SEQ ID NO: 510 - protein, 21 aa, synthetic construct
  REGION 1..21: note = Synthetic
TSRTLSYYKLGASQRVAGDSG

SEQ ID NO: 511 - protein, 15 aa, synthetic construct
  REGION 1..15: note = Synthetic
TDHSSSSDNIALLVQ

SEQ ID NO: 512 - protein, 16 aa, synthetic construct
  REGION 1..16: note = Synthetic
SDNGPQNQRNAPRITF

SEQ ID NO: 513 - protein, 52 aa, synthetic construct
  REGION 1..52: note = Synthetic
GLPNNTASWFTALTQHGKEDLKFPRGQGVPINTNSSPDDQIGYYRRATRRIR

SEQ ID NO: 514 - protein, 15 aa, synthetic construct
  REGION 1..15: note = Synthetic
TGPEAGLPYGANKDG

SEQ ID NO: 515 - protein, 38 aa, synthetic construct; identical to SEQ ID NO: 164
  REGION 1..38: note = Synthetic

SEQ ID NO: 516 - protein, 38 aa, synthetic construct; identical to SEQ ID NO: 34
  REGION 1..38: note = Synthetic

SEQ ID NO: 517 - protein, 47 aa, synthetic construct
  REGION 1..47: note = Synthetic
AAEASKKPRQKRTATKAYNVTQAFGRRGPEQTQGNFGDQELIRQGTD

SEQ ID NO: 518 - protein, 13 aa, synthetic construct
  REGION 1..13: note = Synthetic
IAQFAPSASAFFG

SEQ ID NO: 519 - protein, 47 aa, synthetic construct
  REGION 1..47: note = Synthetic
RIGMEVTPSGTWLTYTGAIKLDDKDPNFKDQVILLNKHIDAYKTFPP

SEQ ID NO: 520 - protein, 47 aa, synthetic construct
  REGION 1..47: note = Synthetic
KKKADETQALPQRQKKQQTVTLLPAADLDDFSKQLQQSMSSADSTQA

SEQ ID NO: 521 - protein, 16 aa, synthetic construct
  REGION 1..16: note = Synthetic
DGSEDNQTTTIQTIVE

SEQ ID NO: 522 - protein, 17 aa, synthetic construct
  REGION 1..17: note = Synthetic
SPDAVTAYNGYLTSSSK

SEQ ID NO: 523 - protein, 18 aa, synthetic construct
  REGION 1..18: note = Synthetic
MDGSIIQFPNTYLEGSVR

SEQ ID NO: 524 - protein, 28 aa, synthetic construct
  REGION 1..28: note = Synthetic
MDLFMRIFTIGTVTLKQGEIKDATPSDF

SEQ ID NO: 525 - protein, 14 aa, synthetic construct
  REGION 1..14: note = Synthetic
ALHFLLFFRALPKS

SEQ ID NO: 526 - protein, 13 aa, synthetic construct
  REGION 1..13: note = Synthetic
YIINLIIKNLSKS

SEQ ID NO: 527 - protein, 29 aa, synthetic construct
  REGION 1..29: note = Synthetic
MDPKISEMHPALRLVDPQIQLAVTRMENA

SEQ ID NO: 528 - protein, 17 aa, synthetic construct
  REGION 1..17: note = Synthetic
VGPKVYPIILRLGSPLS

SEQ ID NO: 529 - protein, 39 aa, synthetic construct
  REGION 1..39: note = Synthetic
TQLPPAYTNSFTRGVYYPDKVFRSSVLHSTQDLFLPFFS

SEQ ID NO: 530 - protein, 31 aa, synthetic construct
  REGION 1..31: note = Synthetic
FKNLREFVFKNIDGYFKIYSKHTPINLVRDL

SEQ ID NO: 531 - protein, 17 aa, synthetic construct
  REGION 1..17: note = Synthetic
INITRFQTLLALHRSYL

SEQ ID NO: 532 - protein, 19 aa, synthetic construct
  REGION 1..19: note = Synthetic
TVEKGIYQTSNFRVQPTES

SEQ ID NO: 533 - protein, 17 aa, synthetic construct
  REGION 1..17: note = Synthetic
ATRFASVYAWNRKRISN

SEQ ID NO: 534 - protein, 16 aa, synthetic construct
  REGION 1..16: note = Synthetic
DSFVIRGDEVRQIAPG

SEQ ID NO: 535 - protein, 25 aa, synthetic construct
  REGION 1..25: note = Synthetic
NYKLPDDFTGCVIAWNSNNLDSKVG

SEQ ID NO: 536 - protein, 16 aa, synthetic construct
  REGION 1..16: note = Synthetic
TEIYQAGSTPCNGVEG

SEQ ID NO: 537 - protein, 28 aa, synthetic construct
  REGION 1..28: note = Synthetic
ESNKKFLPFQQFGRDIADTTDAVRDPQT

SEQ ID NO: 538 - protein, 19 aa, synthetic construct
  REGION 1..19: note = Synthetic
TPTWRVYSTGSNVFQTRAG

SEQ ID NO: 539 - protein, 15 aa, synthetic construct
  REGION 1..15: note = Synthetic
ICASYQTQTNSPRRA

SEQ ID NO: 540 - protein, 18 aa, synthetic construct
  REGION 1..18: note = Synthetic
SVASQSIIAYTMSLGAEN

SEQ ID NO: 541 - protein, 32 aa, synthetic construct
  REGION 1..32: note = Synthetic
CSNLLLQYGSFCTQLNRALTGIAVEQDKNTQE

SEQ ID NO: 542 - protein, 24 aa, synthetic construct
  REGION 1..24: note = Synthetic
VLPPLLTDEMIAQYTSALLAGTIT

SEQ ID NO: 543 - protein, 19 aa, synthetic construct
  REGION 1..19: note = Synthetic
AALQIPFAMQMAYRFNGIG

SEQ ID NO: 544 - protein, 17 aa, synthetic construct
  REGION 1..17: note = Synthetic
TQQLIRAAEIRASANLA

SEQ ID NO: 545 - protein, 15 aa, synthetic construct
  REGION 1..15: note = Synthetic
EPQIITTDNTFVSGN

SEQ ID NO: 546 - protein, 9 aa, synthetic construct
  REGION 1..9: note = Synthetic
LLFFRALPK

SEQ ID NO: 547 - protein, 9 aa, synthetic construct
  REGION 1..9: note = Synthetic
PKVYPIILR

SEQ ID NO: 548 - protein, 9 aa, synthetic construct
  REGION 1..9: note = Synthetic
ISEMHPALR

SEQ ID NO: 549 - protein, 9 aa, synthetic construct
  REGION 1..9: note = Synthetic
VTAYNGYLT

SEQ ID NO: 550 - protein, 9 aa, synthetic construct
  REGION 1..9: note = Synthetic
IIQFPNTYL

SEQ ID NO: 551 - protein, 9 aa, synthetic construct
  REGION 1..9: note = Synthetic
IKDATPSDF

SEQ ID NO: 552 - protein, 9 aa, synthetic construct
  REGION 1..9: note = Synthetic
FTIGTVTLK

SEQ ID NO: 553 - protein, 9 aa, synthetic construct
  REGION 1..9: note = Synthetic
INLIIKNLS

SEQ ID NO: 554 - protein, 9 aa, synthetic construct
  REGION 1..9: note = Synthetic
YTNSFTRGV

SEQ ID NO: 555 - protein, 9 aa, synthetic construct
  REGION 1..9: note = Synthetic
FKNIDGYFK

SEQ ID NO: 556 - protein, 9 aa, synthetic construct
  REGION 1..9: note = Synthetic
FQTLLALHR

SEQ ID NO: 557 - protein, 9 aa, synthetic construct
  REGION 1..9: note = Synthetic
IYQTSNFRV

SEQ ID NO: 558 - protein, 9 aa, synthetic construct
  REGION 1..9: note = Synthetic
FASVYAWNR

SEQ ID NO: 559 - protein, 9 aa, synthetic construct
  REGION 1..9: note = Synthetic
FVIRGDEVR

SEQ ID NO: 560 - protein, 9 aa, synthetic construct
  REGION 1..9: note = Synthetic
VIAWNSNNL

SEQ ID NO: 561 - protein, 9 aa, synthetic construct
  REGION 1..9: note = Synthetic
IAWNSNNLD

SEQ ID NO: 562 - protein, 9 aa, synthetic construct
  REGION 1..9: note = Synthetic
YQAGSTPCN

SEQ ID NO: 563 - protein, 9 aa, synthetic construct
  REGION 1..9: note = Synthetic
FLPFQQFGR

SEQ ID NO: 564 - protein, 9 aa, synthetic construct
  REGION 1..9: note = Synthetic
VYSTGSNVF

SEQ ID NO: 565 - protein, 9 aa, synthetic construct
  REGION 1..9: note = Synthetic
YQTQTNSPR

SEQ ID NO: 566 - protein, 9 aa, synthetic construct
  REGION 1..9: note = Synthetic
YTMSLGAEN

SEQ ID NO: 567 - protein, 9 aa, synthetic construct
  REGION 1..9: note = Synthetic
LLQYGSFCT

SEQ ID NO: 568 - protein, 9 aa, synthetic construct
  REGION 1..9: note = Synthetic
IAQYTSALL

SEQ ID NO: 569 - protein, 9 aa, synthetic construct
  REGION 1..9: note = Synthetic
LQIPFAMQM

SEQ ID NO: 570 - protein, 9 aa, synthetic construct
  REGION 1..9: note = Synthetic
FAMQMAYRF

SEQ ID NO: 571 - protein, 9 aa, synthetic construct
  REGION 1..9: note = Synthetic
LIRAAEIRA

SEQ ID NO: 572 - protein, 9 aa, synthetic construct
  REGION 1..9: note = Synthetic
IITTDNTFV

SEQ ID NO: 573 - protein, 9 aa, synthetic construct
  REGION 1..9: note = Synthetic
LHGTILTRP

SEQ ID NO: 574 - protein, 9 aa, synthetic construct
  REGION 1..9: note = Synthetic
YYKLGASQR

SEQ ID NO: 575 - protein, 9 aa, synthetic construct
  REGION 1..9: note = Synthetic
FTALTQHGK

SEQ ID NO: 576 - protein, 9 aa, synthetic construct
  REGION 1..9: note = Synthetic
TGPEAGLPY

SEQ ID NO: 577 - protein, 9 aa, synthetic construct
  REGION 1..9: note = Synthetic
LPQGTTLPK

SEQ ID NO: 578 - protein, 9 aa, synthetic construct
  REGION 1..9: note = Synthetic
LLLLDRLNQ

SEQ ID NO: 579 - protein, 9 aa, synthetic construct
  REGION 1..9: note = Synthetic
VTQAFGRRG

SEQ ID NO: 580 - protein, 9 aa, synthetic construct
  REGION 1..9: note = Synthetic
FAPSASAFF

SEQ ID NO: 581 - protein, 9 aa, synthetic construct
  REGION 1..9: note = Synthetic
VTPSGTWLT

SEQ ID NO: 582 - protein, 9 aa, synthetic construct
  REGION 1..9: note = Synthetic
TQALPQRQK

SEQ ID NO: 583 - protein, 20 aa, synthetic construct
  REGION 1..20: note = Synthetic
METPAQLLFLLLLWLPDTTG

SEQ ID NO: 584 - protein, 18 aa, synthetic construct
  REGION 1..18: note = Synthetic
MDWTWILFLVAAATRVHS

SEQ ID NO: 585 - protein, 24 aa, synthetic construct
  REGION 1..24: note = Synthetic
MLGSNSGQRVVFTILLLLVAPAYS

SEQ ID NO: 586 - protein, 17 aa, synthetic construct
  REGION 1..17: note = Synthetic
MKCLLYLAFLFIGVNCA

SEQ ID NO: 587 - protein, 15 aa, synthetic construct
  REGION 1..15: note = Synthetic
MWLVSLAIVTACAGA

SEQ ID NO: 588 - protein, 22 aa, synthetic construct
  REGION 1..22: note = Synthetic
GSGATNFSLLKQAGDVEENPGP

SEQ ID NO: 589 - RNA, 66 nt, synthetic construct
  source 1..66: mol_type = other RNA
  misc_feature 1..66: note = Synthetic
ggaagcggagctactaacttcagcctgctgaagcaggctggagacgtggaggagaaccctggacct

(19) United States
(12) Patent Application Publication
(10) Pub. No.: US 2026/0097115 A1
(43) Pub. Date: Apr. 9, 2026

(54) IMMUNOGENIC COMPOSITIONS AND USE THEREOF

(71) Applicants: The Broad Institute, Inc., Cambridge, MA (US); The General Hospital Corporation, Boston, MA (US); Trustees of Boston University, Boston, MA (US); President and Fellows of Harvard College, Cambridge, MA (US)

(72) Inventors: Shira Weingarten-Gabbay, Cambridge, MA (US); Da-Yuan Chen, Boston, MA (US); Siranush Sarkizova, Cambridge, MA (US); Karl Clauser, Cambridge, MA (US); Nir Hacohen, Boston, MA (US); Steven Carr, Cambridge, MA (US); Jennifer Abelin, Cambridge, MA (US); Mohsan Saeed, Boston, MA (US); Pardis Sabeti, Cambridge, MA (US)

(21) Appl. No.: 19/413,044

(22) Filed: Dec. 9, 2025

Related U.S. Application Data
(63) Continuation of application No. PCT/US2024/032913, filed on Jun. 7, 2024.
(60) Provisional application No. 63/507,324, filed on Jun. 9, 2023.

(51) Int. Cl. A61K 39/215; A61K 39/00; A61P 37/04; G01N 33/68

(52) U.S. Cl. CPC A61K 39/215; A61P 37/04; G01N 33/6818; G01N 33/6848; A61K 2039/53; G01N 2333/165

(57) ABSTRACT

Immunogenic compositions comprising one or more peptides, wherein the one or more peptides: are capable of binding to Major Histocompatibility Complex (MHC) class II, and are derived from one or more translation products of SARS-CoV-2. Also provided include methods of treating and preventing diseases using the immunogenic compositions.

### CROSS-REFERENCE TO RELATED APPLICATIONS

This application is a Continuation application of PCT/US2024/032913, filed Jun. 7, 2024, which claims the benefit of and priority to U.S. Provisional Application No. 63/507,324, filed on Jun. 9, 2023, the content of which is incorporated by reference herein in its entirety.

### STATEMENT REGARDING FEDERALLY SPONSORED RESEARCH

This invention was made with government support under Grant Nos. U19AI110818, U2CA271075, U24CA270823, U24CA210986, and U24CA210979 awarded by the National Institutes of Health, and Grant Number 58-3022-2-031 awarded by the United States Department of Agriculture. The government has certain rights in the invention.

### SEQUENCE LISTING

This application contains a sequence listing filed in electronic form as an xml file entitled BROD-5860US_ST26.xml, created on Oct. 21, 2025 and having a size of 714,812 bytes. The content of the sequence listing is incorporated herein in its entirety.

### TECHNICAL FIELD

The subject matter disclosed herein is generally directed to immunogenic compositions and use thereof.

### BACKGROUND

Severe acute respiratory syndrome coronavirus 2 (SARS-CoV-2) is the virus causing the ongoing Coronavirus Disease 19 (COVID19) pandemic. Deciphering how infected host cells interact with the immune system is important for the development of effective vaccines and therapeutics. When viruses infect cells, their proteins are being processed and presented on the host cell surface by class II Major Histocompatibility Complex (MHC-II). Cytotoxic T cells recognize the foreign antigens and initiate an immune response resulting in the infected cell death. Thus, there is a need to identify the repertoire of SARS-CoV-2 derived MHC-II peptides, which will allow development of novel vaccines, therapeutics, and assays based on the viral signature as seen by cytotoxic T cells in infected patients.

### SUMMARY

Described in exemplary embodiments herein are compositions for activating T cell-mediated immunity targeting cells infected by a virus (e.g., SARS-CoV-2) in a subject and the application thereof. In one aspect, the present invention provides for an immunogenic composition comprising one or more peptides, wherein the one or more peptides: are capable of binding to Major Histocompatibility Complex (MHC) class II, and are derived from one or more translation products of SARS-CoV-2. In certain example embodiments, the MHC class II is Human Leukocyte Antigen class II (HLA-II). In certain example embodiments, the one or more peptides have a peptide-HLA-II binding affinity of less than 500 nMa, as determined by a machine learning predictor of HLA-II epitope binding.

In certain example embodiments, the HLA-II is encoded by an HLA allele selected from the group consisting of: HLA-DRB1*07:01, HLA-DRB1*11:04, HLA-DRB1*15:01, HLA-DRB3*02:02, HLA-DRB4*01:01, HLA-DRB5*01:01, HLA-DPB1*03:01/HLA-DPA1*01:03, HLA-DPB1*04:02/HLA-DPA1*01:03, HLA-DPB1*06:01/HLA-DPA1*01:03, HLA-DQB1*02:02/HLA-DQA1*02:01, HLA-DQB1*02:02/HLA-DQA1*05:05, HLA-DQB1*03:01/HLA-DQA1*02:01, HLA-DQB1*03:01/HLA-DQA1*05:05, and HLA-DQB1*06:02/HLA-DQA1*01:02. In certain example embodiments, the HLA-II is encoded by an HLA allele selected from the group consisting of: HLA-DRB1*07:01, HLA-DRB1*11:04, HLA-DRB3*02:02, HLA-DRB4*01:01, DQB1*03:01/HLA-DQA1*05:05, HLA-DQB1*02:02/HLA-DQA1*02:01, HLA-DRB1*15:01, HLA-DRB5*01:01, HLA-DPB1*03:01/HLA-DPA1*01:03, DPB1*06:01/HLA-DPA1*01:03, HLA-DPB1*04:02/HLA-DPA1*01:03, and HLA-DQB1*06:02/HLA-DQA1*01:02.

In certain example embodiments, at least one of the peptides is derived from translation of one or more internal out-of-frame open reading frames (ORFs) of SARS-CoV-2, one or more canonical ORFs of SARS-CoV-2, or any combination thereof. In certain example embodiments, at least one of the peptides comprises a peptide sequence selected from the group consisting of internal ORF protein peptide sequences, canonical ORF protein peptide sequences, any subsequence thereof, and any combination thereof. In certain example embodiments, at least one of the peptide sequences is selected from the group consisting of peptide sequences of Table 1, Table 2, and Table 4, any subsequence thereof, and any combination thereof.

In certain example embodiments, at least one of the peptides is derived from translation of an internal out-of-frame open reading frame (ORF) of SARS-CoV-2. In certain example embodiments, at least one of the internal out-of-frame ORFs is selected from the group consisting of ORF3c (ORF3a.iORF1) and/or ORF9b (N.iORF1). In certain example embodiments, the ORF3c overlaps with the ORF3a. In certain example embodiments, the ORF9b overlaps with the N protein ORF.

In certain example embodiments, at least one of the peptides derived from translation of ORF3c comprises a peptide sequence of LLFFRALPK (SEQ ID NO: 546) or any subsequence thereof. In certain example embodiments, at least one of the peptides derived from translation of ORF3c comprises a peptide sequence of ALHFLLFFRALPKS (SEQ ID NO: 374) or any subsequence thereof. In certain example embodiments, at least one of the peptides derived from translation of ORF9b comprises a peptide sequence selected from the group consisting of PKVYPIILR (SEQ ID NO: 547), ISEMHPALR (SEQ ID NO: 548), any subsequence thereof, and any combination thereof. In certain example embodiments, at least one of the peptides derived from translation of ORF9b comprises a peptide sequence selected from the group consisting of VGPKVYPIILRLGSPLS (SEQ ID NO: 384), MDPKISEMHPALRLVDPQIQLAVTRMENA (SEQ ID NO: 382), any subsequence thereof, and any combination thereof.

In certain example embodiments, at least one of the peptides is derived from translation of canonical ORF of SARS-CoV-2. In certain example embodiments, at least one of the canonical ORFs is selected from the group consisting of non-structural protein 3 (nsp3) ORF, non-structural protein 4 (nsp4) ORF, ORF3a, ORF6, S protein ORF, M protein ORF, N protein ORF, and any combination thereof.

In certain example embodiments, at least one of the peptides derived from translation of the nsp3 ORF comprises a peptide sequence of VTAYNGYLT (SEQ ID NO: 549) or any subsequence thereof. In certain example embodiments, at least one of the peptides derived from translation of the nsp3 ORF comprises a peptide sequence selected from the group consisting of DGSEDNQTTTIQTIVE (SEQ ID NO: 363), SPDAVTAYNGYLTSSSK (SEQ ID NO: 364), any subsequence thereof, and any combination thereof.

In certain example embodiments, at least one of the peptides derived from translation of the nsp4 ORF comprises a peptide sequence of IIQFPNTYL (SEQ ID NO: 550) or any subsequence thereof. In certain example embodiments, at least one of the peptides derived from translation of the nsp4 ORF comprises a peptide sequence of MDGSIIQFPNTYLEGSVR (SEQ ID NO: 365) or any subsequence thereof.

In certain example embodiments, at least one of the peptides derived from translation of ORF3a comprises a peptide sequence selected from the group consisting of IKDATPSDF (SEQ ID NO: 551), FTIGTVTLK (SEQ ID NO: 552), any subsequence thereof, and any combination thereof. In certain example embodiments, at least one of the peptides derived from translation of ORF3a comprises a peptide sequence of MDLFMRIFTIGTVTLKQGEIKDATPSDF (SEQ ID NO: 99) or any subsequence thereof.

In certain example embodiments, at least one of the peptides derived from translation of ORF6 comprises a peptide sequence of INLIIKNLS (SEQ ID NO: 553) or any subsequence thereof. In certain example embodiments, at least one of the peptides derived from translation of ORF6 comprises a peptide sequence of YIINLIIKNLSKS (SEQ ID NO: 100) or any subsequence thereof.

In certain example embodiments, at least one of the peptides derived from translation of the S protein ORF comprises a peptide sequence selected from the group consisting of YTNSFTRGV (SEQ ID NO: 554), FKNIDGYFK (SEQ ID NO: 555), FQTLLALHR (SEQ ID NO: 556), IYQTSNFRV (SEQ ID NO: 557), FASVYAWNR (SEQ ID NO: 558), FVIRGDEVR (SEQ ID NO: 559), VIAWNSNNL (SEQ ID NO: 560), IAWNSNNLD (SEQ ID NO: 561), YQAGSTPCN (SEQ ID NO: 562), FLPFQQFGR (SEQ ID NO: 563), VYSTGSNVF (SEQ ID NO: 564), YQTQTNSPR (SEQ ID NO: 565), YTMSLGAEN (SEQ ID NO: 566), LLQYGSFCT (SEQ ID NO: 567), IAQYTSALL (SEQ ID NO: 568), LQIPFAMQM (SEQ ID NO: 569), FAMQMAYRF (SEQ ID NO: 570), LIRAAEIRA (SEQ ID NO: 571), IITTDNTFV (SEQ ID NO: 572), any subsequence thereof, and any combination thereof. In certain example embodiments, at least one of the peptides derived from translation of the S protein ORF comprises a peptide sequence selected from the group consisting of: TQLPPAYTNSFTRGVYYPDKVFRSSVLHSTQDLFLPFFS (SEQ ID NO: 529), FKNLREFVFKNIDGYFKIYSKHTPINLVRDL (SEQ ID NO: 530), INITRFQTLLALHRSYL (SEQ ID NO: 418), TVEKGIYQTSNFRVQPTES (SEQ ID NO: 532), ATRFASVYAWNRKRISN (SEQ ID NO: 424), DSFVIRGDEVRQIAPG (SEQ ID NO: 425), NYKLPDDFTGCVIAWNSNNLDSKVG (SEQ IDNO: 535), TEIYQAGSTPCNGVEG (SEQ ID NO: 440), ESNKKFLPFQQFGRDIADTTDAVRDPQT (SEQ ID NO: 442),
TPTWRVYSTGSNVFQTRAG (SEQ ID NO: 538), ICASYQTQTNSPRRA (SEQ ID NO: 445), SVASQSIIAYTMSLGAEN (SEQ ID NO: 446), CSNLLLQYGSFCTQLNRALTGIAVEQDKNTQE (SEQ ID NO: 541), EPQIITTDNTFVSGN (SEQ ID NO: 119), VLPPLLTDEMIAQYTSALLAGTIT (SEQ ID NO: 460), AALQIPFAMQMAYRFNGIG (SEQ ID NO: 543), TQQLIRAAEIRASANLA (SEQ ID NO: 498), any subsequence thereof, and any combination thereof.

In certain example embodiments, at least one of the peptides derived from translation of the M protein ORF comprises a peptide sequence selected from the group consisting of LHGTILTRP (SEQ ID NO: 573), YYKLGASQR (SEQ ID NO: 574), any subsequence thereof, and any combination thereof. In certain example embodiments, at least one of the peptides derived from translation of the M protein ORF comprises a peptide sequence selected from the group consisting of: NVPLHGTILTRPLLESELVIGAVILRGHLRIAGHHLGRCDIKDLPKEITVA (SEQ ID NO: 509), TSRTLSYYKLGASQRVAGDSG (SEQ ID NO: 139), TDHSSSSDNIALLVQ (SEQ ID NO: 22), any subsequence thereof, and any combination thereof.

In certain example embodiments, at least one of the peptides derived from translation of the N protein ORF comprises a peptide sequence selected from the group consisting of FTALTQHGK (SEQ ID NO: 575), TGPEAGLPY (SEQ ID NO: 576), LPQGTTLPK (SEQ ID NO: 577), LLLLDRLNQ (SEQ ID NO: 578), VTQAFGRRG (SEQ ID NO: 579), FAPSASAFF (SEQ ID NO: 580), VTPSGTWLT (SEQ ID NO: 581), TQALPQRQK (SEQ ID NO: 582), any subsequence thereof, and any combination thereof. In certain example embodiments, at least one of the peptides derived from translation of the N protein ORF comprises a peptide sequence selected from the group consisting of: GLPNNTASWFTALTQHGKEDLKFPRGQGVPINTNSSPDDQIGYYRRATRRIR (SEQ ID NO: 513), TGPEAGLPYGANKDG (SEQ ID NO: 32), VATEGALNTPKDHIGTRNPANNAAIVLQLPQGTTLPKG (SEQ ID NO: 164), MAGNGGDAALALLLLDRLNQLESKMSGKGQQQQGQTVT (SEQ ID NO: 34),
AAEASKKPRQKRTATKAYNVTQAFGRRGPEQTQGNFGDQELIRQGTD (SEQ ID NO: 517), IAQFAPSASAFFG (SEQ ID NO: 55), SDNGPQNQRNAPRITF (SEQ ID NO: 24), KKKADETQALPQRQKKQQTVTLLPAADLDDFSKQLQQSMSSADSTQA (SEQ ID NO: 520), RIGMEVTPSGTWLTYTGAIKLDDKDPNFKDQVILLNKHIDAYKTFPP (SEQ ID NO: 519), any subsequence thereof, and any combination thereof.

In certain example embodiments, the one or more peptides comprise a peptide sequence of N₁XXN₂XN₃XXN₄, wherein N₁ is F, I, L, M, V, W, or Y, wherein N₂ is A, I, F, L, M, N, T, Q, S, V, W, or Y, wherein N₃ is A, D, E, G, H, K, N, P, R, S, or T and wherein N₄ is A, E, F, G, K, I, L, N, M, R, S, V, or Q. In certain example embodiments, N₁ is F, I, L, M, V, W, or Y; N₂ is N, T, S, or V; or Y; N₃ is A, G, N, P, S, or T; and N₄ is F, I, L, N, M, or V; or N₁ is I, L, M, or V; N₂ is I, L, M, or V; N₃ is H, K, or R; and N₄ is A, G, S, or Q; or N₁ is F, I, L, V, W, or Y; N₂ is F, I, N, M, W, or Y; N₃ is D, G, N, or S; and N₄ is K, L, N, S, or V; or N₁ is F, I, L, M, V, W, or Y; N₂ is A, E, I, L, M, Q, or V; N₃ is A, E, G, or S; and N₄ is E, K, or R.

In one aspect, the present invention provides for a polynucleotide encoding one or more peptides of the present invention. In one aspect, the present invention provides for a vector comprising a polynucleotide of the present invention. In certain example embodiments, the vector is a synthetic mRNA vaccine.

In one aspect, the present invention provides for an immunogenic composition comprising: (a) one or more peptides of the present invention, one or more polynucleotides of the present invention, a vector of the present invention, or any combination thereof, and (b) one or more antigenic components capable of stimulating production of an antibody targeting SARS-CoV-2. In certain example embodiments, the one or more antigenic components comprises one or more antigenic peptides from a nucleocapsid phosphoprotein of SARS-CoV-2, a spike glycoprotein of SARS-CoV-2, or any combination thereof, one or more polynucleotides encoding the one or more antigenic peptides, or any combination thereof.

In one aspect, the present invention provides for a therapeutic composition comprising an immunogenic composition of the present invention, and an anti-viral therapeutic. In certain example embodiments, the one or more polynucleotides encoding the one or more antigenic peptides are a synthetic mRNA vaccine.

In one aspect, the present invention provides for a method of inducing a T cell response, and optionally an antibody response, to SARS-CoV-2 in a subject in need thereof comprising administering, to the subject, an immunogenic composition of the present invention, a vector of the present invention, or any combination thereof.

In one aspect, the present invention provides for a method of treating a SARS-CoV-2 infection in a subject in need thereof comprising administering a therapeutic composition of the present invention to the subject in need thereof.

In one aspect, the present invention provides for a method of determining an infection status of a subject comprising contacting immune cells derived from a subject with the immunogenic composition of the present invention; and detecting cross-reactivity of the immune cells to the immunogenic composition.

In one aspect, the present invention provides for a method of identifying immunogenic peptides comprising: (a) lysing cells having a potential to express the immunogenic peptides of interest with a lysis buffer comprising a cell membrane disrupting detergent; (b) enzymatic shearing of nucleic acids in the lysed cells; (c) isolating HLA-II from the lysed cells, wherein the HLA-II is in complex with one or more peptides or proteins from the lysed cells; and (d) determining sequences of the one or more peptides or proteins in complex with the HLA-II from (c). In certain example embodiments, the method further comprises (e) identifying HLA-II alleles encoding HLA-II that bind the peptides identified in using a HLA-II epitope binding predictor, and (f) selecting a subset of peptides that bind HLA-II encoded by a defined percentage of HLA-II alleles. In certain example embodiments, the method further comprises selecting immunogenic peptides demonstrating a relative abundance above a defined threshold as determined by analysis of the complete cellular transcriptome and or proteome. In certain example embodiments, the method further comprises ribosome sequencing to identify actively translated peptides and selecting immunogenic peptides that are being actively translated at one or more time points. In certain example embodiments, the nucleic acids in the lysed cells are enzymatically sheared using an endonuclease from Serratia marcescens and MgCl₂. In certain example embodiments, the cell membrane disrupting agent is an octylphenol ethoxylate surfactant. In certain example embodiments, (d) is performed by liquid chromatography tandem mass spectrometry analysis. In certain example embodiments, isolating HLA-II comprises immunoprecipitation of the HLA-II complex with an anti-HLA-II antibody. In certain example embodiments, the immunogenic peptides of interest are expressed by a pathogen and wherein the cells have been infected with the pathogen. In certain example embodiments, the infected cells are engineered to express one or more cell surface receptors used by the pathogen to infect the cells. In certain example embodiments, the cells are treated with one or more cell signaling molecules related to infection by the pathogen. In certain example embodiments, the pathogen is a virus. In certain example embodiments, the virus is SARS-Cov-2. In certain example embodiments, the cells are engineered to express class II transactivator (CIITA), angiotensin-converting enzyme 2 (ACE2), and transmembrane serine protease 2 (TMPRSS2). In certain example embodiments, the cells are engineered to increase or decrease HLA presentation. In certain example embodiments, the cells are engineered to increase or decrease expression of one or more of CIITA, proteasome subunits, tissue Plasminogen Activator (tPA), proteasome maturation protein (POMP), or ubiquitin-proteasome genes.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1G—HLA-II immunopeptidome profiling of SARS-CoV-2 infected cells. (FIG. 1A) Schematic of the experimental workflow. (FIG. 1B) HLA-II expression measured by flow cytometry using a FITC-conjugated anti HLA-DR/DP/DQ antibody. Gating was based on unstained cells. (FIG. 1C) SARS-CoV-2 infection levels in A549/AT and HEK293T/AT cells with and without CIITA transduction. The infection was quantified using immunofluorescence staining for the nucleocapsid protein. MOI=Multiplicity Of Infection. (FIG. 1D) Length distribution of the eluted peptides in SARS-CoV-2 infected cells. (FIG. 1E) Gibbs Cluster deconvoluted peptide sequences of the eluted peptides. (FIG. 1F) The percentage of peptides originating from Bovine proteins detected in the HLA-I and HLA-II immunopeptidomes. HLA-I data are from Weingarten-Gabbay et al. (Weingarten-Gabbay et al., 2021) (FIG. 1G) Length distribution of human and bovine peptides in the A549 and HEK293T HLA-I and the HLA-II immunopeptidomes.

FIG. 2A-2C—SARS-CoV-2 HLA-II immunopeptidome. (FIG. 2A) Summary of viral proteins presented on HLA-II complexes in infected 549/ATC and HEK293T/ATC cells. (FIG. 2B (SEQ ID NOs: 22, 34, 139, 164, 460, 498, 509, 513, 517, 519, 520, 529, 530, 532, 535, 541, 543)) The location of HLA-II peptides in canonical structural proteins M, N and S. Peptides detected in A549/ATC and HEK293T/AT/C cells are depicted in black and gray, respectively. (FIG. 2C (SEQ ID NOs: 374, 382, 384)) The location of HLA-II peptides in two non-canonical internal ORFs: ORF9b and ORF3c.

FIG. 3A-3E—Systematic comparison between the SARS-CoV-2 HLA-II immunopeptidome and known CD4+ T cell epitopes. (FIG. 3A) A list of nine studies that identified CD4+ T cells epitopes in COVID-19 patients as described in Table 1 of Grifoni et al (Grifoni et al., 2021). Studies that included peptides from all canonical SARS-CoV-2 proteins are highlighted with an asterisk. (FIG. 3B) Comparing the immunogenicity of SARS-CoV-2 proteins that were detected on the HLA-II complex to proteins that were not. For each protein, Applicant plotted the fraction of CD4+ T cell epitopes from all the detected epitopes in four genome-wide studies (denoted with asterisk in panel A). Wilcoxon-rank sum p-value is shown. (FIG. 3C-3E) Comparing the location of HLA-II peptides that were detected by mass spectrometry in infected A459/AT/CIITA and HEK293T/AT/CIITA cells to previously reported CD4+ T cell epitopes. (left) heatmap showing the density of HLA-II peptides and the reported CD4+ T cell epitopes across individual viral proteins. Rows were normalized according to the maximal density in each row. (right) venn diagram showing the number of amino acids that are covered by HLA-II peptides, CD4+ T cell epitopes and both. To reduce background levels, Applicant counted amino acids with a greater coverage than the mean of each row. Hypergeometric p-value is shown for each viral protein.

FIG. 4A-4D—SARS-CoV-2 protein representation on the HLA-I and HLA-II complexes. (FIG. 4A) A bar chart showing the number of HLA-II peptides detected in SARS-CoV-2 infected A549/AT/CIITA and HEK293T/AT/CIITA cells for each viral protein. Inside the frame is a pie chart showing the relative abundance of peptides derived from structural proteins, non-structural proteins, accessory proteins and non-canonical ORFs. (FIG. 4B) Similar to (FIG. 4A) for HLA-I peptides reported in Weingarten-Gabbay et al. (Weingarten-Gabbay et al., 2021). (FIG. 4C) A cartoon illustrating the source proteins for the HLA-II processing and presentation pathway. Viruses are endocytosed by an antigen presenting cell; The viral structural proteins are cleaved within the endosomal-lysosomal compartment and loaded onto HLA-II complexes. (FIG. 4D) A cartoon illustrating the source proteins for the HLA-I processing and presentation pathway. Viral proteins are produced from the translation of genomic and subgenomic viral RNAs in infected cells. These proteins are cleaved and loaded onto HLA-I complexes.

FIG. 5A-5B—Proper induction of the MHC-II locus and SARS-CoV-2 infectivity in CIITA overexpressing cells. (FIG. 5A) Heatmap of log 10 iBAQ values for key proteins in the MHC-II locus, observed in whole proteome datasets of A549/AT and HEK293T/AT cells with and without CIITA transduction. Shown are two biological replicates for each condition. (FIG. 5B) SARS-CoV-2 infectivity assay comparing A549/AT and HEK293T/AT with and without CIITA transduction. Representative immunofluorescence images at 24 hpi. Red, nucleocapsid; Blue, DAPI. Images were captured with an EVOS microscope using 10× objective lens.

FIG. 6A-6B—HLA-II peptides in SARS-CoV-2 non-structural and accessory proteins. (FIG. 6A (SEQ ID NOs: 521-523)) The location of HLA-II peptides across the non-structural proteins nsp3 and nsp4. (FIG. 6B (SEQ ID NOs: 524, 526)) The location of HLA-II peptides across the accessory proteins ORF3a and ORF6. Peptides detected in A549/ATC and HEK293T/ATC cells are depicted in black and gray, respectively.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The term “peptide sequence” or “segment sequence” are used interchangeably herein to mean a portion of an amino acid sequence of a protein, a nucleic acid sequence of a polynucleotide, etc.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The ongoing COVID-19 pandemic has marked the mass employment of synthetic vaccines in which a relatively small portion of the viral genome is administered to provide protection. While the first generation of vaccines target only a single viral protein, this remarkable technology provides an unprecedented opportunity to incorporate multiple highly potent T cell epitopes into a single vaccine. To select the best viral targets, a fundamental understanding of how T cells “see” and interact with the virus is required. Helper CD4+ T cells are essential players in the immune response to SARS-CoV-2, yet critical information is missing on the repertoire of viral peptides that are presented on the HLA-II complex: (1) A genome-wide view of viral peptides that are presented on the HLA-II complex. Knowledge of HLA-II peptides is limited to only five SARS-CoV-2 proteins that were evaluated outside the context of infection using a recombinant spike protein (Knierman et al. Cell Reports 2020 and Parker et al. Cell Reports 2021) or four individual overexpression plasmids (Nagler et al. Cell Reports 2021); (2) If and to what extent, non-canonical ORFs participate in HLA-II antigen presentation. It was recently reported that non-canonical ORFs are a major, unexpected source for HLA-I presentation with some peptides eliciting greater CD8+ T cell responses than canonical peptides (Weingarten-Gabbay et al. Cell 2021). However, no study to date has examined if peptides derived from non-canonical ORFs are presented on the HLA-II complex or elicit a CD4+ T cell response; (3) If the same viral antigens are presented on the HLA-I and the HLA-II complexes. The assumption that a single viral protein can elicit potent responses of CD4+ and CD8+ T cells stands at the basis of the design of current vaccines. Mapping which parts of the SARS-CoV-2 genome are presented to CD4+ and CD8+ T cells is critical to test this assumption and to maximize vaccine effectiveness.

Recent success with synthetic vaccines against viral diseases has demonstrated their promise for future use, but also highlighted the need for a deeper understanding of how the adaptive immune system recognizes and responds to viral infections (Sette & Crotty, 2021; Wherry & Barouch, 2022). In contrast to traditional attenuated vaccines that mimic the natural interaction between viruses and the immune system, rationally designed synthetic vaccines deliver only a small selected portion of the viral genome, often a single protein, to stimulate an immune response. This allows rationally designed vaccines to be developed quickly, produced in large quantities with low-cost manufacturing, and administered safely (Pardi et al., 2018). However, the development of rationally designed vaccines necessitates a comprehensive knowledge of viral immunology and the parts of the viral genome that are recognized by the different arms of the immune system.

Limitations of the COVID-19 vaccines developed during the course of the pandemic highlighted the importance of eliciting a more comprehensive immune response through T cells (Neale et al., 2023). Early vaccines mostly focused on eliciting a humoral immune response through utilizing the viral spike protein (S) (Baden et al., 2021; Martinez-Flores et al., 2021; Sahin et al., 2021). However, challenges with waning immunity led to a greater focus on providing durable immunity by eliciting CD8+ cytotoxic and CD4+ helper T cell responses. Together with the high frequency of mutations in spike and the associated immune evasion of emerging variants, these findings motivated researchers to incorporate T cell targets, such as the nucleocapsid (N), in the next generation of COVID-19 vaccines (Arieta et al., 2023; Dutta Noton K. et al., 2020; Hajnik et al., 2022; Matchett et al., 2021; Oronsky et al., 2022). CD8+ and CD4+ T cells recognize viral peptides that are endogenously processed inside cells and presented on the surface via HLA-I and HLA-II complexes, respectively. These T cell epitopes originate from all viral proteins, in contrast to neutralizing antibodies that are mostly confined to external structural viral proteins. Although the presence of a large number of potential T cell epitopes in the viral genome offer a wide range of candidates, it can also present a challenge in identifying the most effective targets for T cell-based vaccines.

To understand the full range of T cell epitopes, it is important to implement non-targeted, comprehensive approaches in addition to traditional conventional T cell assays. Targeted methods T cell assays require researchers to decide a priori which peptides to use screen in the experiment, based on assumptions about which viral proteins are expressed and processed for HLA presentation. While T cell responses to SARS-CoV-2 have been extensively studied, many studies have focused on only a subset of viral proteins, particularly the structural proteins S, N, membrane (M), and envelope (E), rather than considering the full range of proteins in the SARS-CoV-2 proteome (ref). (Chen et al., 2021; Joag et al., 2021; Keller et al., 2020; Le Bert et al., 2021; Lee et al., 2021; Mahajan et al., 2021; Nielsen et al., 2021; Rha et al., 2021; Shomuradova et al., 2020). A smaller number of studies have looked at T cell responses to peptides from all annotated viral proteins and found significant responses to both structural and non-structural proteins (cite). (Ferretti et al., 2020; Gangaev et al., 2021; Kared et al., 2021; Mateus et al., 2020; Nelde et al., 2021; Prakash et al., 2021; Tarke et al., 2021). Although these studies provide a broader view, they have still generally only included peptides from annotated canonical proteins and have not considered peptides from non-canonical ORFs that were identified through experimental translation measurements (Finkel et al., 2020).

Taking an untargeted approach of HLA immunopeptidome profiling by mass spectrometry, Applicant recently revealed surprising insights about HLA-I presentation in SARS-CoV-2 infected cells. Immunopeptidome profiling utilizes mass spectrometry to detect peptides that are endogenously presented on the HLA complex in different disease contexts (Abelin et al., 2017; Bassani-Sternberg & Gfeller, 2016; Chong et al., 2018; Croft et al., 2013; McMurtrey et al., 2008; Rucevic et al., 2016; Sarkizova et al., 2020; Schellens et al., 2015; Ternette et al., 2016). By applying it to SARS-CoV-2 infected cells Applicant uncovered HLA-I peptides from canonical proteins as well as overlapping ORFs in the coding region of N and S that were overlooked by dozens of previous T cell studies (Weingarten-Gabbay et al., 2021). Strikingly, some of the non-canonical peptides were more immunogenic in COVID-19 patients and humanized mice than some of when compared to the strongest epitopes canonical protein derived antigens reported to date. Two of the non-canonical peptides were further supported by an independent HLA-I peptidome study from another group (Nagler et al., 2021). Applicant also uncovered that, in contrast to earlier studies focusing mostly on structural proteins, non-structural proteins constitute a significant portion of the HLA-I peptidome, and that the timing of viral protein expression is a key determinant of HLA-I presentation and immunogenicity. Thus, the list of SARS-CoV-2 HLA-I peptides resulting from Applicant's study was recently used to characterize a T cell-directed mRNA vaccine (BNT162b4) (Arieta et al., 2023). Of the 19 HLA-I peptides observed in cells expressing a multi epitope vaccine, 3 were identical to peptides that Applicant detected in infected cells. Together, HLA immunopeptidome profiling can identify new, highly potent T cell epitopes, inform vaccine design, and deepen the understanding of the determinants of viral antigen presentation.

Immunopeptidome profiling can be utilized to directly characterize yet another critical process in the antiviral immune response: HLA-II presentation to CD4+ helper T cells. In contrast to HLA-I presentation that samples endogenous cytosolic proteins, HLA-II mostly presents peptides from proteins that have been taken up from outside the cell via endocytosis. Hence, different viral proteins may differentially access the HLA-I and HLA-II pathways. Understanding these differences can enable researchers to design vaccines that elicit both CD8+ and CD4+ T cells responses. Previous HLA-II peptidome studies of SARS-CoV-2 researched HLA-II peptides derived only from the spike protein, using a purified recombinant protein (Knierman et al., 2020; Parker et al., 2021), or from four viral proteins (N, M, E and nsp6) using plasmid overexpression (Nagler et al., 2021). Thus, a systematic view of HLA-II peptides from the full SARS-CoV-2 genome in the context of authentic virus infection has not yet been achieved.

Applicant discloses herein the first genome-wide immunopeptidome study of SARS-CoV-2 antigens that are naturally processed and presented on the HLA-II complex. To this end, Applicant induced the HLA-II pathway in two SARS-CoV-2 infected cell lines and performed untargeted profiling of HLA-II peptides using mass spectrometry. Applicant complemented this analysis with thousands of reported CD4+ T cell epitopes from multiple studies to evaluate the contribution of peptides that are presented on the HLA-II complex to the T cell response in COVID-19 patients. Finally, Applicant compared the HLA-II and HLA-I immunopeptidomes and the viral antigens that are presented on each complex to assess which parts of the viral genome are seen by CD4+ and CD8+ T cells. The key highlights from this study are:
(1) This is the first experimental profiling of peptides derived from the entire SARS-CoV-2 genome that are endogenously processed and presented on the HLA-II complex, performed in two human cell lines;
(2) This study is the first to employ CIITA overexpression to research the HLA-II immunopeptidome of a high-containment virus. The adaptation of the protocol to the restrictions associated with working in a biosafety level 3 (BSL3) laboratory will facilitate future characterization of highly pathogenic viruses in the context of native infection;
(3) A striking difference was discovered between viral antigens that are presented on the HLA-II and the HLA-I complexes. These differences suggest that the design of current high-profile vaccines might unknowingly be biased toward CD4+ T cell responses at the cost of dismissing crucial antigens for CD8+ T cells;
(4) This analyses point to an inherent difference in the accessibility of viral proteins to HLA-I and HLA-II presentation pathways that correlates with the viral life cycle. The HLA-II complex preferentially presents peptides from structural proteins forming mature virions, while the HLA-I complex samples viral proteins that are actively translated in infected cells, including many proteins that are not assembled into viral particles. It is proposed that this difference is a global feature of antigen presentation across many viruses, with implications in vaccine design;
(5) It was discovered that out-of-frame ORFs in the coding region of the nucleocapsid and ORF3a contribute to the repertoire of HLA-II peptides. These non-canonical ORFs were not captured by any vaccines and are missing from all T cell studies performed to date;
(6) This study provides the first evidence for the expression of the non-canonical ORF3c protein in SARS-CoV-2 infected cells. Functional characterization of ORF3c hints at an important function in viral immune evasion, however, albeit many attempts, scientists were not able to detect this protein in infected cells; and
(7) Using this new knowledge of HLA-II peptides that are endogenously processed in cells and curated datasets of CD4+ T cell epitopes, it was shown that two immunodominant hotspots in the viral membrane protein are formed due to selective presentation of peptides from these regions.

It is believed that these findings and the list of naturally presented HLA-II peptides will have an immediate and direct impact on the vaccine design and immune monitoring of COVID-19 patients. Importantly, the list of HLA-I peptides resulting from Applicant's previous study was recently used by BioNTech to characterize a new T cell-directed mRNA vaccine (BNT162b4) (Arieta et al. Cell 2023). In addition, the biological insights into how SARS-CoV-2 presents different subsets of proteins to CD4+ and CD8+ T cells, challenging the “one-protein-fits-all” approach, will allow scientists to design broader, more effective vaccines.

### Compositions

The present disclosure provides compositions for activating T cell-mediated immunity targeting cells infected by a virus (e.g., SARS-CoV-2) in a subject and the application thereof. In one aspect, the compositions comprise one or more peptides that are a) capable of binding to Major Histocompatibility Complex (MHC) class II and b) derived from one or more viral nucleic acid translation products (e.g., viral polypeptides or viral proteins). In some embodiments, the one or more peptides may be derived from translation of internal out-of-frame open reading frames (ORFs), canonical ORF, or any combination thereof, of a nucleic acid in the virus. In some embodiments, the compositions may further comprise one or more antigenic components capable of stimulating production of an antibody targeting a virus.

In one aspect, the present disclosure provides immunogenic compositions comprising one or more immunogenic peptides suitable for inducing immunity in a subject, e.g., to protect from or treat the infection of a virus. In some cases, the present disclosure includes one or more polynucleotides encoding the one or more peptides herein. In some embodiments, the immunogenic compositions may elicit an immunological response in the host to which the immunogenic compositions are administered. Such immunological response may be a T cell-mediated (e.g., cytotoxic T cell-mediated) immune response to the immunogenic compositions. In certain embodiments, the immunogenic compositions may be combined with one or more antigenic components and/or anti-viral therapeutics. In some examples, such combination may elicit cellular and/or antibody-mediated immune response, e.g., production or activation of antibodies, B cells, helper T cells, suppressor T cells, and/or cytotoxic T cells and/or gamma-delta T cells.

In one aspect the present disclosure provides therapeutic compositions comprising one or more immunogenic compositions of the present disclosure or one or more elements thereof, and an anti-viral therapeutic. In certain example embodiments, the therapeutic compositions may be used to treat viral infection of a subject. For example, the therapeutic compositions may be used to remove infected cells in the subject. Alternatively or additionally, the therapeutic compositions may be used to prevent viral infection or reduce the impact of viral infection on the subject (e.g., clinical signs normally displayed by an infected host, a quicker recovery time and/or a lowered duration of infectivity or lowered pathogen titer in the tissues or body fluids or excretions of the infected subject). In some cases, the subject displays a protective immunological response such that resistance to new infection may be enhanced and/or the clinical severity of the disease may be reduced.

### Major Histocompatibility Complex (MHC) Class II

Described in exemplary embodiments herein are immunogenic compositions comprising one or more peptides, wherein the one or more peptides: are capable of binding to Major Histocompatibility Complex (MHC) class II, and are derived from one or more translation products of SARS-CoV-2. In certain example embodiments, the MHC class II is Human Leukocyte Antigen class II (HLA-II). In certain example embodiments, after binding, the MHC class II may present the one or more peptides to activate cytotoxic T cells. As used herein, MHC refers to protein complexes capable of binding peptides resulting from the proteolytic cleavage of polypeptide or protein antigens and representing potential T-cell epitopes, transporting them to the cell surface and presenting them there to specific cells, in particular cytotoxic T-lymphocytes or T-helper cells. MHC class II, or MHC-II, function mainly in antigen presentation to CD4+ T lymphocytes or cytotoxic T cells and may be heterodimers comprising two polypeptide chains, an α- and β-chains comprising two domains each (α1 and β1, and α2 and β2).

In some embodiments, the MHC class II may be Human Leukocyte Antigen (HLA) class II, which is the MHC class II in humans. HLA class II may comprise α- and β-chains comprising two domains each (α1 and β1, and α2 and β2). An HLA corresponding to MHC class II may be HLA-DP, HLA-DM, HLA-DO, HLA-DQ, or HLA-DR. The alpha chain may be HLA-DMA, HLA-DOA, HLA-DPA1, HLA-DQA1, HLA-DQA2, or HLA-DRA. The beta chain may be HLA-DMB, HLA-DOB, HLA-DPB1, HLA-DQB1, HLA-DQB2, HLA-DRB1, HLA-DRB3, HLA-DRB4, or HLA-DRB5. In one example, the one or more peptides binds, or is capable of binding, to HLA-DP. In another example, the one or more peptides binds, or is capable of binding, to HLA-DM. In another example, the one or more peptides binds, or is capable of binding, to HLA-DO. In another example, the one or more peptides binds, or is capable of binding, to HLA-DQ. In another example, the one or more peptides binds, or is capable of binding, to HLA-DR.

### HLA Alleles

The one or more peptides may bind, or may be capable of binding, to proteins encoded by certain HLA alleles. HLA genes may be polymorphic and have many different alleles, allowing them to fine-tune the immune system. The nomenclature of HLA genes is well known in the art, e.g., as described in Marsh S G E et al., Nomenclature for factors of the HLA system, 2010, Tissue Antigens. 2010 April; 75(4): 291-455, which is incorporated by reference in its entirety.

The HLA alleles may encode HLA protein capable of epitope binding. The present disclosure may further comprise identifying HLA alleles that bind the peptides using an HLA II epitope binding predictor and selecting a subset of peptides that bind a defined percentage of HLA II alleles in a population. In some cases, the HLA alleles may have binding affinities of less than 500 nM.

Exemplary methods are disclosed in: Abelin, et al. (2019). Defining HLA-II Ligand Processing and Binding Rules with Mass Spectrometry Enhances Cancer Epitope Prediction. Immunity, 51(4), 766-779.e17; Andreatta, et al. (2017). GibbsCluster: unsupervised clustering and alignment of peptide sequences. Nucleic Acids Research, 45(W1), W458-W463.; Lippolis, et al., (2002). Analysis of MHC class II antigen processing by quantitation of peptides that constitute nested sets. Journal of Immunology, 169(9), 5089-5097.; Taylor, et al., (2021); and MS-Based HLA-II Peptidomics Combined With Multiomics Will Aid the Development of Future Immunotherapies. Molecular & Cellular Proteomics: MCP, 20, 100116, the subject matter of which is incorporated herein.

The immunogenic peptides may be selected based on sequencing data. For example, the methods may further comprise selecting immunogenic peptides demonstrating a relative abundance above a defined threshold as determine by analysis of the complete cellular transcriptome and/or proteome. In some cases, the expression level of genes may be determined (e.g., by computational methods based on the sequencing data) and the peptides may be ranked and selected from highly abundant genes (e.g., genes with high expression levels). Alternatively or additionally, ribosomal sequencing may be used (in some cases no RNA-seq data is used) to identify peptides that are being actively translated by the cell at one or more time points, and only those peptides that are actively translated are selected. The datasets from this approach are different from conventional mass-spectrometry search datasets in that they include out-of-frame ORFs, which may include internal out-of-frame ORFs.

In certain example embodiments, the proteins encoded by HLA alleles include HLA proteins encoded by an HLA allele selected from the group consisting of: HLA-DRB1*07:01, HLA-DRB1*11:04, HLA-DRB1*15:01, HLA-DRB3*02:02, HLA-DRB4*01:01, HLA-DRB5*01:01, HLA-DPB1*03:01/HLA-DPA1*01:03, HLA-DPB1*04:02/HLA-DPA1*01:03, HLA-DPB1*06:01/HLA-DPA1*01:03, HLA-DQB1*02:02/HLA-DQA1*02:01, HLA-DQB1*02:02/HLA-DQA1*05:05, HLA-DQB1*03:01/HLA-DQA1*02:01, HLA-DQB1*03:01/HLA-DQA1*05:05, and HLA-DQB1*06:02/HLA-DQA1*01:02. In certain example embodiments, the proteins encoded by HLA alleles include HLA proteins encoded by an HLA allele selected from the group consisting of: HLA-DRB1*07:01, HLA-DRB1*11:04, HLA-DRB3*02:02, HLA-DRB4*01:01, DQB1*03:01/HLA-DQA1*05:05, HLA-DQB1*02:02/HLA-DQA1*02:01, HLA-DRB1*15:01, HLA-DRB5*01:01, HLA-DPB1*03:01/HLA-DPA1*01:03, DPB1*06:01/HLA-DPA1*01:03, HLA-DPB1*04:02/HLA-DPA1*01:03, and HLA-DQB1*06:02/HLA-DQA1*01:02.

In one example embodiment, the one or more peptides bind, or are capable of binding, to an HLA protein encoded by HLA-DRB1*07:01. In one example embodiment, the one or more peptides bind, or are capable of binding, to an HLA protein encoded by HLA-DRB1*11:04. In one example embodiment, the one or more peptides bind, or are capable of binding, to an HLA protein encoded by HLA-DRB3*02:02. In one example embodiment, the one or more peptides bind, or are capable of binding, to an HLA protein encoded by HLA-DRB4*01:01. In one example embodiment, the one or more peptides bind, or are capable of binding, to an HLA protein encoded by DQB1*03:01/HLA-DQA1*05:05. In one example embodiment, the one or more peptides bind, or are capable of binding, to an HLA protein encoded by HLA-DQB1*02:02/HLA-DQA1*02:01. In one example embodiment, the one or more peptides bind, or are capable of binding, to an HLA protein encoded by HLA-DRB1*15:01. In one example embodiment, the one or more peptides bind, or are capable of binding, to an HLA protein encoded by HLA-DRB5*01:01. In one example embodiment, the one or more peptides bind, or are capable of binding, to an HLA protein encoded by HLA-DPB1*03:01/HLA-DPA1*01:03. In one example embodiment, the one or more peptides bind, or are capable of binding, to an HLA protein encoded by DPB1*06:01/HLA-DPA1*01:03. In one example embodiment, the one or more peptides bind, or are capable of binding, to an HLA protein encoded by HLA-DPB1*04:02/HLA-DPA1*01:03. In one example embodiment, the one or more peptides bind, or are capable of binding, to an HLA protein encoded by HLA-DQB1*06:02/HLA-DQA1*01:02.

### Peptides

The immunogenic compositions may comprise various peptides. Each peptide is capable of binding to Major Histocompatibility Complex (MHC) class II and is derived from one or more translation products of a virus (e.g., viral polypeptides or viral proteins). In certain example embodiments, the one or more peptides are derived from a virus that is related to a viral infection targeted for prevention, treatment or reduction of impact in a subject.

As used herein, unless otherwise indicated, when a peptide is “derived from a virus”, it means that the peptide is derived from translation of an ORF in a viral genome (a.k.a., the peptide is derived from a viral polypeptide or a viral protein expressed from an ORF in a viral genome). In certain example embodiments, the term “derived from viral polypeptides or viral proteins” means derived from proteolytic cleavage of said viral polypeptides or viral proteins in a cell. In an aspect, the one or more peptides are derived from a polypeptide or a protein of SARS-Co-V-2, and is optionally be derived from expression of an internal out-of-frame open reading frame (ORF) or a canonical ORF of SARS-CoV-2.

### Viral Polypeptides and Viral Proteins

The one or more peptides may be derived from one or more translation products of a viral nucleic acid (e.g., viral polypeptides or viral proteins). A peptide derived from a polypeptide or protein has an amino acid sequence that is a portion or the full-length of the polypeptide or protein's amino acid sequence. In some examples, the one or more peptides may result from digestion or degradation of a viral polypeptide or a viral protein in a cell infected by a virus. The virus may be a DNA virus, a RNA virus, or a retrovirus.

In certain example embodiments, the virus is a coronavirus. The coronavirus may be a positive-sense single stranded RNA family of viruses, infecting a variety of animals and humans. In one example, the virus is SARS-CoV-2. SARS-CoV is one type of coronavirus infection, as well as MERS-CoV. Example sequences of the SARS-CoV-2 are available at GISAID accession no. EPI_ISL_402124 and EPI_ISL_402127-402130, and described in DOI: 10.1101/2020.01.22.914952. Further deposits of the example SARS-CoV2 are deposited in the GISAID platform include EP_ISL_402119-402121 and EP_ISL_402123-402124; see also GenBank Accession No. MN908947.3.

In certain example embodiments, the virus is selected from the group consisting of Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islands virus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2†.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

### Open Reading Frames (ORFs)

The one or more translation products of a virus (e.g., viral polypeptides or viral proteins) may be expressed from one or more open reading frames (ORFs) in a viral genome. An ORF refers to a polynucleotide that encodes a protein, or a portion of a protein (e.g., a polypeptide). An open reading frame usually begins with a start codon and is read in codon-triplets until the frame ends with a STOP codon.

In some embodiments, the ORFs are canonical ORFs. A canonical ORF is an ORF that is most prevalent, most similar to orthologous sequences found in other species, by virtue of its length or amino acid composition, allows for the clearest description of domains, isoforms, polymorphisms, post-translational modifications, or in the absence of other information is the longest sequence. In some examples, the ORFs are canonical ORFs of SARS-CoV-2, such as nsp3, nsp4, ORF3a, ORF6, S protein, M protein, or N protein. In one example, the ORF is nsp3 ORF. In another example, the ORF is nsp4 ORF. In another example the ORF is ORF3a. In another example, the ORF is ORF6. In another example, the ORF is spike (S) protein ORF. In another example, the ORF is membrane (M) protein ORF. In another example, the ORF is membrane (M) protein ORF. In another example, the ORF is nucleocapsid (N) protein ORF. In other example embodiments, peptides are derived from non-canonical translation of ORF, such as via internal ribosome entry, leaky scanning, non-AUD initiation, ribosome shunting, reinitiation, ribosomal frameshifting and stop-codon readthrough.

In some embodiments, the ORFs are out-of-frame ORFs. As used herein, the term “out-of-frame ORF” refers to ORFs that are out of frame with a canonical ORF. In certain example embodiments, the out-of-frame ORF is an internal out-of-frame ORF, which is an ORF found within a canonical ORF but out of frame with the canonical ORF. In some examples, the ORFs are internal out-of-frame ORFs of SARS-CoV-2, such as ORF9b (overlapping with N, also called N.iORF1), or ORF3c (overlapping with ORF3a, also called 3a.iORF1). In one example, the ORF is ORF9b. In another example, the ORF is ORF3c.

Other examples of the ORFs include those described in Finkel Y et al., The coding capacity of SARS-CoV-2, doi: doi.org/10.1101/2020.05.07.082909, which is incorporated by reference in its entirety.

In some embodiments, the sequence, ORFs, and other annotations are based on the sequence of 2019-nCoV/USA-WA1/2020 isolate (NCBI accession number: MN985325) of SARS-CoV-2.

### Examples of Peptides

In some embodiments, the one or more peptides comprise one or more peptide sequences selected from the group consisting of: peptide sequences of Table 1, Table 2, Table 4; any subsequences thereof, and any combination thereof. In some embodiments, the one or more peptides comprise a peptide sequence of N₁XXN₂XN₃XXN₄, wherein N₁ is F, I, L, M, V, W, or Y, wherein N₂ is A, I, F, L, M, N, T, Q, S, V, W, or Y, wherein N₃ is A, D, E, G, H, K, N, P, R, S, or T and wherein N₄ is A, E, F, G, K, I, L, N, M, R, S, V, or Q. In some embodiments, the one or more peptides comprise a peptide sequence of N₁XXN₂XN₃XXN₄, and wherein: N₁ is F, I, L, M, V, W, or Y; N₂ is N, T, S, or V; or Y; N₃ is A, G, N, P, S, or T; and N₄ is F, I, L, N, M, or V; or N₁ is I, L, M, or V; N₂ is I, L, M, or V; N₃ is H, K, or R; and N₄ is A, G, S, or Q; or N₁ is F, I, L, V, W, or Y; N₂ is F, I, N, M, W, or Y; N₃ is D, G, N, or S; and N₄ is K, L, N, S, or V; or N₁ is F, I, L, M, V, W, or Y; N₂ is A, E, I, L, M, Q, or V; N₃ is A, E, G, or S; and N₄ is E, K, or R. In some embodiments, the one or more peptides comprise a peptide sequence of N₁XXN₂XN₃XXN₄, and wherein: N₁ is F, I, L, M, V, W, or Y; N₂ is N, T, S, or V; or Y; N₃ is A, G, N, P, S, or T; and N₄ is F, I, L, N, M, or V. In some embodiments, the one or more peptides comprise a peptide sequence of N₁XXN₂XN₃XXN₄, and wherein: N₁ is I, L, M, or V; N₂ is I, L, M, or V; N₃ is H, K, or R; and N₄ is A, G, S, or Q. In some embodiments, the one or more peptides comprise a peptide sequence of N₁XXN₂XN₃XXN₄, and wherein: N₁ is F, I, L, V, W, or Y; N₂ is F, I, N, M, W, or Y; N₃ is D, G, N, or S; and N₄ is K, L, N, S, or V. In some embodiments, the one or more peptides comprise a peptide sequence of N₁XXN₂XN₃XXN₄, and wherein: or N₁ is F, I, L, M, V, W, or Y; N₂ is A, E, I, L, M, Q, or V; N₃ is A, E, G, or S; and N₄ is E, K, or R.

In some embodiments, at least one of the peptides is derived from translation of an internal out-of-frame open reading frame (ORF) of SARS-CoV-2. In some embodiments, the internal out-of-frame ORF is ORF3c (ORF3a.iORF1) or ORF9b (N.iORF1). In some embodiments, the internal out-of-frame ORF is ORF3c (ORF3a.iORF1). In some embodiments, ORF3c overlaps with ORF3a. In some embodiments, the sequence of at least one peptide derived from translation of ORF3c comprises a peptide sequence of LLFFRALPK (SEQ ID NO: 546) or any subsequence thereof. In some embodiments, the sequence of at least one peptide derived from translation of ORF3c comprises a peptide sequence of ALHFLLFFRALPKS (SEQ ID NO: 374) or any subsequence thereof.

In some embodiments, the internal out-of-frame ORF is ORF9b (N.iORF1). In some embodiments, ORF9b overlaps with the ORF encoding N protein. In some embodiments, the sequence of at least one peptide derived from translation of ORF9b comprises a peptide sequence selected from the group consisting of PKVYPIILR (SEQ ID NO: 547), ISEMHPALR (SEQ ID NO: 548), any subsequence thereof, and any combination thereof. In some embodiments, the sequence of at least one peptide derived from translation of ORF9b comprises a peptide sequence of PKVYPIILR (SEQ ID NO: 547) or any subsequence thereof. In some embodiments, the sequence of at least one peptide derived from translation of ORF9b comprises a peptide sequence of ISEMHPALR (SEQ ID NO: 548) or any subsequence thereof. In some embodiments, the sequence of at least one peptide derived from translation of ORF9b comprises a peptide sequence selected from the group consisting of VGPKVYPIILRLGSPLS (SEQ ID NO: 384), MDPKISEMHPALRLVDPQIQLAVTRMENA (SEQ ID NO: 382), any subsequence thereof, and any combination thereof. In one example, the one or more peptides derived from translation of ORF9b comprises a peptide sequence of VGPKVYPIILRLGSPLS (SEQ ID NO: 384) or any subsequence thereof. In another example, the one or more peptides derived from translation of ORF9b comprises a peptide sequence of MDPKISEMHPALRLVDPQIQLAVTRMENA (SEQ ID NO: 382) or any subsequence thereof.

In some embodiments, at least one of the peptides is derived from translation of a canonical open reading frame (ORF) of SARS-CoV-2. In some embodiments, the canonical ORF is selected from the group consisting of non-structural protein 3 (nsp3) ORF, non-structural protein 4 (nsp4) ORF, ORF3a, ORF6, S protein ORF, M protein ORF, and N protein ORF. In some embodiments, the canonical ORF is nsp3 ORF. In some embodiments, the sequence of at least one peptide derived from translation of the nsp3 ORF comprises a peptide sequence of VTAYNGYLT (SEQ ID NO: 549) or any subsequence thereof. In some embodiments, the sequence of at least one peptide derived from translation of the nsp3 ORF comprises a peptide sequence selected from the group consisting of DGSEDNQTTTIQTIVE (SEQ ID NO: 363), SPDAVTAYNGYLTSSSK (SEQ ID NO: 364), any subsequence thereof, and any combination thereof. In one example, the one or more peptides derived from translation of the nsp3 ORF comprises a peptide sequence of DGSEDNQTTTIQTIVE (SEQ ID NO: 363) or any subsequence thereof. In another example, the one or more peptides derived from translation of the nsp3 ORF comprises a peptide sequence of SPDAVTAYNGYLTSSSK (SEQ ID NO: 364) or any subsequence thereof.

In some embodiments, the canonical ORF is nsp4 ORF. In some embodiments, the sequence of at least one peptide derived from translation of the nsp4 ORF comprises a peptide sequence of IIQFPNTYL (SEQ ID NO: 550) or any subsequence thereof. In some embodiments, the sequence of at least one peptide derived from translation of the nsp4 ORF comprises a peptide sequence of MDGSIIQFPNTYLEGSVR (SEQ ID NO: 365) or any subsequence thereof.

In some embodiments, the canonical ORF is ORF3a. In some embodiments, the sequence of at least one peptide derived from translation of ORF3a comprises a peptide sequence selected from the group consisting of IKDATPSDF (SEQ ID NO: 551), FTIGTVTLK (SEQ ID NO: 552), any subsequence thereof, and any combination thereof. In one example, the one or more peptides derived from translation of a canonical ORF is a peptide sequence of IKDATPSDF (SEQ ID NO: 551) or any subsequence thereof. In another example, the one or more peptides derived from translation of a canonical ORF is a peptide sequence of FTIGTVTLK (SEQ ID NO: 552) or any subsequence thereof. In some embodiments, the sequence of at least one peptide derived from translation of ORF3a comprises a peptide sequence of MDLFMRIFTIGTVTLKQGEIKDATPSDF (SEQ ID NO: 99) or any subsequence thereof.

In some embodiments, the canonical ORF is ORF6. In some embodiments, the sequence of at least one peptide derived from translation of ORF6 comprises a peptide sequence of INLIIKNLS (SEQ ID NO: 553) or any subsequence thereof. In some embodiments, the sequence of at least one peptide derived from translation of ORF6 comprises a peptide sequence of YIINLIIKNLSKS (SEQ ID NO: 100) or any subsequence thereof.

In some embodiments, the canonical ORF is S protein ORF. In some embodiments, the sequence of at least one peptide derived from translation of the S protein ORF comprises a peptide sequence selected from the group consisting of YTNSFTRGV (SEQ ID NO: 554), FKNIDGYFK (SEQ ID NO: 555), FQTLLALHR (SEQ ID NO: 556), IYQTSNFRV (SEQ ID NO: 557), FASVYAWNR (SEQ ID NO: 558), FVIRGDEVR (SEQ ID NO: 559), VIAWNSNNL (SEQ ID NO: 560), IAWNSNNLD (SEQ ID NO: 561), YQAGSTPCN (SEQ ID NO: 562), FLPFQQFGR (SEQ ID NO: 563), VYSTGSNVF (SEQ ID NO: 564), YQTQTNSPR (SEQ ID NO: 565), YTMSLGAEN (SEQ ID NO: 566), LLQYGSFCT (SEQ ID NO: 567), IAQYTSALL (SEQ ID NO: 568), LQIPFAMQM (SEQ ID NO: 569), FAMQMAYRF (SEQ ID NO: 570), LIRAAEIRA (SEQ ID NO: 571), IITTDNTFV (SEQ ID NO: 572), any subsequence thereof, and any combination thereof.

In some embodiments, the sequence of at least one peptide derived from translation of the S protein ORF comprises a peptide sequence of YTNSFTRGV (SEQ ID NO: 554) or any subsequence thereof. In some embodiments, the sequence of at least one peptide derived from translation of the S protein ORF comprises a peptide sequence of FKNIDGYFK (SEQ ID NO: 555) or any subsequence thereof. In some embodiments, the sequence of at least one peptide derived from translation of the S protein ORF comprises a peptide sequence of FQTLLALHR (SEQ ID NO: 556) or any subsequence thereof. In some embodiments, the sequence of at least one peptide derived from translation of the S protein ORF comprises a peptide sequence of IYQTSNFRV (SEQ ID NO: 557) or any subsequence thereof. In some embodiments, the sequence of at least one peptide derived from translation of the S protein ORF comprises a peptide sequence of FASVYAWNR (SEQ ID NO: 558) or any subsequence thereof. In some embodiments, the sequence of at least one peptide derived from translation of the S protein ORF comprises a peptide sequence of FVIRGDEVR (SEQ ID NO: 559) or any subsequence thereof. In some embodiments, the sequence of at least one peptide derived from translation of the S protein ORF comprises a peptide sequence of VIAWNSNNL (SEQ ID NO: 560) or any subsequence thereof. In some embodiments, the sequence of at least one peptide derived from translation of the S protein ORF comprises a peptide sequence of IAWNSNNLD (SEQ ID NO: 561) or any subsequence thereof. In some embodiments, the sequence of at least one peptide derived from translation of the S protein ORF comprises a peptide sequence of YQAGSTPCN (SEQ ID NO: 562) or any subsequence thereof. In some embodiments, the sequence of at least one peptide derived from translation of the S protein ORF comprises a peptide sequence of FLPFQQFGR (SEQ ID NO: 563) or any subsequence thereof. In some embodiments, the sequence of at least one peptide derived from translation of the S protein ORF comprises a peptide sequence of VYSTGSNVF (SEQ ID NO: 564) or any subsequence thereof. In some embodiments, the sequence of at least one peptide derived from translation of the S protein ORF comprises a peptide sequence of YQTQTNSPR (SEQ ID NO: 565) or any subsequence thereof. In some embodiments, the sequence of at least one peptide derived from translation of the S protein ORF comprises a peptide sequence of YTMSLGAEN (SEQ ID NO: 566) or any subsequence thereof. In some embodiments, the sequence of at least one peptide derived from translation of the S protein ORF comprises a peptide sequence of LLQYGSFCT (SEQ ID NO: 567) or any subsequence thereof. In some embodiments, the sequence of at least one peptide derived from translation of the S protein ORF comprises a peptide sequence of IAQYTSALL (SEQ ID NO: 568) or any subsequence thereof. In some embodiments, the sequence of at least one peptide derived from translation of the S protein ORF comprises a peptide sequence of LQIPFAMQM (SEQ ID NO: 569) or any subsequence thereof. In some embodiments, the sequence of at least one peptide derived from translation of the S protein ORF comprises a peptide sequence of FAMQMAYRF (SEQ ID NO: 570) or any subsequence thereof. In some embodiments, the sequence of at least one peptide derived from translation of the S protein ORF comprises a peptide sequence of LIRAAEIRA (SEQ ID NO: 571) or any subsequence thereof. In some embodiments, the sequence of at least one peptide derived from translation of the S protein ORF comprises a peptide sequence of IITTDNTFV (SEQ ID NO: 572) or any subsequence thereof.

In some embodiments, the sequence of at least one peptide derived from translation of the S protein ORF comprises a peptide sequence selected from the group consisting of: TQLPPAYTNSFTRGVYYPDKVFRSSVLHSTQDLFLPFFS (SEQ ID NO: 529), FKNLREFVFKNIDGYFKIYSKHTPINLVRDL (SEQ ID NO: 530), INITRFQTLLALHRSYL (SEQ ID NO: 418), TVEKGIYQTSNFRVQPTES (SEQ ID NO: 532), ATRFASVYAWNRKRISN (SEQ ID NO: 424), DSFVIRGDEVRQIAPG (SEQ ID NO: 425), NYKLPDDFTGCVIAWNSNNLDSKVG (SEQ ID NO: 535), TEIYQAGSTPCNGVEG (SEQ ID NO: 440), ESNKKFLPFQQFGRDIADTTDAVRDPQT (SEQ ID NO: 442), TPTWRVYSTGSNVFQTRAG (SEQ ID NO: 538), ICASYQTQTNSPRRA (SEQ ID NO: 445), SVASQSIIAYTMSLGAEN (SEQ ID NO: 446), CSNLLLQYGSFCTQLNRALTGIAVEQDKNTQE (SEQ ID NO: 541), EPQIITTDNTFVSGN (SEQ ID NO: 119), VLPPLLTDEMIAQYTSALLAGTIT (SEQ ID NO: 460), AALQIPFAMQMAYRFNGIG (SEQ ID NO: 543), TQQLIRAAEIRASANLA (SEQ ID NO: 498), any subsequence thereof, and any combination thereof.

In some embodiments, the sequence of at least one peptide derived from translation of the S protein ORF comprises a peptide sequence of TQLPPAYTNSFTRGVYYPDKVFRSSVLHSTQDLFLPFFS (SEQ ID NO: 529) or any subsequence thereof. In some embodiments, the sequence of at least one peptide derived from translation of the S protein ORF comprises a peptide sequence of FKNLREFVFKNIDGYFKIYSKHTPINLVRDL (SEQ ID NO: 530) or any subsequence thereof. In some embodiments, the sequence of at least one peptide derived from translation of the S protein ORF comprises a peptide sequence of INITRFQTLLALHRSYL (SEQ ID NO: 418) or any subsequence thereof. In some embodiments, the sequence of at least one peptide derived from translation of the S protein ORF comprises a peptide sequence of TVEKGIYQTSNFRVQPTES (SEQ ID NO: 532) or any subsequence thereof. In some embodiments, the sequence of at least one peptide derived from translation of the S protein ORF comprises a peptide sequence of ATRFASVYAWNRKRISN (SEQ ID NO: 424) or any subsequence thereof. In some embodiments, the sequence of at least one peptide derived from translation of the S protein ORF comprises a peptide sequence of DSFVIRGDEVRQIAPG (SEQ ID NO: 425) or any subsequence thereof. In some embodiments, the sequence of at least one peptide derived from translation of the S protein ORF comprises a peptide sequence of NYKLPDDFTGCVIAWNSNNLDSKVG (SEQ ID NO: 535) or any subsequence thereof. In some embodiments, the sequence of at least one peptide derived from translation of the S protein ORF comprises a peptide sequence of TEIYQAGSTPCNGVEG (SEQ ID NO: 440) or any subsequence thereof. In some embodiments, the sequence of at least one peptide derived from translation of the S protein ORF comprises a peptide sequence of ESNKKFLPFQQFGRDIADTTDAVRDPQT (SEQ ID NO: 442) or any subsequence thereof. In some embodiments, the sequence of at least one peptide derived from translation of the S protein ORF comprises a peptide sequence of TPTWRVYSTGSNVFQTRAG (SEQ ID NO: 538) or any subsequence thereof. In some embodiments, the sequence of at least one peptide derived from translation of the S protein ORF comprises a peptide sequence of ICASYQTQTNSPRRA (SEQ ID NO: 445) or any subsequence thereof. In some embodiments, the sequence of at least one peptide derived from translation of the S protein ORF comprises a peptide sequence of SVASQSIIAYTMSLGAEN (SEQ ID NO: 446) or any subsequence thereof. In some embodiments, the sequence of at least one peptide derived from translation of the S protein ORF comprises a peptide sequence of CSNLLLQYGSFCTQLNRALTGIAVEQDKNTQE (SEQ ID NO: 541) or any subsequence thereof. In some embodiments, the sequence of at least one peptide derived from translation of the S protein ORF comprises a peptide sequence of EPQIITTDNTFVSGN (SEQ ID NO: 119) or any subsequence thereof. In some embodiments, the sequence of at least one peptide derived from translation of the S protein ORF comprises a peptide sequence of VLPPLLTDEMIAQYTSALLAGTIT (SEQ ID NO: 460) or any subsequence thereof. In some embodiments, the sequence of at least one peptide derived from translation of the S protein ORF comprises a peptide sequence of AALQIPFAMQMAYRFNGIG (SEQ ID NO: 543) or any subsequence thereof. In some embodiments, the sequence of at least one peptide derived from translation of the S protein ORF comprises a peptide sequence of TQQLIRAAEIRASANLA (SEQ ID NO: 498) or any subsequence thereof.

In some embodiments, the canonical ORF is M protein ORF. In some embodiments, at least one peptide derived from translation of the M protein ORF comprises a peptide sequence selected from the group consisting of LHGTILTRP (SEQ ID NO: 573), YYKLGASQR (SEQ ID NO: 574), any subsequence thereof, and any combination thereof. In some embodiments, at least one peptide derived from translation of the M protein ORF comprises a peptide sequence of LHGTILTRP (SEQ ID NO: 573) or any subsequence thereof. In some embodiments, at least one peptide derived from translation of the M protein ORF comprises a peptide sequence of YYKLGASQR (SEQ ID NO: 574) or any subsequence thereof.

In some embodiments, the sequence of at least one peptide derived from translation of the M protein ORF comprises a peptide sequence selected from the group consisting of: NVPLHGTILTRPLLESELVIGAVILRGHLRIAGHHLGRCDIKDLPKEITVA (SEQ ID NO: 509), TSRTLSYYKLGASQRVAGDSG (SEQ ID NO: 139), TDHSSSSDNIALLVQ (SEQ ID NO: 22), any subsequence thereof, and any combination thereof. In some embodiments, the sequence of at least one peptide derived from translation of the M protein ORF comprises a peptide sequence of NVPLHGTILTRPLLESELVIGAVILRGHLRIAGHHLGRCDIKDLPKEITVA (SEQ ID NO: 509) or any subsequence thereof. In some embodiments, the sequence of at least one peptide derived from translation of the M protein ORF comprises a peptide sequence of TSRTLSYYKLGASQRVAGDSG (SEQ ID NO: 139) or any subsequence thereof. In some embodiments, the sequence of at least one peptide derived from translation of the M protein ORF comprises a peptide sequence of TDHSSSSDNIALLVQ (SEQ ID NO: 22) or any subsequence thereof.

In some embodiments, the canonical ORF is N protein ORF. In some embodiments, the sequence of at least one peptide derived from translation of the N protein ORF comprises a peptide sequence selected from the group consisting of FTALTQHGK (SEQ ID NO: 575), TGPEAGLPY (SEQ ID NO: 576), LPQGTTLPK (SEQ ID NO: 577), LLLLDRLNQ (SEQ ID NO: 578), VTQAFGRRG (SEQ ID NO: 579), FAPSASAFF (SEQ ID NO: 580), VTPSGTWLT (SEQ ID NO: 581), TQALPQRQK (SEQ ID NO: 582), any subsequence thereof, and any combination thereof. In some embodiments, the sequence of at least one peptide derived from translation of the N protein ORF comprises a peptide sequence of FTALTQHGK (SEQ ID NO: 575) or any subsequence thereof. In some embodiments, the sequence of at least one peptide derived from translation of the N protein ORF comprises a peptide sequence of TGPEAGLPY (SEQ ID NO: 576) or any subsequence thereof. In some embodiments, the sequence of at least one peptide derived from translation of the N protein ORF comprises a peptide sequence of LPQGTTLPK (SEQ ID NO: 577) or any subsequence thereof. In some embodiments, the sequence of at least one peptide derived from translation of the N protein ORF comprises a peptide sequence of LLLLDRLNQ (SEQ ID NO: 578) or any subsequence thereof. In some embodiments, the sequence of at least one peptide derived from translation of the N protein ORF comprises a peptide sequence of VTQAFGRRG (SEQ ID NO: 579) or any subsequence thereof. In some embodiments, the sequence of at least one peptide derived from translation of the N protein ORF comprises a peptide sequence of FAPSASAFF (SEQ ID NO: 580) or any subsequence thereof. In some embodiments, the sequence of at least one peptide derived from translation of the N protein ORF comprises a peptide sequence of VTPSGTWLT (SEQ ID NO: 581) or any subsequence thereof. In some embodiments, the sequence of at least one peptide derived from translation of the N protein ORF comprises a peptide sequence of TQALPQRQK (SEQ ID NO: 582) or any subsequence thereof.

In some embodiments, the sequence of at least one peptide derived from translation of the N protein ORF comprises a peptide sequence selected from the group consisting of: GLPNNTASWFTALTQHGKEDLKFPRGQGVPINTNSSPDDQIGYYRRATRRIR (SEQ ID NO: 513), TGPEAGLPYGANKDG (SEQ ID NO: 32), VATEGALNTPKDHIGTRNPANNAAIVLQLPQGTTLPKG (SEQ ID NO: 164), MAGNGGDAALALLLLDRLNQLESKMSGKGQQQQGQTVT (SEQ ID NO: 34), AAEASKKPRQKRTATKAYNVTQAFGRRGPEQTQGNFGDQELIRQGTD (SEQ ID NO: 517), IAQFAPSASAFFG (SEQ ID NO: 55), SDNGPQNQRNAPRITF (SEQ ID NO: 24), KKKADETQALPQRQKKQQTVTLLPAADLDDFSKQLQQSMSSADSTQA (SEQ ID NO: 520), RIGMEVTPSGTWLTYTGAIKLDDKDPNFKDQVILLNKHIDAYKTFPP (SEQ ID NO: 519), any subsequence thereof, and any combination thereof. In some embodiments, the sequence of at least one peptide derived from translation of the N protein ORF comprises a peptide sequence of GLPNNTASWFTALTQHGKEDLKFPRGQGVPINTNSSPDDQIGYYRRATRRIR (SEQ ID NO: 513) or any subsequence thereof. In some embodiments, the sequence of at least one peptide derived from translation of the N protein ORF comprises a peptide sequence of TGPEAGLPYGANKDG (SEQ ID NO: 32) or any subsequence thereof. In some embodiments, the sequence of at least one peptide derived from translation of the N protein ORF comprises a peptide sequence of VATEGALNTPKDHIGTRNPANNAAIVLQLPQGTTLPKG (SEQ ID NO: 164) or any subsequence thereof. In some embodiments, the sequence of at least one peptide derived from translation of the N protein ORF comprises a peptide sequence of MAGNGGDAALALLLLDRLNQLESKMSGKGQQQQGQTVT (SEQ ID NO: 34) or any subsequence thereof. In some embodiments, the sequence of at least one peptide derived from translation of the N protein ORF comprises a peptide sequence of AAEASKKPRQKRTATKAYNVTQAFGRRGPEQTQGNFGDQELIRQGTD (SEQ ID NO: 517) or any subsequence thereof. In some embodiments, the sequence of at least one peptide derived from translation of the N protein ORF comprises a peptide sequence of IAQFAPSASAFFG (SEQ ID NO: 55) or any subsequence thereof. In some embodiments, the sequence of at least one peptide derived from translation of the N protein ORF comprises a peptide sequence of SDNGPQNQRNAPRITF (SEQ ID NO: 24) or any subsequence thereof. In some embodiments, the sequence of at least one peptide derived from translation of the N protein ORF comprises a peptide sequence of KKKADETQALPQRQKKQQTVTLLPAADLDDFSKQLQQSMSSADSTQA (SEQ ID NO: 520) or any subsequence thereof. In some embodiments, the sequence of at least one peptide derived from translation of the N protein ORF comprises a peptide sequence of RIGMEVTPSGTWLTYTGAIKLDDKDPNFKDQVILLNKHIDAYKTFPP (SEQ ID NO: 519) or any subsequence thereof.

As is demonstrated in the Working Examples below, in some embodiments, one or more of the peptides is expressed and/or bound with an MHC-II molecule almost immediately post infection. In some embodiments, these early expressed and/or MHC-II presented/bound peptides are more highly expressed within about 0, 1, 2, 3, 4, 5, 6, 7, 8, 9, 10, 11, or 12 hours post infection as compared to a later time point (e.g., after 12 hours post infection). In one example embodiment, the one or more peptides are expressed and/or are bound by MHC-II at 0-12, 0-11, 0-10, 0-9, 0-8, 0-7, 0-6, 0-5, 0-4, 0-3, 0-2, or 0-1 hours post infection.

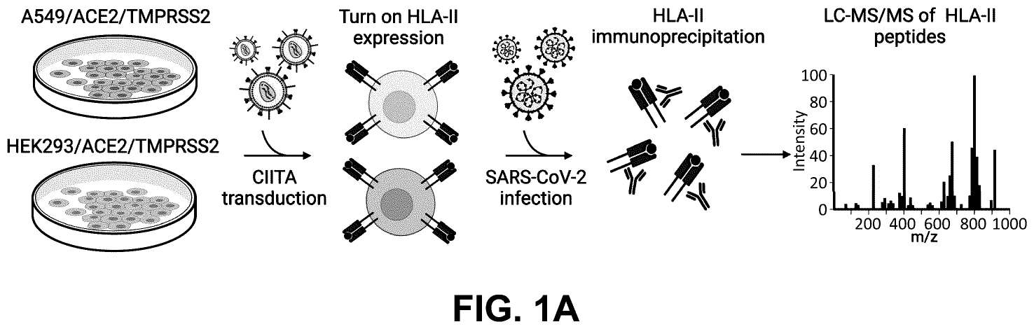

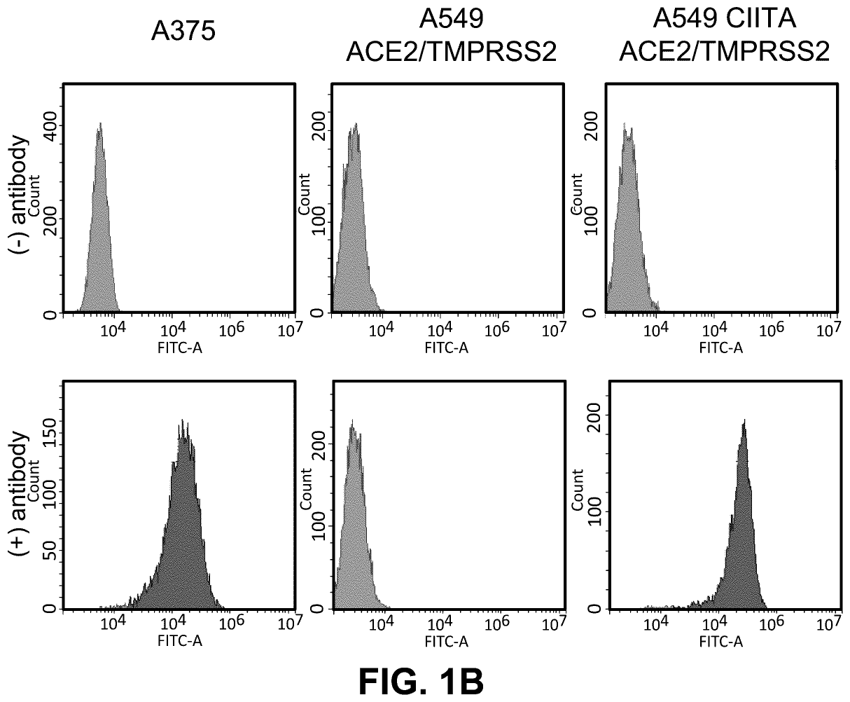

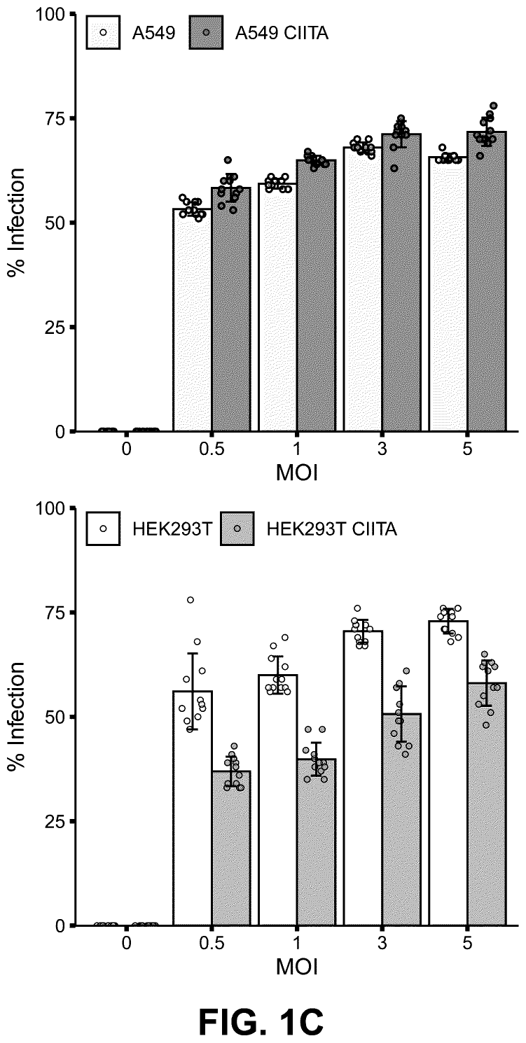

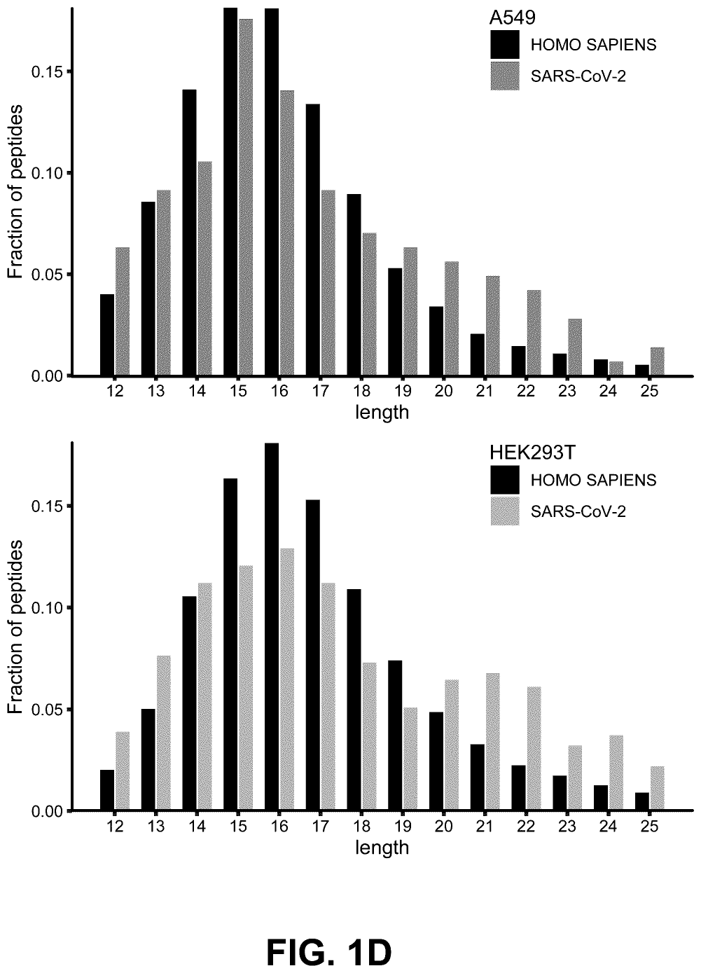

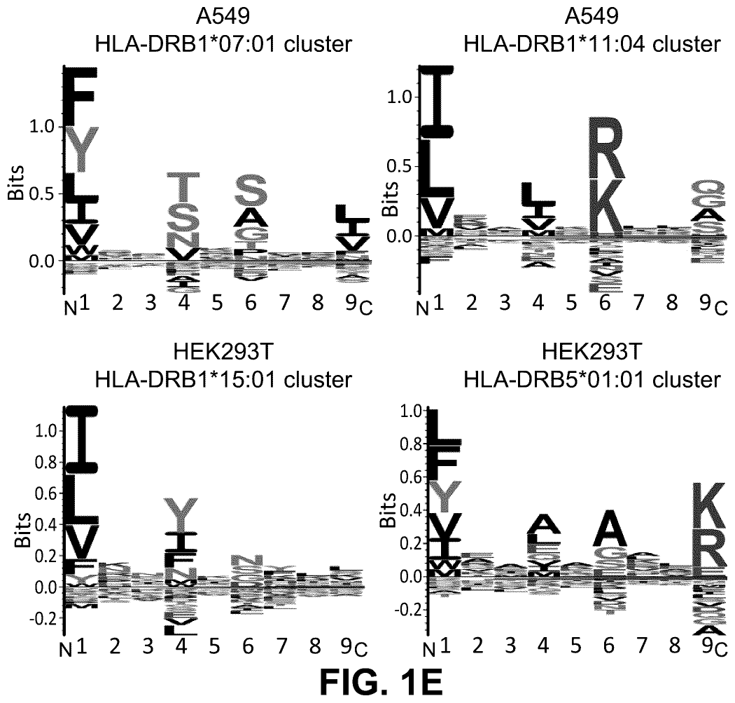

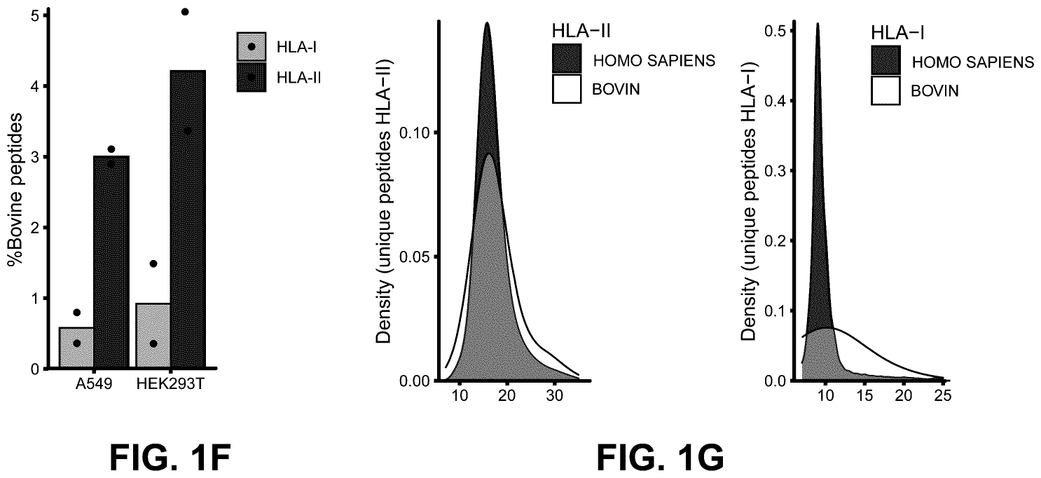

In certain example embodiments, the one or more peptides comprise at least 2, at least 4, at least 6, at least 8, at least 10, at least 12, at least 14, at least 16, at least 18, at least 20, at least 22, at least 24, at least 26, or at least 28, peptide sequences or subsequences thereof of FIGS. 1-6, Table 1, Table 2, and/or Table 4.

The peptides herein may also include those having homology with exemplary peptides herein. For example, the peptides may include those have at least 50%, at least 55%, at least 60%, at least 65%, at least 70%, at least 75%, at least 80%, at least 85%, at least 90%, or at least 95%, at least 96%, at least 97%, at least 98%, or at least 99% sequence identity with the exemplary peptides. The terms “percent (%) sequence identity”, and the like, generally refer to the degree of identity or correspondence between different nucleotide sequences of nucleic acid molecules or amino acid sequences of polypeptides that may or may not share a common evolutionary origin. Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

### Modifications on Peptides

The peptides herein may comprise one or more modifications (e.g., post-translational modifications). In some cases, the peptides may comprise cysteinylated Cysteine. In certain cases, the peptides may comprise oxidized methionine. Other examples of modifications include ubiquitination, phosphorylation, sulfonation, glycosylation, acetylation, methylation, ADP-ribosylation, methionine oxidation, cysteine oxidation, cysteine lipidation, farnesylation, geranylation, pyroglutamation, and deamidation.

### Lengths of Peptides

The peptides may be any length that is reasonable for an epitope. For example, the peptides may have a length of from 5 to 40 amino acids, including all peptide length values and ranges therebetween. For example, the peptides may have a length of from 7 to 39 amino acids, from 9 to 38 amino acids, from 11 to 37 amino acids, from 13 to 30 amino acids, or from 12 to 25 amino acids. For example, the peptides may have 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids. In some embodiments, the optimal length of a peptide may be determined based the immunogenicity of the peptides of different lengths when introduced to a cell or subject.

### Polynucleotides and Vectors

The present disclosure also includes one or more polynucleotides comprising coding sequences of the peptide(s) described herein. As used herein, a polynucleotide may be DNA, RNA, or a hybrid thereof, including without limitation, cDNA, mRNA, genomic DNA, mitochondrial DNA, sgRNA, siRNA, shRNA, miRNA, tRNA, rRNA, snRNA, lncRNA, and synthetic (such as chemically synthesized) DNA or RNA or hybrids thereof. In some examples, a nucleic acid is mRNA. The nucleic acid may be double-stranded or single-stranded. Where single-stranded, the nucleic acid may be the sense strand or the antisense strand. Nucleic acids can include natural nucleotides (such as A, T/U, C, and G), modified nucleotides, analogs of natural nucleotides, such as labeled nucleotides, or any combination thereof.

In certain embodiments, the polynucleotide sequence is recombinant DNA. In further embodiments, the polynucleotide sequence further comprises additional sequences as described elsewhere herein. In certain embodiments, the nucleic acid sequence is synthesized in vitro.

Synthetic mRNA

In some embodiments, the polynucleotide is mRNA, e.g., synthetic mRNA. The mRNA may comprise coding sequence(s) for one or more peptides herein. A synthetic mRNA may be an mRNA produced through an in vitro transcription reaction or through artificial (non-natural) chemical synthesis or through a combination thereof. In some embodiments, the synthetic mRNA further comprises a poly A tail, a Kozak sequence, a 3′ untranslated region, a 5′ untranslated region, or any combination thereof. Poly A tails in particular can be added to a synthetic RNA using a variety of art-recognized techniques, e.g., using poly A polymerase, using transcription directly from PCR products, or by ligating to the 3′ end of a synthetic RNA with RNA ligase.

The mRNA may comprise one or more stabilizing elements that maintain or enhance the stabilities of mRNA, e.g., reducing or preventing degradation of the mRNA. Examples of stabilizing elements include untranslated regions (UTR) at their 5′-end (5′UTR) and/or at their 3′-end (3′UTR), in addition to other structural features, such as a 5′-cap structure or a 3′-poly(A) tail. The stabilizing elements may be a histone stem-loop, e.g., a histone stem loop added by a stem-loop binding protein (SLBP).

### Vectors

The polynucleotides disclosed herein may be in a vector. In some cases, a vector comprises a polynucleotide, the polynucleotide comprising a sequence encoding a barcoding construct operably linked to a first promoter that is an antisense promoter, wherein the barcoding construct comprises a trans-splicing element and a barcode sequence.

The vector may be used for delivering the polynucleotides disclosed herein to cells and/or control the expression of the polynucleotides. A vector refers to a nucleic acid molecule capable of transporting another nucleic acid to which it has been linked. A vector may be a replicon, such as a plasmid, phage, or cosmid, into which another DNA segment may be inserted so as to bring about the replication of the inserted segment. Generally, a vector is capable of replication when associated with the proper control elements. Examples of vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques.

In certain example embodiments, a vector comprising a polynucleotide disclosed herein is a synthetic mRNA vaccine.

Certain vectors may be capable of directing the expression of genes to which they are operatively-linked. Such vectors are referred to herein as “expression vectors.” Common expression vectors of utility in recombinant DNA techniques are often in the form of plasmids. A vector may be a recombinant expression vector that comprises a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. As used herein, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

### Regulatory Elements

A polynucleotide or vector herein may comprise one or more regulatory elements (or sequences encoding thereof), such as transcription control sequences, e.g., sequences which control the initiation, elongation and termination of transcription. The regulatory element(s) may be operably linked to coding sequences of the engineered proteins. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

Exemplary regulatory elements include transcription control sequences, e.g., sequences that control transcription initiation, such as promoter, enhancer, operator and repressor sequences. In some cases, a regulatory element may be a transcription terminator or a sequence encoding thereof. A transcription terminator may comprise a section of nucleic acid sequence that marks the end of a gene or operon in genomic DNA during transcription. This sequence may mediate transcriptional termination by providing signals in the newly synthesized transcript RNA that trigger processes which release the transcript RNA from the transcriptional complex. A regulatory element may be an antisense sequence. In certain case, a regulatory element may be a sense sequence.

### Promoters

In some cases, the promoter may be a constitutive promoter, e.g., U6 and H1 promoters, retroviral Rous sarcoma virus (RSV) LTR promoter, cytomegalovirus (CMV) promoter, SV40 promoter, dihydrofolate reductase promoter, β-actin promoter, phosphoglycerol kinase (PGK) promoter, ubiquitin C, U5 snRNA, U7 snRNA, tRNA promoters or EF1α promoter. In certain cases, the promoter may be a tissue-specific promoter may direct expression primarily in a desired tissue of interest, such as muscle, neuron, bone, skin, blood, specific organs (e.g., liver, pancreas), or particular cell types (e.g., lymphocytes). Examples of tissue-specific promoters include Ick, myogenin, or thy1 promoters. In some embodiments, the promoter may direct expression in a temporal-dependent manner, such as in a cell-cycle dependent or developmental stage-dependent manner, which may or may not also be tissue or cell-type specific. In certain cases, the promoter may be an inducible promoter, e.g., can be activated by a chemical such as doxycycline.

### Codon Optimization

In some embodiments, the polynucleotides herein may be codon optimized, e.g., for expression in a eukaryotic cells such as a mammalian cell or a plant cell. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. In certain embodiments, the polynucleotides are not codon optimized.

Codon optimization a canonical ORF may be guided by the information of internal out-of-frame ORFs of the canonical ORF, e.g., information of the sequences, positions, and expression products of internal out-of-frame ORFs, as well as their functions and activities of the expression products. For example, a canonical ORF of a polynucleotides may be codon optimized in a way that the internal out-of-frame ORF(s) of the canonical ORF is not interrupted, so that the expression of the internal out-of-frame ORFs is maintained. In certain cases, the codon optimization may be performed so that both the canonical ORF and the internal out-of-frame ORF(s) are optimized. In some cases, the present disclosure provides a method of designing an immunogenic composition, comprising: identifying immunogenic peptides derived from translation of out-of-frame ORFs; and codon optimizing nucleic-acid based vaccines directed to immunogenic peptides derived from translation of in-frame ORFs such that expression of immunogenic peptides derived from translation of out-of-frame ORFs is maintained.

### Antigenic Components

The compositions disclosed herein (e.g., immunogenic compositions comprising one or more peptides disclosed herein, polynucleotides disclosed herein, vectors disclosed herein, or or any combination thereof) may further comprise one or more antigenic components. Antigenic components include components that specifically trigger the immune response against the antigen or antigens from which the antigenic components are derived. Examples of the antigenic components include whole virions (e.g., live attenuated or inactive forms), proteins (such as, but not limited to, envelope and capsid proteins), carbohydrates and lipids derived therefrom, polynucleotides encoding such proteins, as well as combinations thereof, and fragments of the same which are capable of eliciting an immune response in a host. Examples of the antigenic components also include non-replicating viral vector, replicating viral vector, proteins, polypeptides, or peptides derived from one or more proteins or polypeptides of the virus, virus-like particles, DNA, and RNA molecules, e.g., DNA or RNA molecules encoding antigenic proteins, polypeptides, or peptides. In one example, the antigenic components may be capable of stimulating production of an antibody targeting SARS-CoV-2

In some examples, the antigenic components are one or more components in a vaccine against SARS-CoV-2 (e.g., a synthetic mRNA vaccine (e.g., LNP-encapsulated mRNA vaccine encoding S protein (e.g., mRNA-1273))), Adenovirus type 5 vector that expresses S protein (e.g., Ad5-nCoV), DNA plasmid encoding S protein delivered by electroporation (e.g., INO-4800), DCs modified with lentiviral vector expressing synthetic minigene based on domains of selected viral proteins (e.g., LV-SMENP-DC), aAPCs modified with lentiviral vector expressing synthetic minigene based on domains of selected viral proteins (e.g., Pathogen specific aAPC). Other examples of the antigenic components include those described in Le T T et al., The COVID-19 vaccine development landscape, Nature Reviews Drug Discovery 19, 305-306 (2020), which is incorporated herein in its entirety. In some embodiments, the antigenic components antigenic peptides from a nucleocapsid phosphoprotein of SARS-CoV-2, a spike glycoprotein of SARS-CoV-2, or a combination thereof, or one or more polynucleotides encoding the one or more antigenic peptides.

### Therapeutic Agents

In some embodiments, the compositions (e.g., immunogenic compositions comprising one or more peptides disclosed herein, polynucleotides disclosed herein, vectors disclosed herein, or any combination thereof) may further comprise one or more therapeutic agents. In some embodiments, the one or more therapeutic agents are anti-viral therapeutics. Such agents may be used together with the immunogenic composition herein for treating virus infection and related health problems. In some cases, the therapeutic agent(s) are drug(s) for treating SARS-CoV-2 and related diseases. Examples of such therapeutic agents include nucleoside analogues (e.g., Remdesivir, Favipiravir, Ribavirin), HIV protease inhibitors (e.g., Kaletra (lopinavir/ritonavir)), agents targeting proinflammatory hypercytokinemia (e.g., Tocilizumab and leronlimab), IFNλ, Antiparasitics (e.g., Ivermectin), antimalarial drugs (e.g., Chloroquine and hydroxychloroquine), agents targeting cardioprotective derivatives (e.g., Colchicine), agents targeting angiotensin-converting enzyme 2 (ACE2), Nicotine, Vitamin D, and Spironolactone. Additional examples of therapeutic agents can be included in the composition include those described in Konstantinidou S K et al., Repurposing current therapeutic regimens against SARS-CoV-2 (Review), Exp Ther Med. 2020 September; 20(3):1845-1855, which is incorporated herein in its entirety.

### Pharmaceutical Formulations

In another aspect, the present disclosure provides pharmaceutical formulations comprising the compositions, or one or more components of the compositions. A pharmaceutical composition may further comprise one or more excipients, such as pharmaceutically acceptable carriers suitable for administration to cells or to a subject. In some examples, the pharmaceutical composition is a vaccine, which elicit protective immunity to a recipient.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. Examples of solvents are e.g. water, alcohols, vegetable or marine oils (e.g., edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof. Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives. The term “pharmaceutically acceptable” as used throughout this specification is consistent with the art and means compatible with the other ingredients of a pharmaceutical composition and not deleterious to the recipient thereof.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical compositions can be applied parenterally, rectally, orally or topically. For example, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. The compositions herein may be administered by the same route or may be administered by a different route. By means of example, and without limitation, cells may be administered parenterally, and other active components may be administered orally. In some cases, the composition or pharmaceutical composition may by intramuscular injection. In some cases, the composition or pharmaceutical composition may by intravascular injection.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure iso-osmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further examples of suitably pharmaceutically acceptable carriers or additives include proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregelatinized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

The pharmaceutical compositions may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

The pharmaceutical composition may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

In some embodiments, the pharmaceutical compositions further comprise one or more adjuvants. Adjuvants may be molecules or compounds that have intrinsic immunomodulatory properties and, when administered in conjunction with an antigen, effectively potentiate the host antigen-specific immune responses compared to responses raised when antigen is given alone. Examples of adjuvants include aluminum hydroxide and aluminum phosphate, saponins e.g., QUIL-A® (commercially available from Brenntag Biosector A/S), QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion may be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g., anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks. Other examples of adjuvants include Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Aluminium salts (e.g. AdjuPhos), Adjuplex, and M1F59, lectins, growth factors, cytokines and lymphokines such as alpha-interferon, gamma interferon, platelet derived growth factor (PDGF), granulocyte-colony stimulating factor (gCSF), granulocyte macrophage colony stimulating factor (gMCSF), tumor necrosis factor (TNF), epidermal growth factor (EGF), IL-I, IL-2, IL-4, IL-6, IL-8, IL-IO, and IL-12 or encoding nucleic acids thereof.

Various delivery systems are known and can be used to administer the pharmacological compositions including encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor or infected tissue), thus requiring only a fraction of the systemic dose. See, for example, Goodson, In: Medical Applications of Controlled Release, 1984. (CRC Press, Boca Raton, Fla.).

### Cells and Organisms

In another aspect, the present disclosure provides cells and organisms comprising the compositions herein. The cells may be in tissue, organ, or isolated cells. Such cells may be of a unique type of cells or a group of different types of cells such as cultured cell lines, primary cells and proliferative cells. The cells may be prokaryotic cells, lower eukaryotic cells such as yeast, and other eukaryotic cells such as insect cells, plant and mammalian (e.g., human or non-human) cells as well as cells capable of producing the vector of the invention (e.g., 293, HER96, PERC.6 cells, Vero, HeLa, CEF, duck cell lines, etc.). The cells may include cells which can be or has been the recipient of the vector described herein as well as progeny of such cells. Host cells can be cultured in conventional fermentation bioreactors, flasks, and petri plates. Culturing can be carried out at a temperature, pH and oxygen content appropriate for a given cell. No attempts will be made here to describe in detail the various prokaryote and eukaryotic host cells and methods known for the production of the peptides and vectors herein.

### Methods of Treatments and Prophylaxis

In another aspect, the present disclosure provides methods of treating and or preventing (e.g., immunizing) an infection (e.g., viral infection) in a subject, and/or disease and conditions related to the infection. Generally, the methods may comprise administering a pharmaceutically effective (e.g., therapeutically effective amount or prophylactically effective amount) amount of the composition herein to a subject, e.g., a subject in need thereof. In some cases, the method comprises administering the composition(s), the polynucleotide(s), and/or the vector(s) herein to a subject. A pharmaceutically effective amount refers to an amount which can elicit a biological, medicinal, or immunological response in a tissue, system, or subject (e.g., animal or human) that can prevent or alleviate one or more of the local or systemic symptoms or features of a disease or condition being treated.

In an aspect, the present invention provides a method for inducing a T cell response and/or antibody response to SARS-CoV-2 in a subject. In some cases, the methods may comprise administering the peptides disclosed herein, the polynucleotides disclosed herein, the vectors disclosed herein, or any combination thereof to a subject (e.g., a subject in need thereof). In some embodiments, the method further comprises administering antigenic agents, polynucleotides encoding thereof, or any combination thereof, to the subject.

In an aspect, the present invention provides a method of treating a SARS-CoV-2 infection, the method comprising administering a composition disclosed herein (e.g., an immunogenic composition comprising one or more peptides disclosed herein, polynucleotides disclosed herein, vectors disclosed herein, or any combination thereof) to a subject (e.g., a subject in need thereof). In some embodiments, the method further comprises administering antigenic agents, anti-viral therapeutics, polynucleotides encoding thereof, or any combination thereof, to the subject.

Methods of administrating to a subject include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment and prophylaxis; this may be achieved by, for example, local infusion during surgery, topical application, by injection, by means of a catheter, by means of a suppository, or by means of an implant.

The term “subject” or “patient” is intended to include mammalian organisms. Examples of subjects/patients include humans and non-human mammals, e.g., non-human primates, dogs, cows, horses, pigs, sheep, goats, cats, mice, rabbits, rats, and transgenic non-human animals. In specific embodiments of the invention, the subject is a human.

In some cases, the methods comprise administering to a subject the pharmaceutical compositions alone or in concert with other therapeutic agents at appropriate dosages defined by routine testing in order to obtain optimal efficacy while minimizing any potential toxicity. The dosage regimen utilizing a pharmaceutical composition may be selected in accordance with a variety of factors including type, species, age, weight, sex, medical condition of the patient; the severity of the condition to be treated; the route of administration; the renal and hepatic function of the patient; and the particular pharmaceutical composition employed.

Optimal precision in achieving concentrations of the therapeutic regimen within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical composition and various therapeutic agents may be independently optimized and combined to achieve a synergistic result wherein the pathology is reduced more than it would be if either was used alone.

In particular, toxicity and therapeutic efficacy of the pharmaceutical composition may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effect is the therapeutic index and it may be expressed as the ratio LD50/ED50. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED50 with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC50 (the concentration of the test composition that achieves a half-maximal inhibition of symptoms) as determined in cell culture. Such information may be used to accurately determine useful doses in humans. Levels in plasma may be measured, for example, by high performance liquid chromatography.

The methods may comprise administering a booster agent in addition to the administration of the composition therein. A booster agent may be an extra administration of the composition herein or a different agent. A booster (or booster vaccine) may be given after an earlier administration of the composition. The time of administration between the initial administration of the composition and the booster may be at least 1 minute, at least 5 minutes, at least 10 minutes, at least 20 minutes, at least 30 minutes, at least 40 minutes, at least 50 minutes, at least 1 hour, at least 2 hours, at least 4 hours, at least 8 hours, at least 12 hours, at least 1 day, at least 1 week, at least 2 week, at least 3 week, at least 1 month, at least 2 months, at least 3 months, at least 4 months, at least 5 months, at least 6 months, at least 1 year, at least 5 years, at least 10 years, and any time period in-between.

### Delivery to Cells and Organisms

The present disclosure also provides delivery systems for introducing the compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

### Cargos

The delivery systems may comprise one or more cargos. A cargo may comprise one or more of the following: i) one or more peptides herein, ii) one or more polynucleotides encoding the peptide(s) or vectors comprising the polynucleotides; iii) mRNA molecules encoding the one or more peptides; iv) cells comprising i), ii) and/or iii). In some examples, a cargo may comprise a plasmid encoding one or more engineered proteins herein.

### Physical Delivery

In some embodiments, the cargos may be introduced to cells by physical delivery methods. Examples of physical methods include microinjection, electroporation, and hydrodynamic delivery. Both nucleic acid and proteins may be delivered using such methods. For example, the peptides and polynucleotides may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Polynucleotides and vectors comprising coding sequences for the peptides may be microinjected. In some cases, microinjection may be used i) to deliver DNA directly to a cell nucleus, and/or ii) to deliver mRNA (e.g., in vitro transcribed) to a cell nucleus or cytoplasm.

Microinjection may be used to generate genetically modified animals. For example, gene editing cargos may be injected into zygotes to allow for efficient germline modification. Such approach can yield normal embryos and full-term mouse pups harboring the desired modification(s).

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may a greatest dimension (e.g., diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA. In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)). In some examples, AAV particles may be created in HEK 293 T cells. Once particles with specific tropism have been created, they are used to infect the target cell line much in the same way that native viral particles do. This may allow for persistent presence of engineered proteins in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of engineered proteins may be packaged directly onto one DNA plasmid vector and delivered via one AAV particle. In some examples, AAVs may be used to deliver gRNAs into cells that have been previously engineered to express the engineered protein. In some examples, coding sequences of two or more engineered proteins may be made into two separate AAV particles, which are used for co-transfection of target cells.

### Lentiviruses

The compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules and/or RNA molecules. In certain cases, LNPs may be use for delivering RNP complexes.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the engineered protein directly, which is then complexed with the gRNA and delivered to cells. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/dObm00427h.

### Method of Identifying Immunogenic Peptides

In another aspect, the present disclosure provides methods for screening and identifying the immunogenic peptides herein. In general, the methods comprise isolating complex(es) of MHC-II (e.g., HLA-II) and binding partners from cells infected by a pathogen such as a virus (e.g., SARS-CoV-2), and characterizing (e.g., determining the sequences) of peptides in the isolated complex, and identifying peptides derived from one or more polypeptides or proteins of the virus. In some embodiments, the method can include identifying peptides derived from one or more open reading frames. In some embodiments, the open reading frames are annotated ORFs, alternative ORFs, canonical ORFs, and/or noncanonical ORFs. ORFs are discussed and described in greater detail elsewhere herein.

In certain examples, the methods of identifying immunogenic peptides comprising: a) lysing cells having a potential to express the immunogenic peptides of interest with a lysis buffer comprising a cell membrane disrupting detergent; b) enzymatic shearing of nucleic acids in the lysed cells; c) isolating HLA-II from the lysed cells, wherein the HLA-II is in complex with one or more peptides from the lysed cells; and d) determining sequences of the one or more polypeptides or proteins in complex with the HLA-II from (c). In certain example embodiments, the methods further comprise (e) identifying HLA alleles encoding HLA that bind the peptides identified in using a HLA-II epitope binding predictor, and (f) selecting a subset of peptides that bind HLA-II of a defined percentage of HLA-II alleles. In certain example embodiments, the methods further comprise selecting immunogenic peptides demonstrating a relative abundance above a defined threshold as determined by analysis of the complete cellular transcriptome and or proteome.

In some embodiments, the methods further comprise ribosome sequencing to identify actively translated peptides and selecting immunogenic peptides that are being actively translated at one or more time points. In certain embodiments, (d) is performed by liquid chromatography tandem mass spectrometry analysis. In certain embodiments, isolating HLA-II comprises immunoprecipitation of the HLA-II complex with an anti-HLA-II antibody. In certain embodiments, the immunogenic peptides of interest are expressed by a pathogen and wherein the cells have been infected with the pathogen.

The cells used in the method may be treated with one or more cell signaling molecules related to infection by the pathogen. In some examples, the pathogen is a virus, e.g., SARS-Cov-2. In some cases, the cells may express (e.g., overexpress by an exogenous gene) one or more proteins regulating or mediating a virus infection. For example, the cells may express one or more receptors involved in a viral infection process, e.g., cell surface receptors used by the pathogen to infect the cells, e.g., ACE-2 and TMPRSS2. In certain cases, the cells may be engineered to alter (e.g., increase or decrease) HLA presentation. For example, the cells may express (e.g., by one or more exogenous genes) one or more of CIITA, proteasome subunits, tPA, POMP, or ubiquitin-proteasome genes.

In some embodiments, the methods comprise lysing the cells infected by the virus with a lysis buffer. The lysis buffer may be capable of breaking the cells while retaining intact complexes of HMC-II and its binding partners. The lysis buffer may comprise one or more membrane disrupting detergents. In certain example embodiments, the membrane disrupting detergent is an alkylphenol ethoxylate surfactant. In one example, the membrane disrupting detergent is an octylphenol ethoxylate surfactant, such as, but not limited to a TRITON® X series surfactant (C₁₄H₂₂O(C₂H₄O)ₙ, where n=7-40). In one example embodiments, the TRITON® X series surfactant is TRITON® X-100 (n˜9.5). The concentration of the TRITON® X in the lysis buffer may be from 0.1% to 5%, e.g., from 0.5% to 3%, from 1% to 2%, from 1.2% to 1.8%, or from 1.4% to 1.6%. In one example, the concentration of the TRITON® X in the lysis buffer may be about 1%, about 1.1%, about 1.2%, about 1.3%, about 1.4%, about 1.5%, about 1.6%, about 1.7%, about 1.8%, about 1.9%, or about 2.0%.

In some embodiments, the methods comprise shearing nucleic acid (e.g., DNA) in the lysed cells. In some examples, the shearing is enzymatic shearing, e.g., no sonication is used. In such cases, the shearing may be performed using an enzyme, e.g., nuclease. The nuclease may be an endonuclease that degrades all forms of DNA and RNA (single stranded, double stranded, linear and circular) while having no proteolytic activity. The endonuclease may be derived from Serratia marcescens or a variant thereof. In one example, the endonuclease is Benzonase. A salt may be used together with the enzyme in shearing the nucleic acid. The salt may be a Magnesium salt, e.g., MgCl₂, MgSO₄, and magnesium acetate. In some embodiments, the nucleic acids in the lysed cells are enzymatically sheared using an endonuclease from Serratia marcescens and MgCl₂.

The methods may further comprise isolating MHC-II from the lysed cells. In an embodiment isolating the MHC-II comprises isolating the HLA-II from the lysed cells, wherein the HLA-II (or other MHC-II) is in complex with one or more peptides derived from polypeptides or proteins from the lysed cells. Such peptides may be derived from the virus that infects the cells. The isolation may be performed using immunoprecipitation, e.g., using a reagent that bind to one or more components of MHC-II or one or more molecules attached to MHC-II.

The methods may also comprise determining the sequences of the one or more peptides in complex with the MHC-II. In some embodiments, the sequences may be determined using mass spectrometry, e.g., liquid chromatography tandem mass spectrometry (LC-MS) analysis.

In some embodiments, the methods may comprise characterizing the nucleic acids, e.g., RNA, in the infected. The results of the characterization may be used to determine the viral abundance in the cells. The determination may be performed using sequencing technologies such as shotgun sequencing, resequencing, de novo assembly, exome sequencing, DNA-Seq, Targeted DNA-Seq, Methyl-Seq, Targeted methyl-Seq, DNase-Seq, Sono-Seq, FAIRE-seq, MAINE-Seq, RNA-Seq, ChIP-Seq, RIP-Seq, CLIP-Seq, HITS-Seq, FRT-Seq, NET-Seq, Hi-C, Chia-PET, Ribo-Seq, TRAP, PARS, synthetic saturation mutagenesis, Immuno-Seq, Deep protein mutagenesis, PhIT-Seq, SMRT, and genome-wide chromatin interaction mapping. In some embodiments, the methods comprise performing RNA-Seq on the RNA in the infected cells.

The methods may further comprise identifying HLA alleles that bind the peptides identified by using Gibbs Cluster deconvolution to identify sequence clusters for these peptides (Andreatta et al., 2017), and comparing these clusters with known preferences of HLA alleles in infected cells.

The immunogenic peptides may be selected based on the sequencing data. For example, the methods may further comprise selecting immunogenic peptides demonstrating a relative abundance above a defined threshold as determine by analysis of the complete cellular transcriptome and/or proteome. In some cases, the expression level of genes may be determined (e.g., by computational methods based on the sequencing data) and the peptides may be ranked and selected from highly abundant genes (e.g., genes with high expression levels). Alternatively or additionally, ribosomal sequencing may be used (in some cases no RNA-seq data is used) to identify peptides that are being actively translated by the cell at one or more time points, and only those peptides that are actively translated are selected. The datasets from this approach are different from conventional mass-spectrometry search datasets in that they include out-of-frame ORFs, which may include internal out-of-frame ORFs.

In some embodiments, the information of HLA-II peptides from internal out-of-frame ORFs may be used to modify the sequence of canonical ORFs, e.g., to ensure the continuous synthesis and presentation of peptides from the optimized sequences.

### Method of Determining Infection Status

In another aspect, the present disclosure provides methods of determining a viral infection status of a subject. In some embodiments, the methods comprise contacting immune cells derived from the subject with a composition of the present invention; and detecting cross-reactivity of the immune cells to the composition. In certain example embodiments, immune cells derived from the subject are contacted with an immunogenic composition described herein or one or more components thereof.

In some embodiments, the methods may be used for performing T cell assay. For example, the methods may determine T cells' response to the compositions such as the peptides. The response may be used to evaluate the infection status of the subject from which the T cells are derived from. In one example, T cells (e.g., CD8+ T cells) may be isolated from PBMCs and incubated with the compositions herein. Proliferation of T cells can be measured by 3H thymidine incorporation. Secretion of cytokines from the T cells may be measured, e.g., by ELISA.

### Method of Preventing or Treating an Infection

In another aspect, the present disclosure provides methods of preventing (e.g., immunizing) or treating a subject against a viral infection or treating a subject infected by a virus. In some embodiments, the methods comprise administering the compositions herein to a subject, e.g., a subject in need thereof.

In some embodiments, the methods may be used for performing T cell assay. For example, the methods may determine T cells' response to the compositions such as the peptides. The response may be used to evaluate the immunity status of the subject from which the T cells are derived from. In one example, T cells (e.g., CD8+ T cells) may be isolated from PBMCs and incubated with the compositions herein. Proliferation of T cells can be measured by 3H thymidine incorporation. Secretion of cytokines from the T cells may be measured, e.g., by ELISA.

mRNA Vaccines

In some embodiments, one or more polynucleotides encoding the one or more immunogenic polypeptides described herein are included in an mRNA vaccine composition. The mRNA vaccine composition can be administered to a subject in need thereof. In some embodiments, the vaccine is administered to a subject in an effective amount to induce an immune response in the subject.

Described herein are pharmaceutical compositions that include one or more isolated messenger ribonucleic (mRNA) polynucleotides encoding at least one SARS-CoV-2 antigenic polypeptide or an immunogenic fragment thereof (e.g., an immunogenic fragment capable of inducing an immune response to the antigenic polypeptide), such as any of those polynucleotides described in greater detail elsewhere herein, where the isolated mRNA is formulated in a lipid nanoparticle. As used herein “antigenic polypeptide” encompasses immunogenic fragments of the antigenic polypeptide (an immunogenic fragment that is induces (or is capable of inducing) an immune response to a SARS-CoV-2 variant. The mRNA encoding at least one SARS-CoV-2 antigenic polypeptide or immunogenic fragment thereof can include an open reading frame that encodes the at least one SARS-CoV-2 antigenic polypeptide or immunogenic fragment thereof. In some embodiments, the open reading frame encodes at least two, at least five, or at least ten SARS-CoV-2 antigenic polypeptides and/or immunogenic fragments thereof. In some embodiments, the open reading frame encodes at least 100 antigenic polypeptides. In some embodiments, the open reading frame encodes 2-100 SARS-CoV-2 antigenic polypeptides and/or immunogenic fragments thereof.

In some embodiments, the pharmaceutical composition comprises a plurality of lipid nanoparticles comprising a cationic lipid, a neutral lipid, a cholesterol, and a PEG lipid, wherein the plurality of lipid nanoparticles optionally has a mean particle size of between 80 nm and 160 nm; and wherein the lipid nanoparticles comprise one or more polynucleotides encoding at least one SARS-CoV-2 antigenic polypeptide or an immunogenic fragment thereof.

In some embodiments, the mRNA vaccine is multivalent. In some embodiments, the mRNA of the mRNA vaccine is codon-optimized. In some embodiments, an RNA (e.g., mRNA) vaccine further includes an adjuvant.

In some embodiments, the isolated mRNA is not self-replicating.

In some embodiment, the isolated mRNA comprises and/or encodes one or more 5′terminal cap (or cap structure), 3′terminal cap, 5′untranslated region, 3′untranslated region, a tailing region, or any combination thereof.

In some embodiments, the capping region of the isolated mRNA region may be from 1 to 10, e.g., 2-9, 3-8, 4-7, 1-5, 5-10, or at least 2, or 10 or fewer nucleotides in length. In some embodiments, the cap is absent.

In some embodiments, a 5′-cap structure is cap0, cap1, ARCA, inosine, N1-methyl-guanosine, 2′-fluoro-guanosine, 7-deaza-guanosine, 8-oxo-guanosine, 2-amino-guanosine, LNA-guanosine, or 2-azido-guanosine.

In some embodiments, the 5′terminal cap is 7mG(5′)ppp(5′)NlmpNp, m7GpppG cap, N⁷-methylguanine. In some embodiments, the 3′terminal cap is a 3′-O-methyl-m7GpppG.

In some embodiments, the 3′-UTR is an alpha-globin 3′-UTR. In some embodiments, the 5′-UTR comprises a Kozak sequence.

In some embodiments, the tailing sequence may range from absent to 500 nucleotides in length (e.g., at least 60, 70, 80, 90, 120, 140, 160, 180, 200, 250, 300, 350, 400, 450, or 500 nucleotides). In some embodiments, the tailing region is or includes a polyA tail. Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional. In some embodiments, the poly-A tail is at least 160 nucleotides in length.

In some embodiments, the at least one SARS-CoV-2 antigenic polypeptide linked to or fused to a signal peptide. In some embodiments, the isolated mRNA encoding a SARS-Cov-2 antigenic polypeptide or immunogenic fragment thereof further includes a polynucleotide sequence encoding a signal peptide. In some embodiments, the signal peptide is selected from: a HuIgGk signal peptide (METPAQLLFLLLLWLPDTTG (SEQ ID NO: 583)); IgE heavy chain epsilon-1 signal peptide (MDWTWILFLVAAATRVHS (SEQ ID NO: 584)); Japanese encephalitis PRM signal sequence (MLGSNSGQRVVFTILLLLVAPAYS (SEQ ID NO: 585)), VSVg protein signal sequence (MKCLLYLAFLFIGVNCA (SEQ ID NO: 586)) and Japanese encephalitis JEV signal sequence (MWLVSLAIVTACAGA (SEQ ID NO: 587)). In some embodiments, the signal peptide is fused to the N-terminus of at least one SARS-CoV-2 antigenic polypeptide. In some embodiments, a signal peptide is fused to the C-terminus of at least one SARS-CoV-2 antigenic polypeptide.

In some embodiments, the polynucleotides of the mRNA vaccine composition are structurally modified and/or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, the polynucleotide, e.g., an mRNA of an mRNA vaccine composition described herein comprises at least one chemical modification. In some embodiments, the polynucleotide, e.g., an mRNA of an mRNA vaccine composition does not comprise a chemical or structural modification.

In some embodiments, the at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-i-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

In some embodiments, about 10%, 15%, 20%, 24%, 30%, 35%, 40%, 45%, 50%, 55%, 60%, 65%, 70%, 75%, 80%, 85%, 90%, 95%, to/or about 100% of the uracil in of the SARS-CoV-2 antigenic polypeptide or immunogenic fragment thereof encoding polynucleotide, such in the open reading frame, have a chemical modification, In some embodiments, 100% of the uracil in the open reading frame of the SARS-CoV-2 antigenic polypeptide or immunogenic fragment thereof encoding polynucleotide have a N1-methyl pseudouridine in the 5-position of the uracil.

In some embodiments, the mRNA polynucleotide includes a stabilization element. In some embodiments, the stabilization element is a histone stem-loop. In some embodiments, the stabilization element is a nucleic acid sequence having increased GC content relative to wild type sequence.

In one embodiment, the mRNA polynucleotide may include a sequence encoding a self-cleaving peptide. The self-cleaving peptide may be, but is not limited to, a 2A peptide. As a non-limiting example, the 2A peptide may have the protein sequence: GSGATNFSLLKQAGDVEENPGP (SEQ ID NO: 588), fragments or variants thereof. In one embodiment, the 2A peptide cleaves between the last glycine and last proline. As another non-limiting example, the polynucleotides of the present invention may include a polynucleotide sequence encoding the 2A peptide having the protein sequence GSGATNFSLLKQAGDVEENPGP (SEQ ID NO: 588) fragments or variants thereof.

One such polynucleotide sequence encoding the 2A peptide is GGAAGCGGAGCTACTAACTTCAGCCTGCTGAAGCAGGCTGGAGACGTGGAG GAGAACCCTGGACCT (SEQ ID NO: 589). The polynucleotide sequence of the 2A peptide may be modified or codon optimized by the methods described herein and/or are known in the art.

In one embodiment, this sequence may be used to separate the coding region of two or more polypeptides of interest. As a non-limiting example, the sequence encoding the 2A peptide may be between a first coding region A and a second coding region B (A-2Apep-B). The presence of the 2 A peptide would result in the cleavage of one long protein into protein A, protein B and the 2A peptide. Protein A and protein B may be the same or different peptides or polypeptides of interest. In another embodiment, the 2A peptide may be used in the polynucleotides of the present invention to produce two, three, four, five, six, seven, eight, nine, ten or more proteins.

In some embodiments, the length of an mRNA included in the mRNA vaccine is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the length of an mRNA included in the mRNA vaccine includes from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000).

In some embodiments, the polynucleotides are linear. In yet another embodiment, the polynucleotides of the present invention that are circular are known as “circular polynucleotides” or “circP.” As used herein, “circular polynucleotides” or “circP” means a single stranded circular polynucleotide which acts substantially like, and has the properties of, an R A. The term “circular” is also meant to encompass any secondary or tertiary configuration of the circP.

Other RNA modifications for mRNA vaccines and production of mRNA can be as described e.g., U.S. Pat. Nos. 8,278,036, 8,691,966, 8,748,089, 9,750,824, 10,232,055, 10,703,789, 10,702,600, 10,577,403, 10,442,756, 10,266,485, 10,064,959, 9,868,692, 10,064,959, 10,272,150; U.S. Publications, US20130197068, US20170043037, US20130261172, US20200030460, US20150038558, US20190274968, US20180303925, US20200276300; International Patent Application Publication Nos. WO/2018/081638A1, WO/2016/176330A1, which are incorporated herein by reference.

In some embodiments, the mRNA vaccine includes one or more additional mRNAs that encode a polypeptide adjuvant. In some embodiments, the mRNA vaccine includes one or more additional mRNAs that encode a non SARS-Cov-2 antigen, such as an antigen to another disease causing agent.

In some embodiments, the one or more additional mRNAs that encode a polypeptide adjuvant encode a flagellin polypeptide. In some embodiments, at least one flagellin polypeptide (e.g., encoded flagellin polypeptide) is an immunogenic flagellin fragment. In some embodiments at least one flagellin polypeptide has at least 80%, at least 85%, at least 90%, or at least 95% identity to a flagellin polypeptide having a sequence identified by any one of SEQ ID NO: 54-56 of U.S. Pat. No. 10,272,150.

In some embodiments, at least one flagellin polypeptide and at least one SARS-Cov2 and/or additional antigenic polypeptide are encoded by a single RNA (e.g., mRNA) polynucleotide. In other embodiments, at least one flagellin polypeptide and at least one SARS-Cov2 and/or additional antigenic polypeptide are each encoded by a different RNA polynucleotide. The isolated mRNA(s) can be made in part or using only in vitro transcription. Methods of making polynucleotides by in vitro transcription are known in the art and are described in U.S. Provisional Patent Application Nos. 61/618,862, 61/681,645, 61/737,130, 61/618,866, 61/681,647, 61/737,134, 61/618,868, 61/681,648, 61/737,135, 61/618,873, 61/681,650, 61/737,147, 61/618,878, 61/681,654, 61/737,152, 61/618,885, 61/681,658, 61/737,155, 61/618,896, 61/668,157, 61/681,661, 61/737,160, 61/618,911, 61/681,667, 61/737,168, 61/618,922, 61/681,675, 61/737,174, 61/618,935, 61/681,687, 61/737,184, 61/618,945, 61/681,696, 61/737,191, 61/618,953, 61/681,704 61/737,203; International Publication Nos WO2013151666, WO2013151668, WO2013151663, WO2013151669, WO2013151670, WO2013151664, WO2013151665, WO2013151736, WO2013151672, WO2013151671 WO2013151667, and WO/2020/205793A1; the contents of each of which are herein incorporated by reference in their entireties.

### Lipid Nanoparticles

The isolated mRNAs and other polynucleotides of the mRNa vaccine can be formulated in a lipid nanoparticle. In some embodiments, the lipid nanoparticle is a cationic lipid nanoparticle.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the cationic lipid is a biodegradable cationic lipid. In some embodiments, the biodegradable cationic lipid comprises an ester linkage. In some embodiments, the biodegradable cationic lipid comprises DLin-DMA with an internal ester, DLin-DMA with a terminal ester, DLin-MC3-DMA with an internal ester, or DLin-MC3-DMA with a terminal ester.

In some embodiments, a lipid nanoparticle comprises a cationic lipid, a PEG-modified lipid, a sterol and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530). In some embodiments, the neutral lipid is 1,2-distearoyl-sn-glycero-3-phosphocholine (DSPC), the sterol is cholesterol, and the PEG-modified lipid is 1,2-dimyristoyl-racalycero-3-methoxypolyethylene glycol-2000 (PEG-DMG) or PEG-cDMA.

In some embodiments, the lipid nanoparticle is any nanoparticle described in U.S. Pat. No. 10,442,756, and/or comprises any compound described in U.S. Pat. No. 10,442,756, including but not limited to a nanoparticle according to any one of Formulas (IA) or (II) described therein.

In some embodiments, the lipid nanoparticle is any nanoparticle described in e.g., U.S. Pat. No. 10,266,485, and/or comprises any compound described in U.S. Pat. No. 10,266,485, including but not limited to a nanoparticle according to Formula (II) described therein.

In some embodiments, the lipid nanoparticle is a nanoparticle described in U.S. Pat. No. 9,868,692, and/or comprises a compound described in e.g., U.S. Pat. No. 9,868,692, including but not limited to a nanoparticle according to Formula (I), (1A), (II), (IIa), (IIb), (IIc), (IId), (IIe),

In some embodiments, a lipid nanoparticle comprises compounds of Formula (I) and/or Formula (II) as described in U.S. patent Ser. No. 10/272,150.

In some embodiments, the mRNA vaccine is formulated in a lipid nanoparticle that comprises a compound selected from Compounds 3, 18, 20, 25, 26, 29, 30, 60, 108-112 and 122 of U.S. Pat. No. 10,272,150.

In some embodiments, at least 80% (e.g., 85%, 90%, 95%, 98%, 99%) of the uracil in the open reading frame have a chemical modification, optionally wherein the vaccine is formulated in a lipid nanoparticle (e.g., a lipid nanoparticle comprises a cationic lipid, a PEG-modified lipid, a sterol and a non-cationic lipid).

In some embodiments, the lipid nanoparticle has a mean diameter of 50-200 nm.

In some embodiments, a lipid nanoparticle comprises compounds of Formula (I) and/or Formula (II), as discussed below.

In some embodiments, a lipid nanoparticle comprises Compounds 3, 18, 20, 25, 26, 29, 30, 60, 108-112, or 122 as set forth in U.S. patent Ser. No. 10/272,150.

In some embodiments, the lipid nanoparticle has a polydispersity value of less than 0.4 (e.g., less than 0.3, 0.2 or 0.1).

In some embodiments, a plurality of lipid nanoparticles, such as when contained in a formulation, has a mean PDI of between 0.02 and 0.2.

In some embodiments, a plurality of lipid nanoparticles, such as when contained in a formulation comprising one or more polynucleotide(s), has a mean lipid to polynucleotide ratio (wt/wt) of between 10 and 20.

In some embodiments, the lipid nanoparticle has a net neutral charge at a neutral pH value.

Methods of mRNA Vaccination

The compositions described herein can be used to induce an antigen specific immune response to a SARS-Cov-2 variant.

In some embodiments, the methods of inducing an antigen specific immune response in a subject include administering to the subject any of the RNA (e.g., mRNA) vaccine as provided herein in an amount effective to produce an antigen-specific immune response.

In some embodiments, an antigen-specific immune response comprises a T cell response and/or a B cell response.

In some embodiments, a method of producing an antigen-specific immune response comprises administering to a subject a single dose (no booster dose) of an RNA (e.g., mRNA) vaccine of the present disclosure.

In some embodiments, the RNA (e.g., mRNA) vaccine is a combination vaccine comprising a combination of an mRNA vaccine described herein and at least one other mRNA vaccine. The at least one other mRNA vaccine can be against the same or a different virus or disease-causing agent.

In some embodiments, a method further comprises administering to the subject a second (booster) dose of an RNA (e.g., mRNA) vaccine. Additional doses of an RNA (e.g., mRNA) vaccine may be administered.

In some embodiments, the subject exhibits a seroconversion rate of at least 80% (e.g., at least 85%, at least 90%, or at least 95%) following the first dose or the second (booster) dose of the vaccine. Seroconversion is the time period during which a specific antibody develops and becomes detectable in the blood. After seroconversion has occurred, a virus can be detected in blood tests for the antibody. During an infection or immunization, antigens enter the blood, and the immune system begins to produce antibodies in response. Before seroconversion, the antigen itself may or may not be detectable, but antibodies are considered absent. During seroconversion, antibodies are present but not yet detectable. Any time after seroconversion, the antibodies can be detected in the blood, indicating a prior or current infection.

In some embodiments, an RNA (e.g., mRNA) vaccine described herein is administered to a subject by intradermal, subcutaneous, or intramuscular injection. In some embodiments, the administering step comprises contacting a muscle tissue of the subject with a device suitable for injection of the composition. In some embodiments, the administering step comprises contacting a muscle tissue of the subject with a device suitable for injection of the composition in combination with electroporation.

In some embodiments, the anti-antigenic polypeptide antibody titer produced in the subject is increased by at least 1 log relative to a control. In some embodiments, the anti-antigenic polypeptide antibody titer produced in the subject is increased by 1-3 log relative to a control.

In some embodiments, the anti-antigenic polypeptide antibody titer produced in a subject is increased at least 2 times relative to a control. In some embodiments, the anti-antigenic polypeptide antibody titer produced in the subject is increased at least 5 times relative to a control. In some embodiments, the anti-antigenic polypeptide antibody titer produced in the subject is increased at least 10 times relative to a control. In some embodiments, the anti-antigenic polypeptide antibody titer produced in the subject is increased 2-10 times relative to a control.

In some embodiments, the control is an anti-antigenic polypeptide antibody titer produced in a subject who has not been administered an RNA (e.g., mRNA) vaccine of the present disclosure. In some embodiments, the control is an anti-antigenic polypeptide antibody titer produced in a subject who has been administered a live attenuated or inactivated vaccine against SARS-CoV-2 or wherein the control is an anti-antigenic polypeptide antibody titer produced in a subject who has been administered a recombinant or purified SARS-CoV-2 protein vaccine.

In some embodiments, the control is an anti-antigenic polypeptide antibody titer produced in a subject who has been administered a virus-like particle (VLP) vaccine comprising structural proteins of SARS-CoV-2.

The RNA (e.g., mRNA) vaccine of the present disclosure can be administered to a subject in an effective amount (e.g., an amount effective to induce an immune response in the subject).

In some embodiments, the RNA (e.g., mRNA) vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

In some embodiments, the efficacy (or effectiveness) of an RNA (e.g., mRNA) vaccine is greater than 60%.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas: Efficacy=(ARU−ARV)/ARU×100; and Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100.

In some embodiments, the efficacy (or effectiveness) of an RNA (e.g., mRNA) vaccine is at least 65%, at least 70%, at least 75%, at least 80%, at least 85%, or at least 90%.

In some embodiments, the vaccine immunizes the subject against one or more SARS-Cov-2 variants. Exemplary SARS-CoV-2 variants are described elsewhere herein.

In some embodiments, the subject to which the mRNA vaccine of the present disclosure is administered is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject to which the mRNA vaccine of the present disclosure is administered was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, an RNA (e.g., mRNA) vaccine may be administered later in life, for example, at the age of about 6 months to about 5 years, or older.

In some embodiments, the subject to which the mRNA vaccine of the present disclosure is administered the subject to which the mRNA vaccine of the present disclosure is administered is pregnant (e.g., in the first, second or third trimester) when administered an RNA (e.g., mRNA) vaccine.

In some embodiments, the subject to which the mRNA vaccine of the present disclosure is administered is a young adult between the ages of about 20 years and about 50 years (e.g., about 20, 25, 30, 35, 40, 45 or 50 years old).

In some embodiments, the subject to which the mRNA vaccine of the present disclosure is administered is an elderly subject about 60 years old, about 70 years old, or older (e.g., about 60, 65, 70, 75, 80, 85, 90, or about 100 or more years old).

In some embodiments, the subject to which the mRNA vaccine of the present disclosure is administered has a chronic pulmonary disease (e.g., chronic obstructive pulmonary disease (COPD) or asthma). Two forms of COPD include chronic bronchitis, which involves a long-term cough with mucus, and emphysema, which involves damage to the lungs overtime. Thus, a subject administered an RNA (e.g., mRNA) vaccine may have chronic bronchitis or emphysema.

In some embodiments, the subject to which the mRNA vaccine of the present disclosure is administered is immunocompromised (has an impaired immune system, e.g., has an immune disorder or autoimmune disorder).

In some embodiments, the mRNA vaccine of the present disclosure is delivered to a subject at a dosage of between 10 μg/kg and 400 μg/kg of the nucleic acid vaccine is administered to the subject. In some embodiments the dosage of the RNA polynucleotide is 1-5 μg, 5-10 μg, 10-μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the subject can receive 1, 2, 3, 4, 5, 6, 7, or more doses. After the initial dose (given at day zero) the subject can receive one or more (e.g., 1, 2, 3, 4, 5, 6, 7, 8, 9, 10, or more additional doses, referred to in the art as “booster” doses. The booster doses can follow the initial dose at any suitable time interval such as within days, weeks, months, or even years. In some embodiments, multiple booster doses are needed close in time after the initial dose (such as within 1, 2, 3, or 4 weeks after the initial dose) followed by a larger gap in time (e.g., months or years before subsequent booster doses are needed). In some embodiments, a first dose of the mRNA vaccine is administered to the subject on day zero. In some embodiments, a second dose of the mRNA vaccine ais administered to the subject on day 14, 21, 28, 35, 42, 49, 56, 63, 70, 77, 84 or more days after the first dose. In some embodiments, a third dose of the mRNA vaccine is administered to the subject on day 14, 21, 28, 35, 42, 49, 56, 63, 70, 77, 84 or more days after the first and/or second dose.

In some embodiments, the mRNA vaccine confers an antibody titer superior to the criterion for seroprotection for a SARS-CoV-2 variant for an acceptable percentage of human subjects. In some embodiments, the antibody titer produced by the mRNA vaccines of the invention is a neutralizing antibody titer. In some embodiments, the neutralizing antibody titer is greater than a protein vaccine. In other embodiments, the neutralizing antibody titer produced by the mRNA vaccines of the invention is greater than an adjuvanted protein vaccine. In yet other embodiments, the neutralizing antibody titer produced by the mRNA vaccines of the invention is 1,000-10,000, 1,200-10,000, 1,400-10,000, 1,500-10,000, 1,000-5,000, 1,000-4,000, 1,800-10,000, 2000-10,000, 2,000-5,000, 2,000-3,000, 2,000-4,000, 3,000-5,000, 3,000-4,000, or 2,000-2,500. A neutralization titer is typically expressed as the highest serum dilution required to achieve a 50% reduction in the number of plaques.

In some embodiments, a unit of use vaccine comprises between 10 ug and 400 ug of one or more RNA polynucleotides encoding the SARS-Cov-2 antigenic polypeptide(s) and/or immunogenic fragment(s) thereof and a pharmaceutically acceptable carrier or excipient, formulated for delivery to a human subject. In some embodiments, the vaccine further comprises a cationic lipid nanoparticle.

Aspects of the invention provide methods of creating, maintaining or restoring antigenic memory to a SARS-CoV-2 variant in an individual or population of individuals comprising administering to said individual or population an mRNA vaccine described herein.

In some embodiments, the methods of vaccinating a subject comprising administering to the subject a single dosage of between 25 ug/kg and 400 ug/kg of an mRNA vaccine comprising one or more RNA polynucleotides encoding a SARS-CoV-2 antigenic polypeptide and/or an immunogenic fragment thereof in an effective amount to vaccinate the subject.

In some embodiments, the mRNA vaccines comprising one or more RNA polynucleotides encoding a SARS-CoV-2 antigenic polypeptide and/or an immunogenic fragment thereof, wherein the RNA comprises at least one chemical modification, wherein the vaccine has at least 10 fold less RNA polynucleotide than is required for an unmodified mRNA vaccine to produce an equivalent antibody titer. In some embodiments, the RNA polynucleotide is present in a dosage of 25-100 micrograms.

In some embodiments, the mRNA vaccine comprises an LNP formulated RNA polynucleotide having an open reading frame comprising no nucleotide modifications (unmodified), the open reading frame one or more RNA polynucleotides encoding a SARS-CoV-2 antigenic polypeptide and/or an immunogenic fragment thereof, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

In some embodiments, the mRNA vaccine comprises an LNP formulated RNA polynucleotide having an open reading frame comprising one or more modifications, the open reading frame one or more RNA polynucleotides encoding a SARS-CoV-2 antigenic polypeptide and/or an immunogenic fragment thereof, wherein the vaccine has at least 10 fold less RNA polynucleotide than is required for an unmodified mRNA vaccine not formulated in a LNP to produce an equivalent antibody titer. In some embodiments, the RNA polynucleotide is present in a dosage of 25-100 micrograms.

In some embodiments, the method includes vaccinating a subject with a combination vaccine including at least two nucleic acid sequences encoding respiratory antigens, wherein at least one encodes a SARS-CoV-2 antigen wherein the dosage for the vaccine is a combined therapeutic dosage wherein the dosage of each individual nucleic acid encoding an antigen is a sub therapeutic dosage. In some embodiments, the combined dosage is 25 micrograms of the RNA polynucleotide in the nucleic acid vaccine administered to the subject. In some embodiments, the combined dosage is 100 micrograms of the RNA polynucleotide in the nucleic acid vaccine administered to the subject. In some embodiments the combined dosage is 50 micrograms of the RNA polynucleotide in the nucleic acid vaccine administered to the subject. In some embodiments, the combined dosage is 75 micrograms of the RNA polynucleotide in the nucleic acid vaccine administered to the subject. In some embodiments, the combined dosage is 150 micrograms of the RNA polynucleotide in the nucleic acid vaccine administered to the subject. In some embodiments, the combined dosage is 400 micrograms of the RNA polynucleotide in the nucleic acid vaccine administered to the subject. In some embodiments, the sub therapeutic dosage of each individual nucleic acid encoding an antigen is 1, 2, 3, 4, 5, 6, 7, 8, 9, 10, 11, 12, 13, 14, 15, 16, 17, 18, 19, or 20 micrograms.

In some embodiments, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In some embodiments, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In some embodiments, antigen-specific antibodies are measured in units of g/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

### SARS-CoV-2 Variants

The present disclosure relates to and/or involves SARS-CoV-2. More particularly the disclosure describes, inter alia, SARS-CoV-2 variant immunogenic polypeptides and encoding polynucleotides. As described herein are vaccines that include the SARS-CoV-2 variant immunogenic polypeptides and/or encoding polynucleotides. Such vaccines can be effective against one or more SARS-CoV-2 variants.

As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from. While there are many thousands of variants of SARS-CoV-2, (Koyama, Takahiko Koyama; Platt, Daniela; Parida, Laxmi (June 2020). “Variant analysis of SARS-CoV-2 genomes”. Bulletin of the World Health Organization. 98: 495-504) there are also much larger groupings called clades. Several different clade nomenclatures for SARS-CoV-2 have been proposed. As of December 2020, GISAID, referring to SARS-CoV-2 as hCoV-19 identified seven clades (0, S, L, V, G, GH, and GR) (Alm E, Broberg E K, Connor T, et al. Geographical and temporal distribution of SARS-CoV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, S84, V251, I378 and D392) (Guan Q, Sadykov M, Mfarrej S, et al. A genetic barcode of SARS-CoV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, A.; et al. “A dynamic nomenclature proposal for SARS-CoV-2 lineages to assist genomic epidemiology”. 5: 1403-1407).

Genetic variants of SARS-CoV-2 have been emerging and circulating around the world throughout the COVID-19 pandemic (see, e.g., The US Centers for Disease Control and Prevention; www.cdc.gov/coronavirus/2019-ncov/variants/variant-info.html). Disclosed herein are exemplary, non-limiting variants applicable to the present disclosure include variants of SARS-CoV-2, particularly those having substitutions of therapeutic concern, e.g., spike protein substitutions. In certain example embodiments, a SARS-CoV-2 variant includes one or more substitutions, optionally spike protein substitutions, disclosed herein.

In some embodiments, the SARS-Cov-2 variant is classified and/or otherwise identified as a Variant of Interest (VOI) by the World Health Organization and/or the U.S. Centers for Disease Control. A VOI will exhibit changes to receptor binding domain (RBD), reduced neutralization by antibodies generated against previous infection or vaccination, reduced efficacy of treatments or tests, and predicted increase in transmissibility or disease severity. A VOI will demonstrate specific genetic markers that are predicted to affect transmission, diagnostics, therapeutics, or immune escape, evidence that it is the cause of an increased proportion of cases or unique outbreak clusters, and limited prevalence or expansion in the US or in other countries.

In some embodiments, the SARS-CoV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures. A VOC may require increased public health action as compared to a VOI.

In some embodiments, the SARS-Cov-2 variant is classified and/or otherwise identified as a Variant of High Consequence (VOHC) by the World Health Organization and/or the U.S. Centers for Disease Control. A variant of high consequence has clear evidence that prevention measures or medical countermeasures (MCMs) have significantly reduced effectiveness relative to previously circulating variants.

In some embodiments, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In some embodiments, the SARS-Cov-2 variant is classified and/or is otherwise identified as a Variant Being Monitored (VBM). As used herein, VBM includes lineages whose data indicates a potential or clear impact on available medical countermeasures, lineages that cause more serious disease or increased transmission but are no longer detected, lineages previously designated as “variants of interest”, “variants of concern”, or “variants of high consequence”. A VOI or VOC may be downgraded to a VBM after it is no longer circulating at sustained levels and no longer poses significant risk to public health.

In certain example embodiments, a SARS-CoV-2 variant Table 5 shows exemplary, non-limiting genetic substitutions in SARS-CoV-2 variants. In certain example embodiments, a SARS-CoV-2 variant has one or more of the substitutions of Table 5.

TABLE 5

| Shared Spike Protein | |
|---|---|
| Substitutions | Common Pango Lineages |
| 69del-70del | B.1.1.7, B.1.525, B.1.1.529 |
| 144del | B.1.1.7, B.1.525, B.1.1.529 |
| E484K | B.1.1.7, B.1.351, B.1.525, B.1.526, B.1.621, |
| | B.1.623, P.1, P.2, P.3, R.1 |
| N501Y | B.1.1.7, B.1.351, B.1.1.529, P.1, P.2, P.3 |
| D614G | B.1.1.7, B.1.351, B.1.427, B.1.525, B.1.526, |
| | B.1.1.529, B.1.617.1, B.1.617.2, B.1.621, P.2, |
| | P.3 |
| A701V | B.1.351, B.1.526 |
| K417N | B.1.351, B.1.1.529 |
| H655Y | P.1, B.1.1.529 |
| T19R | B.1.617.1, B.1.617.2 |
| G142D | B.1.617.1, B.1.617.2, |
| L452R | A.2.5, B.1, B.1.429, B.1.427, B.1.526, |
| | B.1.617.1, B.1.617.2, C.36.3 |
| T478K | B.1.1.529, B.1.617.2 |
| P681R | B.1.617.1, B.1.617.2, A23.1 |
| P681H | B.1.1.7, B.1.1.529, B.1.621, P.3 |
| D950N | B.1.617.2, B.1.621 |
| A67V | B.1.1.529, B.1.525 |
| T95I | B.1.1.529. B.1.526, B.1.617.1, B.1.621 |
| S477N | B.1.1.529, B.1.526 |
| T859N | B.1.526, C.37 |
| V1176F | P.2, P.3 |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-CoV-2 lineages, known as the PANGO nomenclature. It is available at cov-lineages.org.

In some embodiments, the SARS-CoV-2 variant is or includes an Alpha (WHO) or UK variant (e.g., Pango lineage B.1.1.7 and Q lineages and subgroups and sublineages thereof) (Spike protein substitutions: 69del, 70del, 144del, (E484K*), (S494P*), N501Y, A570D, D614G, P681H, T716I, S982A, and D1118H (K1191N*)); a Beta (WHO) or South Africa variant (e.g., Pango lineage B.1.351, and subgroups and sublineages thereof, e.g., B.1.351.1, B.1.351.2, and/or B.1.351.3) (Spike protein substitutions: D80A, D215G, 241del, 242del, 243del, K417N, E484K, N501Y, D614G, L18F, R246I, and A701V); a Gamma (WHO) or Japan/Brazil variant (e.g., Pango lineage P.1 (alias, B.1.1.28.1), e.g., as described in Rambaut et al. 2020. Nat. Microbiol. 5:1403-1407) and subgroups and sublineages thereof (e.g., P.1.1, P.1.2, P.1.4, P.1.6, and/or P.1.7)) (spike protein substitutions: T20N, P26S, D138Y, R190S, K417T, E484K, N501Y, H655Y, TI027I)); a Delta (WHO) or India variant (e.g., Pango lineage B.1.617.2 and subgroups and sublineages thereof (e.g., AY.1 (or Delta plus), AY.2, AY.3, and AY.3.1)) (Spike protein substitutions: T19R, (G142D), 156del, 157del, R158G, L452R, T478K, D614G, P681R, and D950N); a Epsilon (WHO) or US California variant (e.g., B.1.427, and subgroups and sublineages thereof (spike protein substitutions: L452R, and D614G) and B.1.429, and subgroups and sublineages thereof (Spike protein substitutions: S131, W152C, L452R, and D614G)); an Eta variant (e.g., Pango lineage B.1.525 and subgroups and sublineages thereof) (Spike protein substitutions A67V, 69del, 70del, 144del, E484K, D614G, Q677H, F888L)); an Iota variant (e.g., Pango lineage B.1.526 and subgroups and sublineages thereof (Spike protein substitutions L5F, (D80G*), T95I, (Y144-*), (F157S*), D253G, (L452R*), (S477N*), E484K, D614G, A701V, (T859N*), (D950H*), (Q957R*))); a Kappa variant (e.g., Pango lineage B.1.617.1 and subgroups and sublineages thereof) (Spike protein substitutions (T95I), G142D, E154K, L452R, E484Q, D614G, P681R, Q1071H)); a Lambda variant (e.g., Pango lineage C.37 and subgroups and sublineages thereof) (Spike protein substitutions: G75V, T76I, Δ246-252, L452Q, F490S, D614G, and T859N); a Omicon (WHO) variant (B.1.1.529 and subgroups and sublineages thereof) (Spike protein substitutions: A67V, del69-70, T95I, del142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F); a Zeta variant (e.g., Pango lineage P.2, and subgroups and sublineages thereof) (Spike protein substitutions E484K, D614G, and VI 176F); a Mu variant (B.1.621, and subgroups and sublineages thereof, e.g., B1.621.1) (Spike protein substitutions: T95I, Y144S, Y145N, R346K, E484K, N501Y, D614G, P681H, and D950N); Pango lineage variant A.23.1, and subgroups and sublineages thereof, as described in Bugembe et al. medRxiv. 2021. doi: doi.org/10.1101/2021.02.08.21251393, (spike protein substitutions: F157L, V367F, Q613H, P681R); a Theta variant (e.g., Pango lineage P.3 and subgroups and sublineages thereof) (Spike protein substitutions: E484K, N501Y, D614G, P681H, E1092K, H1101Y, V1176F, K2Q); a descendant thereof, or any combination thereof.

The steps of the method described in the various examples disclosed herein are sufficient to carry out the methods of the present disclosure. Thus, in an example, a method consists essentially of a combination of the steps of the methods disclosed herein. In another example, a method consists of such steps.

The following Statements describe various examples of methods, products and systems of the present disclosure and are not intended to be in any way limiting:
1. An immunogenic composition comprising one or more peptides, wherein the one or more peptides are: a. capable of binding to Major Histocompatibility Complex (MHC) class II, and b. derived from translation products of SARS-CoV-2.
2. The immunogenic composition according to Statement 1, wherein the MHC class II is Human Leukocyte Antigen class II (HLA-II).
3. The immunogenic composition according to Statement 2, wherein the one or more peptides have a peptide-HLA-II binding affinity of less than 500 nMa.
4. The immunogenic composition according to Statement 2 or Statement 3, wherein the HLA-II is encoded by an HLA allele selected from the group consisting of: HLA-DRB1*07:01, HLA-DRB1*11:04, HLA-DRB1*15:01, HLA-DRB3*02:02, HLA-DRB4*01:01, HLA-DRB5*01:01, HLA-DPB1*03:01/HLA-DPA1*01:03, HLA-DPB1*04:02/HLA-DPA1*01:03, HLA-DPB1*06:01/HLA-DPA1*01:03, HLA-DQB1*02:02/HLA-DQA1*02:01, HLA-DQB1*02:02/HLA-DQA1*05:05, HLA-DQB1*03:01/HLA-DQA1*02:01, HLA-DQB1*03:01/HLA-DQA1*05:05, and HLA-DQB1*06:02/HLA-DQA1*01:02.
5. The immunogenic composition according to any one of Statements 1 to 4, wherein the HLA-II is encoded by an HLA allele selected from the group consisting of: HLA-DRB1*07:01, HLA-DRB1*11:04, HLA-DRB3*02:02, HLA-DRB4*01:01, DQB1*03:01/HLA-DQA1*05:05, HLA-DQB1*02:02/HLA-DQA1*02:01, HLA-DPB1*03:01/HLA-DPA1*01:03, DPB1*06:01/HLA-DPA1*01:03, HLA-DRB1*15:01, HLA-DRB5*01:01, HLA-DPB1*04:02/HLA-DPA1*01:03, and HLA-DQB1*06:02/HLA-DQA1*01:02.
6. The immunogenic composition according to any one of Statements 1 to 5, wherein at least one of the peptides is derived from translation of one or more internal out-of-frame open reading frames (ORFs) of SARS-CoV-2, one or more canonical ORFs of SARS-CoV-2, or any combination thereof.
7. The immunogenic composition according to Statement 6, wherein at least one of the internal out-of-frame ORFs is selected from the group consisting of ORF3c (ORF3a.iORF1) and/or ORF9b (N.iORF1).
8. The immunogenic composition according to Statement 6 or Statement 7, wherein at least one of the canonical ORFs is selected from the group consisting of non-structural protein 3 (nsp3) ORF, non-structural protein 4 (nsp4) ORF, ORF3a, ORF6, S protein ORF, M protein ORF, N protein ORF, and any combination thereof.
9. The immunogenic composition according to any one of Statements 1 to 8, wherein at least one of the peptides comprises a peptide sequence selected from the group consisting of internal ORF protein peptide sequences, canonical ORF protein peptide sequences, any subsequence thereof, and any combination thereof, and wherein at least one of the peptide sequences is selected from the group consisting of peptide sequences of Table 1, Table 2, and Table 4, any subsequence thereof, and any combination thereof.
10. The immunogenic composition according to any one of Statements 7 to 9, wherein the ORF3c overlaps with the ORF3a.
11. The immunogenic composition according to any one of Statements 7 to 10, wherein the ORF9b overlaps with the N protein ORF.
12. The immunogenic composition according to any one of Statements 7 to 11, wherein at least one of the peptides derived from translation of ORF3c comprises a peptide sequence of LLFFRALPK (SEQ ID NO: 546) or any subsequence thereof.
13. The immunogenic composition according to any one of Statements 7 to 12, wherein at least one of the peptides derived from translation of ORF3c comprises a peptide sequence of ALHFLLFFRALPKS (SEQ ID NO: 374) or any subsequence thereof.
14. The immunogenic composition according to any one of Statements 7 to 13, wherein at least one of the peptides derived from translation of ORF9b comprises a peptide sequence selected from the group consisting of PKVYPIILR (SEQ ID NO: 547), ISEMHPALR (SEQ ID NO: 548), any subsequence thereof, and any combination thereof.
15. The immunogenic composition according to any one of Statements 7 to 14, wherein at least one of the peptides derived from translation of ORF9b comprises a peptide sequence selected from the group consisting of VGPKVYPIILRLGSPLS (SEQ ID NO: 384), MDPKISEMHPALRLVDPQIQLAVTRMENA (SEQ ID NO: 382), any subsequence thereof, and any combination thereof.
16. The immunogenic composition according to any one of Statements 8 to 15, wherein at least one of the peptides derived from translation of the nsp3 ORF comprises a peptide sequence of VTAYNGYLT (SEQ ID NO: 549) or any subsequence thereof.
17. The immunogenic composition according to any one of Statements 8 to 16, wherein at least one of the peptides derived from translation of the nsp3 ORF comprises a peptide sequence selected from the group consisting of DGSEDNQTTTIQTIVE (SEQ ID NO: 363), SPDAVTAYNGYLTSSSK (SEQ ID NO: 364), any subsequence thereof, and any combination thereof.
18. The immunogenic composition according to any one of Statements 8 to 17, wherein at least one of the peptides derived from translation of the nsp4 ORF comprises a peptide sequence of IIQFPNTYL (SEQ ID NO: 550) or any subsequence thereof.
19. The immunogenic composition according to any one of Statements 8 to 18, wherein at least one of the peptides derived from translation of the nsp4 ORF comprises a peptide sequence of MDGSIIQFPNTYLEGSVR (SEQ ID NO: 365) or any subsequence thereof.
20. The immunogenic composition according to any one of Statements 8 to 19, wherein at least one of the peptides derived from translation of ORF3a comprises a peptide sequence selected from the group consisting of IKDATPSDF (SEQ ID NO: 551), FTIGTVTLK (SEQ ID NO: 552), any subsequence thereof, and any combination thereof.
21. The immunogenic composition according to any one of Statements 8 to 20, wherein at least one of the peptides derived from translation of ORF3a comprises a peptide sequence of MDLFMRIFTIGTVTLKQGEIKDATPSDF (SEQ ID NO: 99) or any subsequence thereof.
22. The immunogenic composition according to any one of Statements 8 to 21, wherein at least one of the peptides derived from translation of ORF6 comprises a peptide sequence of INLIIKNLS (SEQ ID NO: 553) or any subsequence thereof.
23. The immunogenic composition according to any one of Statements 8 to 22, wherein at least one of the peptides derived from translation of ORF6 comprises a peptide sequence of YIINLIIKNLSKS (SEQ ID NO: 100) or any subsequence thereof.
24. The immunogenic composition according to any one of Statements 8 to 23, wherein at least one of the peptides derived from translation of the S protein ORF comprises a peptide sequence selected from the group consisting of YTNSFTRGV (SEQ ID NO: 554), FKNIDGYFK (SEQ ID NO: 555), FQTLLALHR (SEQ ID NO: 556), IYQTSNFRV (SEQ ID NO: 557), FASVYAWNR (SEQ ID NO: 558), FVIRGDEVR (SEQ ID NO: 559), VIAWNSNNL (SEQ ID NO: 560), IAWNSNNLD (SEQ ID NO: 561), YQAGSTPCN (SEQ ID NO: 562), FLPFQQFGR (SEQ ID NO: 563), VYSTGSNVF (SEQ ID NO: 564), YQTQTNSPR (SEQ ID NO: 565), YTMSLGAEN (SEQ ID NO: 566), LLQYGSFCT (SEQ ID NO: 567), IAQYTSALL (SEQ ID NO: 568), LQIPFAMQM (SEQ ID NO: 569), FAMQMAYRF (SEQ ID NO: 570), LIRAAEIRA (SEQ ID NO: 571), IITTDNTFV (SEQ ID NO: 572), any subsequence thereof, and any combination thereof.
25. The immunogenic composition according to any one of Statements 8 to 24, wherein at least one of the peptides derived from translation of the S protein ORF comprises a peptide sequence selected from the group consisting of: TQLPPAYTNSFTRGVYYPDKVFRSSVLHSTQDLFLPFFS (SEQ ID NO: 529), FKNLREFVFKNIDGYFKIYSKHTPINLVRDL (SEQ ID NO: 530), INITRFQTLLALHRSYL (SEQ ID NO: 418), TVEKGIYQTSNFRVQPTES (SEQ ID NO: 532), ATRFASVYAWNRKRISN (SEQ ID NO: 424), DSFVIRGDEVRQIAPG (SEQ ID NO: 425), NYKLPDDFTGCVIAWNSNNLDSKVG (SEQ ID NO: 535), TEIYQAGSTPCNGVEG (SEQ ID NO: 440), ESNKKFLPFQQFGRDIADTTDAVRDPQT (SEQ ID NO: 442), TPTWRVYSTGSNVFQTRAG (SEQ ID NO: 538), ICASYQTQTNSPRRA (SEQ IDNO: 445), SVASQSIIAYTMSLGAEN (SEQ ID NO: 446), CSNLLLQYGSFCTQLNRALTGIAVEQDKNTQE (SEQ ID NO: 541), EPQIITTDNTFVSGN (SEQ ID NO: 119), VLPPLLTDEMIAQYTSALLAGTIT (SEQ ID NO: 460), AALQIPFAMQMAYRFNGIG (SEQ ID NO: 543), TQQLIRAAEIRASANLA (SEQ ID NO: 498), any subsequence thereof, and any combination thereof.
26. The immunogenic composition according to any one of Statements 8 to 25, wherein at least one of the peptides derived from translation of the M protein ORF comprises a peptide sequence selected from the group consisting of LHGTILTRP (SEQ ID NO: 573), YYKLGASQR (SEQ ID NO: 574), any subsequence thereof, and any combination thereof.
27. The immunogenic composition according to any one of Statements 8 to 26, wherein at least one of the peptides derived from translation of the M protein ORF comprises a peptide sequence selected from the group consisting of: NVPLHGTILTRPLLESELVIGAVILRGHLRIAGHHLGRCDIKDLPKEITVA (SEQ ID NO: 509), TSRTLSYYKLGASQRVAGDSG (SEQ ID NO: 139), TDHSSSSDNIALLVQ (SEQ ID NO: 22), any subsequence thereof, and any combination thereof.
28. The immunogenic composition according to any one of Statements 8 to 27, wherein at least one of the peptides derived from translation of the N protein ORF comprises a peptide sequence selected from the group consisting of FTALTQHGK (SEQ ID NO: 575), TGPEAGLPY (SEQ ID NO: 576), LPQGTTLPK (SEQ ID NO: 577), LLLLDRLNQ (SEQ ID NO: 578), VTQAFGRRG (SEQ ID NO: 579), FAPSASAFF (SEQ ID NO: 580), VTPSGTWLT (SEQ ID NO: 581), TQALPQRQK (SEQ ID NO: 582), any subsequence thereof, and any combination thereof.
29. The immunogenic composition according to any one of Statements 8 to 28, wherein at least one of the peptides derived from translation of the N protein ORF comprises a peptide sequence selected from the group consisting of: GLPNNTASWFTALTQHGKEDLKFPRGQGVPINTNSSPDDQIGYYRRATRRIR (SEQ ID NO: 513), TGPEAGLPYGANKDG (SEQ ID NO: 32), VATEGALNTPKDHIGTRNPANNAAIVLQLPQGTTLPKG (SEQ ID NO: 164), MAGNGGDAALALLLLDRLNQLESKMSGKGQQQQGQTVT (SEQ ID NO: 34), AAEASKKPRQKRTATKAYNVTQAFGRRGPEQTQGNFGDQELIRQGTD (SEQ ID NO: 517), IAQFAPSASAFFG (SEQ ID NO: 55), SDNGPQNQRNAPRITF (SEQ ID NO: 24), KKKADETQALPQRQKKQQTVTLLPAADLDDFSKQLQQSMSSADSTQA (SEQ ID NO: 520), RIGMEVTPSGTWLTYTGAIKLDDKDPNFKDQVILLNKHIDAYKTFPP (SEQ ID NO: 519), any subsequence thereof, and any combination thereof.
30. The immunogenic composition according to any one of Statements 1 to 29, wherein at least one of the peptides comprises a peptide sequence selected from the group consisting of N1XXN2XN3XXN4, any subsequence thereof, and any combination thereof, and wherein: N1 is F, I, L, M, V, W, or Y; N2 is A, I, F, L, M, N, T, Q, S, V, W, or Y; N3 is A, D, E, G, H, K, N, P, R, S, or T; and N4 is A, E, F, G, K, I, L, N, M, R, S, V, or Q.
31. The immunogenic composition according to Statement 30, wherein: N1 is F, I, L, M, V, W, or Y; N2 is N, T, S, or V; or Y; N3 is A, G, N, P, S, or T; and N4 is F, I, L, N, M, or V; N1 is I, L, M, or V; N2 is I, L, M, or V; N3 is H, K, or R; and N4 is A, G, S, or Q; N1 is F, I, L, V, W, or Y; N2 is F, I, N, M, W, or Y; N3 is D, G, N, or S; and N4 is K, L, N, S, or V; or N1 is F, I, L, M, V, W, or Y; N2 is A, E, I, L, M, Q, or V; N3 is A, E, G, or S; and N4 is E, K, or R.
32. A polynucleotide encoding one or more of the peptides of any one of Statements 1 to 31.
33. A vector comprising a polynucleotide of Statement 30.
34. The vector according to Statement 33, wherein the vector is a synthetic mRNA vaccine.
35. An immunogenic composition comprising: a. one or more peptides of any one of Statements 1 to 31, one or more polynucleotides of Statement 32, a vector of Statement 33 or Statement 34, or any combination thereof, and b. one or more antigenic components capable of stimulating production of an antibody targeting SARS-CoV-2.
36. The immunogenic composition according to Statement 35, wherein the one or more antigenic components comprises one or more antigenic peptides from a nucleocapsid phosphoprotein of SARS-CoV-2, a spike glycoprotein of SARS-CoV-2, or any combination thereof, one or more polynucleotides encoding the one or more antigenic peptides, or any combination thereof.
37. A therapeutic composition comprising an immunogenic composition of any one of Statements 1 to 31, Statement 35, or Statement 36, and an anti-viral therapeutic.
38. The therapeutic composition according to Statement 37, wherein the one or more polynucleotides encoding the one or more antigenic peptides are a synthetic mRNA vaccine.
39. A method of inducing a T cell response, and optionally an antibody response, to SARS-CoV-2 in a subject in need thereof comprising administering, to the subject, an immunogenic composition of any one of Statements 1 to 31, Statement 35, or Statement 36, a vector of Statement 33 or Statement 34, or any combination thereof.
40. A method of treating a SARS-CoV-2 infection in a subject in need thereof comprising administering a therapeutic composition of Statement 37 or Statement 38 to the subject in need thereof.
41. A method of determining an infection status of a subject comprising contacting immune cells derived from a subject with the immunogenic composition of any one of Statements 1 to 31, Statement 35, or Statement 36; and detecting cross-reactivity of the immune cells to the immunogenic composition.
42. A method of identifying immunogenic peptides comprising: a. lysing cells having a potential to express the immunogenic peptides of interest with a lysis buffer comprising a cell membrane disrupting detergent; b. enzymatic shearing of nucleic acids in the lysed cells; c. isolating HLA-II from the lysed cells, wherein the HLA-II is in complex with one or more peptides from the lysed cells; and d. determining sequences of the one or more peptides in complex with the HLA-II from (c).
43. The method according to Statement 42, further comprising (e) identifying HLA alleles that bind the peptides identified in using a HLA-II epitope binding predictor, and (f) selecting a subset of peptides that bind a defined percentage of HLA-II alleles.
44. The method according to Statement 43, further comprising selecting immunogenic peptides demonstrating a relative abundance above a defined threshold as determined by analysis of the complete cellular transcriptome and or proteome.
45. The method according to Statement 43 or Statement 44, further comprising ribosome sequencing to identify actively translated peptides and selecting immunogenic peptides that are being actively translated at one or more time points.
46. The method according to any one of Statements 42 to 45, wherein the nucleic acids in the lysed cells are enzymatically sheared using an endonuclease from Serratia marcescens and MgCl₂.
47. The method according to any one of Statements 42 to 46, wherein the cell membrane disrupting agent is a nonylphenol ethoxylate surfactant.
48. The method according to any one of Statements 42 to 47, wherein (d) is performed by liquid chromatography tandem mass spectrometry analysis.
49. The method according to any one of Statements 42 to 48, wherein isolating HLA-II comprises immunoprecipitation of the HLA-II complex with an anti-HLA-II antibody.
50. The method according to any one of Statements 42 to 49, wherein the immunogenic peptides of interest are expressed by a pathogen and wherein the cells have been infected with the pathogen.
51. The method according to any one of Statement 50, wherein the infected cells are engineered to express one or more cell surface receptors used by the pathogen to infect the cells.
52. The method according to Statement 50 or Statement 51, wherein the cells are treated with one or more cell signaling molecules related to infection by the pathogen.
53. The method according to any one of Statements 50 to 52, wherein the pathogen is a virus.
54. The method according to Statement 53, wherein the virus is SARS-Cov-2.
55. The method according to any one of Statements 42 to 54, wherein the cells are engineered to express CIITA, ACE2, and TMPRSS2.
56. The method according to any one of Statements 42 to 55, wherein the cells are engineered to increase or decrease HLA presentation.
57. The method according to any one of Statements 42 to 56, wherein the cells are engineered to increase or decrease expression of one or more of CIITA, proteasome subunits, tPA, POMP, or ubiquitin-proteasome genes.

### EXAMPLES

### Example 1

Targeted synthetic vaccines have the potential to transform the response to viral outbreaks; yet the design of these vaccines requires a comprehensive knowledge of viral immunogens, including T-cell epitopes. Having previously mapped the SARS-CoV-2 HLA-I landscape, here Applicant reports viral peptides that are naturally processed and loaded onto HLA-II complexes in infected cells. Applicant has identified over 500 unique viral peptides from canonical proteins, as well as overlapping internal open reading frames (ORFs), revealing, for the first time, the contribution of internal ORFs to the HLA-II peptide repertoire. Most HLA-II peptides co-localized with the known CD4+ T cell epitopes in COVID-19 patients. Applicant also observed that two reported immunodominant regions in the SARS-CoV-2 membrane protein are formed at the level of HLA-II presentation. Overall, these analyses showed that the HLA-I and HLA-II pathways target distinct viral proteins, with the structural proteins accounting for most of the HLA-II peptidome and non-structural and non-canonical proteins accounting for the majority of the HLA-I peptidome. These findings highlight the need for a vaccine design that incorporates multiple viral elements harboring CD4+ and CD8+ T cell epitopes to maximize the vaccine effectiveness.

In this study, Applicant set out to achieve a comprehensive map of SARS-CoV-2 peptides that are processed and presented by HLA-II complexes. Using these data, Applicant dissected the source viral proteins and the processed regions within each viral protein that are presented by HLA-II. Applicant contextualized these findings by using thousands of reported CD4+ T cell epitopes, inferring the contribution of antigen processing and presentation steps to T cell responses observed in COVID-19 patients. Applicant then compared the immunopeptidome of HLA-I and HLA-II complexes, revealing important differences between SARS-CoV-2 presentation to CD4+ and CD8+ T cells. The newly identified CD4+ T cell targets and the insights of this study rendered about viral HLA-II presentation will enable a more precise selection of peptides for the next generation of COVID-19 vaccines that aim to target multiple viral proteins. The concepts learned from this study can also be applied to the design of synthetic vaccines against other viral pathogens.

### Results

Immunopeptidome Profiling of HLA-II peptides in SARS-CoV-2 Infected Cells

To interrogate the HLA-II immunopeptidome of SARS-CoV-2, Applicant infected A549 and HEK293T cells with the virus after inducing the HLA-II presentation pathway, immunoprecipitated (IP) HLA-II-peptide complexes, and identified bound peptides by liquid chromatography-tandem mass spectrometry (LC-MS/MS) (FIG. 1A). Both cell lines stably expressed ACE2 and TMPRSS2, two important SARS-CoV-2 entry factors. To induce the HLA-II pathway, Applicant transduced the cells with the MHC class II transactivator (CIITA) using a lentiviral vector. Overexpression of CIITA, a master transcriptional regulator, facilitates cell-surface expression of HLA-II complexes and the peptide-loading machinery and has been previously used to interrogate the HLA-II immunopeptidome of virus-infected cells (Becerra-Artiles et al., 2019, 2022) and tumors (Forlani et al., 2021; Hos et al., 2022). In addition, some lung epithelial cells express HLA-II (Neuwelt et al., 2020; Wosen et al., 2018) and thus, studying the HLA-II immunopeptidome of infected cells mimics HLA-II presentation that can occur in-vivo during the course of infection with a respiratory virus.

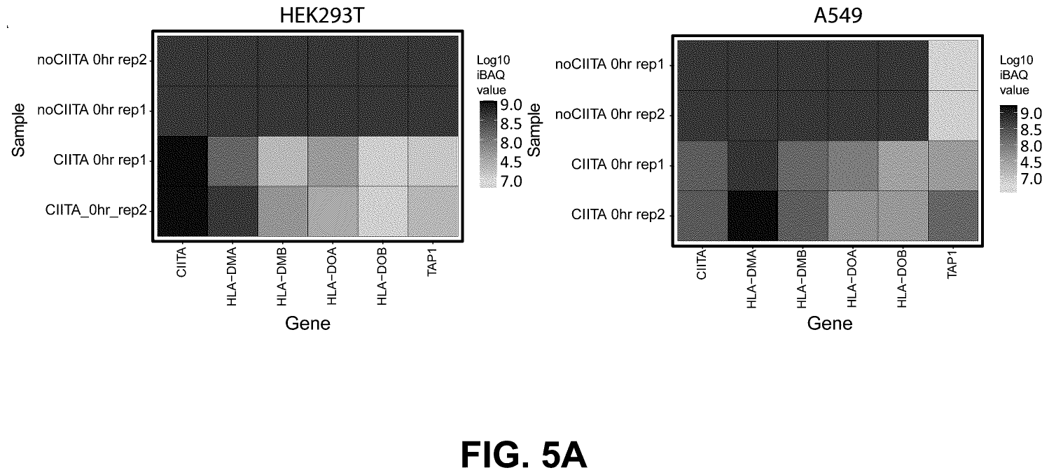

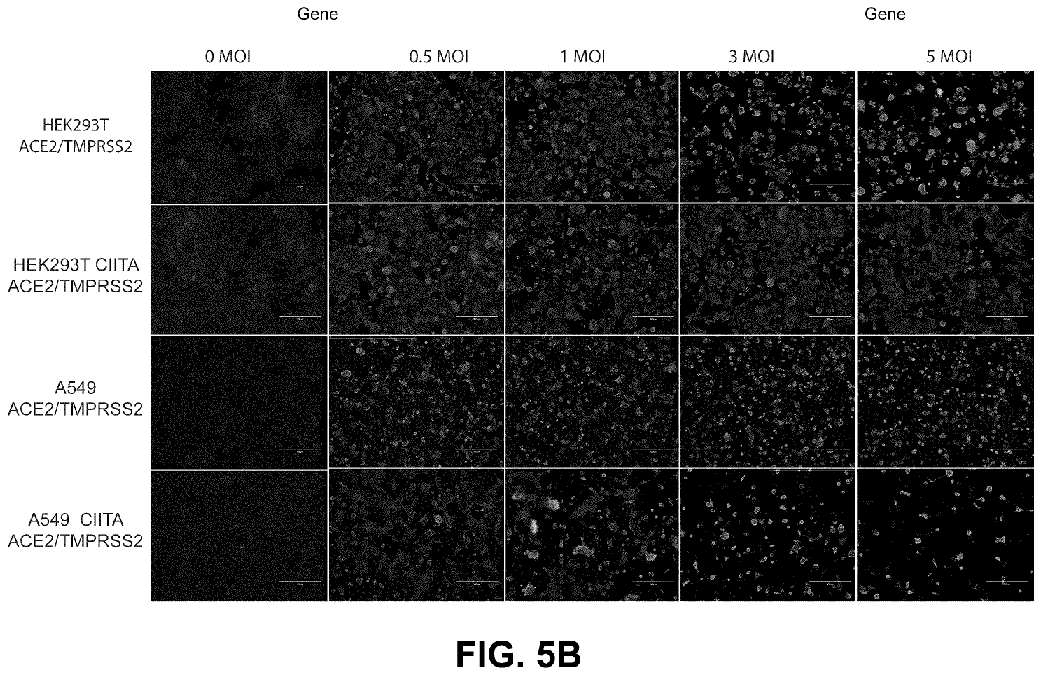

Applicant characterized the CIITA-transduced cells to ensure proper induction of proteins in the HLA-II pathway. First, Applicant compared cell surface levels of HLA-II in A549 cells with those in a positive control human melanoma A375 cell line that endogenously expresses HLA-II (Deffrennes et al., 2001). The cell surface flow cytometry revealed strong induction (˜80-fold) of HLA-II expression in A549/ACE2/TMPRSS2 (A549/AT) cells upon CIITA transduction, with a similar fluorescence intensity as in A375 cells (FIG. 1B). To monitor the expression of additional proteins in the HLA-II pathway, Applicant examined the whole proteome of A549/AT and HEK293T/AT cells by analyzing lysates after the HLA-II IP using LC-MS/MS. Applicant observed the expected increase in CIITA-induced proteins that are localized in the MHC-II region of the MHC locus including HLA-DM, HLA-DO, and TAP1 (FIG. 5A).

Applicant also ensured that CIITA overexpression did not affect the cell susceptibility to SARS-CoV-2 infection, since a recent study reported that CIITA can restrict SARS-like coronaviruses in U2OS osteosarcoma cells (Bruchez et al. 2020). Applicant quantified SARS-CoV-2 infection by immunofluorescence (IF) staining of cells with an anti-N antibody at 24 hours post infection (hpi) (FIGS. 1C, 5B). When infected at different multiplicities of infection (MOIs), and A549/AT cells exhibited similar infection levels regardless of CIITA expression. In contrast, HEK293T/AT cells showed reduced infection upon CIITA expression, although a substantial number of cells were still positive, with ˜50% infected cells at MOI of 3 (the MOI used for Applicant's HLA-II IP experiment).

To gauge the technical performance of Applicant's experimental system, Applicant examined if the peptides detected by LC-MS/MS match known characteristics of HLA-II peptides. Applicant performed HLA-II IP of non-infected and infected cells in two biological replicates was performed at 24 hpi using a mixture of antibodies targeting the three HLA-II loci (HLA-DR, HLA-DP, and HLA-DQ). Applicant recovered 21,541 and 29,600 unique HLA-II peptides from A549/ACE2/TMPRSS2/CIITA (A549/ATC) and HEK293T/ACE2/TMPRSS2/CIITA (HEK293T/ATC) cells, respectively. Of those, 0.5% (n=119) of A549 (Table 1) and 1.3% (n=389) of HEK293T peptides (Table 2) were derived from SARS-CoV-2 proteins.

TABLE 1
All SARS-CoV-2 peptides detected in infected A549 cells, annotated by experiments in which they are observed, viral segment, and
netMHCIIpan-4.1 binding prediction.

| Segment_ | | | netMHCIIpan-4.1 predictions | | | | | | | | | |
|---|---|---|---|---|---|---|---|---|---|---|---|---|
| id | Peptide | | DRB1_ | DRB1_ | DRB3_ | DRB4_ | DQA10201_ | DQA10505_ | DPA10103_ | DPA10103_ | Best | Best_allele |
| Gene: | length | Peptide | 0701 | 1104 | 0202 | 0101 | DQB10202 | DQB10301 | DPB10301 | DPB10601 | Rank | (Rank <= 2) |
| M: 121-171 | 20 | VPLHGTILT | 90.48 | 27.99 | 86.12 | 37.86 | 86.87 | 95 | 83.44 | 78.73 | 27.99 | unassigned |
| | | RPLLESELV | | | | | | | | | | |
| | | IG (SEQ | | | | | | | | | | |
| | | ID NO: 1) | | | | | | | | | | |
| M: 121-171 | 16 | VILRGHLRI | 23.88 | 1.17 | 17.73 | 9.46 | 71.64 | 48.12 | 15.55 | 11.42 | 1.17 | DRB1_1104 |
| | | AGHHLGR | | | | | | | | | | |
| | | (SEQ ID | | | | | | | | | | |
| | | NO: 2) | | | | | | | | | | |
| M: 121-171 | 18 | VILRGHLRI | 40.35 | 1.23 | 21.91 | 11.45 | 80.73 | 66.25 | 35.7 | 22.48 | 1.23 | DRB1_1104 |
| | | AGHHLGRCD | | | | | | | | | | |
| | | (SEQ ID | | | | | | | | | | |
| | | NO: 3) | | | | | | | | | | |
| M: 121-171 | 15 | ILRGHLRIA | 18.31 | 0.84 | 13.55 | 6.02 | 63.77 | 52.44 | 22.93 | 15.82 | 0.84 | DRB1_1104 |
| | | GHHLGR | | | | | | | | | | |
| | | (SEQ ID | | | | | | | | | | |
| | | NO: 4) | | | | | | | | | | |
| M: 121-171 | 16 | ILRGHLRIA | 35.17 | 1.55 | 23.25 | 13.04 | 80.82 | 68.4 | 32.93 | 21.46 | 1.55 | DRB1_1104 |
| | | GHHLGRC | | | | | | | | | | |
| | | (SEQ ID | | | | | | | | | | |
| | | NO: 5) | | | | | | | | | | |
| M: 121-171 | 17 | ILRGHLRIA | 32.74 | 0.9 | 16.65 | 7.98 | 74.96 | 59.21 | 37.46 | 22.86 | 0.9 | DRB1_1104 |
| | | GHHLGRCD | | | | | | | | | | |
| | | (SEQ ID | | | | | | | | | | |
| | | NO: 6) | | | | | | | | | | |
| M: 121-171 | 20 | ILRGHLRIA | 67.43 | 3.68 | 48.52 | 24.76 | 94.58 | 81.18 | 68.55 | 40.83 | 3.68 | unassigned |
| | | GHHLGRCDI | | | | | | | | | | |
| | | KD (SEQ | | | | | | | | | | |
| | | ID NO: 7) | | | | | | | | | | |
| M: 121-171 | 14 | LRGHLRIAG | 17.65 | 0.64 | 11.85 | 5.02 | 58.24 | 43.56 | 26.7 | 19.33 | 0.64 | DRB1_1104 |
| | | HHLGR | | | | | | | | | | |
| | | (SEQ ID | | | | | | | | | | |
| | | NO: 8) | | | | | | | | | | |
| M: 121-171 | 15 | LRGHLRIAG | 29.71 | 1.21 | 18.53 | 8.78 | 73.39 | 61.71 | 45.52 | 28.71 | 1.21 | DRB1_1104 |
| | | HHLGRC | | | | | | | | | | |
| | | (SEQ ID | | | | | | | | | | |
| | | NO: 9) | | | | | | | | | | |
| M: 121-171 | 16 | LRGHLRIAG | 30.56 | 0.76 | 14.7 | 6.54 | 70.9 | 46.23 | 34.21 | 22.2 | 0.76 | DRB1_1104 |
| | | HHLGRCD | | | | | | | | | | |
| | | (SEQ ID | | | | | | | | | | |
| | | NO: 10) | | | | | | | | | | |
| M: 121-171 | 13 | RGHLRIAGH | 18.07 | 0.62 | 10.83 | 4.59 | 57.48 | 40.38 | 28.89 | 20.13 | 0.62 | DRB1_1104 |
| | | HLGR (SEQ | | | | | | | | | | |
| | | ID NO: | | | | | | | | | | |
| | | 11) | | | | | | | | | | |
| M: 121-171 | 15 | RGHLRIAGH | 27.56 | 0.73 | 12.66 | 4.91 | 64.26 | 40.7 | 33.59 | 22.13 | 0.73 | DRB1_1104 |
| | | HLGRCD | | | | | | | | | | |
| | | (SEQ ID | | | | | | | | | | |
| | | NO: 12) | | | | | | | | | | |
| M: 121-171 | 18 | RGHLRIAGH | 55.05 | 2.62 | 35.65 | 16.32 | 83.78 | 63.33 | 48.1 | 30.73 | 2.62 | unassigned |
| | | HLGRCDIKD | | | | | | | | | | |
| | | (SEQ ID | | | | | | | | | | |
| | | NO: 13) | | | | | | | | | | |
| M: 121-171 | 22 | RGHLRIAGH | 88.39 | 10.52 | 72.98 | 44.7 | 92.7 | 89.75 | 82.42 | 66.91 | 10.52 | unassigned |
| | | HLGRCDIKD | | | | | | | | | | |
| | | LPKE (SEQ | | | | | | | | | | |
| | | ID NO: | | | | | | | | | | |
| | | 14) | | | | | | | | | | |
| M: 121-171 | 12 | GHLRIAGHH | 45.9 | 2.78 | 28.48 | 15.58 | 77.73 | 66.72 | 52.64 | 35.8 | 2.78 | unassigned |
| | | LGR (SEQ | | | | | | | | | | |
| | | ID NO: | | | | | | | | | | |
| | | 15) | | | | | | | | | | |
| M: 121-171 | 14 | RCDIKDLPK | 91.76 | 2.25 | 69 | 28.64 | 93.45 | 94.09 | 80.4 | 72.72 | 2.25 | unassigned |
| | | EITVA | | | | | | | | | | |
| | | (SEQ ID | | | | | | | | | | |
| | | NO: 16) | | | | | | | | | | |
| M: 172-192 | 19 | RTLSYYKLG | 2.93 | 6.11 | 5.9 | 52.14 | 41.53 | 6.77 | 46.42 | 35.34 | 2.93 | unassigned |
| | | ASQRVAGDS | | | | | | | | | | |
| | | G (SEQ ID | | | | | | | | | | |
| | | NO: 17) | | | | | | | | | | |
| M: 172-192 | 15 | TLSYYKLGA | 1.31 | 31.14 | 2.94 | 45.27 | 14.91 | 2.07 | 25.93 | 22.81 | 1.31 | DRB1_0701 |
| | | SQRVAG | | | | | | | | | | |
| | | (SEQ ID | | | | | | | | | | |
| | | NO: 18) | | | | | | | | | | |
| M: 172-192 | 17 | TLSYYKLGA | 1.28 | 5.92 | 2.78 | 43.35 | 19.52 | 2.35 | 27.91 | 24.41 | 1.28 | DRB1_0701 |
| | | SQRVAGDS | | | | | | | | | | |
| | | (SEQ ID | | | | | | | | | | |
| | | NO: 19) | | | | | | | | | | |
| M: 172-192 | 14 | LSYYKLGAS | 1.14 | 31.82 | 2.61 | 46.99 | 12.48 | 1.48 | 28.76 | 27.27 | 1.14 | DRB1_0701 |
| | | QRVAG | | | | | | | | | | |
| | | (SEQ ID | | | | | | | | | | |
| | | NO: 20) | | | | | | | | | | |
| M: 172-192 | 16 | LSYYKLGAS | 1.19 | 4.73 | 2.51 | 39.1 | 14.58 | 1.64 | 23.32 | 21.28 | 1.19 | DRB1_0701 |
| | | QRVAGDS | | | | | | | | | | |
| | | (SEQ ID | | | | | | | | | | |
| | | NO: 21) | | | | | | | | | | |
| M: 208-222 | 15 | TDHSSSSDN | 51.82 | 95 | 37.95 | 84.63 | 3.25 | 26.09 | 81.38 | 80.63 | 3.25 | unassigned |
| | | IALLVQ | | | | | | | | | | |
| | | (SEQ ID | | | | | | | | | | |
| | | NO: 22) | | | | | | | | | | |
| M: 208-222 | 13 | HSSSSDNIA | 67.29 | 95 | 39.72 | 92.71 | 6.46 | 37.33 | 82.08 | 85.38 | 6.46 | unassigned |
| | | LLVQ (SEQ | | | | | | | | | | |
| | | ID NO: | | | | | | | | | | |
| | | 23) | | | | | | | | | | |
| N: 2-17 | 16 | SDNGPQNQR | 82 | 68.55 | 45.88 | 47.24 | 78.63 | 64.2 | 79.59 | 63.89 | 45.88 | unassigned |
| | | NAPRITF | | | | | | | | | | |
| | | (SEQ ID | | | | | | | | | | |
| | | NO: 24) | | | | | | | | | | |
| N: 44-95 | 17 | NTASWFTAL | 8.68 | 12.89 | 3.46 | 11.53 | 6.92 | 2.74 | 18.68 | 24.92 | 2.74 | unassigned |
| | | TQHGKEDL | | | | | | | | | | |
| | | (SEQ ID | | | | | | | | | | |
| | | NO: 25) | | | | | | | | | | |
| N: 44-95 | 18 | NTASWFTAL | 12.95 | 18.37 | 5.52 | 18.52 | 12.48 | 4.76 | 26.79 | 31.76 | 4.76 | unassigned |
| | | TQHGKEDLK | | | | | | | | | | |
| | | (SEQ ID | | | | | | | | | | |
| | | NO: 26) | | | | | | | | | | |
| N: 44-95 | 15 | TASWFTALT | 5.8 | 8.45 | 2.13 | 7.07 | 3.17 | 1.29 | 13.37 | 18.19 | 1.29 | DQA10505_D |
| | | QHGKED | | | | | | | | | | QB10301 |
| | | (SEQ ID | | | | | | | | | | |
| | | NO: 27) | | | | | | | | | | |
| N: 44-95 | 16 | TASWFTALT | 8.26 | 11.84 | 3.28 | 10.81 | 5.22 | 1.94 | 17.06 | 21.94 | 1.94 | DQA10505_D |
| | | QHGKEDL | | | | | | | | | | QB10301 |
| | | (SEQ ID | | | | | | | | | | |
| | | NO: 28) | | | | | | | | | | |
| N: 44-95 | 17 | TASWFTALT | 11.55 | 15.76 | 4.72 | 15.59 | 9.22 | 3.54 | 21.64 | 27.49 | 3.54 | unassigned |
| | | QHGKEDLK | | | | | | | | | | |
| | | (SEQ ID | | | | | | | | | | |
| | | NO: 29) | | | | | | | | | | |
| N: 44-95 | 14 | ASWETALTQ | 9.65 | 10.36 | 4.04 | 11.97 | 4.53 | 1.93 | 17.32 | 21.6 | 1.93 | DQA10505_D |
| | | HGKED | | | | | | | | | | QB10301 |
| | | (SEQ ID | | | | | | | | | | |
| | | NO: 30) | | | | | | | | | | |
| N: 44-95 | 15 | ASWETALTQ | 12.94 | 13.34 | 5.68 | 16.89 | 7.81 | 3.04 | 20.25 | 24.86 | 3.04 | unassigned |
| | | HGKEDL | | | | | | | | | | |
| | | (SEQ ID | | | | | | | | | | |
| | | NO: 31) | | | | | | | | | | |
| N: 115-129 | 15 | TGPEAGLPY | 79.27 | 71.92 | 79.15 | 83.03 | 7.33 | 0.17 | 43.09 | 43.7 | 0.17 | DQA10505_D |
| | | GANKDG | | | | | | | | | | QB10301 |
| | | (SEQ ID | | | | | | | | | | |
| | | NO: 32) | | | | | | | | | | |
| N: 210-247 | 34 | MAGNGGDAA | 59.84 | 5.97 | 50.08 | 18.05 | 83.25 | 78.87 | 51.63 | 52.84 | 5.97 | unassigned |
| | | LALLLLDRL | | | | | | | | | | |
| | | NQLESKMSG | | | | | | | | | | |
| | | KGQQQQG | | | | | | | | | | |
| | | (SEQ ID | | | | | | | | | | |
| | | NO: 33) | | | | | | | | | | |
| N: 210-247 | 38 | MAGNGGDAA | 57.35 | 5.22 | 46.41 | 16.49 | 82.02 | 74.88 | 36.62 | 41.71 | 5.22 | unassigned |
| | | LALLLLDRL | | | | | | | | | | |
| | | NQLESKMSG | | | | | | | | | | |
| | | KGQQQQGQT | | | | | | | | | | |
| | | VT (SEQ | | | | | | | | | | |
| | | ID NO: | | | | | | | | | | |
| | | 34) | | | | | | | | | | |
| N: 210-247 | 22 | GNGGDAALA | 95 | 12.36 | 81.4 | 32.33 | 88.86 | 95 | 95 | 95 | 12.36 | unassigned |
| | | LLLLDRLNQ | | | | | | | | | | |
| | | LESK (SEQ | | | | | | | | | | |
| | | ID NO: | | | | | | | | | | |
| | | 35) | | | | | | | | | | |
| N: 210-247 | 36 | GNGGDAALA | 59.11 | 5.63 | 48.43 | 18.51 | 85.08 | 80.42 | 37.92 | 42.99 | 5.63 | unassigned |
| | | LLLLDRLNQ | | | | | | | | | | |
| | | LESKMSGKG | | | | | | | | | | |
| | | QQQQGQTVT | | | | | | | | | | |
| | | (SEQ ID | | | | | | | | | | |
| | | NO: 36) | | | | | | | | | | |
| N: 210-247 | 21 | NGGDAALAL | 95 | 13.44 | 83.06 | 35.85 | 90.36 | 95 | 95 | 95 | 13.44 | unassigned |
| | | LLLDRLNQL | | | | | | | | | | |
| | | ESK (SEQ | | | | | | | | | | |
| | | ID NO: | | | | | | | | | | |
| | | 37) | | | | | | | | | | |
| N: 210-247 | 20 | GGDAALALL | 95 | 9.03 | 75.41 | 25.75 | 87.64 | 95 | 93.02 | 91.93 | 9.03 | unassigned |
| | | LLDRLNQLE | | | | | | | | | | |
| | | SK (SEQ | | | | | | | | | | |
| | | ID NO: | | | | | | | | | | |
| | | 38) | | | | | | | | | | |
| N: 210-247 | 26 | GGDAALALL | 70.94 | 11.02 | 61.48 | 29.83 | 89.88 | 92.75 | 67.73 | 70.73 | 11.02 | unassigned |
| | | LLDRLNQLE | | | | | | | | | | |
| | | SKMSGKGQ | | | | | | | | | | |
| | | (SEQ ID | | | | | | | | | | |
| | | NO: 39) | | | | | | | | | | |
| N: 210-247 | 30 | GGDAALALL | 64.6 | 7.35 | 55.21 | 24.59 | 89.08 | 88.25 | 56.31 | 56.22 | 7.35 | unassigned |
| | | LLDRLNQLE | | | | | | | | | | |
| | | SKMSGKGQQ | | | | | | | | | | |
| | | QQG (SEQ | | | | | | | | | | |
| | | ID NO: | | | | | | | | | | |
| | | 40) | | | | | | | | | | |
| N: 210-247 | 32 | GGDAALALL | 62.84 | 6.69 | 52.21 | 23.02 | 88.32 | 83.99 | 42.96 | 47.3 | 6.69 | unassigned |
| | | LLDRLNQLE | | | | | | | | | | |
| | | SKMSGKGQQ | | | | | | | | | | |
| | | QQGQT | | | | | | | | | | |
| | | (SEQ ID | | | | | | | | | | |
| | | NO: 41) | | | | | | | | | | |
| N: 210-247 | 34 | GGDAALALL | 61.48 | 6.28 | 51.03 | 21.89 | 87.27 | 82.42 | 39.7 | 44.46 | 6.28 | unassigned |
| | | LLDRLNQLE | | | | | | | | | | |
| | | SKMSGKGQQ | | | | | | | | | | |
| | | QQGQTVT | | | | | | | | | | |
| | | (SEQ ID | | | | | | | | | | |
| | | NO: 42) | | | | | | | | | | |
| N: 210-247 | 19 | GDAALALLL | 94.5 | 6.63 | 66 | 20.28 | 85.05 | 95 | 91.94 | 90.41 | 6.63 | unassigned |
| | | LDRLNQLES | | | | | | | | | | |
| | | K (SEQ ID | | | | | | | | | | |
| | | NO: 43) | | | | | | | | | | |
| N: 210-247 | 18 | DAALALLLL | 91.28 | 4.89 | 54.74 | 18.02 | 81.77 | 95 | 89.44 | 86.82 | 4.89 | unassigned |
| | | DRLNQLESK | | | | | | | | | | |
| | | (SEQ ID | | | | | | | | | | |
| | | NO: 44) | | | | | | | | | | |
| N: 210-247 | 13 | AALALLLLD | 93.77 | 6.27 | 89.08 | 5.68 | 56.32 | 82.32 | 88.21 | 90.91 | 5.68 | unassigned |
| | | RLNQ (SEQ | | | | | | | | | | |
| | | ID NO: | | | | | | | | | | |
| | | 45) | | | | | | | | | | |
| N: 210-247 | 17 | AALALLLLD | 85.64 | 3.4 | 42.06 | 22.33 | 78.02 | 93.77 | 85.61 | 80.4 | 3.4 | unassigned |
| | | RLNQLESK | | | | | | | | | | |
| | | (SEQ ID | | | | | | | | | | |
| | | NO: 46) | | | | | | | | | | |
| N: 210-247 | 12 | ALALLLLDR | 95 | 9.95 | 95 | 30.61 | 85.04 | 95 | 95 | 95 | 9.95 | unassigned |
| | | LNQ (SEQ | | | | | | | | | | |
| | | ID NO: | | | | | | | | | | |
| | | 47) | | | | | | | | | | |
| N: 210-247 | 14 | ALALLLLDR | 88.72 | 2.21 | 57.91 | 30.64 | 69.05 | 92.79 | 88.49 | 87.61 | 2.21 | unassigned |
| | | LNQLE | | | | | | | | | | |
| | | (SEQ ID | | | | | | | | | | |
| | | NO: 48) | | | | | | | | | | |
| N: 210-247 | 15 | ALALLLLDR | 84.54 | 1.73 | 42.65 | 21.73 | 74.09 | 92.41 | 85.14 | 82.7 | 1.73 | DRB1_1104 |
| | | LNQLES | | | | | | | | | | |
| | | (SEQ ID | | | | | | | | | | |
| | | NO: 49) | | | | | | | | | | |
| N: 210-247 | 16 | ALALLLLDR | 79.95 | 3.15 | 34.12 | 22.25 | 69.34 | 92.54 | 81.35 | 75.68 | 3.15 | unassigned |
| | | LNQLESK | | | | | | | | | | |
| | | (SEQ ID | | | | | | | | | | |
| | | NO: 50) | | | | | | | | | | |
| N: 210-247 | 15 | LALLLLDRL | 77.23 | 4.71 | 26.75 | 17.77 | 67.36 | 90 | 76.52 | 73.77 | 4.71 | unassigned |
| | | NQLESK | | | | | | | | | | |
| | | (SEQ ID | | | | | | | | | | |
| | | NO: 51) | | | | | | | | | | |
| N: 210-247 | 14 | ALLLLDRLN | 82.71 | 9.97 | 32.69 | 20.79 | 62.22 | 89.81 | 74.65 | 73.04 | 9.97 | unassigned |
| | | QLESK | | | | | | | | | | |
| | | (SEQ ID | | | | | | | | | | |
| | | NO: 52) | | | | | | | | | | |
| N: 210-247 | 12 | LLLDRLNQL | 95 | 64.69 | 95 | 84.99 | 95 | 95 | 93.28 | 95 | 64.69 | unassigned |
| | | ESK (SEQ | | | | | | | | | | |
| | | ID NO: | | | | | | | | | | |
| | | 53) | | | | | | | | | | |
| N: 251-297 | 19 | TATKAYNVT | 3.67 | 26.09 | 5.65 | 35.57 | 51.81 | 34.43 | 27.15 | 21.4 | 3.67 | unassigned |
| | | QAFGRRGPE | | | | | | | | | | |
| | | Q (SEQ ID | | | | | | | | | | |
| | | NO: 54) | | | | | | | | | | |
| N: 304-316 | 13 | IAQFAPSAS | 0.56 | 73.25 | 11.75 | 82.69 | 20.21 | 12.57 | 67.64 | 53.63 | 0.56 | DRB1_0701 |
| | | AFFG (SEQ | | | | | | | | | | |
| | | ID NO: | | | | | | | | | | |
| | | 55) | | | | | | | | | | |
| N: 319-365 | 30 | IGMEVTPSG | 0.16 | 9.23 | 8.69 | 3.72 | 31.98 | 40.07 | 28.72 | 30.35 | 0.16 | DRB1_0701 |
| | | TWLTYTGAI | | | | | | | | | | |
| | | KLDDKDPNE | | | | | | | | | | |
| | | KDQ (SEQ | | | | | | | | | | |
| | | ID NO: | | | | | | | | | | |
| | | 56) | | | | | | | | | | |
| N: 319-365 | 35 | IGMEVTPSG | 0.15 | 7.88 | 7.48 | 2.95 | 30.67 | 37.85 | 26.51 | 28.39 | 0.15 | DRB1_0701 |
| | | TWLTYTGAI | | | | | | | | | | |
| | | KLDDKDPNE | | | | | | | | | | |
| | | KDQVILLN | | | | | | | | | | |
| | | (SEQ ID | | | | | | | | | | |
| | | NO: 57) | | | | | | | | | | |
| N: 319-365 | 23 | EVTPSGTWL | 0.25 | 28.09 | 17.21 | 58.07 | 40.55 | 51.92 | 39.81 | 39.65 | 0.25 | DRB1_0701 |
| | | TYTGAIKLD | | | | | | | | | | |
| | | DKDPN | | | | | | | | | | |
| | | (SEQ ID | | | | | | | | | | |
| | | NO: 58) | | | | | | | | | | |
| N: 319-365 | 22 | VTPSGTWLT | 0.28 | 29.71 | 18.88 | 59.95 | 43.26 | 54.34 | 41.78 | 41.74 | 0.28 | DRB1_0701 |
| | | YTGAIKLDD | | | | | | | | | | |
| | | KDPN (SEQ | | | | | | | | | | |
| | | ID NO: | | | | | | | | | | |
| | | 59) | | | | | | | | | | |
| N: 319-365 | 23 | VTPSGTWLT | 0.27 | 25.54 | 17.1 | 28.1 | 42.46 | 53 | 40.24 | 40.55 | 0.27 | DRB1_0701 |
| | | YTGAIKLDD | | | | | | | | | | |
| | | KDPNF | | | | | | | | | | |
| | | (SEQ ID | | | | | | | | | | |
| | | NO: 60) | | | | | | | | | | |
| N: 319-365 | 24 | VTPSGTWLT | 0.27 | 17.52 | 14.68 | 9.68 | 41.3 | 50.91 | 37.75 | 38.95 | 0.27 | DRB1_0701 |
| | | YTGAIKLDD | | | | | | | | | | |
| | | KDPNEK | | | | | | | | | | |
| | | (SEQ ID | | | | | | | | | | |
| | | NO: 61) | | | | | | | | | | |
| N: 319-365 | 26 | VTPSGTWLT | 0.26 | 10.21 | 10.54 | 4.08 | 38.35 | 47.32 | 32.56 | 35.2 | 0.26 | DRB1_0701 |
| | | YTGAIKLDD | | | | | | | | | | |
| | | KDPNFKDQ | | | | | | | | | | |
| | | (SEQ ID | | | | | | | | | | |
| | | NO: 62) | | | | | | | | | | |
| N: 319-365 | 18 | TPSGTWLTY | 0.05 | 26.6 | 8.29 | 46.72 | 20.25 | 29.84 | 19.35 | 18.43 | 0.05 | DRB1_0701 |
| | | TGAIKLDDK | | | | | | | | | | |
| | | (SEQ ID | | | | | | | | | | |
| | | NO: 63) | | | | | | | | | | |
| N: 319-365 | 20 | TPSGTWLTY | 0.22 | 25.29 | 14.2 | 63.09 | 35.09 | 42.74 | 28.25 | 25.55 | 0.22 | DRB1_0701 |
| | | TGAIKLDDK | | | | | | | | | | |
| | | DP (SEQ | | | | | | | | | | |
| | | ID NO: | | | | | | | | | | |
| | | 64) | | | | | | | | | | |
| N: 319-365 | 21 | TPSGTWLTY | 0.35 | 31.8 | 21.29 | 62.41 | 46.7 | 56.89 | 44.52 | 44.24 | 0.35 | DRB1_0701 |
| | | TGAIKLDDK | | | | | | | | | | |
| | | DPN (SEQ | | | | | | | | | | |
| | | ID NO: | | | | | | | | | | |
| | | 65) | | | | | | | | | | |
| N: 319-365 | 15 | GTWLTYTGA | 0.03 | 15.59 | 3.87 | 26.65 | 6.7 | 10.78 | 10.89 | 8.86 | 0.03 | DRB1_0701 |
| | | IKLDDK | | | | | | | | | | |
| | | (SEQ ID | | | | | | | | | | |
| | | NO: 66) | | | | | | | | | | |
| N: 319-365 | 13 | WLTYTGAIK | 0.54 | 19.56 | 17.02 | 61.38 | 18.82 | 20.23 | 30.05 | 27.98 | 0.54 | DRB1_0701 |
| | | LDDK (SEQ | | | | | | | | | | |
| | | ID NO: | | | | | | | | | | |
| | | 67) | | | | | | | | | | |
| N: 319-365 | 12 | LTYTGAIKL | 23.13 | 29.93 | 26.79 | 79 | 43.61 | 40.57 | 61.19 | 59.47 | 23.13 | unassigned |
| | | DDK (SEQ | | | | | | | | | | |
| | | ID NO: | | | | | | | | | | |
| | | 68) | | | | | | | | | | |
| N: 319-365 | 15 | LTYTGAIKL | 20.43 | 12.87 | 14.21 | 7.74 | 32.7 | 27.51 | 32.98 | 30.82 | 7.74 | unassigned |
| | | DDKDPN | | | | | | | | | | |
| | | (SEQ ID | | | | | | | | | | |
| | | NO: 69) | | | | | | | | | | |
| N: 319-365 | 11 | TYTGAIKLD | 74.94 | 62.17 | 60.02 | 92.86 | 88.19 | 77.86 | 88.4 | 86.84 | 60.02 | unassigned |
| | | DK (SEQ | | | | | | | | | | |
| | | ID NO: | | | | | | | | | | |
| | | 70) | | | | | | | | | | |
| N: 319-365 | 11 | YTGAIKLDD | 95 | 95 | 95 | 95 | 95 | 95 | 95 | 95 | 95 | unassigned |
| | | KD (SEQ | | | | | | | | | | |
| | | ID NO: | | | | | | | | | | |
| | | 71) | | | | | | | | | | |
| N: 319-365 | 27 | LDDKDPNEK | 53.94 | 1.04 | 4.43 | 7.67 | 72.32 | 68.92 | 24.5 | 35.56 | 1.04 | DRB1_1104 |
| | | DQVILLNKH | | | | | | | | | | |
| | | IDAYKTFPP | | | | | | | | | | |
| | | (SEQ ID | | | | | | | | | | |
| | | NO: 72) | | | | | | | | | | |
| N: 319-365 | 21 | DKDPNF KDQ | 65.29 | 1.84 | 11.58 | 17.04 | 80.74 | 75.98 | 30.74 | 44.41 | 1.84 | DRB1_1104 |
| | | VILLNKHID | | | | | | | | | | |
| | | AYK (SEQ | | | | | | | | | | |
| | | ID NO: | | | | | | | | | | |
| | | 73) | | | | | | | | | | |
| N: 319-365 | 22 | DKDPNF KDQ | 62.08 | 1.57 | 6.65 | 12.25 | 80.22 | 74.94 | 30.16 | 42.85 | 1.57 | DRB1_1104 |
| | | VILLNKHID | | | | | | | | | | |
| | | AYKT (SEQ | | | | | | | | | | |
| | | ID NO: | | | | | | | | | | |
| | | 74) | | | | | | | | | | |
| N: 319-365 | 25 | DKDPNF KDQ | 57.59 | 1.2 | 4.99 | 9.33 | 78.84 | 73.28 | 29.03 | 40.34 | 1.2 | DRB1_1104 |
| | | VILLNKHID | | | | | | | | | | |
| | | AYKTFPP | | | | | | | | | | |
| | | (SEQ ID | | | | | | | | | | |
| | | NO: 75) | | | | | | | | | | |
| N: 319-365 | 19 | DPNFKDQVI | 49.69 | 0.76 | 5.35 | 8.86 | 72.03 | 66.61 | 20.54 | 26.46 | 0.76 | DRB1_1104 |
| | | LLNKHIDAY | | | | | | | | | | |
| | | K (SEQ ID | | | | | | | | | | |
| | | NO: 76) | | | | | | | | | | |
| N: 319-365 | 20 | DPNFKDQVI | 60 | 1.16 | 4.93 | 10.35 | 80.84 | 74.55 | 27.27 | 32.52 | 1.16 | DRB1_1104 |
| | | LLNKHIDAY | | | | | | | | | | |
| | | KT (SEQ | | | | | | | | | | |
| | | ID NO: | | | | | | | | | | |
| | | 77) | | | | | | | | | | |
| N: 319-365 | 23 | DPNFKDQVI | 63.85 | 1.54 | 5.92 | 12 | 87.21 | 80.88 | 42.5 | 50.98 | 1.54 | DRB1_1104 |
| | | LLNKHIDAY | | | | | | | | | | |
| | | KTFPP | | | | | | | | | | |
| | | (SEQ ID | | | | | | | | | | |
| | | NO: 78) | | | | | | | | | | |
| N: 319-365 | 16 | FKDQVILLN | 32.72 | 0.2 | 1.72 | 3.81 | 75.77 | 67.34 | 39.82 | 32.45 | 0.2 | DRB1_1104 |
| | | KHIDAYK | | | | | | | | | | |
| | | (SEQ ID | | | | | | | | | | |
| | | NO: 79) | | | | | | | | | | |
| N: 319-365 | 17 | FKDQVILLN | 38.99 | 0.29 | 1.59 | 3.64 | 81.36 | 73.75 | 51.18 | 37.27 | 0.29 | DRB1_1104 |
| | | KHIDAYKT | | | | | | | | | | |
| | | (SEQ ID | | | | | | | | | | |
| | | NO: 80) | | | | | | | | | | |
| N: 319-365 | 20 | FKDQVILLN | 69.44 | 1.8 | 5.74 | 13 | 95 | 93.8 | 75.84 | 66.07 | 1.8 | DRB1_1104 |
| | | KHIDAYKTE | | | | | | | | | | |
| | | PP (SEQ | | | | | | | | | | |
| | | ID NO: | | | | | | | | | | |
| | | 81) | | | | | | | | | | |
| N: 319-365 | 13 | KDQVILLNK | 31.84 | 0.41 | 5.75 | 10.56 | 75.72 | 72.86 | 48.52 | 43.73 | 0.41 | DRB1_1104 |
| | | HIDA (SEQ | | | | | | | | | | |
| | | ID NO: | | | | | | | | | | |
| | | 82) | | | | | | | | | | |
| N: 319-365 | 14 | KDQVILLNK | 32.74 | 0.29 | 2.32 | 5.34 | 74.42 | 69.17 | 42.66 | 38.43 | 0.29 | DRB1_1104 |
| | | HIDAY | | | | | | | | | | |
| | | (SEQ ID | | | | | | | | | | |
| | | NO: 83) | | | | | | | | | | |
| N: 319-365 | 15 | KDQVILLNK | 32.28 | 0.16 | 1.32 | 2.78 | 71.66 | 63.76 | 39.2 | 30.14 | 0.16 | DRB1_1104 |
| | | HIDAYK | | | | | | | | | | |
| | | (SEQ ID | | | | | | | | | | |
| | | NO: 84) | | | | | | | | | | |
| N: 319-365 | 16 | KDQVILLNK | 36.3 | 0.25 | 1.22 | 2.88 | 75.6 | 66.74 | 44.47 | 32.48 | 0.25 | DRB1_1104 |
| | | HIDAYKT | | | | | | | | | | |
| | | (SEQ ID | | | | | | | | | | |
| | | NO: 85) | | | | | | | | | | |
| N: 319-365 | 19 | KDQVILLNK | 62 | 1.6 | 4.34 | 10.06 | 93.44 | 90.9 | 73.92 | 62.99 | 1.6 | DRB1_1104 |
| | | HIDAYKTEP | | | | | | | | | | |
| | | P (SEQ ID | | | | | | | | | | |
| | | NO: 86) | | | | | | | | | | |
| N: 319-365 | 12 | DQVILLNKH | 61.57 | 2.13 | 8.86 | 29.78 | 91.05 | 88.5 | 80.78 | 72.93 | 2.13 | unassigned |
| | | IDA (SEQ | | | | | | | | | | |
| | | ID NO: | | | | | | | | | | |
| | | 87) | | | | | | | | | | |
| N: 319-365 | 14 | DQVILLNKH | 39.76 | 0.38 | 1.08 | 2.53 | 71.85 | 64.78 | 49.14 | 37.29 | 0.38 | DRB1_1104 |
| | | IDAYK | | | | | | | | | | |
| | | (SEQ ID | | | | | | | | | | |
| | | NO: 88) | | | | | | | | | | |
| N: 319-365 | 15 | DQVILLNKH | 43.11 | 0.58 | 1.1 | 2.43 | 77.06 | 70.65 | 57.06 | 38.36 | 0.58 | DRB1_1104 |
| | | IDAYKT | | | | | | | | | | |
| | | (SEQ ID | | | | | | | | | | |
| | | NO: 89) | | | | | | | | | | |
| N: 319-365 | 11 | ILLNKHIDA | 95 | 81.47 | 95 | 41 | 95 | 95 | 95 | 95 | 41 | unassigned |
| | | YK (SEQ | | | | | | | | | | |
| | | ID NO: | | | | | | | | | | |
| | | 90) | | | | | | | | | | |
| N: 319-365 | 10 | LLNKHI DAY | 95 | 95 | 95 | 95 | 95 | 95 | 95 | 95 | 95 | unassigned |
| | | K (SEQ ID | | | | | | | | | | |
| | | NO: 91) | | | | | | | | | | |
| N: 373-419 | 19 | QRQKKQQTV | 26.21 | 33.03 | 46.96 | 26.92 | 17.34 | 45.61 | 18.11 | 24.91 | 17.34 | unassigned |
| | | TLLPAADLD | | | | | | | | | | |
| | | D (SEQ ID | | | | | | | | | | |
| | | NO: 92) | | | | | | | | | | |
| N: 373-419 | 21 | QRQKKQQTV | 40.47 | 42.78 | 62.72 | 42.78 | 37.47 | 67.73 | 34.96 | 45.75 | 34.96 | unassigned |
| | | TLLPAADLD | | | | | | | | | | |
| | | DES (SEQ | | | | | | | | | | |
| | | ID NO: | | | | | | | | | | |
| | | 93) | | | | | | | | | | |
| N: 373-419 | 25 | QRQKKQQTV | 36.25 | 36.51 | 54.77 | 34.61 | 33.14 | 59.5 | 31.91 | 42.35 | 31.91 | unassigned |
| | | TLLPAADLD | | | | | | | | | | |
| | | DF SKQLQ | | | | | | | | | | |
| | | (SEQ ID | | | | | | | | | | |
| | | NO: 94) | | | | | | | | | | |
| N: 373-419 | 35 | QRQKKQQTV | 22.03 | 11.38 | 33.44 | 25.4 | 31.12 | 54.7 | 25.18 | 35.02 | 11.38 | unassigned |
| | | TLLPAADLD | | | | | | | | | | |
| | | DE SKQLQQS | | | | | | | | | | |
| | | MS SADSTQ | | | | | | | | | | |
| | | (SEQ ID | | | | | | | | | | |
| | | NO: 95) | | | | | | | | | | |
| N: 373-419 | 36 | QRQKKQQTV | 20.47 | 11.11 | 32.28 | 24.94 | 30.99 | 53.36 | 24.31 | 33.96 | 11.11 | unassigned |
| | | TLLPAADLD | | | | | | | | | | |
| | | DE SKQLQQS | | | | | | | | | | |
| | | MS SADSTQA | | | | | | | | | | |
| | | (SEQ ID | | | | | | | | | | |
| | | NO: 96) | | | | | | | | | | |
| NA | 13 | KELITNIGR | NA | NA | NA | NA | NA | NA | NA | NA | NA | NA |
| | | KLHN (SEQ | | | | | | | | | | |
| | | ID NO: | | | | | | | | | | |
| | | 97) | | | | | | | | | | |
| NA | 15 | EVFVKITGF | NA | NA | NA | NA | NA | NA | NA | NA | NA | NA |
| | | DSLKSN | | | | | | | | | | |
| | | (SEQ ID | | | | | | | | | | |
| | | NO: 98) | | | | | | | | | | |
| ORF3a: 1-28 | 28 | MDLFMRIFT | 62.28 | 34.18 | 44.97 | 30.56 | 48.69 | 29.5 | 34.04 | 31.98 | 29.5 | unassigned |
| | | IGTVTLKQG | | | | | | | | | | |
| | | EIKDATPSD | | | | | | | | | | |
| | | F (SEQ ID | | | | | | | | | | |
| | | NO: 99) | | | | | | | | | | |
| ORF6: 31-43 | 13 | YIINLIIKN | 85.04 | 1.28 | 36.07 | 22.42 | 82.77 | 84.01 | 68.7 | 65.07 | 1.28 | DRB1_1104 |
| | | LSKS (SEQ | | | | | | | | | | |
| | | ID NO: | | | | | | | | | | |
| | | 100) | | | | | | | | | | |
| S: 22-60 | 32 | TQLPPAYTN | 7.11 | 17.25 | 2.48 | 8.87 | 34.35 | 13.55 | 17.47 | 14.22 | 2.48 | unassigned |
| | | SFTRGVYYP | | | | | | | | | | |
| | | DKVFRSSVL | | | | | | | | | | |
| | | HSTQD | | | | | | | | | | |
| | | (SEQ ID | | | | | | | | | | |
| | | NO: 101) | | | | | | | | | | |
| S: 22-60 | 17 | LPPAYTNSF | 2.02 | 41.21 | 12.58 | 63.33 | 56.62 | 20.28 | 57.45 | 60.13 | 2.02 | unassigned |
| | | TRGVYYPD | | | | | | | | | | |
| | | (SEQ ID | | | | | | | | | | |
| | | NO: 102) | | | | | | | | | | |
| S: 22-60 | 18 | SFTRGVYYP | 25.16 | 27.79 | 1.37 | 17.68 | 36.23 | 28.79 | 25.92 | 26.99 | 1.37 | DRB3_0202 |
| | | DKVFRSSVL | | | | | | | | | | |
| | | (SEQ ID | | | | | | | | | | |
| | | NO: 103) | | | | | | | | | | |
| S: 22-60 | 16 | FTRGVYYPD | 16.69 | 20.42 | 0.8 | 10.47 | 18.43 | 14.89 | 16.1 | 21.46 | 0.8 | DRB3_0202 |
| | | KVERSSV | | | | | | | | | | |
| | | (SEQ ID | | | | | | | | | | |
| | | NO: 104) | | | | | | | | | | |
| S: 22-60 | 17 | FTRGVYYPD | 20.72 | 22.58 | 1.07 | 13.24 | 26.45 | 21.52 | 19.78 | 27.93 | 1.07 | DRB3_0202 |
| | | KVFRSSVL | | | | | | | | | | |
| | | (SEQ ID | | | | | | | | | | |
| | | NO: 105) | | | | | | | | | | |
| S: 22-60 | 15 | TRGVYYPDK | 13.66 | 17.61 | 0.62 | 8.44 | 14.42 | 11.98 | 14.5 | 22.92 | 0.62 | DRB3_0202 |
| | | VERSSV | | | | | | | | | | |
| | | (SEQ ID | | | | | | | | | | |
| | | NO: 106) | | | | | | | | | | |
| S: 22-60 | 14 | RGVYYPDKV | 13.53 | 18.92 | 0.57 | 7.53 | 11.84 | 9.94 | 13.7 | 26.81 | 0.57 | DRB3_0202 |
| | | FRSSV | | | | | | | | | | |
| | | (SEQ ID | | | | | | | | | | |
| | | NO: 107) | | | | | | | | | | |
| S: 22-60 | 14 | GVYYPDKVE | 28.04 | 35.65 | 1.51 | 18.31 | 22.47 | 17.97 | 24.54 | 41.88 | 1.51 | DRB3_0202 |
| | | RSSVL | | | | | | | | | | |
| | | (SEQ ID | | | | | | | | | | |
| | | NO: 108) | | | | | | | | | | |
| S: 22-60 | 16 | YPDKVERSS | 5.93 | 6.66 | 5.96 | 2.08 | 6.54 | 1 | 5.58 | 3.72 | 1 | DQA10505_D |
| | | VLHSTQD | | | | | | | | | | QB10301 |
| | | (SEQ ID | | | | | | | | | | |
| | | NO: 109) | | | | | | | | | | |
| S: 22-60 | 17 | YPDKVERSS | 7.79 | 8.4 | 7.93 | 3.11 | 11.84 | 2.05 | 7.1 | 5.74 | 2.05 | unassigned |
| | | VLHSTQDL | | | | | | | | | | |
| | | (SEQ ID | | | | | | | | | | |
| | | NO: 110) | | | | | | | | | | |
| S: 22-60 | 23 | YPDKVERSS | 24.77 | 30.9 | 38.06 | 22.13 | 43.61 | 24.12 | 25.81 | 25.08 | 22.13 | unassigned |
| | | VLHSTQDLF | | | | | | | | | | |
| | | LPFFS | | | | | | | | | | |
| | | (SEQ ID | | | | | | | | | | |
| | | NO: 111) | | | | | | | | | | |
| S: 22-60 | 16 | RSSVLHSTQ | 6.9 | 73.36 | 77.35 | 27.89 | 12.3 | 52.77 | 89.32 | 89.23 | 6.9 | unassigned |
| | | DLFLPFF | | | | | | | | | | |
| | | (SEQ ID | | | | | | | | | | |
| | | NO: 112) | | | | | | | | | | |
| S: 22-60 | 17 | RSSVLHSTQ | 11.08 | 79.62 | 85.68 | 38.57 | 20.41 | 67.96 | 93.34 | 93.04 | 11.08 | unassigned |
| | | DLFLPFFS | | | | | | | | | | |
| | | (SEQ ID | | | | | | | | | | |
| | | NO: 113) | | | | | | | | | | |
| S: 186-216 | 22 | KNIDGYFKI | 7.95 | 23.69 | 29.13 | 63.59 | 91.69 | 89.83 | 63.71 | 40.75 | 7.95 | unassigned |
| | | YSKHTPINL | | | | | | | | | | |
| | | VRDL (SEQ | | | | | | | | | | |
| | | ID NO: | | | | | | | | | | |
| | | 114) | | | | | | | | | | |
| S: 307-325 | 18 | TVEKGI YQT | 0.24 | 13.7 | 7.82 | 23.02 | 30.97 | 43.6 | 36.81 | 13.62 | 0.24 | DRB1_0701 |
| | | SNFRVQPTE | | | | | | | | | | |
| | | (SEQ ID | | | | | | | | | | |
| | | NO: 115) | | | | | | | | | | |
| S: 307-325 | 16 | VEKGIYQTS | 0.09 | 9.63 | 4.28 | 13.01 | 17.9 | 27.71 | 27.94 | 10.52 | 0.09 | DRB1_0701 |
| | | NFRVQPT | | | | | | | | | | |
| | | (SEQ ID | | | | | | | | | | |
| | | NO: 116) | | | | | | | | | | |
| S: 307-325 | 18 | VEKGIYQTS | 0.28 | 15.45 | 8.88 | 27.18 | 34.28 | 46.82 | 38.59 | 14.29 | 0.28 | DRB1_0701 |
| | | NERVQPTES | | | | | | | | | | |
| | | (SEQ ID | | | | | | | | | | |
| | | NO: 117) | | | | | | | | | | |
| S: 630-648 | 17 | TPTWRVYST | 1.55 | 33.3 | 15.18 | 54.04 | 11.34 | 22.81 | 1.76 | 1.8 | 1.55 | DRB1_0701 |
| | | GSNVFQTR | | | | | | | | | | |
| | | (SEQ ID | | | | | | | | | | |
| | | NO: 118) | | | | | | | | | | |
| S: 1111-1125 | 15 | EPQIITTDN | 1.1 | 10.88 | 2.72 | 2.69 | 1.32 | 5.18 | 35.37 | 53.97 | 1.1 | DRB1_0701 |
| | | TFVSGN | | | | | | | | | | |
| | | (SEQ ID | | | | | | | | | | |
| | | NO: 119) | | | | | | | | | | |

TABLE 2
All SARS-CoV-2 peptides detected in infected HEK293T cells, annotated by experiments in which
they are observed, viral segment, and netMHCIIpan-4.1 binding prediction.

| | | | netMHCIIpan-4.1 predictions | | | | | |
|---|---|---|---|---|---|---|---|---|
| Segment_ | | | | | | | | Best_allele |
| id | Peptide | | DRB1_ | DRB5_ | DPA10103_ | DQA10102_ | Best | (Rank |
| Gene: | length | Peptide | 1501 | 0101 | DPB10402 | DQB10602 | Rank | <= 2) |
| M: 121-171 | 14 | NVPLHGT | 85.06 | 86.75 | 65.82 | 15.34 | 15.34 | unassigned |
| | | ILTRPLL | | | | | | |
| | | (SEQ ID | | | | | | |
| | | NO: 120) | | | | | | |
| M: 121-171 | 14 | ESELVIG | 24.96 | 3.95 | 21.8 | 0.41 | 0.41 | DQA10102_ |
| | | AVILRGH | | | | | | DQB10602 |
| | | (SEQ ID | | | | | | |
| | | NO: 121) | | | | | | |
| M: 121-171 | 15 | ESELVIG | 30.56 | 5.76 | 29.17 | 0.62 | 0.62 | DQA10102_ |
| | | AVILRGH | | | | | | DQB10602 |
| | | L | | | | | | |
| | | (SEQ ID | | | | | | |
| | | NO: 122) | | | | | | |
| M: 121-171 | 16 | ESELVIG | 36.77 | 10.71 | 25.18 | 0.35 | 0.35 | DQA10102_ |
| | | AVILRGH | | | | | | DQB10602 |
| | | LR | | | | | | |
| | | (SEQ ID | | | | | | |
| | | NO: 123) | | | | | | |
| M: 121-171 | 11 | SELVIGA | 92.73 | 87.77 | 95 | 95 | 87.77 | unassigned |
| | | VILR | | | | | | |
| | | (SEQ ID | | | | | | |
| | | NO: 124) | | | | | | |
| M: 121-171 | 13 | SELVIGA | 37.02 | 10.32 | 26.38 | 0.33 | 0.33 | DQA10102_ |
| | | VILRGH | | | | | | DQB10602 |
| | | (SEQ ID | | | | | | |
| | | NO: 125) | | | | | | |
| M: 121-171 | 14 | SELVIGA | 46.26 | 19.03 | 38.95 | 0.67 | 0.67 | DQA10102_ |
| | | VILRGHL | | | | | | DQB10602 |
| | | (SEQ ID | | | | | | |
| | | NO: 126) | | | | | | |
| M: 121-171 | 15 | SELVIGA | 39.44 | 16.12 | 21.01 | 0.23 | 0.23 | DQA10102_ |
| | | VILRGHL | | | | | | DQB10602 |
| | | R | | | | | | |
| | | (SEQ ID | | | | | | |
| | | NO: 127) | | | | | | |
| M: 121-171 | 12 | ELVIGAV | 69.86 | 58.25 | 63.98 | 1.68 | 1.68 | DQA10102_ |
| | | ILRGH | | | | | | DQB10602 |
| | | (SEQ ID | | | | | | |
| | | NO: 128) | | | | | | |
| M: 121-171 | 14 | ELVIGAV | 51.62 | 24.68 | 34.62 | 0.63 | 0.63 | DQA10102_ |
| | | ILRGHLR | | | | | | DQB10602 |
| | | (SEQ ID | | | | | | |
| | | NO: 129) | | | | | | |
| M: 121-171 | 11 | LVIGAVI | 95 | 95 | 95 | 17.38 | 17.38 | unassigned |
| | | LRGH | | | | | | |
| | | (SEQ ID | | | | | | |
| | | NO: 130) | | | | | | |
| M: 121-171 | 12 | LVIGAVI | 93.4 | 95 | 95 | 13.13 | 13.13 | unassigned |
| | | LRGHL | | | | | | |
| | | (SEQ ID | | | | | | |
| | | NO: 131) | | | | | | |
| M: 121-171 | 13 | LVIGAVI | 58.17 | 19.37 | 59.81 | 4.35 | 4.35 | unassigned |
| | | LRGHLR | | | | | | |
| | | (SEQ ID | | | | | | |
| | | NO: 132) | | | | | | |
| M: 121-171 | 10 | VIGAVIL | 95 | 95 | 95 | 85.25 | 85.25 | unassigned |
| | | RGH | | | | | | |
| | | (SEQ ID | | | | | | |
| | | NO: 133) | | | | | | |
| M: 121-171 | 9 | IGAVILR | 95 | 95 | 95 | 95 | 95 | unassigned |
| | | GH | | | | | | |
| | | (SEQ ID | | | | | | |
| | | NO: 134) | | | | | | |
| M: 121-171 | 1C | IGAVILR | 95 | 95 | 95 | 95 | 95 | unassigned |
| | | GHL | | | | | | |
| | | (SEQ ID | | | | | | |
| | | NO: 135) | | | | | | |
| M: 121-171 | 11 | IGAVILR | 89.8 | 51.3 | 92.89 | 95 | 51.3 | unassigned |
| | | GHLR | | | | | | |
| | | (SEQ ID | | | | | | |
| | | NO: 136) | | | | | | |
| M: 121-171 | 10 | GAVILRG | 95 | 89.46 | 95 | 95 | 89.46 | unassigned |
| | | HLR | | | | | | |
| | | (SEQ ID | | | | | | |
| | | NO: 137) | | | | | | |
| M: 172-192 | 18 | TSRTLSY | 6.08 | 0.41 | 26.52 | 40.43 | 0.41 | DRB5_ |
| | | YKLGASQ | | | | | | 0101 |
| | | RVAG | | | | | | |
| | | (SEQ ID | | | | | | |
| | | NO: 138) | | | | | | |
| M: 172-192 | 21 | TSRTLSY | 18.93 | 2.16 | 61.88 | 56.84 | 2.16 | unassigned |
| | | YKLGASQ | | | | | | |
| | | RVAGDSG | | | | | | |
| | | (SEQ ID | | | | | | |
| | | NO: 139) | | | | | | |
| M: 172-192 | 16 | SRTLSYY | 4.82 | 0.41 | 17.36 | 25.01 | 0.41 | DRB5_ |
| | | KLGASQR | | | | | | 0101 |
| | | VA | | | | | | |
| | | (SEQ ID | | | | | | |
| | | NO: 140) | | | | | | |
| M: 172-192 | 17 | SRTLSYY | 6.26 | 0.26 | 21.94 | 32.25 | 0.26 | DRB5_ |
| | | KLGASQR | | | | | | 0101 |
| | | VAG | | | | | | |
| | | (SEQ ID | | | | | | |
| | | NO: 141) | | | | | | |
| M: 172-192 | 18 | SRTLSYY | 7.4 | 0.45 | 26.92 | 28.36 | 0.45 | DRB5_ |
| | | KLGASQR | | | | | | 0101 |
| | | VAGD | | | | | | |
| | | (SEQ ID | | | | | | |
| | | NO: 142) | | | | | | |
| M: 172-192 | 20 | SRTLSYY | 18.45 | 1.54 | 55.4 | 43.17 | 1.54 | DRB5_ |
| | | KLGASQR | | | | | | 0101 |
| | | VAGDSG | | | | | | |
| | | (SEQ ID | | | | | | |
| | | NO: 143) | | | | | | |
| M: 172-192 | 15 | RTLSYYK | 8.53 | 0.29 | 25.05 | 39.85 | 0.29 | DRB5_ |
| | | LGASQRV | | | | | | 0101 |
| | | A | | | | | | |
| | | (SEQ ID | | | | | | |
| | | NO: 144) | | | | | | |
| M: 172-192 | 16 | RTLSYYK | 10.43 | 0.17 | 22.65 | 33.65 | 0.17 | DRB5_ |
| | | LGASQRV | | | | | | 0101 |
| | | AG | | | | | | |
| | | (SEQ ID | | | | | | |
| | | NO: 145) | | | | | | |
| M: 172-192 | 17 | RTLSYYK | 10.48 | 0.32 | 23.95 | 21.85 | 0.32 | DRB5_ |
| | | LGASQRV | | | | | | 0101 |
| | | AGD | | | | | | |
| | | (SEQ ID | | | | | | |
| | | NO: 146) | | | | | | |
| M: 172-192 | 19 | RTLSYYK | 20.91 | 1.14 | 49.99 | 32.59 | 1.14 | DRB5_ |
| | | LGASQRV | | | | | | 0101 |
| | | AGDSG | | | | | | |
| | | (SEQ ID | | | | | | |
| | | NO: 147) | | | | | | |
| M: 172-192 | 14 | TLSYYKL | 18.89 | 0.2 | 31.29 | 54.45 | 0.2 | DRB5_ |
| | | GASQRVA | | | | | | 0101 |
| | | (SEQ ID | | | | | | |
| | | NO: 148) | | | | | | |
| M: 172-192 | 15 | TLSYYKL | 15.79 | 0.16 | 21.8 | 32.81 | 0.16 | DRB5_ |
| | | GASQRVA | | | | | | 0101 |
| | | G | | | | | | |
| | | (SEQ ID | | | | | | |
| | | NO: 149) | | | | | | |
| M: 172-192 | 16 | TLSYYKL | 11.15 | 0.26 | 20.6 | 16.94 | 0.26 | DRB5_ |
| | | GASQRVA | | | | | | 0101 |
| | | GD | | | | | | |
| | | (SEQ ID | | | | | | |
| | | NO: 150) | | | | | | |
| M: 208-222 | 15 | TDHSSSS | 87.89 | 92.56 | 82.87 | 49.48 | 49.48 | unassigned |
| | | DNIALLV | | | | | | |
| | | Q | | | | | | |
| | | (SEQ ID | | | | | | |
| | | NO: 151) | | | | | | |
| M: 208-222 | 13 | HSSSSDN | 95 | 95 | 80.9 | 60.27 | 60.27 | unassigned |
| | | IALLVQ | | | | | | |
| | | (SEQ ID | | | | | | |
| | | NO: 152) | | | | | | |
| N: 44-95 | 22 | GLPNNTA | 52.38 | 2.26 | 40.34 | 29.38 | 2.26 | unassigned |
| | | SWFTALT | | | | | | |
| | | QHGKEDL | | | | | | |
| | | K | | | | | | |
| | | (SEQ ID | | | | | | |
| | | NO: 153) | | | | | | |
| N: 44-95 | 25 | GLPNNTA | 43.76 | 1.85 | 37.38 | 26.63 | 1.85 | DRB5_ |
| | | SWFTALT | | | | | | 0101 |
| | | QHGKEDL | | | | | | |
| | | KFPR | | | | | | |
| | | (SEQ ID | | | | | | |
| | | NO: 154) | | | | | | |
| N: 44-95 | 30 | GLPNNTA | 39.29 | 1.65 | 33.66 | 24.19 | 1.65 | DRB5_ |
| | | SWFTALT | | | | | | 0101 |
| | | QHGKEDL | | | | | | |
| | | KFPRGQG | | | | | | |
| | | VP | | | | | | |
| | | (SEQ ID | | | | | | |
| | | NO: 155) | | | | | | |
| N: 44-95 | 34 | GLPNNTA | 30.17 | 1.58 | 24.01 | 23.56 | 1.58 | DRB5_ |
| | | SWFTALT | | | | | | 0101 |
| | | QHGKEDL | | | | | | |
| | | KFPRGQG | | | | | | |
| | | VPINTN | | | | | | |
| | | (SEQ ID | | | | | | |
| | | NO: 156) | | | | | | |
| N: 44-95 | 28 | NTASWFT | 41.4 | 2.98 | 37.27 | 36.49 | 2.98 | unassigned |
| | | ALTQHGK | | | | | | |
| | | EDLKFPR | | | | | | |
| | | GQGVPIN | | | | | | |
| | | (SEQ ID | | | | | | |
| | | NO: 157) | | | | | | |
| N: 44-95 | 17 | TASWFTA | 33.02 | 0.45 | 15.2 | 9.99 | 0.45 | DRB5_ |
| | | LTQHGKE | | | | | | 0101 |
| | | DLK | | | | | | |
| | | (SEQ ID | | | | | | |
| | | NO: 158) | | | | | | |
| N: 44-95 | 21 | TASWFTA | 63.68 | 4.64 | 63.81 | 49.25 | 4.64 | unassigned |
| | | LTQHGKE | | | | | | |
| | | DLKFPRG | | | | | | |
| | | (SEQ ID | | | | | | |
| | | NO: 159) | | | | | | |
| N: 44-95 | 29 | TASWFTA | 39.24 | 4.02 | 37.21 | 44.66 | 4.02 | unassigned |
| | | LTQHGKE | | | | | | |
| | | DLKFPRG | | | | | | |
| | | QGVPINT | | | | | | |
| | | N | | | | | | |
| | | (SEQ ID | | | | | | |
| | | NO: 160) | | | | | | |
| N: 44-95 | 14 | ASWFTAL | 27.11 | 0.12 | 9.53 | 8.77 | 0.12 | DRB5_ |
| | | TQHGKED | | | | | | 0101 |
| | | (SEQ ID | | | | | | |
| | | NO: 161) | | | | | | |
| N: 44-95 | 16 | ASWETAL | 38.2 | 0.42 | 19.94 | 13.75 | 0.42 | DRB5_ |
| | | TQHGKED | | | | | | 0101 |
| | | LK | | | | | | |
| | | (SEQ ID | | | | | | |
| | | NO: 162) | | | | | | |
| N: 44-95 | 27 | GQGVPIN | 17.61 | 29.57 | 49.73 | 59.66 | 17.61 | unassigned |
| | | TNSSPDD | | | | | | |
| | | QIGYYRR | | | | | | |
| | | ATRRIR | | | | | | |
| | | (SEQ ID | | | | | | |
| | | NO: 163) | | | | | | |
| N: 133-170 | 38 | VATEGAL | 39.02 | 43.59 | 38.9 | 0.71 | 0.71 | DQA10102_ |
| | | NTPKDHI | | | | | | DQB10602 |
| | | GTRNPAN | | | | | | |
| | | NAAIVLQ | | | | | | |
| | | LPQGTTL | | | | | | |
| | | PKG | | | | | | |
| | | (SEQ ID | | | | | | |
| | | NO: 164) | | | | | | |
| N: 133-170 | 37 | ATEGALN | 39.83 | 44.25 | 39.68 | 0.73 | 0.73 | DQA10102_ |
| | | TPKDHIG | | | | | | DQB10602 |
| | | TRNPANN | | | | | | |
| | | AAIVLQL | | | | | | |
| | | PQGTTLP | | | | | | |
| | | KG | | | | | | |
| | | (SEQ ID | | | | | | |
| | | NO: 165) | | | | | | |
| N: 133-170 | 35 | TEGALNT | 43.03 | 46.38 | 41.72 | 0.8 | 0.8 | DQA10102_ |
| | | PKDHIGT | | | | | | DQB10602 |
| | | RNPANNA | | | | | | |
| | | AIVLQLP | | | | | | |
| | | QGTTLPK | | | | | | |
| | | (SEQ ID | | | | | | |
| | | NO: 166) | | | | | | |
| N: 133-170 | 36 | TEGALNT | 40.66 | 45.05 | 40.52 | 0.76 | 0.76 | DQA10102_ |
| | | PKDHIGT | | | | | | DQB10602 |
| | | RNPANNA | | | | | | |
| | | AIVLQLP | | | | | | |
| | | QGTTLPK | | | | | | |
| | | G | | | | | | |
| | | (SEQ ID | | | | | | |
| | | NO: 167) | | | | | | |
| N: 133-170 | 32 | LNTPKDH | 43.34 | 49.03 | 44.5 | 0.9 | 0.9 | DQA10102_ |
| | | IGTRNPA | | | | | | DQB10602 |
| | | NNAAIVL | | | | | | |
| | | QLPQGTT | | | | | | |
| | | LPKG | | | | | | |
| | | (SEQ ID | | | | | | |
| | | NO: 168) | | | | | | |
| N: 133-170 | 27 | NTPKDHI | 68.5 | 68.37 | 59.99 | 1.29 | 1.29 | DQA10102_ |
| | | GTRNPAN | | | | | | DQB10602 |
| | | NAAIVLQ | | | | | | |
| | | LPQGTT | | | | | | |
| | | (SEQ ID | | | | | | |
| | | NO: 169) | | | | | | |
| N: 133-170 | 31 | NTPKDHI | 44.09 | 50.11 | 45.87 | 0.96 | 0.96 | DQA10102_ |
| | | GTRNPAN | | | | | | DQB10602 |
| | | NAAIVLQ | | | | | | |
| | | LPQGTTL | | | | | | |
| | | PKG | | | | | | |
| | | (SEQ ID | | | | | | |
| | | NO: 170) | | | | | | |
| N: 133-170 | 24 | TPKDHIG | 77.02 | 73.46 | 67.09 | 2.43 | 2.43 | unassigned |
| | | TRNPANN | | | | | | |
| | | AAIVLQL | | | | | | |
| | | PQG | | | | | | |
| | | (SEQ ID | | | | | | |
| | | NO: 171) | | | | | | |
| N: 133-170 | 26 | TPKDHIG | 70.56 | 70.48 | 62.44 | 1.4 | 1.4 | DQA10102_ |
| | | TRNPANN | | | | | | DQB10602 |
| | | AAIVLQL | | | | | | |
| | | PQGTT | | | | | | |
| | | (SEQ ID | | | | | | |
| | | NO: 172) | | | | | | |
| N: 133-170 | 28 | TPKDHIG | 52.49 | 56 | 51.65 | 1.14 | 1.14 | DQA10102_ |
| | | TRNPANN | | | | | | DQB10602 |
| | | AAIVLQL | | | | | | |
| | | PQGTTLP | | | | | | |
| | | (SEQ ID | | | | | | |
| | | NO: 173) | | | | | | |
| N: 133-170 | 29 | TPKDHIG | 47.61 | 53.17 | 49.08 | 1.07 | 1.07 | DQA10102_ |
| | | TRNPANN | | | | | | DQB10602 |
| | | AAIVLQL | | | | | | |
| | | PQGTTLP | | | | | | |
| | | K | | | | | | |
| | | (SEQ ID | | | | | | |
| | | NO: 174) | | | | | | |
| N: 133-170 | 30 | TPKDHIG | 44.96 | 51.43 | 47.54 | 1.03 | 1.03 | DQA10102_ |
| | | TRNPANN | | | | | | DQB10602 |
| | | AAIVLQL | | | | | | |
| | | PQGTTLP | | | | | | |
| | | KG | | | | | | |
| | | (SEQ ID | | | | | | |
| | | NO: 175) | | | | | | |
| N: 133-170 | 20 | HIGTRNP | 84.12 | 89.16 | 79.54 | 1.45 | 1.45 | DQA10102_ |
| | | ANNAAIV | | | | | | DQB10602 |
| | | LQLPQG | | | | | | |
| | | (SEQ ID | | | | | | |
| | | NO: 176) | | | | | | |
| N: 133-170 | 22 | HIGTRNP | 80.25 | 86.1 | 80.18 | 2.09 | 2.09 | unassigned |
| | | ANNAAIV | | | | | | |
| | | LQLPQGT | | | | | | |
| | | T | | | | | | |
| | | (SEQ ID | | | | | | |
| | | NO: 177) | | | | | | |
| N: 133-170 | 19 | TRNPANN | 65.71 | 74.67 | 66.29 | 0.57 | 0.57 | DQA10102_ |
| | | AAIVLQL | | | | | | DQB10602 |
| | | PQGTT | | | | | | |
| | | (SEQ ID | | | | | | |
| | | NO: 178) | | | | | | |
| N: 133-170 | 22 | TRNPANN | 58.06 | 65.21 | 69.96 | 3.12 | 3.12 | unassigned |
| | | AAIVLQL | | | | | | |
| | | PQGTTLP | | | | | | |
| | | K | | | | | | |
| | | (SEQ ID | | | | | | |
| | | NO: 179) | | | | | | |
| N: 133-170 | 23 | TRNPANN | 54.91 | 62.93 | 68.44 | 2.97 | 2.97 | unassigned |
| | | AAIVLQL | | | | | | |
| | | PQGTTLP | | | | | | |
| | | KG | | | | | | |
| | | (SEQ ID | | | | | | |
| | | NO: 180) | | | | | | |
| N: 133-170 | 15 | RNPANNA | 67.66 | 80.07 | 64.89 | 0.17 | 0.17 | DQA10102_ |
| | | AIVLQLP | | | | | | DQB10602 |
| | | Q | | | | | | |
| | | (SEQ ID | | | | | | |
| | | NO: 181) | | | | | | |
| N: 133-170 | 16 | RNPANNA | 65.22 | 81.52 | 53.92 | 0.06 | 0.06 | DQA10102_ |
| | | AIVLQLP | | | | | | DQB10602 |
| | | QG | | | | | | |
| | | (SEQ ID | | | | | | |
| | | NO: 182) | | | | | | |
| N: 133-170 | 18 | RNPANNA | 57.7 | 67 | 56.87 | 0.27 | 0.27 | DQA10102_ |
| | | AIVLQLP | | | | | | DQB10602 |
| | | QGTT | | | | | | |
| | | (SEQ ID | | | | | | |
| | | NO: 183) | | | | | | |
| N: 133-170 | 21 | RNPANNA | 60.5 | 67.31 | 74.35 | 4.54 | 4.54 | unassigned |
| | | AIVLQLP | | | | | | |
| | | QGTTLPK | | | | | | |
| | | (SEQ ID | | | | | | |
| | | NO: 184) | | | | | | |
| N: 133-170 | 22 | RNPANNA | 57.64 | 64.85 | 72.8 | 4.32 | 4.32 | unassigned |
| | | AIVLQLP | | | | | | |
| | | QGTTLPK | | | | | | |
| | | G | | | | | | |
| | | (SEQ ID | | | | | | |
| | | NO: 185) | | | | | | |
| N: 133-170 | 11 | NPANNAA | 95 | 95 | 95 | 73.39 | 73.39 | unassigned |
| | | IVLQ | | | | | | |
| | | (SEQ ID | | | | | | |
| | | NO: 186) | | | | | | |
| N: 133-170 | 14 | NPANNAA | 73.18 | 86.7 | 60.82 | 0.1 | 0.1 | DQA10102_ |
| | | IVLQLPQ | | | | | | DQB10602 |
| | | (SEQ ID | | | | | | |
| | | NO: 187) | | | | | | |
| N: 133-170 | 15 | NPANNAA | 62.16 | 76.75 | 54.11 | 0.07 | 0.07 | DQA10102_ |
| | | IVLQLPQ | | | | | | DQB10602 |
| | | G | | | | | | |
| | | (SEQ ID | | | | | | |
| | | NO: 188) | | | | | | |
| N: 133-170 | 17 | NPANNAA | 51.47 | 56.91 | 49.68 | 0.15 | 0.15 | DQA10102_ |
| | | IVLQLPQ | | | | | | DQB10602 |
| | | GTT | | | | | | |
| | | (SEQ ID | | | | | | |
| | | NO: 189) | | | | | | |
| N: 133-170 | 20 | NPANNAA | 49.66 | 56.68 | 62.03 | 3.14 | 3.14 | unassigned |
| | | IVLQLPQ | | | | | | |
| | | GTTLPK | | | | | | |
| | | (SEQ ID | | | | | | |
| | | NO: 190) | | | | | | |
| N: 133-170 | 21 | NPANNAA | 60.65 | 67.37 | 76.53 | 6.93 | 6.93 | unassigned |
| | | IVLQLPQ | | | | | | |
| | | GTTLPKG | | | | | | |
| | | (SEQ ID | | | | | | |
| | | NO: 191) | | | | | | |
| N: 133-170 | 13 | PANNAAI | 83.19 | 94.83 | 73.04 | 0.21 | 0.21 | DQA10102_ |
| | | VLQLPQ | | | | | | DQB10602 |
| | | (SEQ ID | | | | | | |
| | | NO: 192) | | | | | | |
| N: 133-170 | 14 | PANNAAI | 70.86 | 89.08 | 61.45 | 0.09 | 0.09 | DQA10102_ |
| | | VLQLPQG | | | | | | DQB10602 |
| | | (SEQ ID | | | | | | |
| | | NO: 193) | | | | | | |
| N: 251-297 | 31 | AAEASKK | 12.86 | 0.06 | 6.79 | 25.85 | 0.06 | DRB5_ |
| | | PRQKRTA | | | | | | 0101 |
| | | TKAYNVT | | | | | | |
| | | QAFGRRG | | | | | | |
| | | PEQ | | | | | | |
| | | (SEQ ID | | | | | | |
| | | NO: 194) | | | | | | |
| N: 251-297 | 29 | EASKKPR | 13.7 | 0.07 | 7.52 | 27.44 | 0.07 | DRB5_ |
| | | QKRTATK | | | | | | 0101 |
| | | AYNVTQA | | | | | | |
| | | FGRRGPE | | | | | | |
| | | Q | | | | | | |
| | | (SEQ ID | | | | | | |
| | | NO: 195) | | | | | | |
| N: 251-297 | 20 | QKRTATK | 17.17 | 0.08 | 12.42 | 44.88 | 0.08 | DRB5_ |
| | | AYNVTQA | | | | | | 0101 |
| | | FGRRGP | | | | | | |
| | | (SEQ ID | | | | | | |
| | | NO: 196) | | | | | | |
| N: 251-297 | 21 | QKRTATK | 22.97 | 0.18 | 19.47 | 54.02 | 0.18 | DRB5_ |
| | | AYNVTQA | | | | | | 0101 |
| | | FGRRGPE | | | | | | |
| | | (SEQ ID | | | | | | |
| | | NO: 197) | | | | | | |
| N: 251-297 | 22 | QKRTATK | 21.48 | 0.15 | 18.41 | 41.57 | 0.15 | DRB5_ |
| | | AYNVTQA | | | | | | 0101 |
| | | FGRRGPE | | | | | | |
| | | Q | | | | | | |
| | | (SEQ ID | | | | | | |
| | | NO: 198) | | | | | | |
| N: 251-297 | 23 | QKRTATK | 20.53 | 0.12 | 17.64 | 38.97 | 0.12 | DRB5_ |
| | | AYNVTQA | | | | | | 0101 |
| | | FGRRGPE | | | | | | |
| | | QT | | | | | | |
| | | (SEQ ID | | | | | | |
| | | NO: 199) | | | | | | |
| N: 251-297 | 26 | QKRTATK | 18.74 | 0.1 | 15.97 | 34.96 | 0.1 | DRB5_ |
| | | AYNVTQA | | | | | | 0101 |
| | | FGRRGPE | | | | | | |
| | | QTQGN | | | | | | |
| | | (SEQ ID | | | | | | |
| | | NO: 200) | | | | | | |
| N: 251-297 | 17 | KRTATKA | 10.43 | 0.24 | 12.11 | 19.87 | 0.24 | DRB5_ |
| | | YNVTQAF | | | | | | 0101 |
| | | GRR | | | | | | |
| | | (SEQ ID | | | | | | |
| | | NO: 201) | | | | | | |
| N: 251-297 | 19 | KRTATKA | 14.45 | 0.03 | 9.86 | 34.59 | 0.03 | DRB5_ |
| | | YNVTQAF | | | | | | 0101 |
| | | GRRGP | | | | | | |
| | | (SEQ ID | | | | | | |
| | | NO: 202) | | | | | | |
| N: 251-297 | 20 | KRTATKA | 18.67 | 0.08 | 13.56 | 42.42 | 0.08 | DRB5_ |
| | | YNVTQAF | | | | | | 0101 |
| | | GRRGPE | | | | | | |
| | | (SEQ ID | | | | | | |
| | | NO: 203) | | | | | | |
| N: 251-297 | 21 | KRTATKA | 24.27 | 0.19 | 21.82 | 44.69 | 0.19 | DRB5_ |
| | | YNVTQAF | | | | | | 0101 |
| | | GRRGPEQ | | | | | | |
| | | (SEQ ID | | | | | | |
| | | NO: 204) | | | | | | |
| N: 251-297 | 25 | KRTATKA | 21.07 | 0.12 | 19.01 | 37.45 | 0.12 | DRB5_ |
| | | YNVTQAF | | | | | | 0101 |
| | | GRRGPEQ | | | | | | |
| | | TQGN | | | | | | |
| | | (SEQ ID | | | | | | |
| | | NO: 205) | | | | | | |
| N: 251-297 | 16 | RTATKAY | 10.29 | 0.11 | 10.38 | 17.31 | 0.11 | DRB5_ |
| | | NVTQAFG | | | | | | 0101 |
| | | RR | | | | | | |
| | | (SEQ ID | | | | | | |
| | | NO: 206) | | | | | | |
| N: 251-297 | 17 | RTATKAY | 11.41 | 0.03 | 8.31 | 19.14 | 0.03 | DRB5_ |
| | | NVTQAFG | | | | | | 0101 |
| | | RRG | | | | | | |
| | | (SEQ ID | | | | | | |
| | | NO: 207) | | | | | | |
| N: 251-297 | 18 | RTATKAY | 12.69 | 0.02 | 7.36 | 25.68 | 0.02 | DRB5_ |
| | | NVTQAFG | | | | | | 0101 |
| | | RRGP | | | | | | |
| | | (SEQ ID | | | | | | |
| | | NO: 208) | | | | | | |
| N: 251-297 | 19 | RTATKAY | 16.45 | 0.03 | 10.21 | 31.65 | 0.03 | DRB5_ |
| | | NVTQAFG | | | | | | 0101 |
| | | RRGPE | | | | | | |
| | | (SEQ ID | | | | | | |
| | | NO: 209) | | | | | | |
| N: 251-297 | 20 | RTATKAY | 20 | 0.09 | 14.67 | 32.72 | 0.09 | DRB5_ |
| | | NVTQAFG | | | | | | 0101 |
| | | RRGPEQ | | | | | | |
| | | (SEQ ID | | | | | | |
| | | NO: 210) | | | | | | |
| N: 251-297 | 21 | RTATKAY | 26.87 | 0.22 | 23.52 | 45.65 | 0.22 | DRB5_ |
| | | NVTQAFG | | | | | | 0101 |
| | | RRGPEQT | | | | | | |
| | | (SEQ ID | | | | | | |
| | | NO: 211) | | | | | | |
| N: 251-297 | 22 | RTATKAY | 25.86 | 0.2 | 22.74 | 43.43 | 0.2 | DRB5_ |
| | | NVTQAFG | | | | | | 0101 |
| | | RRGPEQT | | | | | | |
| | | Q | | | | | | |
| | | (SEQ ID | | | | | | |
| | | NO: 212) | | | | | | |
| N: 251-297 | 23 | RTATKAY | 25.11 | 0.19 | 22.09 | 41.93 | 0.19 | DRB5_ |
| | | NVTQAFG | | | | | | 0101 |
| | | RRGPEQT | | | | | | |
| | | QG | | | | | | |
| | | (SEQ ID | | | | | | |
| | | NO: 213) | | | | | | |
| N: 251-297 | 24 | RTATKAY | 24.5 | 0.17 | 21.43 | 40.82 | 0.17 | DRB5_ |
| | | NVTQAFG | | | | | | 0101 |
| | | RRGPEQT | | | | | | |
| | | QGN | | | | | | |
| | | (SEQ ID | | | | | | |
| | | NO: 214) | | | | | | |
| N: 251-297 | 25 | RTATKAY | 23.98 | 0.17 | 21.01 | 40.01 | 0.17 | DRB5_ |
| | | NVTQAFG | | | | | | 0101 |
| | | RRGPEQT | | | | | | |
| | | QGNF | | | | | | |
| | | (SEQ ID | | | | | | |
| | | NO: 215) | | | | | | |
| N: 251-297 | 26 | RTATKAY | 23.57 | 0.16 | 20.64 | 39.33 | 0.16 | DRB5_ |
| | | NVTQAFG | | | | | | 0101 |
| | | RRGPEQT | | | | | | |
| | | QGNFG | | | | | | |
| | | (SEQ ID | | | | | | |
| | | NO: 216) | | | | | | |
| N: 251-297 | 32 | RTATKAY | 21.87 | 0.13 | 19.39 | 36.49 | 0.13 | DRB5_ |
| | | NVTQAFG | | | | | | 0101 |
| | | RRGPEQT | | | | | | |
| | | QGNFGDQ | | | | | | |
| | | ELIR | | | | | | |
| | | (SEQ ID | | | | | | |
| | | NO: 217) | | | | | | |
| N: 251-297 | 15 | TATKAYN | 14.89 | 0.06 | 8.5 | 19.33 | 0.06 | DRB5_ |
| | | VTQAFGR | | | | | | 0101 |
| | | R | | | | | | |
| | | (SEQ ID | | | | | | |
| | | NO: 218) | | | | | | |
| N: 251-297 | 16 | TATKAYN | 15.14 | 0.02 | 6.6 | 16.86 | 0.02 | DRB5_ |
| | | VTQAFGR | | | | | | 0101 |
| | | RG | | | | | | |
| | | (SEQ ID | | | | | | |
| | | NO: 219) | | | | | | |
| N: 251-297 | 17 | TATKAYN | 12.56 | 0 | 4.94 | 20.01 | 0 | DRB5_ |
| | | VTQAFGR | | | | | | 0101 |
| | | RGP | | | | | | |
| | | (SEQ ID | | | | | | |
| | | NO: 220) | | | | | | |
| N: 251-297 | 18 | TATKAYN | 16.13 | 0.02 | 7.9 | 22.76 | 0.02 | DRB5_ |
| | | VTQAFGR | | | | | | 0101 |
| | | RGPE | | | | | | |
| | | (SEQ ID | | | | | | |
| | | NO: 221) | | | | | | |
| N: 251-297 | 19 | TATKAYN | 18.26 | 0.04 | 11.56 | 24.24 | 0.04 | DRB5_ |
| | | VTQAFGR | | | | | | 0101 |
| | | RGPEQ | | | | | | |
| | | (SEQ ID | | | | | | |
| | | NO: 222) | | | | | | |
| N: 251-297 | 20 | TATKAYN | 23.98 | 0.1 | 16.71 | 34.51 | 0.1 | DRB5_ |
| | | VTQAFGR | | | | | | 0101 |
| | | RGPEQT | | | | | | |
| | | (SEQ ID | | | | | | |
| | | NO: 223) | | | | | | |
| N: 251-297 | 21 | TATKAYN | 31.51 | 0.29 | 26.03 | 49.41 | 0.29 | DRB5_ |
| | | VTQAFGR | | | | | | 0101 |
| | | RGPEQTQ | | | | | | |
| | | (SEQ ID | | | | | | |
| | | NO: 224) | | | | | | |
| N: 251-297 | 22 | TATKAYN | 30.58 | 0.27 | 25.35 | 47.63 | 0.27 | DRB5_ |
| | | VTQAFGR | | | | | | 0101 |
| | | RGPEQTQ | | | | | | |
| | | G | | | | | | |
| | | (SEQ ID | | | | | | |
| | | NO: 225) | | | | | | |
| N: 251-297 | 23 | TATKAYN | 29.81 | 0.25 | 24.64 | 46.3 | 0.25 | DRB5_ |
| | | VTQAFGR | | | | | | 0101 |
| | | RGPEQTQ | | | | | | |
| | | GN | | | | | | |
| | | (SEQ ID | | | | | | |
| | | NO: 226) | | | | | | |
| N: 251-297 | 24 | TATKAYN | 29.19 | 0.24 | 24.22 | 45.28 | 0.24 | DRB5_ |
| | | VTQAFGR | | | | | | 0101 |
| | | RGPEQTQ | | | | | | |
| | | GNF | | | | | | |
| | | (SEQ ID | | | | | | |
| | | NO: 227) | | | | | | |
| N: 251-297 | 25 | TATKAYN | 28.7 | 0.23 | 23.75 | 44.52 | 0.23 | DRB5_ |
| | | VTQAFGR | | | | | | 0101 |
| | | RGPEQTQ | | | | | | |
| | | GNEG | | | | | | |
| | | (SEQ ID | | | | | | |
| | | NO: 228) | | | | | | |
| N: 251-297 | 26 | TATKAYN | 28.29 | 0.23 | 23.26 | 43.85 | 0.23 | DRB5_ |
| | | VTQAFGR | | | | | | 0101 |
| | | RGPEQTQ | | | | | | |
| | | GNFGD | | | | | | |
| | | (SEQ ID | | | | | | |
| | | NO: 229) | | | | | | |
| N: 251-297 | 27 | TATKAYN | 27.9 | 0.22 | 23 | 43.09 | 0.22 | DRB5_ |
| | | VTQAFGR | | | | | | 0101 |
| | | RGPEQTQ | | | | | | |
| | | GNEGDQ | | | | | | |
| | | (SEQ ID | | | | | | |
| | | NO: 230) | | | | | | |
| N: 251-297 | 28 | TATKAYN | 27.44 | 0.22 | 22.79 | 42.28 | 0.22 | DRB5_ |
| | | VTQAFGR | | | | | | 0101 |
| | | RGPEQTQ | | | | | | |
| | | GNFGDQE | | | | | | |
| | | (SEQ ID | | | | | | |
| | | NO: 231) | | | | | | |
| N: 251-297 | 29 | TATKAYN | 27.14 | 0.21 | 22.62 | 41.91 | 0.21 | DRB5_ |
| | | VTQAFGR | | | | | | 0101 |
| | | RGPEQTQ | | | | | | |
| | | GNFGDQE | | | | | | |
| | | L | | | | | | |
| | | (SEQ ID | | | | | | |
| | | NO: 232) | | | | | | |
| N: 251-297 | 30 | TATKAYN | 26.88 | 0.21 | 22.46 | 41.52 | 0.21 | DRB5_ |
| | | VTQAFGR | | | | | | 0101 |
| | | RGPEQTQ | | | | | | |
| | | GNEGDQE | | | | | | |
| | | LI | | | | | | |
| | | (SEQ ID | | | | | | |
| | | NO: 233) | | | | | | |
| N: 251-297 | 31 | TATKAYN | 26.66 | 0.2 | 22.32 | 40.96 | 0.2 | DRB5_ |
| | | VTQAFGR | | | | | | 0101 |
| | | RGPEQTQ | | | | | | |
| | | GNFGDQE | | | | | | |
| | | LIR | | | | | | |
| | | (SEQ ID | | | | | | |
| | | NO: 234) | | | | | | |
| N: 251-297 | 32 | TATKAYN | 26.45 | 0.2 | 22.14 | 39.31 | 0.2 | DRB5_ |
| | | VTQAFGR | | | | | | 0101 |
| | | RGPEQTQ | | | | | | |
| | | GNEGDQE | | | | | | |
| | | LIRQ | | | | | | |
| | | (SEQ ID | | | | | | |
| | | NO: 235) | | | | | | |
| N: 251-297 | 33 | TATKAYN | 26.27 | 0.2 | 21.63 | 38.41 | 0.2 | DRB5_ |
| | | VTQAFGR | | | | | | 0101 |
| | | RGPEQTQ | | | | | | |
| | | GNFGDQE | | | | | | |
| | | LIRQG | | | | | | |
| | | (SEQ ID | | | | | | |
| | | NO: 236) | | | | | | |
| N: 251-297 | 34 | TATKAYN | 26.1 | 0.2 | 20.87 | 37.02 | 0.2 | DRB5_ |
| | | VTQAFGR | | | | | | 0101 |
| | | RGPEQTQ | | | | | | |
| | | GNFGDQE | | | | | | |
| | | LIRQGT | | | | | | |
| | | (SEQ ID | | | | | | |
| | | NO: 237) | | | | | | |
| N: 251-297 | 35 | TATKAYN | 25.95 | 0.19 | 17.91 | 34.88 | 0.19 | DRB5_ |
| | | VTQAFGR | | | | | | 0101 |
| | | RGPEQTQ | | | | | | |
| | | GNFGDQE | | | | | | |
| | | LIRQGTD | | | | | | |
| | | (SEQ ID | | | | | | |
| | | NO: 238) | | | | | | |
| N: 251-297 | 14 | ATKAYNV | 19.19 | 0.03 | 8.13 | 21.22 | 0.03 | DRB5_ |
| | | TQAFGRR | | | | | | 0101 |
| | | (SEQ ID | | | | | | |
| | | NO: 239) | | | | | | |
| N: 251-297 | 15 | ATKAYNV | 14.03 | 0.01 | 5.59 | 17.77 | 0.01 | DRB5_ |
| | | TQAFGRR | | | | | | 0101 |
| | | G | | | | | | |
| | | (SEQ ID | | | | | | |
| | | NO: 240) | | | | | | |
| N: 251-297 | 16 | ATKAYNV | 11.83 | 0 | 4.36 | 16.94 | 0 | DRB5_ |
| | | TQAFGRR | | | | | | 0101 |
| | | GP | | | | | | |
| | | (SEQ ID | | | | | | |
| | | NO: 241) | | | | | | |
| N: 251-297 | 17 | ATKAYNV | 13.82 | 0.01 | 5.9 | 17.12 | 0.01 | DRB5_ |
| | | TQAFGRR | | | | | | 0101 |
| | | GPE | | | | | | |
| | | (SEQ ID | | | | | | |
| | | NO: 242) | | | | | | |
| N: 251-297 | 18 | ATKAYNV | 15.88 | 0.03 | 9.73 | 16.72 | 0.03 | DRB5_ |
| | | TQAFGRR | | | | | | 0101 |
| | | GPEQ | | | | | | |
| | | (SEQ ID | | | | | | |
| | | NO: 243) | | | | | | |
| N: 251-297 | 19 | ATKAYNV | 20.59 | 0.06 | 14.26 | 25.67 | 0.06 | DRB5_ |
| | | TQAFGRR | | | | | | 0101 |
| | | GPEQT | | | | | | |
| | | (SEQ ID | | | | | | |
| | | NO: 244) | | | | | | |
| N: 251-297 | 20 | ATKAYNV | 27.56 | 0.17 | 20.24 | 36.94 | 0.17 | DRB5_ |
| | | TQAFGRR | | | | | | 0101 |
| | | GPEQTQ | | | | | | |
| | | (SEQ ID | | | | | | |
| | | NO: 245) | | | | | | |
| N: 251-297 | 22 | ATKAYNV | 35.32 | 0.43 | 29.23 | 51.67 | 0.43 | DRB5_ |
| | | TQAFGRR | | | | | | 0101 |
| | | GPEQTQG | | | | | | |
| | | N | | | | | | |
| | | (SEQ ID | | | | | | |
| | | NO: 246) | | | | | | |
| N: 251-297 | 24 | ATKAYNV | 33.98 | 0.4 | 28.19 | 49.67 | 0.4 | DRB5_ |
| | | TQAFGRR | | | | | | 0101 |
| | | GPEQTQG | | | | | | |
| | | NFG | | | | | | |
| | | (SEQ ID | | | | | | |
| | | NO: 247) | | | | | | |
| N: 251-297 | 13 | TKAYNVT | 18.67 | 0.03 | 8.78 | 32.44 | 0.03 | DRB5_ |
| | | QAFGRR | | | | | | 0101 |
| | | (SEQ ID | | | | | | |
| | | NO: 248) | | | | | | |
| N: 251-297 | 14 | TKAYNVT | 14.69 | 0 | 5.74 | 24.3 | 0 | DRB5_ |
| | | QAFGRRG | | | | | | 0101 |
| | | (SEQ ID | | | | | | |
| | | NO: 249) | | | | | | |
| N: 251-297 | 15 | TKAYNVT | 10.14 | 0 | 3.91 | 19.38 | 0 | DRB5_ |
| | | QAFGRRG | | | | | | 0101 |
| | | P | | | | | | |
| | | (SEQ ID | | | | | | |
| | | NO: 250) | | | | | | |
| N: 251-297 | 16 | TKAYNVT | 13.45 | 0.01 | 5.78 | 14.09 | 0.01 | DRB5_ |
| | | QAFGRRG | | | | | | 0101 |
| | | PE | | | | | | |
| | | (SEQ ID | | | | | | |
| | | NO: 251) | | | | | | |
| N: 251-297 | 17 | TKAYNVT | 13.88 | 0.02 | 8.03 | 12.6 | 0.02 | DRB5_ |
| | | QAFGRRG | | | | | | 0101 |
| | | PEQ | | | | | | |
| | | (SEQ ID | | | | | | |
| | | NO: 252) | | | | | | |
| N: 251-297 | 21 | TKAYNVT | 44.01 | 0.83 | 36.33 | 57.82 | 0.83 | DRB5_ |
| | | QAFGRRG | | | | | | 0101 |
| | | PEQTQGN | | | | | | |
| | | (SEQ ID | | | | | | |
| | | NO: 253) | | | | | | |
| N: 251-297 | 12 | KAYNVTQ | 40.38 | 0.24 | 25.46 | 77.41 | 0.24 | DRB5_ |
| | | AFGRR | | | | | | 0101 |
| | | (SEQ ID | | | | | | |
| | | NO: 254) | | | | | | |
| N: 251-297 | 13 | KAYNVTQ | 22.46 | 0.03 | 11.64 | 41.21 | 0.03 | DRB5_ |
| | | AFGRRG | | | | | | 0101 |
| | | (SEQ ID | | | | | | |
| | | NO: 255) | | | | | | |
| N: 251-297 | 14 | KAYNVTQ | 17.55 | 0.02 | 7.85 | 20.97 | 0.02 | DRB5_ |
| | | AFGRRGP | | | | | | 0101 |
| | | (SEQ ID | | | | | | |
| | | NO: 256) | | | | | | |
| N: 251-297 | 15 | KAYNVTQ | 16.61 | 0.03 | 9.84 | 14 | 0.03 | DRB5_ |
| | | AFGRRGP | | | | | | 0101 |
| | | E | | | | | | |
| | | (SEQ ID | | | | | | |
| | | NO: 257) | | | | | | |
| N: 251-297 | 16 | KAYNVTQ | 16.18 | 0.08 | 13.88 | 9.77 | 0.08 | DRB5_ |
| | | AFGRRGP | | | | | | 0101 |
| | | EQ | | | | | | |
| | | (SEQ ID | | | | | | |
| | | NO: 258) | | | | | | |
| N: 251-297 | 17 | KAYNVTQ | 20.17 | 0.2 | 18.42 | 14.12 | 0.2 | DRB5_ |
| | | AFGRRGP | | | | | | 0101 |
| | | EQT | | | | | | |
| | | (SEQ ID | | | | | | |
| | | NO: 259) | | | | | | |
| N: 251-297 | 20 | KAYNVTQ | 48.65 | 2.24 | 48.72 | 47.86 | 2.24 | unassigned |
| | | AFGRRGP | | | | | | |
| | | EQTQGN | | | | | | |
| | | (SEQ ID | | | | | | |
| | | NO: 260) | | | | | | |
| N: 251-297 | 13 | AYNVTQA | 34.79 | 0.39 | 30.08 | 22.33 | 0.39 | DRB5_ |
| | | FGRRGP | | | | | | 0101 |
| | | (SEQ ID | | | | | | |
| | | NO: 261) | | | | | | |
| N: 251-297 | 15 | AYNVTQA | 16.42 | 1.65 | 35.41 | 10.42 | 1.65 | DRB5_ |
| | | FGRRGPE | | | | | | 0101 |
| | | Q | | | | | | |
| | | (SEQ ID | | | | | | |
| | | NO: 262) | | | | | | |
| N: 251-297 | 19 | AYNVTQA | 50.22 | 14.81 | 70.11 | 42.73 | 14.81 | unassigned |
| | | FGRRGPE | | | | | | |
| | | QTQGN | | | | | | |
| | | (SEQ ID | | | | | | |
| | | NO: 263) | | | | | | |
| N: 251-297 | 12 | NVTQAFG | 78.88 | 66.73 | 95 | 63.94 | 63.94 | unassigned |
| | | RRGPE | | | | | | |
| | | (SEQ ID | | | | | | |
| | | NO: 264) | | | | | | |
| N: 251-297 | 13 | NVTQAFG | 59.75 | 46.5 | 89.84 | 40.78 | 40.78 | unassigned |
| | | RRGPEQ | | | | | | |
| | | (SEQ ID | | | | | | |
| | | NO: 265) | | | | | | |
| N: 251-297 | 10 | VTQAFGR | 95 | 95 | 95 | 95 | 95 | unassigned |
| | | RGP | | | | | | |
| | | (SEQ ID | | | | | | |
| | | NO: 266) | | | | | | |
| N: 251-297 | 11 | VTQAFGR | 95 | 95 | 95 | 95 | 95 | unassigned |
| | | RGPE | | | | | | |
| | | (SEQ ID | | | | | | |
| | | NO: 267) | | | | | | |
| N: 251-297 | 12 | VTQAFGR | 95 | 95 | 95 | 95 | 95 | unassigned |
| | | RGPEQ | | | | | | |
| | | (SEQ ID | | | | | | |
| | | NO: 268) | | | | | | |
| N: 251-297 | 9 | TQAFGRR | 95 | 95 | 95 | 95 | 95 | unassigned |
| | | GP | | | | | | |
| | | (SEQ ID | | | | | | |
| | | NO: 269) | | | | | | |
| N: 251-297 | 10 | TQAFGRR | 95 | 95 | 95 | 95 | 95 | unassigned |
| | | GPE | | | | | | |
| | | (SEQ ID | | | | | | |
| | | NO: 270) | | | | | | |
| N: 251-297 | 11 | TQAFGRR | 95 | 95 | 95 | 95 | 95 | unassigned |
| | | GPEQ | | | | | | |
| | | (SEQ ID | | | | | | |
| | | NO: 271) | | | | | | |
| N: 251-297 | 15 | TQAFGRR | 72.29 | 36.58 | 45.7 | 84.75 | 36.58 | unassigned |
| | | GPEQTQG | | | | | | |
| | | N | | | | | | |
| | | (SEQ ID | | | | | | |
| | | NO: 272) | | | | | | |
| N: 251-297 | 9 | QAFGRRG | 95 | 95 | 95 | 95 | 95 | unassigned |
| | | PE | | | | | | |
| | | (SEQ ID | | | | | | |
| | | NO: 273) | | | | | | |
| N: 251-297 | 1C | QAFGRRG | 95 | 95 | 95 | 95 | 95 | unassigned |
| | | PEQ | | | | | | |
| | | (SEQ ID | | | | | | |
| | | NO: 274) | | | | | | |
| N: 319-365 | 31 | RIGMEVT | 6.99 | 8.32 | 11.41 | 48.72 | 6.99 | unassigned |
| | | PSGTWLT | | | | | | |
| | | YTGAIKL | | | | | | |
| | | DDKDPNF | | | | | | |
| | | KDQ | | | | | | |
| | | (SEQ ID | | | | | | |
| | | NO: 275) | | | | | | |
| N: 319-365 | 30 | IGMEVTP | 7.34 | 8.76 | 11.92 | 50.67 | 7.34 | unassigned |
| | | SGTWLTY | | | | | | |
| | | TGAIKLD | | | | | | |
| | | DKDPNEK | | | | | | |
| | | DQ | | | | | | |
| | | (SEQ ID | | | | | | |
| | | NO: 276) | | | | | | |
| N: 319-365 | 35 | IGMEVTP | 6.8 | 7.87 | 10.96 | 48.39 | 6.8 | unassigned |
| | | SGTWLTY | | | | | | |
| | | TGAIKLD | | | | | | |
| | | DKDPNEK | | | | | | |
| | | DQVILLN | | | | | | |
| | | (SEQ ID | | | | | | |
| | | NO: 277) | | | | | | |
| N: 319-365 | 25 | MEVTPSG | 9.13 | 10.94 | 15.78 | 58.98 | 9.13 | unassigned |
| | | TWLTYTG | | | | | | |
| | | AIKLDDK | | | | | | |
| | | DPNF | | | | | | |
| | | (SEQ ID | | | | | | |
| | | NO: 278) | | | | | | |
| N: 319-365 | 24 | EVTPSGT | 9.97 | 12.04 | 17.27 | 61.32 | 9.97 | unassigned |
| | | WLTYTGA | | | | | | |
| | | IKLDDKD | | | | | | |
| | | PNF | | | | | | |
| | | (SEQ ID | | | | | | |
| | | NO: 279) | | | | | | |
| N: 319-365 | 27 | EVTPSGT | 9.06 | 11.15 | 14.53 | 57.24 | 9.06 | unassigned |
| | | WLTYTGA | | | | | | |
| | | IKLDDKD | | | | | | |
| | | PNEKDQ | | | | | | |
| | | (SEQ ID | | | | | | |
| | | NO: 280) | | | | | | |
| N: 319-365 | 30 | EVTPSGT | 8.58 | 10.21 | 13.65 | 55.56 | 8.58 | unassigned |
| | | WLTYTGA | | | | | | |
| | | IKLDDKD | | | | | | |
| | | PNEKDQV | | | | | | |
| | | IL | | | | | | |
| | | (SEQ ID | | | | | | |
| | | NO: 281) | | | | | | |
| N: 319-365 | 32 | EVTPSGT | 8.36 | 9.84 | 13.29 | 54.81 | 8.36 | unassigned |
| | | WLTYTGA | | | | | | |
| | | IKLDDKD | | | | | | |
| | | PNEKDQV | | | | | | |
| | | ILLN | | | | | | |
| | | (SEQ ID | | | | | | |
| | | NO: 282) | | | | | | |
| N: 319-365 | 22 | VTPSGTW | 11.86 | 13.94 | 20.24 | 65.57 | 11.86 | unassigned |
| | | LTYTGAI | | | | | | |
| | | KLDDKDP | | | | | | |
| | | N4 | | | | | | |
| | | (SEQ ID | | | | | | |
| | | NO: 283) | | | | | | |
| N: 319-365 | 23 | VTPSGTW | 11.25 | 13.5 | 19.19 | 64.09 | 11.25 | unassigned |
| | | LTYTGAI | | | | | | |
| | | KLDDKDP | | | | | | |
| | | NE | | | | | | |
| | | (SEQ ID | | | | | | |
| | | NO: 284) | | | | | | |
| N: 319-365 | 24 | VTPSGTW | 10.79 | 13.14 | 18.08 | 62.71 | 10.79 | unassigned |
| | | LTYTGAI | | | | | | |
| | | KLDDKDP | | | | | | |
| | | NEK | | | | | | |
| | | (SEQ ID | | | | | | |
| | | NO: 285) | | | | | | |
| N: 319-365 | 26 | VTPSGTW | 10.13 | 12.54 | 16.01 | 59.77 | 10.13 | unassigned |
| | | LTYTGAI | | | | | | |
| | | KLDDKDP | | | | | | |
| | | NEKDQ | | | | | | |
| | | (SEQ ID | | | | | | |
| | | NO: 286) | | | | | | |
| N: 319-365 | 27 | VTPSGTW | 9.9 | 12.05 | 15.63 | 59.17 | 9.9 | unassigned |
| | | LTYTGAI | | | | | | |
| | | KLDDKDP | | | | | | |
| | | NEKDQV | | | | | | |
| | | (SEQ ID | | | | | | |
| | | NO: 287) | | | | | | |
| N: 319-365 | 18 | TPSGTWL | 4.78 | 4.13 | 5.87 | 35.11 | 4.13 | unassigned |
| | | TYTGAIK | | | | | | |
| | | LDDK | | | | | | |
| | | (SEQ ID | | | | | | |
| | | NO: 288) | | | | | | |
| N: 319-365 | 20 | TPSGTWL | 9.29 | 10.74 | 11.6 | 53.31 | 9.29 | unassigned |
| | | TYTGAIK | | | | | | |
| | | LDDKDP | | | | | | |
| | | (SEQ ID | | | | | | |
| | | NO: 289) | | | | | | |
| N: 319-365 | 21 | TPSGTWL | 13.95 | 16.11 | 22.95 | 68.72 | 13.95 | unassigned |
| | | TYTGAIK | | | | | | |
| | | LDDKDPN | | | | | | |
| | | (SEQ ID | | | | | | |
| | | NO: 290) | | | | | | |
| N: 319-365 | 22 | TPSGTWL | 13.23 | 15.58 | 21.76 | 67.28 | 13.23 | unassigned |
| | | TYTGAIK | | | | | | |
| | | LDDKDPN | | | | | | |
| | | E | | | | | | |
| | | (SEQ ID | | | | | | |
| | | NO: 291) | | | | | | |
| N: 373-419 | 32 | KKKADET | 7.84 | 26.31 | 41.16 | 1.87 | 1.87 | DQA10102_ |
| | | QALPQRQ | | | | | | DQB10602 |
| | | KKQQTVT | | | | | | |
| | | LLPAADL | | | | | | |
| | | DDES | | | | | | |
| | | (SEQ ID | | | | | | |
| | | NO: 292) | | | | | | |
| N: 373-419 | 29 | KKADETQ | 15.3 | 38 | 48.7 | 2.25 | 2.25 | unassigned |
| | | ALPQRQK | | | | | | |
| | | KQQTVTL | | | | | | |
| | | LPAADLD | | | | | | |
| | | D | | | | | | |
| | | (SEQ ID | | | | | | |
| | | NO: 293) | | | | | | |
| N: 373-419 | 27 | KADETQA | 39.88 | 54.96 | 59.34 | 2.67 | 2.67 | unassigned |
| | | LPQRQKK | | | | | | |
| | | QQTVTLL | | | | | | |
| | | PAADLD | | | | | | |
| | | (SEQ ID | | | | | | |
| | | NO: 294) | | | | | | |
| N: 373-419 | 28 | KADETQA | 15.69 | 40.32 | 49.92 | 2.41 | 2.41 | unassigned |
| | | LPQRQKK | | | | | | |
| | | QQTVTLL | | | | | | |
| | | PAADLDD | | | | | | |
| | | (SEQ ID | | | | | | |
| | | NO: 295) | | | | | | |
| N: 373-419 | 30 | KADETQA | 8.23 | 28.7 | 43.46 | 2.13 | 2.13 | unassigned |
| | | LPQRQKK | | | | | | |
| | | QQTVTLL | | | | | | |
| | | PAADLDD | | | | | | |
| | | ES | | | | | | |
| | | (SEQ ID | | | | | | |
| | | NO: 296) | | | | | | |
| N: 373-419 | 34 | KADETQA | 6.36 | 23.6 | 38.7 | 1.86 | 1.86 | DQA10102_ |
| | | LPQRQKK | | | | | | DQB10602 |
| | | QQTVTLL | | | | | | |
| | | PAADLDD | | | | | | |
| | | ESKQLQ | | | | | | |
| | | (SEQ ID | | | | | | |
| | | NO: 297) | | | | | | |
| N: 373-419 | 26 | ADETQAL | 40.66 | 60.38 | 60.93 | 2.9 | 2.9 | unassigned |
| | | PQRQKKQ | | | | | | |
| | | QTVTLLP | | | | | | |
| | | AADLD | | | | | | |
| | | (SEQ ID | | | | | | |
| | | NO: 298) | | | | | | |
| N: 373-419 | 27 | ADETQAL | 16.14 | 42.65 | 51.3 | 2.61 | 2.61 | unassigned |
| | | PQRQKKQ | | | | | | |
| | | QTVTLLP | | | | | | |
| | | AADLDD | | | | | | |
| | | (SEQ ID | | | | | | |
| | | NO: 299) | | | | | | |
| N: 373-419 | 29 | ADETQAL | 8.48 | 30 | 44.54 | 2.31 | 2.31 | unassigned |
| | | PQRQKKQ | | | | | | |
| | | QTVTLLP | | | | | | |
| | | AADLDDE | | | | | | |
| | | S | | | | | | |
| | | (SEQ ID | | | | | | |
| | | NO: 300) | | | | | | |
| N: 373-419 | 31 | ADETQAL | 7.18 | 26.51 | 41.35 | 2.13 | 2.13 | unassigned |
| | | PQRQKKQ | | | | | | |
| | | QTVTLLP | | | | | | |
| | | AADLDDE | | | | | | |
| | | SKQ | | | | | | |
| | | (SEQ ID | | | | | | |
| | | NO: 301) | | | | | | |
| N: 373-419 | 32 | ADETQAL | 6.82 | 25.43 | 40.39 | 2.06 | 2.06 | unassigned |
| | | PQRQKKQ | | | | | | |
| | | QTVTLLP | | | | | | |
| | | AADLDDE | | | | | | |
| | | SKQL | | | | | | |
| | | (SEQ ID | | | | | | |
| | | NO: 302) | | | | | | |
| N: 373-419 | 33 | ADETQAL | 6.54 | 24.57 | 39.64 | 2.01 | 2.01 | unassigned |
| | | PQRQKKQ | | | | | | |
| | | QTVTLLP | | | | | | |
| | | AADLDDE | | | | | | |
| | | SKQLQ | | | | | | |
| | | (SEQ ID | | | | | | |
| | | NO: 303) | | | | | | |
| N: 373-419 | 35 | ADETQAL | 6.11 | 23.46 | 37.99 | 1.94 | 1.94 | DQA10102_ |
| | | PQRQKKQ | | | | | | DQB10602 |
| | | QTVTLLP | | | | | | |
| | | AADLDDE | | | | | | |
| | | SKQLQQS | | | | | | |
| | | (SEQ ID | | | | | | |
| | | NO: 304) | | | | | | |
| N: 373-419 | 25 | DETQALP | 41.53 | 64.09 | 62.48 | 3.03 | 3.03 | unassigned |
| | | QRQKKQQ | | | | | | |
| | | TVTLLPA | | | | | | |
| | | ADLD | | | | | | |
| | | (SEQ ID | | | | | | |
| | | NO: 305) | | | | | | |
| N: 373-419 | 26 | DETQALP | 16.66 | 44.13 | 52.58 | 2.73 | 2.73 | unassigned |
| | | QRQKKQQ | | | | | | |
| | | TVTLLPA | | | | | | |
| | | ADLDD | | | | | | |
| | | (SEQ ID | | | | | | |
| | | NO: 306) | | | | | | |
| N: 373-419 | 30 | DETQALP | 7.4 | 27.36 | 42.44 | 2.22 | 2.22 | unassigned |
| | | QRQKKQQ | | | | | | |
| | | TVTLLPA | | | | | | |
| | | ADLDDES | | | | | | |
| | | KQ | | | | | | |
| | | (SEQ ID | | | | | | |
| | | NO: 307) | | | | | | |
| N: 373-419 | 32 | DETQALP | 6.75 | 25.36 | 40.66 | 2.11 | 2.11 | unassigned |
| | | QRQKKQQ | | | | | | |
| | | TVTLLPA | | | | | | |
| | | ADLDDES | | | | | | |
| | | KQLQ | | | | | | |
| | | (SEQ ID | | | | | | |
| | | NO: 308) | | | | | | |
| N: 373-419 | 34 | DETQALP | 6.32 | 24.17 | 38.93 | 2.03 | 2.03 | unassigned |
| | | QRQKKQQ | | | | | | |
| | | TVTLLPA | | | | | | |
| | | ADLDDES | | | | | | |
| | | KQLQQS | | | | | | |
| | | (SEQ ID | | | | | | |
| | | NO: 309) | | | | | | |
| N: 373-419 | 25 | QALPQRQ | 9.94 | 34.12 | 50.07 | 2.9 | 2.9 | unassigned |
| | | KKQQTVT | | | | | | |
| | | LLPAADL | | | | | | |
| | | DDES | | | | | | |
| | | (SEQ ID | | | | | | |
| | | NO: 310) | | | | | | |
| N: 373-419 | 21 | ALPQRQK | 46.27 | 70.54 | 70.26 | 4.11 | 4.11 | unassigned |
| | | KQQTVTL | | | | | | |
| | | LPAADLD | | | | | | |
| | | (SEQ ID | | | | | | |
| | | NO: 311) | | | | | | |
| N: 373-419 | 22 | ALPQRQK | 19.78 | 48.63 | 59.38 | 3.65 | 3.65 | unassigned |
| | | KQQTVTL | | | | | | |
| | | LPAADLD | | | | | | |
| | | D | | | | | | |
| | | (SEQ ID | | | | | | |
| | | NO: 312) | | | | | | |
| N: 373-419 | 24 | ALPQRQK | 10.56 | 35.59 | 52.2 | 3.18 | 3.18 | unassigned |
| | | KQQTVTL | | | | | | |
| | | LPAADLD | | | | | | |
| | | DES | | | | | | |
| | | (SEQ ID | | | | | | |
| | | NO: 313) | | | | | | |
| N: 373-419 | 25 | ALPQRQK | 9.5 | 33.29 | 49.79 | 3.02 | 3.02 | unassigned |
| | | KQQTVTL | | | | | | |
| | | LPAADLD | | | | | | |
| | | DESK | | | | | | |
| | | (SEQ ID | | | | | | |
| | | NO: 314) | | | | | | |
| N: 373-419 | 26 | ALPQRQK | 8.86 | 31.69 | 48.44 | 2.91 | 2.91 | unassigned |
| | | KQQTVTL | | | | | | |
| | | LPAADLD | | | | | | |
| | | DESKQ | | | | | | |
| | | (SEQ ID | | | | | | |
| | | NO: 315) | | | | | | |
| N: 373-419 | 27 | ALPQRQK | 8.38 | 30.47 | 47.4 | 2.84 | 2.84 | unassigned |
| | | KQQTVTL | | | | | | |
| | | LPAADLD | | | | | | |
| | | DESKQL | | | | | | |
| | | (SEQ ID | | | | | | |
| | | NO: 316) | | | | | | |
| N: 373-419 | 28 | ALPQRQK | 8 | 29.46 | 46.63 | 2.77 | 2.77 | unassigned |
| | | KQQTVTL | | | | | | |
| | | LPAADLD | | | | | | |
| | | DESKQLQ | | | | | | |
| | | (SEQ ID | | | | | | |
| | | NO: 317) | | | | | | |
| N: 373-419 | 29 | ALPQRQK | 7.77 | 28.73 | 45.56 | 2.72 | 2.72 | unassigned |
| | | KQQTVTL | | | | | | |
| | | LPAADLD | | | | | | |
| | | DESKQLQ | | | | | | |
| | | Q | | | | | | |
| | | (SEQ ID | | | | | | |
| | | NO: 318) | | | | | | |
| N: 373-419 | 30 | ALPQRQK | 7.52 | 28.13 | 44.58 | 2.67 | 2.67 | unassigned |
| | | KQQTVTL | | | | | | |
| | | LPAADLD | | | | | | |
| | | DESKQLQ | | | | | | |
| | | QS | | | | | | |
| | | (SEQ ID | | | | | | |
| | | NO: 319) | | | | | | |
| N: 373-419 | 31 | ALPQRQK | 7.27 | 27.64 | 43.75 | 2.63 | 2.63 | unassigned |
| | | KQQTVTL | | | | | | |
| | | LPAADLD | | | | | | |
| | | DESKQLQ | | | | | | |
| | | QSM | | | | | | |
| | | (SEQ ID | | | | | | |
| | | NO: 320) | | | | | | |
| N: 373-419 | 32 | ALPQRQK | 7.09 | 27.22 | 43.1 | 2.6 | 2.6 | unassigned |
| | | KQQTVTL | | | | | | |
| | | LPAADLD | | | | | | |
| | | DESKQLQ | | | | | | |
| | | QSMS | | | | | | |
| | | (SEQ ID | | | | | | |
| | | NO: 321) | | | | | | |
| N: 373-419 | 33 | ALPQRQK | 6.94 | 26.77 | 42.34 | 2.56 | 2.56 | unassigned |
| | | KQQTVTL | | | | | | |
| | | LPAADLD | | | | | | |
| | | DESKQLQ | | | | | | |
| | | QSMSS | | | | | | |
| | | (SEQ ID | | | | | | |
| | | NO: 322) | | | | | | |
| N: 373-419 | 34 | ALPQRQK | 6.83 | 25.86 | 40.72 | 2.53 | 2.53 | unassigned |
| | | KQQTVTL | | | | | | |
| | | LPAADLD | | | | | | |
| | | DESKQLQ | | | | | | |
| | | QSMSSA | | | | | | |
| | | (SEQ ID | | | | | | |
| | | NO: 323) | | | | | | |
| N: 373-419 | 35 | ALPQRQK | 6.73 | 25.42 | 39.94 | 2.5 | 2.5 | unassigned |
| | | KQQTVTL | | | | | | |
| | | LPAADLD | | | | | | |
| | | DESKQLQ | | | | | | |
| | | QSMSSAD | | | | | | |
| | | (SEQ ID | | | | | | |
| | | NO: 324) | | | | | | |
| N: 373-419 | 20 | LPQRQKK | 35.35 | 62.21 | 58.51 | 2.34 | 2.34 | unassigned |
| | | QQTVTLL | | | | | | |
| | | PAADLD | | | | | | |
| | | (SEQ ID | | | | | | |
| | | NO: 325) | | | | | | |
| N: 373-419 | 21 | LPQRQKK | 20.94 | 50.71 | 61.73 | 4.14 | 4.14 | unassigned |
| | | QQTVTLL | | | | | | |
| | | PAADLDD | | | | | | |
| | | (SEQ ID | | | | | | |
| | | NO: 326) | | | | | | |
| N: 373-419 | 16 | QRQKKQQ | 57.13 | 55.89 | 18.81 | 0.33 | 0.33 | DQA10102_ |
| | | TVTLLPA | | | | | | DQB10602 |
| | | AD | | | | | | |
| | | (SEQ ID | | | | | | |
| | | NO: 327) | | | | | | |
| N: 373-419 | 17 | QRQKKQQ | 50.41 | 58.56 | 25.89 | 0.51 | 0.51 | DQA10102_ |
| | | TVTLLPA | | | | | | DQB10602 |
| | | ADL | | | | | | |
| | | (SEQ ID | | | | | | |
| | | NO: 328) | | | | | | |
| N: 373-419 | 18 | QRQKKQQ | 17.75 | 42.35 | 34.73 | 0.76 | 0.76 | DQA10102_ |
| | | TVTLLPA | | | | | | DQB10602 |
| | | ADLD | | | | | | |
| | | (SEQ ID | | | | | | |
| | | NO: 329) | | | | | | |
| N: 373-419 | 19 | QRQKKQQ | 9.31 | 29.97 | 40.34 | 1.52 | 1.52 | DQA10102_ |
| | | TVTLLPA | | | | | | DQB10602 |
| | | ADLDD | | | | | | |
| | | (SEQ ID | | | | | | |
| | | NO: 330) | | | | | | |
| N: 373-419 | 20 | QRQKKQQ | 9.38 | 32.07 | 47.33 | 2.78 | 2.78 | unassigned |
| | | TVTLLPA | | | | | | |
| | | ADLDDE | | | | | | |
| | | (SEQ ID | | | | | | |
| | | NO: 331) | | | | | | |
| N: 373-419 | 21 | QRQKKQQ | 13.54 | 41.76 | 60.16 | 5.23 | 5.23 | unassigned |
| | | TVTLLPA | | | | | | |
| | | ADLDDES | | | | | | |
| | | (SEQ ID | | | | | | |
| | | NO: 332) | | | | | | |
| N: 373-419 | 22 | QRQKKQQ | 12.24 | 39.5 | 57.62 | 4.95 | 4.95 | unassigned |
| | | TVTLLPA | | | | | | |
| | | ADLDDES | | | | | | |
| | | K | | | | | | |
| | | (SEQ ID | | | | | | |
| | | NO: 333) | | | | | | |
| N: 373-419 | 23 | QRQKKQQ | 11.38 | 37.91 | 56.24 | 4.75 | 4.75 | unassigned |
| | | TVTLLPA | | | | | | |
| | | ADLDDES | | | | | | |
| | | KQ | | | | | | |
| | | (SEQ ID | | | | | | |
| | | NO: 334) | | | | | | |
| N: 373-419 | 24 | QRQKKQQ | 10.75 | 36.66 | 55.14 | 4.6 | 4.6 | unassigned |
| | | TVTLLPA | | | | | | |
| | | ADLDDES | | | | | | |
| | | KQL | | | | | | |
| | | (SEQ ID | | | | | | |
| | | NO: 335) | | | | | | |
| N: 373-419 | 25 | QRQKKQQ | 10.3 | 35.57 | 54.33 | 4.47 | 4.47 | unassigned |
| | | TVTLLPA | | | | | | |
| | | ADLDDES | | | | | | |
| | | KQLQ | | | | | | |
| | | (SEQ ID | | | | | | |
| | | NO: 336) | | | | | | |
| N: 373-419 | 26 | QRQKKQQ | 9.92 | 34.75 | 53.03 | 4.37 | 4.37 | unassigned |
| | | TVTLLPA | | | | | | |
| | | ADLDDES | | | | | | |
| | | KQLQQ | | | | | | |
| | | (SEQ ID | | | | | | |
| | | NO: 337) | | | | | | |
| N: 373-419 | 27 | QRQKKQQ | 9.55 | 34.06 | 51.8 | 4.28 | 4.28 | unassigned |
| | | TVTLLPA | | | | | | |
| | | ADLDDFS | | | | | | |
| | | KQLQQS | | | | | | |
| | | (SEQ ID | | | | | | |
| | | NO: 338) | | | | | | |
| N: 373-419 | 28 | QRQKKQQ | 9.14 | 33.53 | 50.74 | 4.21 | 4.21 | unassigned |
| | | TVTLLPA | | | | | | |
| | | ADLDDES | | | | | | |
| | | KQLQQSM | | | | | | |
| | | (SEQ ID | | | | | | |
| | | NO: 339) | | | | | | |
| N: 373-419 | 29 | QRQKKQQ | 8.9 | 33.05 | 49.92 | 4.15 | 4.15 | unassigned |
| | | TVTLLPA | | | | | | |
| | | ADLDDES | | | | | | |
| | | KQLQQSM | | | | | | |
| | | S | | | | | | |
| | | (SEQ ID | | | | | | |
| | | NO: 340) | | | | | | |
| N: 373-419 | 30 | QRQKKQQ | 8.71 | 32.5 | 48.97 | 4.07 | 4.07 | unassigned |
| | | TVTLLPA | | | | | | |
| | | ADLDDES | | | | | | |
| | | KQLQQSM | | | | | | |
| | | SS | | | | | | |
| | | (SEQ ID | | | | | | |
| | | NO: 341) | | | | | | |
| N: 373-419 | 31 | QRQKKQQ | 8.55 | 31.13 | 46.94 | 4.01 | 4.01 | unassigned |
| | | TVTLLPA | | | | | | |
| | | ADLDDES | | | | | | |
| | | KQLQQSM | | | | | | |
| | | SSA | | | | | | |
| | | (SEQ ID | | | | | | |
| | | NO: 342) | | | | | | |
| N: 373-419 | 32 | QRQKKQQ | 8.42 | 30.56 | 45.95 | 3.97 | 3.97 | unassigned |
| | | TVTLLPA | | | | | | |
| | | ADLDDES | | | | | | |
| | | KQLQQSM | | | | | | |
| | | SSAD | | | | | | |
| | | (SEQ ID | | | | | | |
| | | NO: 343) | | | | | | |
| N: 373-419 | 33 | QRQKKQQ | 8.3 | 30.09 | 45.12 | 3.93 | 3.93 | unassigned |
| | | TVTLLPA | | | | | | |
| | | ADLDDES | | | | | | |
| | | KQLQQSM | | | | | | |
| | | SSADS | | | | | | |
| | | (SEQ ID | | | | | | |
| | | NO: 344) | | | | | | |
| N: 373-419 | 34 | QRQKKQQ | 8.2 | 29.74 | 44.55 | 3.9 | 3.9 | unassigned |
| | | TVTLLPA | | | | | | |
| | | ADLDDES | | | | | | |
| | | KQLQQSM | | | | | | |
| | | SSADST | | | | | | |
| | | (SEQ ID | | | | | | |
| | | NO: 345) | | | | | | |
| N: 373-419 | 35 | QRQKKQQ | 8.11 | 29.37 | 43.5 | 3.86 | 3.86 | unassigned |
| | | TVTLLPA | | | | | | |
| | | ADLDDES | | | | | | |
| | | KQLQQSM | | | | | | |
| | | SSADSTQ | | | | | | |
| | | (SEQ ID | | | | | | |
| | | NO: 346) | | | | | | |
| N: 373-419 | 36 | QRQKKQQ | 8.03 | 29.04 | 43.13 | 3.81 | 3.81 | unassigned |
| | | TVTLLPA | | | | | | |
| | | ADLDDES | | | | | | |
| | | KQLQQSM | | | | | | |
| | | SSADSTQ | | | | | | |
| | | A | | | | | | |
| | | (SEQ ID | | | | | | |
| | | NO: 347) | | | | | | |
| N: 373-419 | 15 | RQKKQQT | 57.81 | 57.47 | 15.29 | 0.36 | 0.36 | DQA10102_ |
| | | VTLLPAA | | | | | | DQB10602 |
| | | D | | | | | | |
| | | (SEQ ID | | | | | | |
| | | NO: 348) | | | | | | |
| N: 373-419 | 16 | RQKKQQT | 45.96 | 57.63 | 19.53 | 0.36 | 0.36 | DQA10102_ |
| | | VTLLPAA | | | | | | DQB10602 |
| | | DL | | | | | | |
| | | (SEQ ID | | | | | | |
| | | NO: 349) | | | | | | |
| N: 373-419 | 17 | RQKKQQT | 12.59 | 33.52 | 26.12 | 0.56 | 0.56 | DQA10102_ |
| | | VTLLPAA | | | | | | DQB10602 |
| | | DLD | | | | | | |
| | | (SEQ ID | | | | | | |
| | | NO: 350) | | | | | | |
| N: 373-419 | 18 | RQKKQQT | 6.48 | 22.78 | 31.85 | 0.91 | 0.91 | DQA10102_ |
| | | VTLLPAA | | | | | | DQB10602 |
| | | DLDD | | | | | | |
| | | (SEQ ID | | | | | | |
| | | NO: 351) | | | | | | |
| N: 373-419 | 19 | RQKKQQT | 6.55 | 23.12 | 38.67 | 1.85 | 1.85 | DQA10102_ |
| | | VTLLPAA | | | | | | DQB10602 |
| | | DLDDE | | | | | | |
| | | (SEQ ID | | | | | | |
| | | NO: 352) | | | | | | |
| N: 373-419 | 20 | RQKKQQT | 9.13 | 31.73 | 49.01 | 3.52 | 3.52 | unassigned |
| | | VTLLPAA | | | | | | |
| | | DLDDES | | | | | | |
| | | (SEQ ID | | | | | | |
| | | NO: 353) | | | | | | |
| N: 373-419 | 22 | RQKKQQT | 12.87 | 40.77 | 60.31 | 6.43 | 6.43 | unassigned |
| | | VTLLPAA | | | | | | |
| | | DLDDESK | | | | | | |
| | | Q | | | | | | |
| | | (SEQ ID | | | | | | |
| | | NO: 354) | | | | | | |
| N: 373-419 | 24 | RQKKQQT | 11.68 | 38.53 | 58.42 | 6.03 | 6.03 | unassigned |
| | | VTLLPAA | | | | | | |
| | | DLDDESK | | | | | | |
| | | QLQ | | | | | | |
| | | (SEQ ID | | | | | | |
| | | NO: 355) | | | | | | |
| N: 373-419 | 14 | QKKQQTV | 56.59 | 60.66 | 14.75 | 0.25 | 0.25 | DQA10102_ |
| | | TLLPAAD | | | | | | DQB10602 |
| | | (SEQ ID | | | | | | |
| | | NO: 356) | | | | | | |
| N: 373-419 | 15 | QKKQQTV | 34.8 | 48.99 | 18.83 | 0.44 | 0.44 | DQA10102_ |
| | | TLLPAAD | | | | | | DQB10602 |
| | | L | | | | | | |
| | | (SEQ ID | | | | | | |
| | | NO: 357) | | | | | | |
| N: 373-419 | 16 | QKKQQTV | 9.84 | 26.84 | 22.68 | 0.44 | 0.44 | DQA10102_ |
| | | TLLPAAD | | | | | | DQB10602 |
| | | LD | | | | | | |
| | | (SEQ ID | | | | | | |
| | | NO: 358) | | | | | | |
| N: 373-419 | 18 | TVTLLPA | 58.52 | 70.82 | 80.39 | 50.8 | 50.8 | unassigned |
| | | ADLDDES | | | | | | |
| | | KQLQ | | | | | | |
| | | (SEQ ID | | | | | | |
| | | NO: 359) | | | | | | |
| N: 373-419 | 15 | LLPAADL | 53.96 | 83.22 | 79.11 | 88.44 | 53.96 | unassigned |
| | | DDESKQL | | | | | | |
| | | Q | | | | | | |
| | | (SEQ ID | | | | | | |
| | | NO: 360) | | | | | | |
| NA | 19 | THNSIKG | NA | NA | NA | NA | NA | NA |
| | | ADFLAPT | | | | | | |
| | | LIYHL | | | | | | |
| | | (SEQ ID | | | | | | |
| | | NO: 361) | | | | | | |
| NA | 24 | QSPYCLM | NA | NA | NA | NA | NA | NA |
| | | KPASASV | | | | | | |
| | | TLLKSRS | | | | | | |
| | | SIN | | | | | | |
| | | (SEQ ID | | | | | | |
| | | NO: 362) | | | | | | |
| nsp3: 174-189 | 16 | DGSEDNQ | 82.77 | 85.56 | 43.34 | 0.82 | 0.82 | DQA10102_ |
| | | TTTIQTI | | | | | | DQB10602 |
| | | VE | | | | | | |
| | | (SEQ ID | | | | | | |
| | | NO: 363) | | | | | | |
| nsp3: 661-677 | 17 | SPDAVTA | 0.03 | 25.57 | 13.04 | 47.03 | 0.03 | DRB1_ |
| | | YNGYLTS | | | | | | 1501 |
| | | SSK | | | | | | |
| | | (SEQ ID | | | | | | |
| | | NO: 364) | | | | | | |
| nsp4: 194-211 | 18 | MDGSIIQ | 0.01 | 13.41 | 32.43 | 63.78 | 0.01 | DRB1_ |
| | | FPNTYLE | | | | | | 1501 |
| | | GSVR | | | | | | |
| | | (SEQ ID | | | | | | |
| | | NO: 365) | | | | | | |
| nsp4: 194-211 | 15 | DGSIIQF | 0 | 5.46 | 17.81 | 47.38 | 0 | DRB1_ |
| | | PNTYLEG | | | | | | 1501 |
| | | S | | | | | | |
| | | (SEQ ID | | | | | | |
| | | NO: 366) | | | | | | |
| nsp4: 194-211 | 17 | DGSIIQF | 0 | 11.6 | 29.19 | 56.07 | 0 | DRB1_ |
| | | PNTYLEG | | | | | | 1501 |
| | | SVR | | | | | | |
| | | (SEQ ID | | | | | | |
| | | NO: 367) | | | | | | |
| nsp4: 194-211 | 14 | GSIIQFP | 0 | 10.49 | 27.38 | 53.5 | 0 | DRB1_ |
| | | NTYLEGS | | | | | | 1501 |
| | | (SEQ ID | | | | | | |
| | | NO: 368) | | | | | | |
| nsp4: 194-211 | 16 | GSIIQFP | 0.07 | 23.19 | 36.78 | 57.87 | 0.07 | DRB1_ |
| | | NTYLEGS | | | | | | 1501 |
| | | VR | | | | | | |
| | | (SEQ ID | | | | | | |
| | | NO: 369) | | | | | | |
| ORF3a: 1-28 | 28 | MDLFMRI | 38.22 | 8.09 | 60.15 | 12.81 | 8.09 | unassigned |
| | | FTIGTVT | | | | | | |
| | | LKQGEIK | | | | | | |
| | | DATPSDF | | | | | | |
| | | (SEQ ID | | | | | | |
| | | NO: 370) | | | | | | |
| ORF3a: 1-28 | 24 | MRIFTIG | 57.05 | 13.99 | 70.15 | 19 | 13.99 | unassigned |
| | | TVTLKQG | | | | | | |
| | | EIKDATP | | | | | | |
| | | SDF | | | | | | |
| | | (SEQ ID | | | | | | |
| | | NO: 371) | | | | | | |
| ORF3a: 1-28 | 22 | IFTIGTV | 65.59 | 19.38 | 78.56 | 26.32 | 19.38 | unassigned |
| | | TLKQGEI | | | | | | |
| | | KDATPSD | | | | | | |
| | | F | | | | | | |
| | | (SEQ ID | | | | | | |
| | | NO: 372) | | | | | | |
| ORF3a: 1-28 | 19 | IGTVTLK | 67.16 | 17.59 | 75.55 | 14.58 | 14.58 | unassigned |
| | | QGEIKDA | | | | | | |
| | | TPSDF | | | | | | |
| | | (SEQ ID | | | | | | |
| | | NO: 373) | | | | | | |
| ORF3a.iORF1: | 14 | ALHFLLF | 10.09 | 4.47 | 22.86 | 94.22 | 4.47 | unassigned |
| 28-41 | | FRALPKS | | | | | | |
| | | (SEQ ID | | | | | | |
| | | NO: 374) | | | | | | |
| ORF9b: 1-29 | 16 | MDPKISE | 15.51 | 0.59 | 16.11 | 27.41 | 0.59 | DRB5_ |
| | | MHPALRL | | | | | | 0101 |
| | | VD | | | | | | |
| | | (SEQ ID | | | | | | |
| | | NO: 375) | | | | | | |
| ORF9b: 1-29 | 17 | MDPKISE | 15.69 | 0.83 | 15.36 | 34.25 | 0.83 | DRB5_ |
| | | MHPALRL | | | | | | 0101 |
| | | VDP | | | | | | |
| | | (SEQ ID | | | | | | |
| | | NO: 376) | | | | | | |
| ORF9b: 1-29 | 18 | MDPKISE | 20.89 | 1.41 | 20.03 | 43.7 | 1.41 | DRB5_ |
| | | MHPALRL | | | | | | 0101 |
| | | VDPQ | | | | | | |
| | | (SEQ ID | | | | | | |
| | | NO: 377) | | | | | | |
| ORF9b: 1-29 | 20 | MDPKISE | 37.04 | 4.11 | 38.92 | 71.19 | 4.11 | unassigned |
| | | MHPALRL | | | | | | |
| | | VDPQIQ | | | | | | |
| | | (SEQ ID | | | | | | |
| | | NO: 378) | | | | | | |
| ORF9b: 1-29 | 21 | MDPKISE | 45.9 | 7.15 | 53.28 | 79.35 | 7.15 | unassigned |
| | | MHPALRL | | | | | | |
| | | VDPQIQL | | | | | | |
| | | (SEQ ID | | | | | | |
| | | NO: 379) | | | | | | |
| ORF9b: 1-29 | 22 | MDPKISE | 43.2 | 6.91 | 49.75 | 71.6 | 6.91 | unassigned |
| | | MHPALRL | | | | | | |
| | | VDPQIQL | | | | | | |
| | | A | | | | | | |
| | | (SEQ ID | | | | | | |
| | | NO: 380) | | | | | | |
| ORF9b: 1-29 | 28 | MDPKISE | 34.26 | 6.02 | 36.16 | 40.22 | 6.02 | unassigned |
| | | MHPALRL | | | | | | |
| | | VDPQIQL | | | | | | |
| | | AVTRMEN | | | | | | |
| | | (SEQ ID | | | | | | |
| | | NO: 381) | | | | | | |
| ORF9b: 1-29 | 29 | MDPKISE | 30.43 | 5.93 | 34.74 | 29.46 | 5.93 | unassigned |
| | | MHPALRL | | | | | | |
| | | VDPQIQL | | | | | | |
| | | AVTRMEN | | | | | | |
| | | A | | | | | | |
| | | (SEQ ID | | | | | | |
| | | NO: 382) | | | | | | |
| ORF9b: 37-53 | 16 | VGPKVYP | 24.83 | 39.11 | 8.07 | 1.88 | 1.88 | DQA10102_ |
| | | IILRLGS | | | | | | DQB10602 |
| | | PL | | | | | | |
| | | (SEQ ID | | | | | | |
| | | NO: 383) | | | | | | |
| ORF9b: 37-53 | 17 | VGPKVYP | 19.88 | 42.7 | 10.88 | 3.12 | 3.12 | unassigned |
| | | IILRLGS | | | | | | |
| | | PLS | | | | | | |
| | | (SEQ ID | | | | | | |
| | | NO: 384) | | | | | | |
| S: 186-216 | 26 | FKNLREF | 19.79 | 4.91 | 36.31 | 84.09 | 4.91 | unassigned |
| | | VEKNIDG | | | | | | |
| | | YFKIYSK | | | | | | |
| | | HTPIN | | | | | | |
| | | (SEQ ID | | | | | | |
| | | NO: 385) | | | | | | |
| S: 186-216 | 15 | NLREFVE | 22.79 | 6.92 | 5.2 | 66.96 | 5.2 | unassigned |
| | | KNIDGYF | | | | | | |
| | | K | | | | | | |
| | | (SEQ ID | | | | | | |
| | | NO: 386) | | | | | | |
| S: 186-216 | 17 | NLREFVE | 32.28 | 2.17 | 11.5 | 77.69 | 2.17 | unassigned |
| | | KNIDGYF | | | | | | |
| | | KIY | | | | | | |
| | | (SEQ ID | | | | | | |
| | | NO: 387) | | | | | | |
| S: 186-216 | 18 | NLREFVE | 37.22 | 1.76 | 22.41 | 84.07 | 1.76 | DRB5_ |
| | | KNIDGYF | | | | | | 0101 |
| | | KIYS | | | | | | |
| | | (SEQ ID | | | | | | |
| | | NO: 388) | | | | | | |
| S: 186-216 | 19 | NLREFVE | 36.02 | 2.83 | 38.64 | 90.73 | 2.83 | unassigned |
| | | KNIDGYF | | | | | | |
| | | KIYSK | | | | | | |
| | | (SEQ ID | | | | | | |
| | | NO: 389) | | | | | | |
| S: 186-216 | 20 | NLREFVE | 31.25 | 4.5 | 49.82 | 93.88 | 4.5 | unassigned |
| | | KNIDGYF | | | | | | |
| | | KIYSKH | | | | | | |
| | | (SEQ ID | | | | | | |
| | | NO: 390) | | | | | | |
| S: 186-216 | 21 | NLREFVE | 29.7 | 8.22 | 63.01 | 94.19 | 8.22 | unassigned |
| | | KNIDGYF | | | | | | |
| | | KIYSKHT | | | | | | |
| | | (SEQ ID | | | | | | |
| | | NO: 391) | | | | | | |
| S: 186-216 | 22 | NLREFVE | 27.36 | 7.74 | 56.6 | 89.39 | 7.74 | unassigned |
| | | KNIDGYF | | | | | | |
| | | KIYSKHT | | | | | | |
| | | P | | | | | | |
| | | (SEQ ID | | | | | | |
| | | NO: 392) | | | | | | |
| S: 186-216 | 23 | NLREFVE | 25.47 | 7.33 | 54.2 | 88.34 | 7.33 | unassigned |
| | | KNIDGYF | | | | | | |
| | | KIYSKHT | | | | | | |
| | | PI | | | | | | |
| | | (SEQ ID | | | | | | |
| | | NO: 393) | | | | | | |
| S: 186-216 | 24 | NLREFVE | 21.72 | 6.59 | 48.4 | 86.83 | 6.59 | unassigned |
| | | KNIDGYF | | | | | | |
| | | KIYSKHT | | | | | | |
| | | PIN | | | | | | |
| | | (SEQ ID | | | | | | |
| | | NO: 394) | | | | | | |
| S: 186-216 | 14 | LREFVEK | 28.33 | 5.47 | 3.56 | 61.13 | 3.56 | unassigned |
| | | NIDGYFK | | | | | | |
| | | (SEQ ID | | | | | | |
| | | NO: 395) | | | | | | |
| S: 186-216 | 16 | LREFVEK | 33.75 | 1.4 | 9.62 | 75.84 | 1.4 | DRB5_ |
| | | NIDGYFK | | | | | | 0101 |
| | | IY | | | | | | |
| | | (SEQ ID | | | | | | |
| | | NO: 396) | | | | | | |
| S: 186-216 | 17 | LREFVEK | 36.18 | 1.08 | 18.49 | 83.97 | 1.08 | DRB5_ |
| | | NIDGYFK | | | | | | 0101 |
| | | IYS | | | | | | |
| | | (SEQ ID | | | | | | |
| | | NO: 397) | | | | | | |
| S: 186-216 | 18 | LREFVEK | 27.7 | 1.89 | 31.38 | 89.43 | 1.89 | DRB5_ |
| | | NIDGYFK | | | | | | 0101 |
| | | IYSK | | | | | | |
| | | (SEQ ID | | | | | | |
| | | NO: 398) | | | | | | |
| S: 186-216 | 19 | LREFVEK | 25.22 | 3.25 | 46.26 | 90.71 | 3.25 | unassigned |
| | | NIDGYFK | | | | | | |
| | | IYSKH | | | | | | |
| | | (SEQ ID | | | | | | |
| | | NO: 399) | | | | | | |
| S: 186-216 | 21 | LREFVEK | 28.94 | 9.6 | 61.59 | 90.95 | 9.6 | unassigned |
| | | NIDGYFK | | | | | | |
| | | IYSKHTP | | | | | | |
| | | (SEQ ID | | | | | | |
| | | NO: 400) | | | | | | |
| S: 186-216 | 23 | LREFVEK | 23.03 | 8.09 | 52.52 | 88.2 | 8.09 | unassigned |
| | | NIDGYFK | | | | | | |
| | | IYSKHTP | | | | | | |
| | | IN | | | | | | |
| | | (SEQ ID | | | | | | |
| | | NO: 401) | | | | | | |
| S: 186-216 | 13 | REFVEKN | 30.23 | 3.76 | 4.36 | 62.14 | 3.76 | unassigned |
| | | IDGYFK | | | | | | |
| | | (SEQ ID | | | | | | |
| | | NO: 402) | | | | | | |
| S: 186-216 | 14 | REFVFKN | 30.16 | 1.09 | 5.3 | 66.63 | 1.09 | DRB5_ |
| | | IDGYFKI | | | | | | 0101 |
| | | (SEQ ID | | | | | | |
| | | NO: 403) | | | | | | |
| S: 186-216 | 15 | REFVEKN | 31.91 | 0.86 | 13.34 | 78.95 | 0.86 | DRB5_ |
| | | IDGYFKI | | | | | | 0101 |
| | | Y | | | | | | |
| | | (SEQ ID | | | | | | |
| | | NO: 404) | | | | | | |
| S: 186-216 | 16 | REFVEKN | 34.98 | 0.75 | 18.98 | 84.67 | 0.75 | DRB5_ |
| | | IDGYFKI | | | | | | 0101 |
| | | YS | | | | | | |
| | | (SEQ ID | | | | | | |
| | | NO: 405) | | | | | | |
| S: 186-216 | 17 | REFVEKN | 24.45 | 1.32 | 30.09 | 88.92 | 1.32 | DRB5_ |
| | | IDGYFKI | | | | | | 0101 |
| | | YSK | | | | | | |
| | | (SEQ ID | | | | | | |
| | | NO: 406) | | | | | | |
| S: 186-216 | 18 | REFVEKN | 17.78 | 2.44 | 42.87 | 87.02 | 2.44 | unassigned |
| | | IDGYFKI | | | | | | |
| | | YSKH | | | | | | |
| | | (SEQ ID | | | | | | |
| | | NO: 407) | | | | | | |
| S: 186-216 | 20 | REFVEKN | 22.97 | 7.13 | 52.49 | 86.53 | 7.13 | unassigned |
| | | IDGYFKI | | | | | | |
| | | YSKHTP | | | | | | |
| | | (SEQ ID | | | | | | |
| | | NO: 408) | | | | | | |
| S: 186-216 | 22 | REFVEKN | 24.73 | 10.52 | 56.51 | 89.31 | 10.52 | unassigned |
| | | IDGYFKI | | | | | | |
| | | YSKHTPI | | | | | | |
| | | N | | | | | | |
| | | (SEQ ID | | | | | | |
| | | NO: 409) | | | | | | |
| S: 186-216 | 12 | EFVEKNI | 66.83 | 6.86 | 41.29 | 92.47 | 6.86 | unassigned |
| | | DGYFK | | | | | | |
| | | (SEQ ID | | | | | | |
| | | NO: 410) | | | | | | |
| S: 186-216 | 14 | EFVEKNI | 42.6 | 0.72 | 31.54 | 89.69 | 0.72 | DRB5_ |
| | | DGYFKIY | | | | | | 0101 |
| | | (SEQ ID | | | | | | |
| | | NO: 411) | | | | | | |
| S: 186-216 | 15 | EFVEKNI | 33.55 | 0.58 | 36.83 | 92.35 | 0.58 | DRB5_ |
| | | DGYFKIY | | | | | | 0101 |
| | | S | | | | | | |
| | | (SEQ ID | | | | | | |
| | | NO: 412) | | | | | | |
| S: 186-216 | 16 | EFVEKNI | 20.08 | 1.09 | 37.68 | 92.51 | 1.09 | DRB5_ |
| | | DGYFKIY | | | | | | 0101 |
| | | SK | | | | | | |
| | | (SEQ ID | | | | | | |
| | | NO: 413) | | | | | | |
| S: 186-216 | 17 | EFVEKNI | 15.16 | 2.01 | 43.33 | 84.72 | 2.01 | unassigned |
| | | DGYFKIY | | | | | | |
| | | SKH | | | | | | |
| | | (SEQ ID | | | | | | |
| | | NO: 414) | | | | | | |
| S: 186-216 | 21 | EFVEKNI | 27 | 14.77 | 63.73 | 90.9 | 14.77 | unassigned |
| | | DGYFKIY | | | | | | |
| | | SKHTPIN | | | | | | |
| | | (SEQ ID | | | | | | |
| | | NO: 415) | | | | | | |
| S: 186-216 | 16 | FVFKNID | 12.15 | 5.52 | 42.51 | 79.68 | 5.52 | unassigned |
| | | GYFKIYS | | | | | | |
| | | KH | | | | | | |
| | | (SEQ ID | | | | | | |
| | | NO: 416) | | | | | | |
| S: 186-216 | 20 | FVFKNID | 21.33 | 15.48 | 52.41 | 85.73 | 15.48 | unassigned |
| | | GYFKIYS | | | | | | |
| | | KHTPIN | | | | | | |
| | | (SEQ ID | | | | | | |
| | | NO: 417) | | | | | | |
| S: 233-249 | 17 | INITRFQ | 5.69 | 0.95 | 16.07 | 27.51 | 0.95 | DRB5_ |
| | | TLLALHR | | | | | | 0101 |
| | | SYL | | | | | | |
| | | (SEQ ID | | | | | | |
| | | NO: 418) | | | | | | |
| S: 307-325 | 16 | VEKGIYQ | 0.45 | 5.02 | 0.3 | 19.67 | 0.3 | DPA10103_ |
| | | TSNERVQ | | | | | | DPB10402 |
| | | PT | | | | | | |
| | | (SEQ ID | | | | | | |
| | | NO: 419) | | | | | | |
| S: 307-325 | 17 | VEKGIYQ | 0.58 | 6.58 | 0.22 | 18.36 | 0.22 | DPA10103_ |
| | | TSNERVQ | | | | | | DPB10402 |
| | | PTE | | | | | | |
| | | (SEQ ID | | | | | | |
| | | NO: 420) | | | | | | |
| S: 307-325 | 18 | VEKGIYQ | 0.77 | 9.86 | 0.4 | 21.6 | 0.4 | DPA10103_ |
| | | TSNERVQ | | | | | | DPB10402 |
| | | PTES | | | | | | |
| | | (SEQ ID | | | | | | |
| | | NO: 421) | | | | | | |
| S: 344-360 | 14 | ATRFASV | 12.02 | 0.03 | 3.84 | 36.34 | 0.03 | DRB5_ |
| | | YAWNRKR | | | | | | 0101 |
| | | (SEQ ID | | | | | | |
| | | NO: 422) | | | | | | |
| S: 344-360 | 16 | ATREASV | 13.08 | 0.13 | 7.12 | 52.85 | 0.13 | DRB5_ |
| | | YAWNRKR | | | | | | 0101 |
| | | IS | | | | | | |
| | | (SEQ ID | | | | | | |
| | | NO: 423) | | | | | | |
| S: 344-360 | 17 | ATRFASV | 12.61 | 0.29 | 11.87 | 66.44 | 0.29 | DRB5_ |
| | | YAWNRKR | | | | | | 0101 |
| | | ISN | | | | | | |
| | | (SEQ ID | | | | | | |
| | | NO: 424) | | | | | | |
| S: 398-413 | 16 | DSFVIRG | 13.7 | 9.95 | 6.79 | 8.3 | 6.79 | unassigned |
| | | DEVRQIA | | | | | | |
| | | PG | | | | | | |
| | | (SEQ ID | | | | | | |
| | | NO: 425) | | | | | | |
| S: 422-446 | 20 | NYKLPDD | 44.83 | 59.89 | 56.18 | 24.68 | 24.68 | unassigned |
| | | ETGCVIA | | | | | | |
| | | WNSNNL | | | | | | |
| | | (SEQ ID | | | | | | |
| | | NO: 426) | | | | | | |
| S: 422-446 | 24 | NYKLPDD | 0.49 | 45.56 | 56.02 | 34.5 | 0.49 | DRB1_ |
| | | ETGCVIA | | | | | | 1501 |
| | | WNSNNLD | | | | | | |
| | | SKV | | | | | | |
| | | (SEQ ID | | | | | | |
| | | NO: 427) | | | | | | |
| S: 422-446 | 22 | YKLPDDE | 0.66 | 51.8 | 64.38 | 38.65 | 0.66 | DRB1_ |
| | | TGCVIAW | | | | | | 1501 |
| | | NSNNLDS | | | | | | |
| | | K | | | | | | |
| | | (SEQ ID | | | | | | |
| | | NO: 428) | | | | | | |
| S: 422-446 | 24 | YKLPDDF | 0.47 | 44.31 | 54.66 | 36.87 | 0.47 | DRB11501 |
| | | TGCVIAW | | | | | | |
| | | NSNNLDS | | | | | | |
| | | KVG | | | | | | |
| | | (SEQ ID | | | | | | |
| | | NO: 429) | | | | | | |
| S: 422-446 | 21 | KLPDDFT | 0.74 | 54.24 | 68.84 | 42.77 | 0.74 | DRB1_ |
| | | GCVIAWN | | | | | | 1501 |
| | | SNNLDSK | | | | | | |
| | | (SEQ ID | | | | | | |
| | | NO: 430) | | | | | | |
| S: 422-446 | 23 | KLPDDFT | 0.53 | 46.64 | 58.61 | 40.94 | 0.53 | DRB1_ |
| | | GCVIAWN | | | | | | 1501 |
| | | SNNLDSK | | | | | | |
| | | VG | | | | | | |
| | | (SEQ ID | | | | | | |
| | | NO: 431) | | | | | | |
| S: 422-446 | 18 | LPDDETG | 2.05 | 47.56 | 35.02 | 13.99 | 2.05 | unassigned |
| | | CVIAWNS | | | | | | |
| | | NNLD | | | | | | |
| | | (SEQ ID | | | | | | |
| | | NO: 432) | | | | | | |
| S: 422-446 | 20 | LPDDETG | 0.45 | 44.42 | 51.32 | 31.94 | 0.45 | DRB1_ |
| | | CVIAWNS | | | | | | 1501 |
| | | NNLDSK | | | | | | |
| | | (SEQ ID | | | | | | |
| | | NO: 433) | | | | | | |
| S: 422-446 | 22 | LPDDETG | 0.6 | 49.33 | 62.89 | 46.46 | 0.6 | DRB1_ |
| | | CVIAWNS | | | | | | 1501 |
| | | NNLDSKV | | | | | | |
| | | G | | | | | | |
| | | (SEQ ID | | | | | | |
| | | NO: 434) | | | | | | |
| S: 422-446 | 17 | DETGCVI | 0.1 | 19.58 | 29.28 | 57.84 | 0.1 | DRB1_ |
| | | AWNSNNL | | | | | | 1501 |
| | | DSK | | | | | | |
| | | (SEQ ID | | | | | | |
| | | NO: 435) | | | | | | |
| S: 422-446 | 16 | FTGCVIA | 0.08 | 16.1 | 23.55 | 61.17 | 0.08 | DRB1_ |
| | | WNSNNLD | | | | | | 1501 |
| | | SK | | | | | | |
| | | (SEQ ID | | | | | | |
| | | NO: 436) | | | | | | |
| S: 422-446 | 15 | TGCVIAW | 0.07 | 13.61 | 22.22 | 59.22 | 0.07 | DRB1_ |
| | | NSNNLDS | | | | | | 1501 |
| | | K | | | | | | |
| | | (SEQ ID | | | | | | |
| | | NO: 437) | | | | | | |
| S: 422-446 | 14 | GCVIAWN | 0.22 | 22.23 | 31.7 | 58.79 | 0.22 | DRB1_ |
| | | SNNLDSK | | | | | | 1501 |
| | | (SEQ ID | | | | | | |
| | | NO: 438) | | | | | | |
| S: 422-446 | 16 | GCVIAWN | 0.72 | 34.02 | 36.95 | 65.04 | 0.72 | DRB1_ |
| | | SNNLDSK | | | | | | 1501 |
| | | VG | | | | | | |
| | | (SEQ ID | | | | | | |
| | | NO: 439) | | | | | | |
| S: 470-485 | 16 | TEIYQAG | 34.99 | 33.65 | 85.37 | 7.82 | 7.82 | unassigned |
| | | STPCNGV | | | | | | |
| | | EG | | | | | | |
| | | (SEQ ID | | | | | | |
| | | NO: 440) | | | | | | |
| S: 470-485 | 15 | EIYQAGS | 61.52 | 52 | 93.36 | 7.28 | 7.28 | unassigned |
| | | TPCNGVE | | | | | | |
| | | G | | | | | | |
| | | (SEQ ID | | | | | | |
| | | NO: 441) | | | | | | |
| S: 554-581 | 28 | ESNKKEL | 9.87 | 7.69 | 26.69 | 32.74 | 7.69 | unassigned |
| | | PFQQFGR | | | | | | |
| | | DIADTTD | | | | | | |
| | | AVRDPQT | | | | | | |
| | | (SEQ ID | | | | | | |
| | | NO: 442) | | | | | | |
| S: 630-648 | 15 | WRVYSTG | 43.94 | 33.18 | 69.21 | 1.04 | 1.04 | DQA10102_ |
| | | SNVFQTR | | | | | | DQB10602 |
| | | A | | | | | | |
| | | (SEQ ID | | | | | | |
| | | NO: 443) | | | | | | |
| S: 630-648 | 16 | WRVYSTG | 56.28 | 45.7 | 68.92 | 0.51 | 0.51 | DQA10102_ |
| | | SNVFQTR | | | | | | DQB10602 |
| | | AG | | | | | | |
| | | (SEQ ID | | | | | | |
| | | NO: 444) | | | | | | |
| S: 670-684 | 15 | ICASYQT | 40.44 | 0.64 | 63.51 | 59.39 | 0.64 | DRB5_ |
| | | QTNSPRR | | | | | | 0101 |
| | | A | | | | | | |
| | | (SEQ ID | | | | | | |
| | | NO: 445) | | | | | | |
| S: 686-703 | 18 | SVASQSI | 2.74 | 49.07 | 41.29 | 30.16 | 2.74 | unassigned |
| | | IAYTMSL | | | | | | |
| | | GAEN | | | | | | |
| | | (SEQ ID | | | | | | |
| | | NO: 446) | | | | | | |
| S: 686-703 | 13 | ASQSIIA | 12.29 | 82.64 | 83.95 | 62.71 | 12.29 | unassigned |
| | | YTMSLG | | | | | | |
| | | (SEQ ID | | | | | | |
| | | NO: 447) | | | | | | |
| S: 686-703 | 16 | ASQSIIA | 1.61 | 26.04 | 19.32 | 14.13 | 1.61 | DRB1_ |
| | | YTMSLGA | | | | | | 1501 |
| | | EN | | | | | | |
| | | (SEQ ID | | | | | | |
| | | NO: 448) | | | | | | |
| S: 749-780 | 14 | CSNLLLQ | 7.32 | 90.22 | 81.23 | 95 | 7.32 | unassigned |
| | | YGSFCTQ | | | | | | |
| | | (SEQ ID | | | | | | |
| | | NO: 449) | | | | | | |
| S: 749-780 | 13 | SNLLLQY | 6.24 | 87.34 | 76.28 | 95 | 6.24 | unassigned |
| | | GSFCTQ | | | | | | |
| | | (SEQ ID | | | | | | |
| | | NO: 450) | | | | | | |
| S: 749-780 | 14 | SNLLLQY | 7.92 | 90.65 | 77.19 | 95 | 7.92 | unassigned |
| | | GSFCTQL | | | | | | |
| | | (SEQ ID | | | | | | |
| | | NO: 451) | | | | | | |
| S: 749-780 | 15 | SNLLLQY | 4.93 | 81.63 | 76.31 | 95 | 4.93 | unassigned |
| | | GSFCTQL | | | | | | |
| | | N | | | | | | |
| | | (SEQ ID | | | | | | |
| | | NO: 452) | | | | | | |
| S: 749-780 | 16 | SNLLLQY | 8.39 | 73.28 | 60.26 | 89.74 | 8.39 | unassigned |
| | | GSFCTQL | | | | | | |
| | | NR | | | | | | |
| | | (SEQ ID | | | | | | |
| | | NO: 453) | | | | | | |
| S: 749-780 | 18 | LNRALTG | 23.72 | 48.58 | 24.05 | 3.13 | 3.13 | unassigned |
| | | IAVEQDK | | | | | | |
| | | NTQE | | | | | | |
| | | (SEQ ID | | | | | | |
| | | NO: 454) | | | | | | |
| S: 749-780 | 14 | NRALTGI | 35.63 | 35.99 | 9.64 | 1.23 | 1.23 | DQA10102_ |
| | | AVEQDKN | | | | | | DQB10602 |
| | | (SEQ ID | | | | | | |
| | | NO: 455) | | | | | | |
| S: 749-780 | 15 | NRALTGI | 28.94 | 32.2 | 8.69 | 1.14 | 1.14 | DQA10102_ |
| | | AVEQDKN | | | | | | DQB10602 |
| | | T | | | | | | |
| | | (SEQ ID | | | | | | |
| | | NO: 456) | | | | | | |
| S: 749-780 | 16 | NRALTGI | 26.71 | 38.97 | 12.25 | 1.4 | 1.4 | DQA10102_ |
| | | AVEQDKN | | | | | | DQB10602 |
| | | TQ | | | | | | |
| | | (SEQ ID | | | | | | |
| | | NO: 457) | | | | | | |
| S: 749-780 | 14 | RALTGIA | 48.2 | 47.15 | 18.53 | 2.15 | 2.15 | unassigned |
| | | VEQDKNT | | | | | | |
| | | (SEQ ID | | | | | | |
| | | NO: 458) | | | | | | |
| S: 860-883 | 20 | VLPPLLT | 2.94 | 79.92 | 80.74 | 90.51 | 2.94 | unassigned |
| | | DEMIAQY | | | | | | |
| | | TSALLA | | | | | | |
| | | (SEQ ID | | | | | | |
| | | NO: 459) | | | | | | |
| S: 860-883 | 24 | VLPPLLT | 0.6 | 63.16 | 73.74 | 47.42 | 0.6 | DRB11501 |
| | | DEMIAQY | | | | | | |
| | | TSALLAG | | | | | | |
| | | TIT | | | | | | |
| | | (SEQ ID | | | | | | |
| | | NO: 460) | | | | | | |
| S: 860-883 | 21 | LPPLLTD | 0.81 | 68.97 | 86.61 | 74.97 | 0.81 | DRB1_ |
| | | EMIAQYT | | | | | | 1501 |
| | | SALLAGT | | | | | | |
| | | (SEQ ID | | | | | | |
| | | NO: 461) | | | | | | |
| S: 860-883 | 23 | LPPLLTD | 0.64 | 64.98 | 75.88 | 49.21 | 0.64 | DRB1_ |
| | | EMIAQYT | | | | | | 1501 |
| | | SALLAGT | | | | | | |
| | | IT | | | | | | |
| | | (SEQ ID | | | | | | |
| | | NO: 462) | | | | | | |
| S: 860-883 | 20 | PPLLTDE | 0.43 | 57.21 | 72.28 | 53.38 | 0.43 | DRB1_ |
| | | MIAQYTS | | | | | | 1501 |
| | | ALLAGT | | | | | | |
| | | (SEQ ID | | | | | | |
| | | NO: 463) | | | | | | |
| S: 860-883 | 15 | LTDEMIA | 0.22 | 32.43 | 56.07 | 53.97 | 0.22 | DRB1_ |
| | | QYTSALL | | | | | | 1501 |
| | | A | | | | | | |
| | | (SEQ ID | | | | | | |
| | | NO: 464) | | | | | | |
| S: 860-883 | 16 | LTDEMIA | 0.11 | 26.47 | 38.58 | 24.11 | 0.11 | DRB1_ |
| | | QYTSALL | | | | | | 1501 |
| | | AG | | | | | | |
| | | (SEQ ID | | | | | | |
| | | NO: 465) | | | | | | |
| S: 860-883 | 17 | LTDEMIA | 0.08 | 25.52 | 34.64 | 14.5 | 0.08 | DRB1_ |
| | | QYTSALL | | | | | | 1501 |
| | | AGT | | | | | | |
| | | (SEQ ID | | | | | | |
| | | NO: 466) | | | | | | |
| S: 860-883 | 19 | LTDEMIA | 0.28 | 51.54 | 54.4 | 22.53 | 0.28 | DRB1_ |
| | | QYTSALL | | | | | | 1501 |
| | | AGTIT | | | | | | |
| | | (SEQ ID | | | | | | |
| | | NO: 467) | | | | | | |
| S: 860-883 | 13 | TDEMIAQ | 1.09 | 46.95 | 73.18 | 75.43 | 1.09 | DRB1_ |
| | | YTSALL | | | | | | 1501 |
| | | (SEQ ID | | | | | | |
| | | NO: 468) | | | | | | |
| S: 860-883 | 14 | TDEMIAQ | 0.17 | 31.46 | 47.84 | 46.01 | 0.17 | DRB1_ |
| | | YTSALLA | | | | | | 1501 |
| | | (SEQ ID | | | | | | |
| | | NO: 469) | | | | | | |
| S: 860-883 | 15 | TDEMIAQ | 0.05 | 18.76 | 29.95 | 16.71 | 0.05 | DRB1_ |
| | | YTSALLA | | | | | | 1501 |
| | | G | | | | | | |
| | | (SEQ ID | | | | | | |
| | | NO: 470) | | | | | | |
| S: 860-883 | 16 | TDEMIAQ | 0.06 | 23.43 | 28.22 | 10.05 | 0.06 | DRB1_ |
| | | YTSALLA | | | | | | 1501 |
| | | GT | | | | | | |
| | | (SEQ ID | | | | | | |
| | | NO: 471) | | | | | | |
| S: 860-883 | 17 | TDEMIAQ | 0.09 | 29.41 | 30.07 | 11.55 | 0.09 | DRB1_ |
| | | YTSALLA | | | | | | 1501 |
| | | GTI | | | | | | |
| | | (SEQ ID | | | | | | |
| | | NO: 472) | | | | | | |
| S: 860-883 | 18 | TDEMIAQ | 0.2 | 43.48 | 39.6 | 14.42 | 0.2 | DRB1_ |
| | | YTSALLA | | | | | | 1501 |
| | | GTIT | | | | | | |
| | | (SEQ ID | | | | | | |
| | | NO: 473) | | | | | | |
| S: 860-883 | 12 | DEMIAQY | 1.76 | 66.39 | 88.42 | 92.8 | 1.76 | DRB1_ |
| | | TSALL | | | | | | 1501 |
| | | (SEQ ID | | | | | | |
| | | NO: 474) | | | | | | |
| S: 860-883 | 13 | DEMIAQY | 0.13 | 33.37 | 46.08 | 41.35 | 0.13 | DRB1_ |
| | | TSALLA | | | | | | 1501 |
| | | (SEQ ID | | | | | | |
| | | NO: 475) | | | | | | |
| S: 860-883 | 14 | DEMIAQY | 0.05 | 24.14 | 31.1 | 14.46 | 0.05 | DRB1_ |
| | | TSALLAG | | | | | | 1501 |
| | | (SEQ ID | | | | | | |
| | | NO: 476) | | | | | | |
| S: 860-883 | 15 | DEMIAQY | 0.03 | 18.22 | 22.92 | 6.92 | 0.03 | DRB1_ |
| | | TSALLAG | | | | | | 1501 |
| | | T | | | | | | |
| | | (SEQ ID | | | | | | |
| | | NO: 477) | | | | | | |
| S: 860-883 | 16 | DEMIAQY | 0.08 | 31.68 | 23.98 | 8.46 | 0.08 | DRB1_ |
| | | TSALLAG | | | | | | 1501 |
| | | TI | | | | | | |
| | | (SEQ ID | | | | | | |
| | | NO: 478) | | | | | | |
| S: 860-883 | 17 | DEMIAQY | 0.12 | 38.94 | 23.85 | 8.47 | 0.12 | DRB1_ |
| | | TSALLAG | | | | | | 1501 |
| | | TIT | | | | | | |
| | | (SEQ ID | | | | | | |
| | | NO: 479) | | | | | | |
| S: 860-883 | 12 | EMIAQYT | 0.72 | 67.39 | 83.52 | 56.37 | 0.72 | DRB1_ |
| | | SALLA | | | | | | 1501 |
| | | (SEQ ID | | | | | | |
| | | NO: 480) | | | | | | |
| S: 860-883 | 13 | EMIAQYT | 0.19 | 41.16 | 47.46 | 12.42 | 0.19 | DRB1_ |
| | | SALLAG | | | | | | 1501 |
| | | (SEQ ID | | | | | | |
| | | NO: 481) | | | | | | |
| S: 860-883 | 14 | EMIAQYT | 0.15 | 39.95 | 32.14 | 5.9 | 0.15 | DRB1_ |
| | | SALLAGT | | | | | | 1501 |
| | | (SEQ ID | | | | | | |
| | | NO: 482) | | | | | | |
| S: 860-883 | 16 | EMIAQYT | 0.54 | 54.58 | 18.5 | 6.56 | 0.54 | DRB1_ |
| | | SALLAGT | | | | | | 1501 |
| | | IT | | | | | | |
| | | (SEQ ID | | | | | | |
| | | NO: 483) | | | | | | |
| S: 892-910 | 14 | AALQIPF | 60.43 | 72.13 | 57.87 | 15.28 | 15.28 | unassigned |
| | | AMQMAYR | | | | | | |
| | | (SEQ ID | | | | | | |
| | | NO: 484) | | | | | | |
| S: 892-910 | 16 | AALQIPF | 26.81 | 7.71 | 24.81 | 26.66 | 7.71 | unassigned |
| | | AMQMAYR | | | | | | |
| | | EN | | | | | | |
| | | (SEQ ID | | | | | | |
| | | NO: 485) | | | | | | |
| S: 892-910 | 14 | LQIPFAM | 16.69 | 3.91 | 12.63 | 69.76 | 3.91 | unassigned |
| | | QMAYREN | | | | | | |
| | | (SEQ ID | | | | | | |
| | | NO: 486) | | | | | | |
| S: 892-910 | 15 | LQIPFAM | 10.57 | 2.44 | 9.03 | 69.17 | 2.44 | unassigned |
| | | QMAYREN | | | | | | |
| | | G | | | | | | |
| | | (SEQ ID | | | | | | |
| | | NO: 487) | | | | | | |
| S: 892-910 | 17 | LQIPFAM | 15.78 | 4.55 | 15.2 | 77.5 | 4.55 | unassigned |
| | | QMAYREN | | | | | | |
| | | GIG | | | | | | |
| | | (SEQ ID | | | | | | |
| | | NO: 488) | | | | | | |
| S: 892-910 | 13 | QIPFAMQ | 14.62 | 3.22 | 9.1 | 79.59 | 3.22 | unassigned |
| | | MAYREN | | | | | | |
| | | (SEQ ID | | | | | | |
| | | NO: 489) | | | | | | |
| S: 892-910 | 14 | QIPFAMQ | 9.73 | 2.09 | 6.24 | 69.46 | 2.09 | unassigned |
| | | MAYRENG | | | | | | |
| | | (SEQ ID | | | | | | |
| | | NO: 490) | | | | | | |
| S: 892-910 | 16 | QIPFAMQ | 15.27 | 4.26 | 12.45 | 72.29 | 4.26 | unassigned |
| | | MAYRENG | | | | | | |
| | | IG | | | | | | |
| | | (SEQ ID | | | | | | |
| | | NO: 491) | | | | | | |
| S: 892-910 | 12 | IPFAMQM | 41.11 | 11.49 | 34.11 | 94.6 | 11.49 | unassigned |
| | | AYREN | | | | | | |
| | | (SEQ ID | | | | | | |
| | | NO: 492) | | | | | | |
| S: 892-910 | 13 | IPFAMQM | 18.43 | 4.07 | 12.92 | 80.82 | 4.07 | unassigned |
| | | AYRENG | | | | | | |
| | | (SEQ ID | | | | | | |
| | | NO: 493) | | | | | | |
| S: 892-910 | 15 | IPFAMQM | 26.66 | 7.65 | 26.27 | 75.73 | 7.65 | unassigned |
| | | AYRENGI | | | | | | |
| | | G | | | | | | |
| | | (SEQ ID | | | | | | |
| | | NO: 494) | | | | | | |
| S: 1009-1025 | 13 | TQQLIRA | 6.93 | 14.93 | 21.47 | 0.15 | 0.15 | DQA10102_ |
| | | AEIRAS | | | | | | DQB10602 |
| | | (SEQ ID | | | | | | |
| | | NO: 495) | | | | | | |
| S: 1009-1025 | 15 | TQQLIRA | 3.15 | 5.91 | 4.8 | 0 | 0 | DQA10102_ |
| | | AEIRASA | | | | | | DQB10602 |
| | | N | | | | | | |
| | | (SEQ ID | | | | | | |
| | | NO: 496) | | | | | | |
| S: 1009-1025 | 16 | TQQLIRA | 4.04 | 8.41 | 5.85 | 0.01 | 0.01 | DQA10102_ |
| | | AEIRASA | | | | | | DQB10602 |
| | | NL | | | | | | |
| | | (SEQ ID | | | | | | |
| | | NO: 497) | | | | | | |
| S: 1009-1025 | 17 | TQQLIRA | 5.39 | 11.61 | 9.37 | 0.03 | 0.03 | DQA10102_ |
| | | AEIRASA | | | | | | DQB10602 |
| | | NLA | | | | | | |
| | | (SEQ ID | | | | | | |
| | | NO: 498) | | | | | | |
| S: 1009-1025 | 12 | QQLIRAA | 20.37 | 34.9 | 42.26 | 0.29 | 0.29 | DQA10102_ |
| | | EIRAS | | | | | | DQB10602 |
| | | (SEQ ID | | | | | | |
| | | NO: 499) | | | | | | |
| S: 1009-1025 | 13 | QQLIRAA | 7.26 | 12.55 | 8.92 | 0 | 0 | DQA10102_ |
| | | EIRASA | | | | | | DQB10602 |
| | | (SEQ ID | | | | | | |
| | | NO: 500) | | | | | | |
| S: 1009-1025 | 14 | QQLIRAA | 5.42 | 8.2 | 5.36 | 0 | 0 | DQA10102_ |
| | | EIRASAN | | | | | | DQB10602 |
| | | (SEQ ID | | | | | | |
| | | NO: 501) | | | | | | |
| S: 1009-1025 | 15 | QQLIRAA | 7 | 10.61 | 6.4 | 0.01 | 0.01 | DQA10102_ |
| | | EIRASAN | | | | | | DQB10602 |
| | | L | | | | | | |
| | | (SEQ ID | | | | | | |
| | | NO: 502) | | | | | | |
| S: 1009-1025 | 16 | QQLIRAA | 9.99 | 16.27 | 9.09 | 0.02 | 0.02 | DQA10102_ |
| | | EIRASAN | | | | | | DQB10602 |
| | | LA | | | | | | |
| | | (SEQ ID | | | | | | |
| | | NO: 503) | | | | | | |
| S: 1009-1025 | 11 | QLIRAAE | 70.75 | 78.59 | 86.12 | 1.9 | 1.9 | DQA10102_ |
| | | IRAS | | | | | | DQB10602 |
| | | (SEQ ID | | | | | | |
| | | NO: 504) | | | | | | |
| S: 1009-1025 | 10 | LIRAAEI | 95 | 95 | 95 | 16.4 | 16.4 | unassigned |
| | | RAS | | | | | | |
| | | (SEQ ID | | | | | | |
| | | NO: 505) | | | | | | |
| S: 1009-1025 | 11 | LIRAAEI | 88.24 | 89.71 | 87.44 | 2.23 | 2.23 | unassigned |
| | | RASA | | | | | | |
| | | (SEQ ID | | | | | | |
| | | NO: 506) | | | | | | |
| S: 1009-1025 | 12 | LIRAAEI | 66.1 | 71.65 | 65.04 | 0.53 | 0.53 | DQA10102_ |
| | | RASAN | | | | | | DQB10602 |
| | | (SEQ ID | | | | | | |
| | | NO: 507) | | | | | | |
| S: 1009-1025 | 13 | LIRAAEI | 55.37 | 63.56 | 54.23 | 0.4 | 0.4 | DQA10102_ |
| | | RASANL | | | | | | DQB10602 |
| | | (SEQ ID | | | | | | |
| | | NO: 508) | | | | | | |

This result aligns with the overall low percentage of viral peptides reported in previous studies that investigated the HLA-II immunopeptidome of EBV-B cells infected with measles virus (Ovsyannikova et al., 2003) and Monocyte-derived DCs (moDC) pulsed with recombinant H1-HA protein of influenza virus (Cassotta et al., 2020). Furthermore, the recovered HLA-II peptides exhibited the expected 12-25 amino acid length distribution (FIG. 1D). Applicant examined the amino acid sequences of detected peptides and checked if these sequences conformed to the binding preferences of the HLA-II alleles expressed in A549 and HEK293T cells. The binding peptide sequences, deconvoluted by Gibbs Cluster (Andreatta et al., 2017), agreed with known preferences of HLA-DR heterodimers (Abelin et al., 2019) expressed in these cell lines (FIG. 1E, Table 3). As expected, the deconvoluted peptide sequences matched more to HLA-DR heterodimers, and less so to HLA-DP and HLA-DQ heterodimers, in both cell lines, as HLA-DR is often expressed at higher levels when compared to HLA-DP and HLA-DQ (Taylor et al., 2021). Notably, some of the HLA-II alleles expressed by the two cell lines, and confirmed by peptide sequence deconvolution are highly prevalent in the European (EUR) and United States (USA) populations including DRB1*07:01 (˜14% EUR, 12% ˜USA) expressed by A549 and DRB1*15:01 (˜14% EUR, ˜11% USA) and DRB5*01:01 (˜16% EUR) expressed by HEK293T (Table 3).

TABLE 3
HLA-II alleles present in A549 and HEK293T cells used in this study.

| Cell | | Population | | | | |
|---|---|---|---|---|---|---|
| line | Allele | EUR | AFA | API | HIS | USA |
| A549 | DRB1*07:01 | 13.8% | 9.8% | 8.2% | 10.5% | 12.3% |
| | DRB1*11:04 | 3.2% | 0.6% | 0.7% | 2.6% | 2.6% |
| | DRB3*02:02 | 16.4% | | | | |
| | DRB4*01:01 | 28.0% | | | | |
| | DPB1*03:01 DPA1*01:03 | 10.0% | | 0.0% | | |
| | DPB1*06:01 DPA1*01:03 | 1.8% | | 1.2% | | |
| | DQB1*02:02 DQA1*02:01 | 11.8% | | 0.0% | | |
| | DQB1*02:02 DQA1*05:05 | | | | | |
| | DQB1*03:01 DQA1*02:01 | | | | | |
| | DQB1*03:01 DQA1*05:05 | 11.6% | | 0.0% | | |
| Expi293T | DRB1*15:01 | 14.4% | 2.9% | 7.9% | 6.7% | 11.1% |
| | DRB5*01:01 | 16.1% | | | | |
| | DPB1*04:02 DPA1*01:03 | 11.4% | | 7.0% | | |
| | DQB1*06:02 DQA1*01:02 | 14.3% | | 3.6% | | |

Finally, Applicant confirmed that CIITA-transduced cells presented peptides derived from extracellular proteins, as should be expected for HLA-II presentation. To this end, Applicant quantified the fraction of HLA-II peptides derived from bovine serum albumin (BSA), a non-human protein present in the cell growth medium, as done previously (Forlani et al., 2021). As a negative control, Applicant examined the HLA-I immunopeptidome of the same cells (Weingarten-Gabbay et al., 2021). Since HLA-I peptides are mostly processed from endogenous proteins, Applicant expected low representation of the exogenous BSA protein in these data. Indeed, Applicant observed 5.2- and 4.6-fold more BSA-derived peptides in the HLA-II peptidome than the HLA-I peptidome in A549/ATC and HEK293T/ATC cells, respectively (FIG. 1G). Moreover, BSA-derived HLA-II peptides across both A549/ATC and HEK293T/ATC experiments had the expected lengths of 12-25 amino acids (˜87% of peptides), matching the distribution of human-derived HLA-II peptides in the same samples (FIG. 1F). In contrast, the BSA-derived peptides in HLA-I samples had longer lengths than canonical HLA-I peptides, suggesting that these peptides might have arisen from exogenous oeotudase trimming and binding to empty surface HLA-I. Altogether, these data indicate that CIITA-transduced A549/AT and HEK293T/AT cells can present HLA-II peptides generated from endocytosed proteins.

SARS-CoV-2 HLA-II Peptides from Canonical and Out-of-Frame Overlapping ORFs

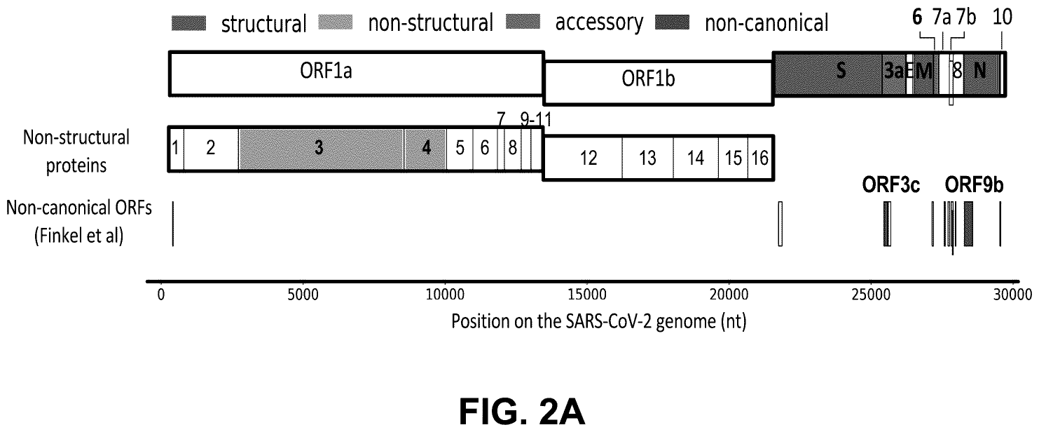

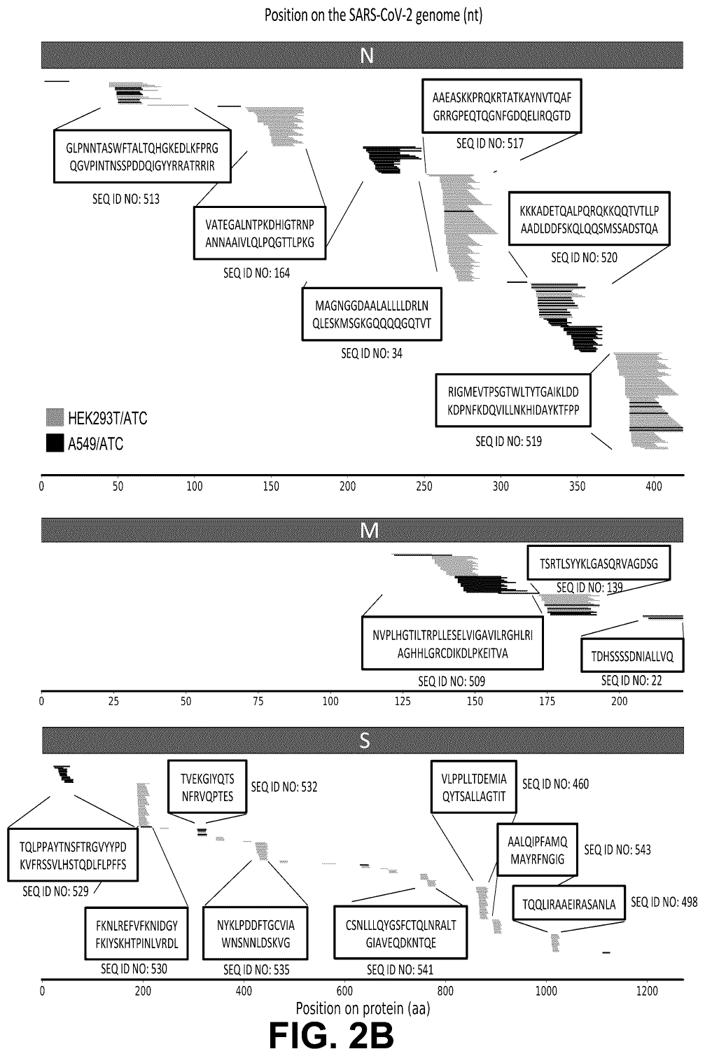

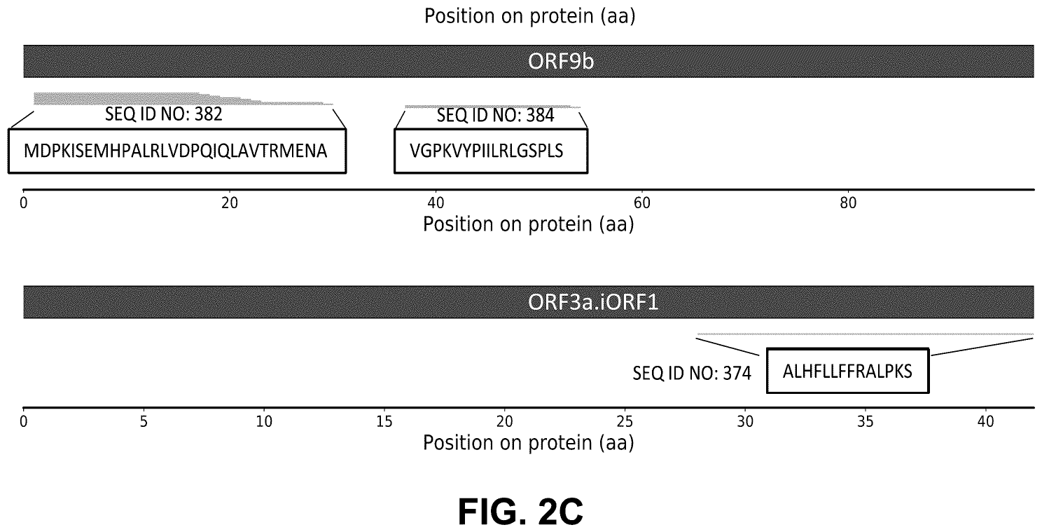

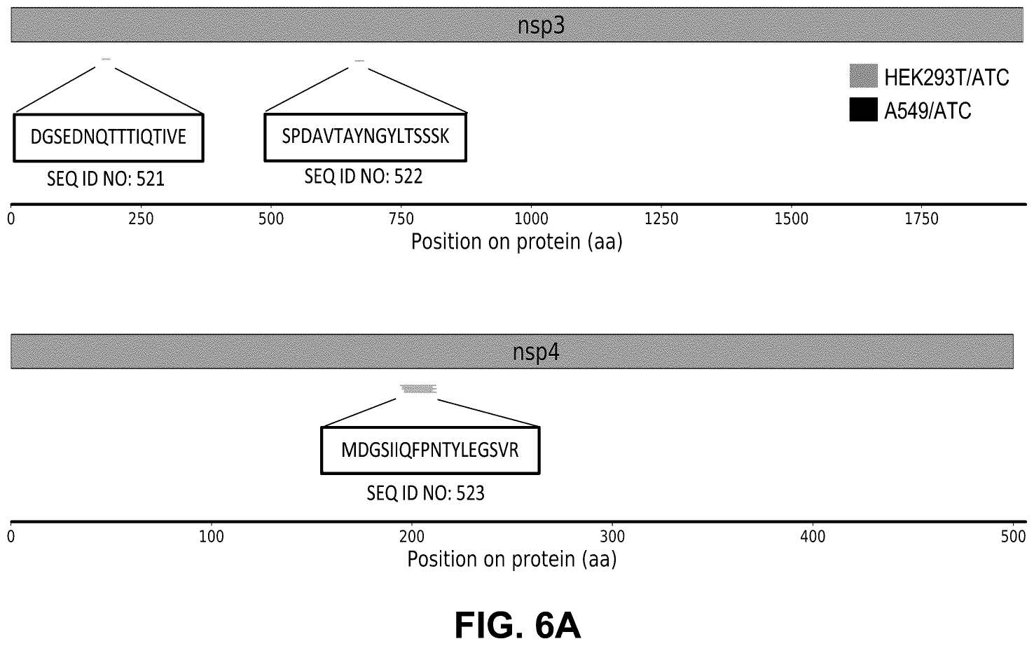

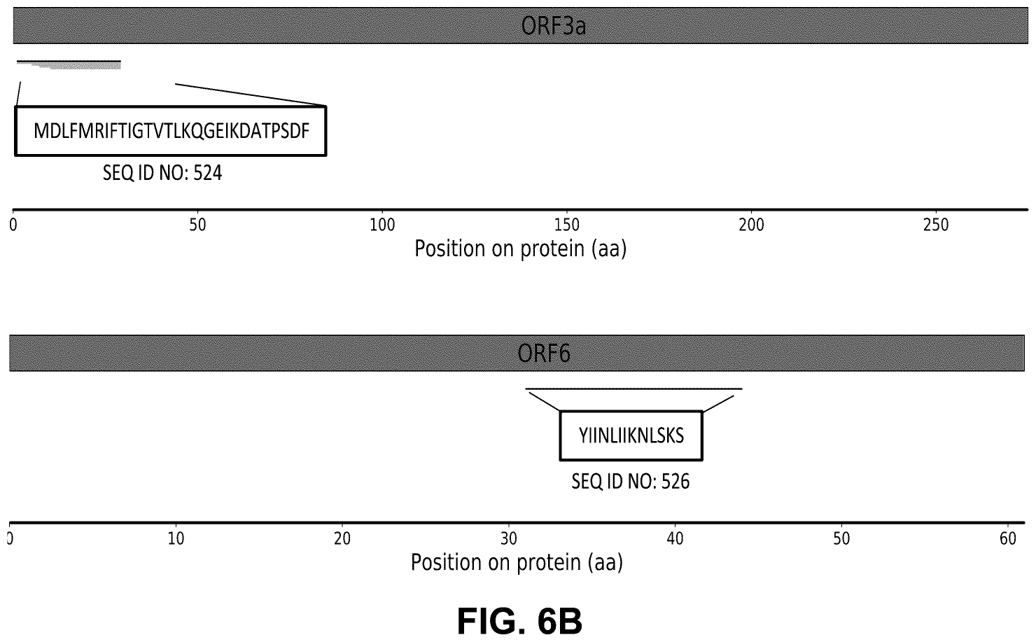

Applicant next analyzed the HLA-II presented peptides derived from SARS-CoV-2 proteins, detecting 469 unique peptides from canonical viral proteins: N, S, M, ORF3a, ORF6, non-structural protein 3 (nsp3) and nsp4 (Tables 1 & 2, FIG. 2A). Examining the distribution of HLA-II peptides across SARS-CoV-2 proteins, Applicant observed the expected clustering of peptides into nested sets, with shared core sequences but different N- or C-terminal ends (FIG. 2B, FIG. 6). This pattern is a hallmark of the HLA class II pathway and differentiates it from the class I presentation. While MHC class I molecules structurally constrain the length of loaded peptides, the open structure of the binding groove of MHC-II molecules allows interaction with peptides of variable length, with parts of the peptides protruding out of the binding groove (Lippolis et al., 2002). Interestingly, although A549/ATC and HEK293T/ATC cells express different HLA-II alleles (Table 3) with distinct binding preferences, some clusters contained peptides from both cell lines. This observation suggests that viral antigen processing steps upstream of HLA-II peptide loading play a key role in shaping the HLA-II immunopeptidome.

The untargeted nature of Applicant's analysis allowed a search for peptides originating from non-canonical ORFs (Finkel et al., 2020) in addition to the canonical SARS-CoV-2 proteins. Overall, Applicant detected 11 peptides from two internal overlapping ORFs: ORF9b (overlapping with N, also called NiORF1) and ORF3c (overlapping with ORF3a, also called 3a.iORF1). ORF9b gave rise to 10 peptides, all of which clustered into two nested sets in the first half of the protein (Tables 1 & 2, FIG. 2C). From each nested set, at least one peptide was predicted to bind one of the HLA-II alleles expressed in HEK293T cells: DRB5*01:01 and DQA1*01:02/DQB1*06:02 in the first and second sets, respectively. Interestingly, HLA-II peptides arose from a different region of the ORF9b protein compared to HLA-I peptides, which originated from the C-terminal region (Weingarten-Gabbay et al., 2021). Applicant identified one HLA-II peptide (ALHFLLFFRALPKS (SEQ ID NO: 374)) from ORF3c. Although it was only one peptide, it was observed in the same fraction (fxn 3) in two biological replicates and was a high scoring peptide, which supports its authenticity. To the best of Applicant's knowledge, this is the first experimental evidence for ORF3c expression at the protein level since it was originally detected using ribosome profiling (Finkel et al., 2020) and computational predictions (Cagliani et al., 2020; Firth, 2020; Jungreis et al., 2021).

SARS-CoV-2 HLA-II Peptides Co-Localize with Epitopes that Elicit CD4+ T Cell Responses in COVID-19 Patients

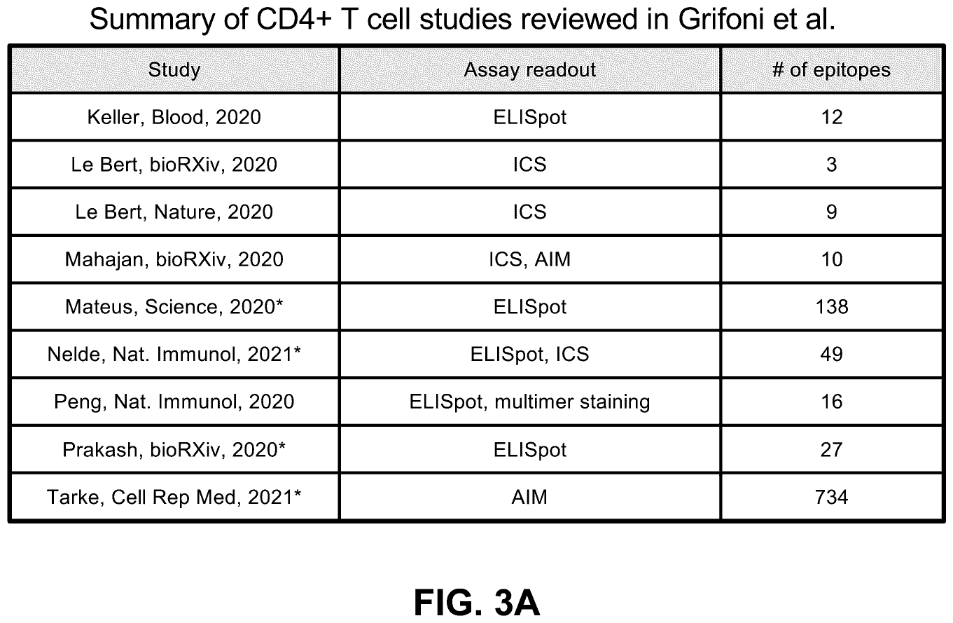

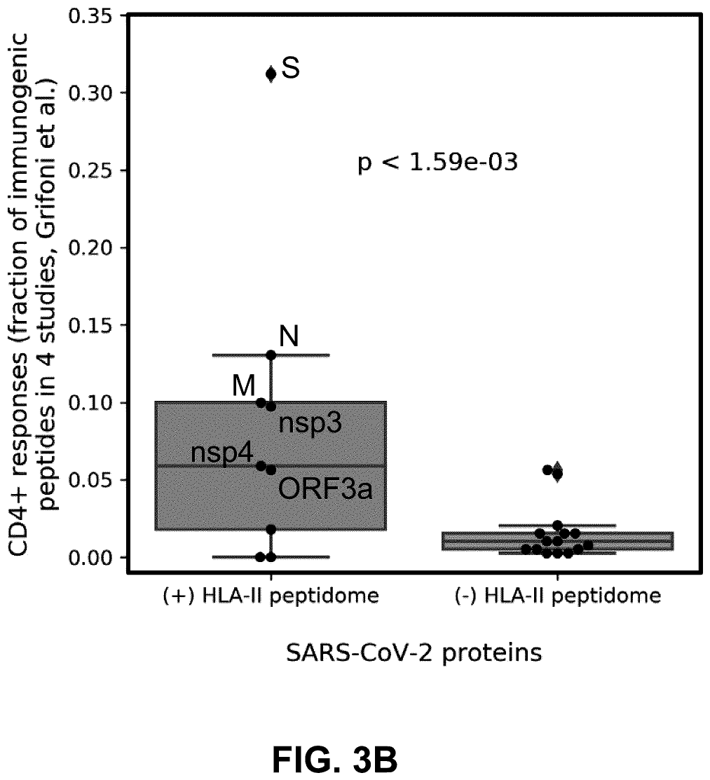

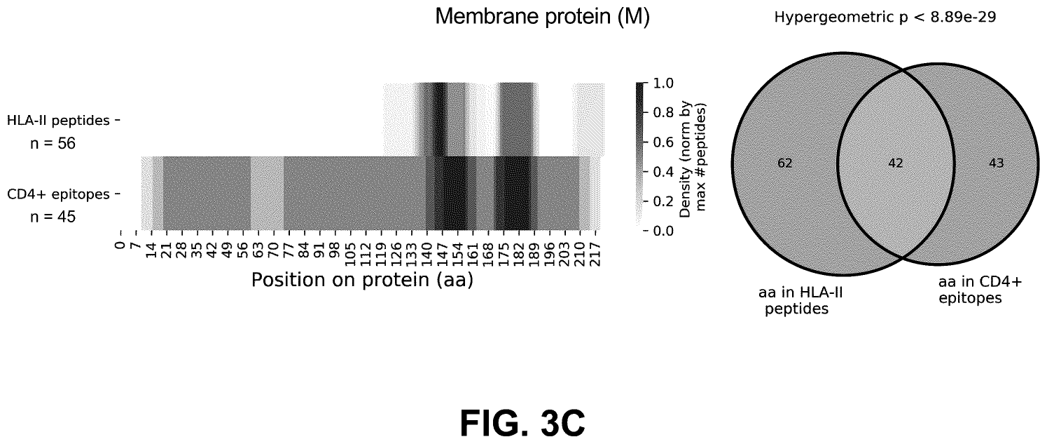

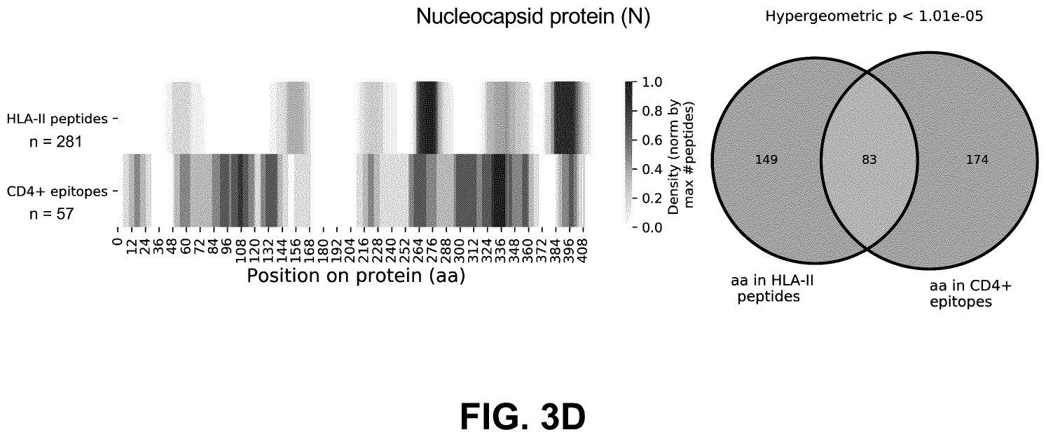

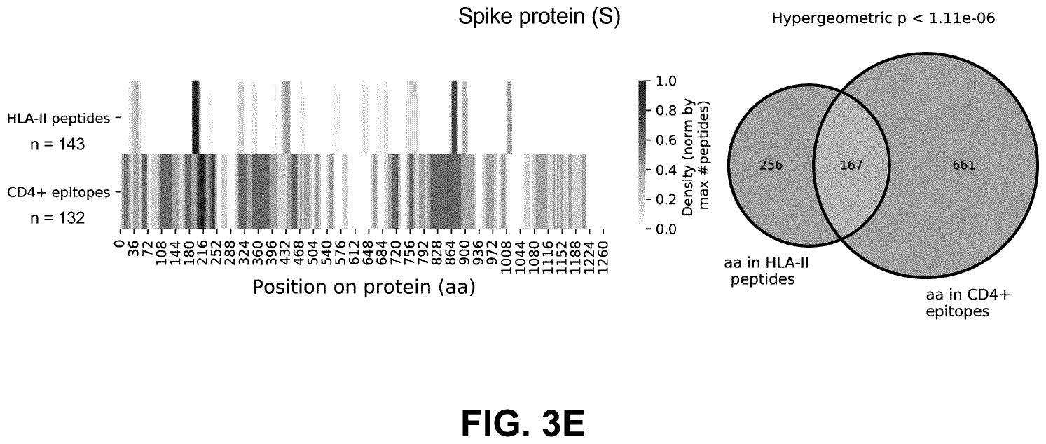

To evaluate if the HLA-II peptides that Applicant detected by mass spectrometry contribute to T cell responses in COVID-19 patients, Applicant compared the HLA-II viral peptides to reported CD4+ epitopes derived from SARS-CoV-2 proteins. Applicant used a curated dataset of T cell epitopes reported by Grifoni et al. (Grifoni et al., 2021). This dataset combines 9 studies that tested CD4+ T cell responses using various assays including ELISpot, Intracellular Cytokine Staining (ICS) and Activation Induced Markers (AIM) (FIG. 3A) (Keller et al., 2020; Le Bert et al., 2020, 2021; Mahajan et al., 2021; Mateus et al., 2020; Nelde et al., 2021; Peng et al., 2020; Prakash et al., n.d.; Tarke et al., 2021).

First, Applicant checked the overlap between viral proteins that generated HLA-II peptides and those known to contain CD4+ T cell epitopes. Applicant computed the fraction of total CD4+ epitopes that were derived from each of the SARS-CoV-2 proteins. To avoid biases stemming from over-representation of highly characterized viral proteins, such as S, N and M, Applicant limited the analysis to four studies that surveyed the entire canonical SARS-CoV-2 proteome (highlighted in asterisk in FIG. 3A) (Mateus et al., 2020; Nelde et al., 2021; Prakash et al., n.d.; Tarke et al., 2021). As expected, viral proteins for which Applicant observed HLA-II peptides elicited T cell responses significantly more frequently than those with no detectable HLA-II peptides (Wilcoxon rank-sum p<10-3, FIG. 3B).

Applicant then considered individual viral proteins and tested if the HLA-II peptides co-localize with regions that elicited CD4+ T cell responses in COVID-19 patients. Applicant focused on the three structural proteins that gave rise to the majority of the detected HLA-II peptides: N (n=281), S (n=143), and M (n=56). These three proteins were also the most abundant source of epitopes in the compiled T cell data, accounting for 54% of the total CD4+ epitopes detected in the canonical SARS-CoV-2 proteome. To determine if HLA-II peptides were derived from regions that were more immunogenic in patients, Applicant counted the number of amino acids that were covered by HLA-II peptides, CD4+ epitopes and both, and computed a hypergeometric p-value that estimates the overlap between these two groups. Since Applicant compared peptide localization within individual proteins, Applicant accounted for epitopes reported in all 9 CD4+ T cell studies listed in FIG. 3A, including studies that examined only a few SARS-CoV-2 proteins. Applicant found a statistically significant enrichment of HLA-II peptides in regions from which CD4+ epitopes were derived with p<10-29, p<10-5, and p<10-5 for M, N and S, respectively (FIG. 3C-3E).

Furthermore, Applicant's analysis showed that HLA-II peptides greatly overlap the two immunodominant regions reported in the M protein. These regions, M:144-163 and M:173-192, were recently identified as hotspots of CD4-restricted epitopes that elicited T cell responses in eight and six COVID-19 convalescent samples, respectively (Keller et al., 2020). The HLA-JJ peptides that Applicant detected by mass spectrometry were also confined to the same region of M with highest density in the two reported hot spots: M:121-171 (n=34 peptides) and M:172-192 (n=16 peptides), both detected in HEK293T/ATC and A549/ATC cells (FIGS. 2B, 3C, Tables 1, 2, & 4). Moreover, the predicted HLA restriction of the two T cell epitopes from M:144-163 described by Keller et al (Keller et al., 2020) matches with two of the BILA-II alleles expressed in A549: DRB1*11:04 and DRB4*01:01. Altogether, these results indicate that the HLA-II immunopeptidome of SARS-CoV-2 co-localizes with reported CD4+ T cell epitopes and defines the immunogenic regions in the M protein.

TABLE 4
Definition of segments across viral genes according to coverage by overlapping
HLA-II presented peptides.

| | | | | Unique | Unique | |
|---|---|---|---|---|---|---|
| | | | Unique | A549 | HEK293T | |
| | | | peptides | peptides | peptides | |
| Gene | Segment | | in | in | in | |
| Symbol | length | Segment_id | segment | segment | segment | Segment sequence |
| M | 51 | M: 121-171 | 34 | 16 | 18 | NVPLHGTILT |
| | | | | | | RPLLESELVI |
| | | | | | | GAVILRGHLR |
| | | | | | | IAGHHLGRCD |
| | | | | | | IKDLPKEITV |
| | | | | | | A |
| | | | | | | (SEQ ID NO: 509) |
| M | 21 | M: 172-192 | 16 | 5 | 13 | TSRTLSYYKL |
| | | | | | | GASQRVAGDS |
| | | | | | | G |
| | | | | | | (SEQ ID NO: 510) |
| M | 15 | M: 208-222 | 2 | 2 | 2 | TDHSSSSDNI |
| | | | | | | ALLVQ |
| | | | | | | (SEQ ID NO: 511) |
| N | 16 | N: 2-17 | 1 | 1 | 0 | SDNGPQNQRN |
| | | | | | | APRITE |
| | | | | | | (SEQ ID NO: 512) |
| N | 52 | N: 44-95 | 16 | 7 | 11 | GLPNNTASWF |
| | | | | | | TALTQHGKED |
| | | | | | | LKFPRGQGVP |
| | | | | | | INTNSSPDDQ |
| | | | | | | IGYYRRATRR |
| | | | | | | IR |
| | | | | | | (SEQ ID NO: 513) |
| N | 15 | N: 115-129 | 1 | 1 | 0 | TGPEAGLPYG |
| | | | | | | ANKDG |
| | | | | | | (SEQ ID NO: 514) |
| N | 38 | N: 133-170 | 30 | 0 | 30 | VATEGALNTP |
| | | | | | | KDHIGTRNPA |
| | | | | | | NNAAIVLQLP |
| | | | | | | QGTTLPKG |
| | | | | | | (SEQ ID NO: 515) |
| N | 38 | N: 210-247 | 21 | 21 | 0 | MAGNGGDAAL |
| | | | | | | ALLLLDRLNQ |
| | | | | | | LESKMSGKGQ |
| | | | | | | QQQGQTVT |
| | | | | | | (SEQ ID NO: 516) |
| N | 47 | N: 251-297 | 81 | 1 | 81 | AAEASKKPRQ |
| | | | | | | KRTATKAYNV |
| | | | | | | TQAFGRRGPE |
| | | | | | | QTQGNFGDQE |
| | | | | | | LIRQGTD |
| | | | | | | (SEQ ID NO: 517) |
| N | 13 | N: 304-316 | 1 | 1 | 0 | IAQFAPSASA |
| | | | | | | FFG |
| | | | | | | (SEQ ID NO: 518) |
| N | 47 | N: 319-365 | 44 | 36 | 17 | RIGMEVTPSG |
| | | | | | | TWLTYTGAIK |
| | | | | | | LDDKDPNEKD |
| | | | | | | QVILLNKHID |
| | | | | | | AYKTEPP |
| | | | | | | (SEQ ID NO: 519) |
| N | 47 | N: 373-419 | 69 | 5 | 69 | KKKADETQAL |
| | | | | | | PQRQKKQQTV |
| | | | | | | TLLPAADLDD |
| | | | | | | ESKQLQQSMS |
| | | | | | | SADSTQA |
| | | | | | | (SEQ ID NO: 520) |
| nsp3 | 16 | nsp3: 174-189 | 1 | 0 | 1 | DGSEDNQTTT |
| | | | | | | IQTIVE |
| | | | | | | (SEQ ID NO: 521) |
| nsp3 | 17 | nsp3: 661-677 | 1 | 0 | 1 | SPDAVTAYNG |
| | | | | | | YLTSSSK |
| | | | | | | (SEQ ID NO: 522) |
| nsp4 | 18 | nsp4: 194-211 | 5 | 0 | 5 | MDGSIIQFPN |
| | | | | | | TYLEGSVR |
| | | | | | | (SEQ ID NO: 523) |
| ORF3a | 28 | ORF3a: 1-28 | 4 | 1 | 4 | MDLFMRIFTI |
| | | | | | | GTVTLKQGEI |
| | | | | | | KDATPSDF |
| | | | | | | (SEQ ID NO: 524) |
| ORF3a. | 14 | ORF3a. | 1 | 0 | 1 | ALHFLLFFRA |
| iORF1 | | iORF1: 28-41 | | | | LPKS |
| | | | | | | (SEQ ID NO: 525) |
| ORF6 | 13 | ORF6: 31-43 | 1 | 1 | 0 | YIINLIIKNL |
| | | | | | | SKS |
| | | | | | | (SEQ ID NO: 526) |
| ORF9b | 29 | ORF9b: 1-29 | 8 | 0 | 8 | MDPKISEMHP |
| | | | | | | ALRLVDPQIQ |
| | | | | | | LAVTRMENA |
| | | | | | | (SEQ ID NO: 527) |
| ORF9b | 17 | ORF9b: 37-53 | 2 | 0 | 2 | VGPKVYPIIL |
| | | | | | | RLGSPLS |
| | | | | | | (SEQ ID NO: 528) |
| S | 39 | S: 22-60 | 13 | 13 | 0 | TQLPPAYTNS |
| | | | | | | FTRGVYYPDK |
| | | | | | | VERSSVLHST |
| | | | | | | QDLFLPFFS |
| | | | | | | (SEQ ID NO: 529) |
| S | 31 | S:186-216 | 34 | 1 | 33 | FKNLREFVEK |
| | | | | | | NIDGYFKIYS |
| | | | | | | KHTPINLVRD |
| | | | | | | L |
| | | | | | | (SEQ ID NO: 530) |
| S | 17 | S: 233-249 | 1 | 0 | 1 | INITRFQTLL |
| | | | | | | ALHRSYL |
| | | | | | | (SEQ ID NO: 531) |
| S | 19 | S:307-325 | 4 | 3 | 3 | TVEKGIYQTS |
| | | | | | | NERVQPTES |
| | | | | | | (SEQ ID NO: 532) |
| S | 17 | S:344-360 | 3 | 0 | 3 | ATREASVYAW |
| | | | | | | NRKRISN |
| | | | | | | (SEQ ID NO: 533) |
| S | 16 | S:398-413 | 1 | 0 | 1 | DSFVIRGDEV |
| | | | | | | RQIAPG |
| | | | | | | (SEQ ID NO: 534) |
| S | 25 | S: 422-446 | 14 | 0 | 14 | NYKLPDDETG |
| | | | | | | CVIAWNSNNL |
| | | | | | | DSKVG |
| | | | | | | (SEQ ID NO: 535) |
| S | 16 | S:470-485 | 2 | 0 | 2 | TEIYQAGSTP |
| | | | | | | CNGVEG |
| | | | | | | (SEQ ID NO: 536) |
| S | 28 | S:554-581 | 1 | 0 | 1 | ESNKKELPFQ |
| | | | | | | QFGRDIADTT |
| | | | | | | DAVRDPQT |
| | | | | | | (SEQ ID NO: 537) |
| S | 19 | S:630-648 | 3 | 1 | 2 | TPTWRVYSTG |
| | | | | | | SNVFQTRAG |
| | | | | | | (SEQ ID NO: 538) |
| S | 15 | S:670-684 | 1 | 0 | 1 | ICASYQTQTN |
| | | | | | | SPRRA |
| | | | | | | (SEQ ID NO: 539) |
| S | 18 | S:686-703 | 3 | 0 | 3 | SVASQSIIAY |
| | | | | | | TMSLGAEN |
| | | | | | | (SEQ ID NO: 540) |
| S | 32 | S:749-780 | 10 | 0 | 10 | CSNLLLQYGS |
| | | | | | | FCTQLNRALT |
| | | | | | | GIAVEQDKNT |
| | | | | | | QE |
| | | | | | | (SEQ ID NO: 541) |
| S | 24 | S:860-883 | 25 | 0 | 25 | VLPPLLTDEM |
| | | | | | | IAQYTSALLA |
| | | | | | | GTIT |
| | | | | | | (SEQ ID NO: 542) |
| S | 19 | S:892-910 | 11 | 0 | 11 | AALQIPFAMQ |
| | | | | | | MAYRENGIG |
| | | | | | | (SEQ ID NO: 543) |
| S | 17 | S:1009-1025 | 14 | 0 | 14 | TQQLIRAAEI |
| | | | | | | RASANLA |
| | | | | | | (SEQ ID NO: 544) |
| S | 15 | S:1111-1125 | 1 | 1 | 0 | EPQIITTDNT |
| | | | | | | FVSGN |
| | | | | | | (SEQ ID NO: 545) |

### Distinct Subsets of Viral Proteins are Presented on the HLA-I and HLA-II Complexes

In the context of vaccine design, it is important to decipher both CD4+ and CD8+ T cell epitopes, since effective vaccines require potent induction of both arms of the adaptive cellular response. Many of the current vaccine strategies rely on the delivery of a single viral protein to invoke both CD8+ and CD4+ T cell responses and mainly focus on the structural proteins S or N (Barouch, 2022; Creech et al., 2021; Dai & Gao, 2021; Krammer, 2020; Kyriakidis et al., 2021; Oronsky et al., 2022; Silva et al., 2022). However, HLA-I and HLA-II presentation, which facilitate CD4+ and CD8+ responses, respectively, have distinct antigen processing steps, raising the likelihood that each of these pathways samples a different subset of viral proteins.

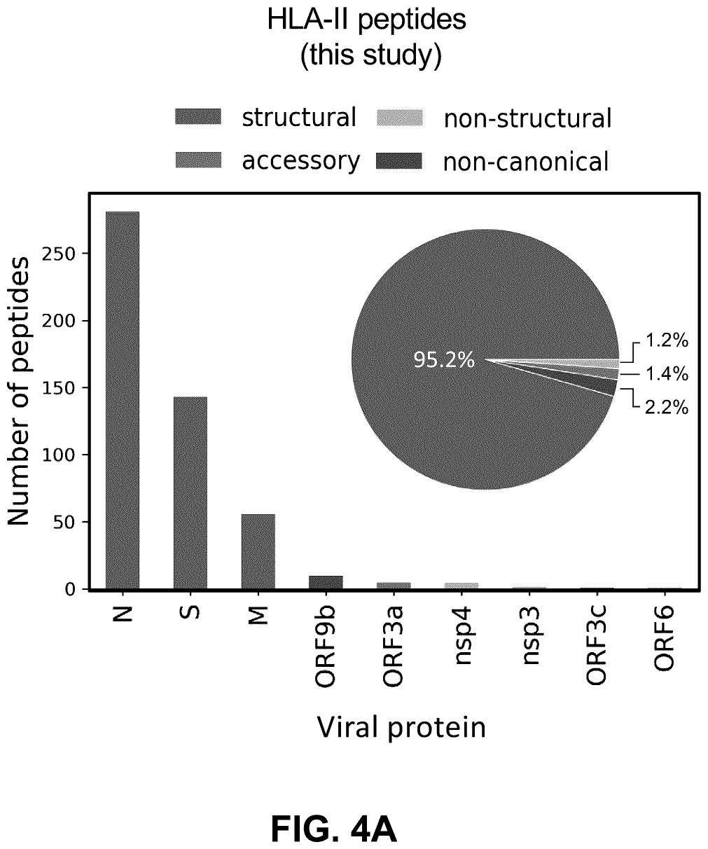

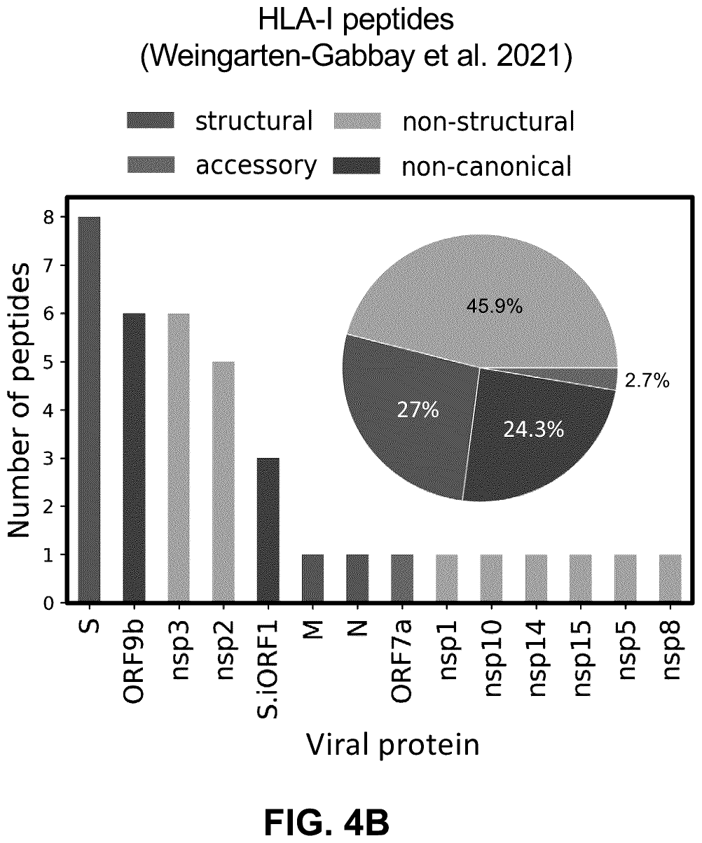

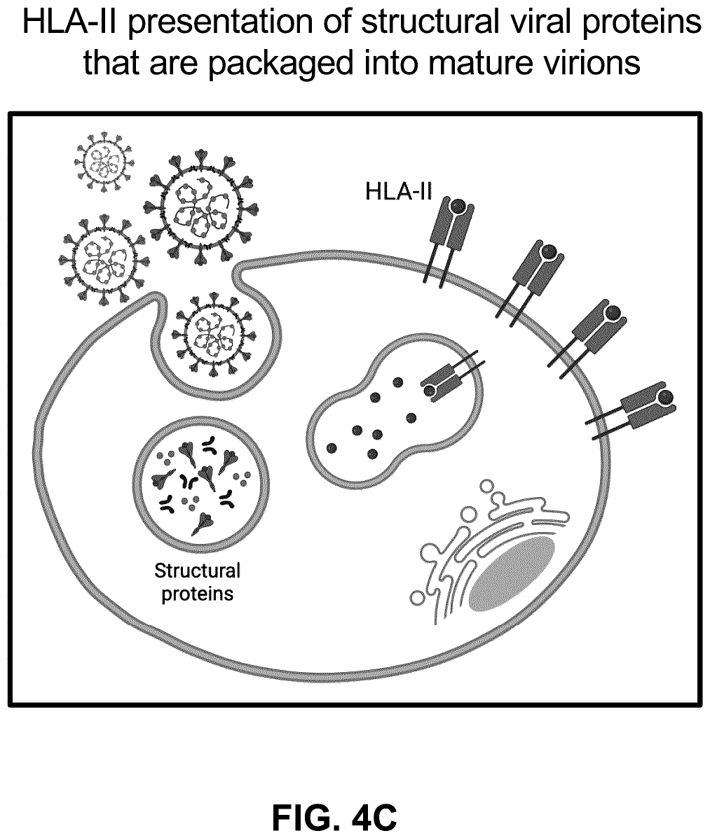

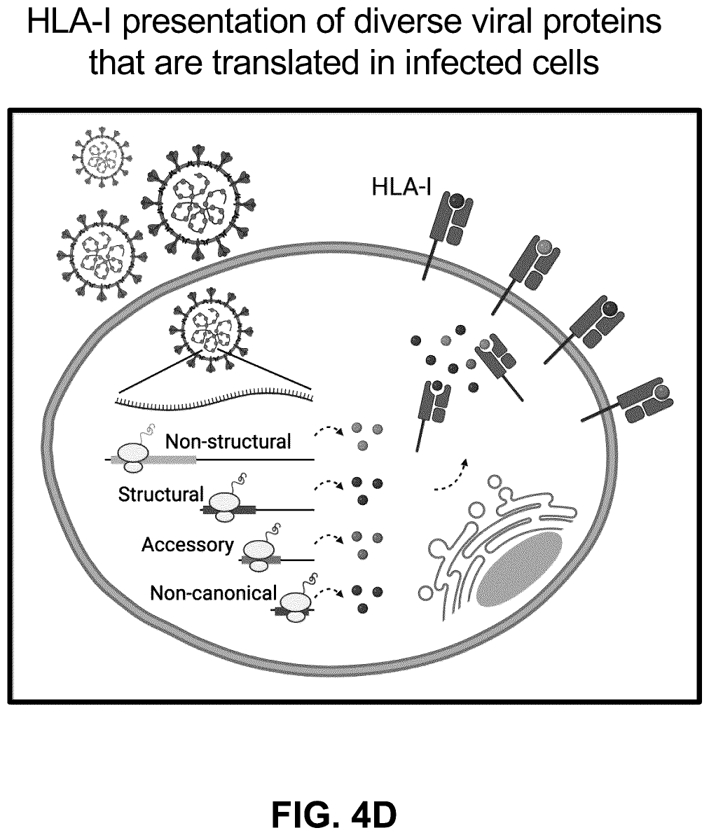

To compare viral proteins presented on HLA-I versus HLA-II complexes, Applicant examined the HLA-I (Weingarten-Gabbay et al., 2021) and HLA-II immunopeptidomes of SARS-CoV-2 infected HEK293T/ATC and A549/ATC cells. Applicant computed the number of peptides observed from each source protein and assessed the representation of proteins in four groups: Non-structural proteins, structural proteins, accessory proteins, and non-canonical ORFs. HLA-II presentation was dominated by the structural proteins N, S, and M, accounting for 95.2% of the detected peptides in HEK293T/ATC and A549/ATC cells, with negligible contribution of the non-structural (1.2%), accessory (1.4%) and non-canonical proteins (2.2%) (FIG. 4A). In contrast, only 27% of the detected HLA-I peptides were derived from structural proteins, with a large fraction of peptides from non-structural proteins (45.9%) and non-canonical ORFs (24.3%) (FIG. 4B). Together, these results point to a different presentation profile of SARS-CoV-2 on the HLA-I and HLA-II complexes and suggest that CD4+ and CD8+ T cells engage with different parts of the virus.

### Discussion

Applicant provides the first genomic landscape of SARS-CoV-2 peptides that are naturally processed and presented on the HLA-II complex. This genome-wide view allowed the systematic comparison of the HLA-II immunopeptidome to the T cell epitopes in COVID-19 patients and to the HLA-I immunopeptidome of SARS-CoV-2 and uncovered new insights into antigen presentation.

Applicant's work adds to a growing list of studies that employ overexpression of the CIITA master regulator to infer the HLA-II immunopeptidome of cancer cells and viruses (Becerra-Artiles et al., 2019, 2022; Forlani et al., 2021; Hos et al., 2022). Although the profiled cells were not professional antigen-presenting cells (APCs), Applicant believes that these measurements uncovered genuine HLA-II peptides: The peptides Applicant detected were in the expected length range and contained sequence peptide sequences that matched with the HLA-II alleles of respective cell lines. In addition, Applicant detected presentation of exogenous proteins derived from extracellular bovine serum. Most importantly, the immunopeptidome data successfully captured the SARS-CoV-2 CD4+ T cell epitopes that were detected in a wide range of independent studies using conventional targeted T cell assays. The source proteins of HLA-II peptides in this study were also found to be the most immunogenic proteins in convalescent COVID-19 patients. Moreover, the HLA-II peptides co-localized with regions in the SARS-CoV-2 proteins that are known to elicit strong T cell responses.

In the context of highly pathogenic viruses, CIITA induction in HLA-II null cell lines provides an efficient platform to probe the entire viral genome within the restrictions of high-containment facilities. To date, HLA-II immunopeptidome studies of SARS-CoV-2 were limited to a single protein, using pulse experiments with a recombinant protein (Knierman et al., 2020; Parker et al., 2021), or four exogenously expressed proteins using plasmid overexpression (Nagler et al., 2021). By inducing the HLA-II pathway in SARS-CoV-2 infected cells, Applicant could detect HLA-II peptides from the entire viral genome. Although HLA-II peptides are predominantly presented by APCs, such as dendritic cells and macrophages, they can also be presented by non-immune cells upon induction of HLA-II expression, such as lung epithelial cells exposed to IFN-gamma (Neuwelt et al., 2020; Wosen et al., 2018). Thus, the CIITA induction system can also recapitulate naturally occurring HLA-II presentation events of infected cells in-vivo. Overexpressing CIITA in the same cell lines in which Applicant profiled the HLA-I immunopeptidome allowed use of the same reagents and inactivation protocol that Applicant optimized for Biosafety Level 3 (BSL3) laboratory (Weingarten-Gabbay et al., 2022). Thus, Applicant believes that this approach can readily be extended to additional high-containment viruses.

This analysis uncovers striking differences in the subset of viral proteins that are presented on HLA-I versus HLA-II complexes. Applicant hypothesizes that the observed differences stem from the different stages of the viral life cycle at which viral proteins are processed and loaded onto HLA molecules (FIG. 4C,D). The HLA class II pathway can present peptides from exogenous sources, e.g. mature virions and infected cell debris, that are cleaved within endosomal-lysosomal compartments and loaded onto HLA-II complexes (FIG. 4C). Thus, the repertoire of HLA-II peptides mostly reflects the viral proteins that are part of mature virus particles. In contrast, the HLA-I pathway samples viral proteins that are actively translated in the cytoplasm of infected cells. Thus, the HLA-I immunopeptidome mirrors the translatome of the virus, including non-structural proteins, accessory proteins, and non-canonical proteins, which are not necessarily incorporated into mature virions (FIG. 4D). These differences may extend to other viruses as well, many of which encode proteins that are required for viral replication in infected cells but are not packaged into mature virions. The distinct repertoire of viral peptides that are presented on HLA-I versus HLA-II complexes suggests that CD4+ and CD8+ T cells recognize different parts of the viral genome. This observation warrants revision of the current “one-protein-fits-all” approach that forms the basis of most synthetic vaccines, including the broadly administered COVID-19 mRNA vaccines. Rather, incorporating targets from different classes of viral proteins, including structural, non-structural and non-canonical proteins, has the potential to invoke a more holistic response of both CD4+ and CD8+ T cells.

An intriguing question in T cell immunology is what determines the immunodominance of a specific protein or a region within a protein. Observing T cell reactivity in convalescent patients to immunodominant epitopes represents the final outcome in a chain of events that determine which peptides will elicit a T cell response. Some factors defining the immunodominance of a given epitope include protein expression levels, accessibility to proteolytic cleavage and antigen processing, loading onto the HLA complex, the presence of a matching T cell receptor (TCR) in the repertoire of naive T cells, and the binding affinity of an HLA-peptide complex with its matching TCR. Since the immunopeptidome represents antigen processing and presentation steps occurring prior to interaction with TCR, it distinguishes between epitope selectivity at the level of presentation versus T cell recognition. A recent study identified that the HLA-II peptidome of influenza virus defines H1-HA immunodominant regions targeted by memory CD4+ T cells (Cassotta et al., 2020). Similarly to this observation, Applicant found that the two immunodominant hotspots that were reported in the M protein (Keller et al., 2020) greatly overlap the HLA-II immunopeptidome of infected cells, suggesting that antigen processing and presentation steps define the immunodominant regions of M. Together, these observations demonstrate the robustness of immunopeptidome analysis for identifying the most relevant protein regions for designing T cell assays and selecting candidates for vaccines.

This work uncovers HLA-II peptides from two non-canonical overlapping ORFs: ORF9b and ORF3c, and provides the first experimental evidence for ORF3c expression at the peptide level. In contrast to HLA-I presentation, in which non-canonical peptides were enriched on the HLA-I complex, non-canonical peptides represented only a small fraction of the HLA-II immunopeptidome. However, the detection of peptides from non-canonical ORFs in both the HLA-I and the HLA-II immunopeptidomes emphasizes the importance of incorporating non-canonical ORFs into T cell studies to achieve a complete picture of the antiviral immune profile. The discovery of a peptide from ORF3c highlights another important aspect of immunopeptidome studies and its potential impact on the understanding of non-canonical ORFs. Although non-canonical ORFs have numerous roles in the viral life cycle, including regulating viral gene expression and modulating virus infection, their detection in tryptic proteome experiments is often challenging due to their small size, shorter half life, and in some cases, lack of observable tryptic peptides. Of the 23 non-canonical ORFs that were identified in the SARS-CoV-2 genome (Finkel et al., 2020), only one (ORF9b) has so far been detected in global tryptic proteomic experiments (Weingarten-Gabbay et al., 2021). The longer half-life of the HLA-peptide complex compared to the non-canonical ORF translation product in the cell may increase the probability of detection by mass spectrometry (Ruiz Cuevas et al., 2021). As an example, although Applicant and others identified three peptides from S.iORF1 (an internal overlapping ORF in spike) on the HLA-I complex (Nagler et al., 2021; Weingarten-Gabbay et al., 2021), this protein was not detected in whole tryptic proteome analysis of the same infected cell lysates. ORF3c was recently shown to inhibit innate immunity by restricting IFN-β production, exposing an important mechanism of SARS-CoV-2 immune evasion (Stewart et al., 2022). Applicant's study provides evidence that this important non-canonical protein is expressed in cells that are infected with SARS-CoV-2. Thus, in addition to enhancing the understanding of viral antigen presentation, immunopeptidome studies contribute to the basic understanding of viruses by illuminating the complete set of canonical and non-canonical viral proteins.

There are limitations to this study. Although CIITA overexpression activated the HLA-II pathway in the cell lines used in this study, allowing the investigation of SARS-CoV-2 HLA-II immunopeptidome, these cells may not capture the unique biology of how APCs subtypes uptake and process mature virions and virus infected cells in vivo. In contrast to the cells profiled in this study, APCs are not naturally infected by SARS-CoV-2. It is possible that productive infection impacts the antigen processing and presentation steps and provides an internal source of viral proteins, in addition to the endocytosed particles, for production of HLA-II peptides. For instance, Ghosh and colleagues (Ghosh et al. 2020) showed that β-Coronaviruses can traffic to lysosomes and egress by Arl8b-dependent lysosomal exocytosis. In this context, lysosomes are deacidified, which can inactivate proteolytic enzymes in infected cells and impair antigen presentation. Further, this study only uncovered the peptides that are presented by the HLA-II alleles expressed in A549 and HEK293T cells. As such, Applicant might have missed SARS-CoV-2 HLA-II peptides that were incompatible with A549 and HEK293T cells. Nonetheless, this study identifies HLA-II peptides that can be presented on virally infected cells and provides valuable insights into which viral proteins are more likely to be presented by SARS-CoV-2 infected cells.

### Methods

### Plasmid

Lentiviral vectors pLOC_hACE2_PuroR and pLOC_hTMPRSS2_BlastR, harboring human ACE2 and TMPRSS2, respectively, have been described (Chen et al., JVI). To generate a lentiviral vector containing human CIITA, Applicant amplified the CIITA cDNA from pcDNA3 myc CIITA (Addgene #14650) and cloned it into pTRIP-SFFV-Hygro-2A (previously described (Gentili et al., 2023)) via GIBSON ASSEMBLY®. The resultant plasmid was named pTRIP-SFFV-Hygro-2A-myc-CIITA.

### Cell Culture

Human embryonic kidney HEK293T cells (female), human lung A549 cells (male), and African green monkey kidney epithelial Vero E6 cells (female) were maintained at 37° C. and 5% CO2 in DMEM containing 10% FBS. To generate HEK293T and A549 cells overexpressing human ACE2, TMPRSS2, and CIITA, Applicant transduced these cells with lentiviral vectors pLOC_hACE2_PuroR, pLOC_hTMPRSS2_BlastR, and pTRIP-SFFV-Hygro-2A-myc-CIITA, and selected for the triple-transduced cells in culture medium supplemented with 1 μg/ml each of puromycin and blasticidin and 320 μg/ml of hygromycin. A375 cells were obtained from ATCC (ATCC® CRL-1619). A375 cells were grown in ATCC-formulated Dulbecco's Modified Eagle's Medium (Catalog No. 30-2002) with fetal bovine serum to a final concentration of 10% using ATCC guidelines. A375 cells were harvested by trypsinization (Trypsin-EDTA 0.25%, Gibco™ 25200056), pelleted and rinsed in PBS twice. Pellets were snap frozen and stored at −80° C.

### Flow Cytometry

1.5*10\hat{ }A549 or A375 cells were incubated with 2 ul FITC anti HLA-DR, DP, DQ antibody (BD Pharmingen #562008) in 100 ul PBS at 4° C. for 45 minutes. Cells were washed three times with PBS, resuspended in 400 ul PBS and analyzed using a CYTOFLEX flow cytometer.

### SARS-CoV-2 Virus Stock Preparation and Titration

The SARS-CoV-2 USA-WA1/2020 isolate (NCBI accession number: MT246667) was deposited by the Centers for Disease Control and Prevention and obtained through BEI Resources, NIAID, NIH (NR-52281). Applicant then passaged the virus twice onto Vero E6 cells to obtain the P2 stock, as previously described (Chen et al., JVI). The virus titration was performed on Vero E6 cells. All experiments in this study utilized the P2 stock.

### Quantification of Virus Infectivity Using Immunofluorescence

A549 and 293T cells stably overexpressing ACE2, TMPRSS2, and CIITA were infected with SARS-CoV-2 at an MOI of 0.5, 1, or 3 for 12, 18, 24, 36, or 48 hours. At indicated times, the culture medium was removed, and the cells were fixed with 4% paraformaldehyde for 60 minutes at room temperature. The cells were then permeabilized with 0.1% of TRITON® X-100 in PBS for 10 minutes and hybridized with anti-SARS-CoV nucleocapsid (rabbit polyclonal) antibody (1:2000, Rockland, #200-401-A50) at 4° C. overnight. ALEXA FLUOR™ 568 goat anti-rabbit antibody (Invitrogen, #A11011) was used as the secondary antibody. Finally, DAPI was used to stain cell nuclei. Images were captured with an EVOS® microscope using a 10× lens, and the percentage of infected cells was calculated with ImageJ.

Immunoprecipitation of HLA-II complexes from cells

Cells from 3×15 cm dishes were scraped into 2.5 ml/dish of cold lysis buffer (20 mM Tris, pH 8.0, 100 mM NaCl, 6 mM MgCl₂, 1 mM EDTA, 60 mM Octyl β-d-glucopyranoside, 0.2 mM Iodoacetamide, 1.5% TRITON® X-100, 50× COMPLETE™ Protease Inhibitor Tablet-EDTA free and PMSF) obtaining a total of ˜9 mL lysate. This lysate was split into 6 eppendorf tubes, with each tube receiving 1.5 mL volume, and incubated on ice for 15 min with 1 ul of Benzonase (Thomas Scientific, E1014-25KU) to degrade nucleic acid. The lysates were then centrifuged at 4,000 rpm for 22 min at 4° C. and the supernatants were transferred to another set of 6 eppendorf tubes containing a mixture of pre-washed beads (Millipore Sigma, GE17-0886-01) and 12.5 uL (12.5 ug) of MHC class II antibodies in a 3:1:1 mixture of TAL-1B5 (Abcam, ab20181), EPR11226 (Abcam, ab157210) and B-K27 (Abcam, ab47342). The immune complexes were captured on the beads by incubating on a rotor at 4° C. for 3 hr in the BSL3 lab. Virus inactivation was confirmed before subsequent samples processing outside the BSL3 using plaque assay (Weingarten-Gabbay et al., 2021, 2022). In total, nine washing steps were performed; one wash with 1 mL of cold lysis wash buffer (20 mM Tris, pH 8.0, 100 mM NaCl, 6 mM MgCl₂, 1 mM EDTA, 60 mM Octyl β-d-glucopyranoside, 0.2 mM Iodoacetamide, 1.5% TRITON® X-100), four washes with 1 mL of cold complete wash buffer (20 mM Tris, pH 8.0, 100 mM NaCl, 1 mM EDTA, 60 mM Octyl β-d-glucopyranoside, 0.2 mM Iodoacetamide), and four washes with 20 mM Tris pH 8.0 buffer. Dry beads were stored at −80° C. until mass-spectrometry analysis was performed.

### HLA-II Peptidome Desalting and LC-MS/MS Data Generation

HLA peptides were eluted and desalted from beads as follows: wells of the tC18 40 mg SEP-PAK® desalting plate (Waters, Milford, MA) were activated with 2×1 mL of methanol (MeOH) and 500 μL of 99.9% acetonitrile (ACN)/0.1% formic acid (FA), then washed with 4×1 mL of 1% FA. A 10 m PE fritted filter plate containing the HLA-IP beads was placed on top of the SEP-PAK® desalting plate. To dissociate peptides from HLA molecules and facilitate peptides binding to the tC18 solid phase, 200 μL of 3% ACN/5% FA was added to the beads in the filter plate. 100 fmol internal retention time (iRT) standards (Biognosys SKU: Ki-3002-2) was spiked into each sample as a loading control and pushed through both the filter plate and 40 mg SEP-PAK® desalting plate. Following sample loading there was one wash with 400 μL of 1% FA. Beads were then incubated with 500 μL of 10% acetic acid (AcOH) three times for 5 min to further dissociate bound peptides from the HLA molecules. The beads were rinsed once with 1 mL 1% FA and the filter plate was removed. The SEP-PAK® desalting plate was rinsed with 1 mL 1% FA an additional three times. The peptides were eluted from the Sep-Pak desalt plate using 250 μL of 15% ACN/1% FA and 2×250 μL of 50% ACN/1% FA. HLA peptides were eluted into 1.5 mL micro tubes (Sarstedt, Numbrecht, Germany), frozen, and dried down via vacuum centrifugation. Dried peptides were stored at −80° C. until microscaled basic reverse phase separation.

Briefly, peptides were loaded on Stage-tips with 2 punches of SDB-XC material (EMPORE™ 3M). HLA-II peptides were eluted in three fractions with increasing concentrations of ACN (5%, 15%, and 40% in 0.1% NH₄OH, pH 10). Peptides were reconstituted in 3% ACN/5% FA prior to loading onto an analytical column (35 cm, 1.9 μm C18 (Dr. Maisch HPLC GmbH), packed in-house PICOFRIT® 75 μm inner diameter, 10 μm emitter (New Objective)). Peptides were eluted with a linear gradient (EASY-NLC™ 1200, Thermo Fisher Scientific) ranging from 6-30% Solvent B (0.1% FA in 90% ACN) over 84 min, 30-90% B over 9 min and held at 90% B for 5 min at 200 nl/min. MS/MS data were acquired on a THERMO SCIENTIFIC™ ORBITRAP EXLORIS™ 480 equipped with (HLA-I) and without (HLA-II) FAIMS (Thermo Fisher Scientific) in data-dependent acquisition. FAIMS compensation voltages (CVs) were set to −50 and −70 with a cycle time of 1.5 s per FAIMS experiment. MS2 fill time was set to 100 ms; collision energy was 30, 34 or 36 CE.

Proteome Analysis from HLA Enrichment Flow-Through

Flow-throughs of the HLA-II IP that were stored as flash-frozen native protein lysates were briefly thawed on ice for ˜15 min. Once thawed, 10% SDS was added for a final concentration of 5% SDS to denature the lysate, resulting in a final volume of ˜1.5 mL lysate which was prepared for S-TRAP™ digestion (Abelin et al., 2023).

Protein concentration was estimated using a BCA assay for scaling of digestion enzymes. Disulfide bonds were reduced in 5 mM DTT for 30 min at 25° C. and 1000 rpm shaking and cysteine residues were alkylated in 10 mM IAA in the dark for 45 min at 25° C. and 1000 rpm shaking. Lysates were then transferred to a 15 mL conical tube to prepare for protein precipitation. 27% phosphoric acid was added at a 1:10 ratio of lysate volume to acidify and proteins were precipitated with 6× sample volume of ice cold S-TRAP™ buffer (90% methanol, 100 mM TEAB). The precipitate was transferred in successive loads of 3 mL to a S-TRAP™ Midi (Protifi) and loaded with 1 min centrifugation at 4000×g, mixing the remaining precipitate thoroughly between transfers. The precipitated proteins were washed 4× with 3 mL S-TRAP™ buffer at 4000× g for 1 min. To digest the deposited protein material, 350 μL digestion buffer (50 mM TEAB) containing both trypsin and endopeptidase C (LysC), each at 1:50 enzyme:substrate, was passed through each S-TRAP™ column with 1 min centrifugation at 4000×g. The digestion buffer was then added back atop the S-TRAP™ and the cartridges were left capped overnight at 25° C.

Peptide digests were eluted from the S-TRAP™, first with 500 μL 50 mM TEAB and next with 500 μL 0.1% FA, each for 30 sec at 1000×g. The final elution of 500 μL 50% ACN/0.1% FA was centrifuged for 1 min at 4000×g to clear the cartridge. Peptide concentration of the pooled elutions was estimated with a BCA assay, and 10 pg peptide was used for stagetip fractionation. Each 25 ug proteome sample was reconstituted in 4.5 mM ammonium formate (pH 10) in 2% (vol/vol) acetonitrile and separated into four fractions using basic reversed phase fractionation on a C-18 Stage-tip. Fractions were eluted at 5%, 12.5%, 15%, and 50% ACN/4.5 mM ammonium formate buffer (pH 10) and dried. Fractions were reconstituted in 3% ACN/5% FA, and 1 μg was used for LC-MS/MS analysis.

Data-dependent acquisition was performed using a THERMO SCIENTIFIC™ ORBITRAP EXLORIS™ 480 V2.0 software in positive ion mode at a spray voltage of 1.8 kV. MS1 spectra were measured with a resolution of 60,000, a normalized AGC target of 300% for, a maximum injection time of 10 ms, and a mass range from 350 to 1800 m/z. The data-dependent mode cycle was set to trigger MS/MS on up to the top 20 most abundant precursors per cycle at an MS2 resolution of 45,000, an AGC target of 30%, an isolation window of 0.7 m/z, a maximum injection time of 105 ms for proteome, and an HCD collision energy of 34%. Peptides that triggered MS/MS scans were dynamically excluded from further MS/MS scans for 20 s in proteome/phosphoproteome/ubiquitylome and for 30 s in acetylome, with a ±10 ppm mass tolerance. Theoretical precursor envelope fit filter was enabled with a fit threshold of 50% and window of 1.2 m/z. Monoisotopic peak determination was set to peptide and charge state screening was enabled to only include precursor charge states 2-6 with an intensity threshold of 5.0e3. Advanced peak determination (APD) was enabled. “Perform dependent scan on single charge state per precursor only” was disabled.

### LC-MS/MS Data Interpretation

Peptide sequences were interpreted from MS/MS spectra using Spectrum Mill (SM) v 7.08 (proteomics.broadinstitute.org) against a RefSeq-based sequence database containing 41,457 proteins mapped to the human reference genome (hg38) obtained via the UCSC Table Browser (genome.ucsc.edu/cgi-bin/hgTables) on Jun. 29, 2018, with the addition of 13 proteins encoded in the human mitochondrial genome, 264 common laboratory contaminant proteins, 553 human non-canonical small open reading frames, 28 SARS-CoV2 proteins obtained from RefSeq derived from original Wuhan-Hu-1 China isolate NC_045512.2 (ncbi.nlm.nih.gov/nuccore/1798174254) (Wu et al., 2020), and 23 novel unannotated virus ORFs whose translation is supported by Ribo-seq (Finkel et al., 2020) for a total of 42,337 proteins. Among the 28 annotated SARS-CoV2 proteins, Applicant opted to omit the full-length polyproteins ORF1a and ORF1ab, to simplify peptide-to-protein assignment, and instead represented ORF1ab as the mature 16 individual non-structural proteins that result from proteolytic processing of the 1a and 1ab polyproteins. Applicant added the D614G variant of the SARS-Cov2 Spike protein that is commonly observed in European and American virus isolates, and also added 2036 entries from 6-frame translation of the SARS-Cov2 genome for all possible ORFs longer than 6 amino acids.

Parameters for the SM MS/MS search module for HLA-11 immunopeptidomes included: no enzyme specificity; precursor and product mass tolerance of ±10 ppm; minimum matched peak intensity of 30%; ESI-QEXACTIVE-HCD-HLA-v3 scoring; fixed modification: carbamidomethylation of cysteine; variable modifications: cysteinylation of cysteine, oxidation of methionine, deamidation of asparagine, acetylation of protein N-termini, and pyroglutamic acid at peptide N-terminal glutamine; and precursor mass shift range of −18 to 81 Da. For tryptic proteomes, parameters included: “trypsin allow P” enzyme specificity with up to 4 missed cleavages, precursor and product mass tolerance of ±20 ppm, and 30% minimum matched peak intensity (40% for acetylome). Scoring parameters were ESI-QEXACTIVE-HCD-v2. Allowed fixed modifications included carbamidomethylation of cysteine and selenocysteine. Allowed variable modifications for whole proteome datasets were acetylation of protein N-termini, oxidized methionine, deamidation of asparagine, hydroxylation of proline in PG peptide sequences, pyro-glutamic acid at peptide N-terminal glutamine, and pyro-carbamidomethylation at peptide N-terminal cysteine with a precursor MH+ shift range of −18 to 97 Da.

### REFERENCES FOR EXAMPLE 1

Abelin, J. G., Bergstrom, E. J., Rivera, K. D., Taylor, H. B., Klaeger, S., Xu, C., Verzani, E. K., Jackson White, C., Woldemichael, H. B., Virshup, M., Olive, M. E., Maynard, M., Vartany, S. A., Allen, J. D., Phulphagar, K., Harry Kane, M., Rachimi, S., Mani, D. R., Gillette, M. A., Satpathy, S., Clauser, K. R., Udeshi, N. D., & Carr, S. A. (2023). Workflow enabling deepscale immunopeptidome, proteome, ubiquitylome, phosphoproteome, and acetylome analyses of sample-limited tissues. Nature Communications, 14(1), 1851.
Abelin, J. G., Harjanto, D., Malloy, M., Suri, P., Colson, T., Goulding, S. P., Creech, A. L., Serrano, L. R., Nasir, G., Nasrullah, Y., McGann, C. D., Velez, D., Ting, Y. S., Poran, A., Rothenberg, D. A., Chhangawala, S., Rubinsteyn, A., Hammerbacher, J., Gaynor, R. B., Fritsch, E. F., Greshock, J., Oslund, R. C., Barthelme, D., Addona, T. A., Arieta, C. M., & Rooney, M. S. (2019). Defining HLA-II Ligand Processing and Binding Rules with Mass Spectrometry Enhances Cancer Epitope Prediction. Immunity, 51(4), 766-779.e17.
Abelin, J. G., Keskin, D. B., Sarkizova, S., Hartigan, C. R., Zhang, W., Sidney, J., Stevens, J., Lane, W., Zhang, G. L., Eisenhaure, T. M., Clauser, K. R., Hacohen, N., Rooney, M. S., Carr, S. A., & Wu, C. J. (2017). Mass Spectrometry Profiling of HLA-Associated Peptidomes in Mono-allelic Cells Enables More Accurate Epitope Prediction. Immunity, 46(2), 315-326.
Andreatta, M., Alvarez, B., & Nielsen, M. (2017). GibbsCluster: unsupervised clustering and alignment of peptide sequences. Nucleic Acids Research, 45(W1), W458-W463.
Arieta, C. M., Xie, Y. J., Rothenberg, D. A., Diao, H., Harjanto, D., Meda, S., Marquart, K., Koenitzer, B., Sciuto, T. E., Lobo, A., & Others. (2023). The T-cell-directed vaccine BNT162b4 encoding conserved non-spike antigens protects animals from severe SARS-CoV-2 infection. Cell. https://www.cell.com/cell/pdf/S0092-8674(23)00403-8.pdf
Baden, L. R., El Sahly, H. M., Essink, B., Kotloff, K., Frey, S., Novak, R., Diemert, D., Spector, S. A., Rouphael, N., Creech, C. B., McGettigan, J., Khetan, S., Segall, N., Solis, J., Brosz, A., Fierro, C., Schwartz, H., Neuzil, K., Corey, L., . . . COVE Study Group. (2021). Efficacy and Safety of the mRNA-1273 SARS-CoV-2 Vaccine. The New England Journal of Medicine, 384(5), 403-416.
Barouch, D. H. (2022). Covid-19 Vaccines—Immunity, Variants, Boosters. In New England Journal of Medicine (Vol. 387, Issue 11, pp. 1011-1020). doi.org/10.1056/nejmra2206573.
Bassani-Sternberg, M., & Gfeller, D. (2016). Unsupervised HLA Peptidome Deconvolution Improves Ligand Prediction Accuracy and Predicts Cooperative Effects in Peptide-HLA Interactions. Journal of Immunology, 197(6), 2492-2499.
Becerra-Artiles, A., Cruz, J., Leszyk, J. D., Sidney, J., Sette, A., Shaffer, S. A., & Stern, L. J. (2019). Naturally processed HLA-DR3-restricted HHV-6B peptides are recognized broadly with polyfunctional and cytotoxic CD4 T-cell responses. European Journal of Immunology, 49(8), 1167-1185.
Becerra-Artiles, A., Nanaware, P. P., Muneeruddin, K., Weaver, G. C., Shaffer, S. A., Calvo-Calle, J. M., & Stern, L. J. (2022). Immunopeptidome profiling of human coronavirus OC43-infected cells identifies CD4 T cell epitopes specific to seasonal coronaviruses or cross-reactive with SARS-CoV-2. bioRxiv: The Preprint Server for Biology. doi.org/10.1101/2022.12.01.518643
Cagliani, R., Forni, D., Clerici, M., & Sironi, M. (2020). Coding potential and sequence conservation of SARS-CoV-2 and related animal viruses. Infection, Genetics and Evolution: Journal of Molecular Epidemiology and Evolutionary Genetics in Infectious Diseases, 83, 104353.
Cassotta, A., Paparoditis, P., Geiger, R., Mettu, R. R., Landry, S. J., Donati, A., Benevento, M., Foglierini, M., Lewis, D. J. M., Lanzavecchia, A., & Sallusto, F. (2020). Deciphering and predicting CD4+ T cell immunodominance of influenza virus hemagglutinin. The Journal of Experimental Medicine, 217(10). doi.org/10.1084/jem.20200206
Chen, Z., Ruan, P., Wang, L., Nie, X., Ma, X., & Tan, Y. (2021). T and B cell Epitope analysis of SARS-CoV-2 S protein based on immunoinformatics and experimental research. Journal of Cellular and Molecular Medicine, 25(2), 1274-1289.
Chong, C., Marino, F., Pak, H., Racle, J., Daniel, R. T., Müller, M., Gfeller, D., Coukos, G., & Bassani-Sternberg, M. (2018). High-throughput and Sensitive Immunopeptidomics Platform Reveals Profound Interferonγ-Mediated Remodeling of the Human Leukocyte Antigen (HLA) Ligandome. Molecular & Cellular Proteomics: MCP, 17(3), 533-548.
Creech, C. B., Walker, S. C., & Samuels, R. J. (2021). SARS-CoV-2 Vaccines. JAMA: The Journal of the American Medical Association, 325(13), 1318-1320.
Croft, N. P., Smith, S. A., Wong, Y. C., Tan, C. T., Dudek, N. L., Flesch, I. E. A., Lin, L. C. W., Tscharke, D. C., & Purcell, A. W. (2013). Kinetics of antigen expression and epitope presentation during virus infection. PLoS Pathogens, 9(1), e1003129.
Dai, L., & Gao, G. F. (2021). Viral targets for vaccines against COVID-19. Nature Reviews. Immunology, 21(2), 73-82.
Deffrennes, V., Vedrenne, J., Stolzenberg, M. C., Piskurich, J., Barbieri, G., Ting, J. P., Charron, D., & Alcaïde-Loridan, C. (2001). Constitutive expression of MHC class II genes in melanoma cell lines results from the transcription of class II transactivator abnormally initiated from its B cell-specific promoter. Journal of Immunology, 167(1), 98-106.
Dutta Noton K., Mazumdar Kaushiki, & Gordy James T. (2020). The Nucleocapsid Protein of SARS-CoV-2: a Target for Vaccine Development. Journal of Virology, 94(13), e00647-20.
Ferretti, A. P., Kula, T., Wang, Y., Nguyen, D. M. V., Weinheimer, A., Dunlap, G. S., Xu, Q., Nabilsi, N., Perullo, C. R., Cristofaro, A. W., Whitton, H. J., Virbasius, A., Olivier, K. J., Jr, Buckner, L. R., Alistar, A. T., Whitman, E. D., Bertino, S. A., Chattopadhyay, S., & MacBeath, G. (2020). Unbiased Screens Show CD8+ T Cells of COVID-19 Patients Recognize Shared Epitopes in SARS-CoV-2 that Largely Reside outside the Spike Protein. Immunity, 53(5), 1095-1107.e3.
Finkel, Y., Mizrahi, O., & Nachshon, A. (2020). The coding capacity of SARS-CoV-2. bioRxiv. www.biorxiv.org/content/10.1101/2020.05.07.082909v1.abstract
Firth, A. E. (2020). A putative new SARS-CoV protein, 3c, encoded in an ORF overlapping ORF3a. The Journal of General Virology, 101(10), 1085-1089.
Forlani, G., Michaux, J., Pak, H., Huber, F., Marie Joseph, E. L., Ramia, E., Stevenson, B. J., Linnebacher, M., Accolla, R. S., & Bassani-Sternberg, M. (2021). CIITA-Transduced Glioblastoma Cells Uncover a Rich Repertoire of Clinically Relevant Tumor-Associated HLA-II Antigens. Molecular & Cellular Proteomics: MCP, 20, 100032.
Gangaev, A., Ketelaars, S. L. C., Isaeva, O. I., Patiwael, S., Dopler, A., Hoefakker, K., De Biasi, S., Gibellini, L., Mussini, C., Guaraldi, G., Girardis, M., Ormeno, C. M. P. T., Hekking, P. J. M., Lardy, N. M., Toebes, M., Balderas, R., Schumacher, T. N., Ovaa, H., Cossarizza, A., & Kvistborg, P. (2021). Identification and characterization of a SARS-CoV-2 specific CD8+ T cell response with immunodominant features. Nature Communications, 12(1), 2593.
Gentili, M., Liu, B., Papanastasiou, M., Dele-Oni, D., Schwartz, M. A., Carlson, R. J., Al'Khafaji, A. M., Krug, K., Brown, A., Doench, J. G., Carr, S. A., & Hacohen, N. (2023). ESCRT-dependent STING degradation inhibits steady-state and cGAMP-induced signalling. Nature Communications, 14(1), 611.
Grifoni, A., Sidney, J., Vita, R., Peters, B., Crotty, S., Weiskopf, D., & Sette, A. (2021). SARS-CoV-2 human T cell epitopes: Adaptive immune response against COVID-19. Cell Host & Microbe, 29(7), 1076-1092.
Hajnik, R. L., Plante, J. A., Liang, Y., Alameh, M.-G., Tang, J., Bonam, S. R., Zhong, C., Adam, A., Scharton, D., Rafael, G. H., Liu, Y., Hazell, N. C., Sun, J., Soong, L., Shi, P.-Y., Wang, T., Walker, D. H., Sun, J., Weissman, D., . . . Hu, H. (2022). Dual spike and nucleocapsid mRNA vaccination confer protection against SARS-CoV-2 Omicron and Delta variants in preclinical models. Science Translational Medicine, 14(662), eabq1945.
Hos, B. J., Tondini, E., Camps, M. G. M., Rademaker, W., van den Bulk, J., Ruano, D., Janssen, G. M. C., de Ru, A. H., van den Elsen, P. J., de Miranda, N. F. C. C., van Veelen, P. A., & Ossendorp, F. (2022). Cancer-specific T helper shared and neo-epitopes uncovered by expression of the MHC class II master regulator CIITA. Cell Reports, 41(2), 111485.
Joag, V., Wijeyesinghe, S., Stolley, J. M., Quarnstrom, C. F., Dileepan, T., Soerens, A. G., Sangala, J. A., O'Flanagan, S. D., Gavil, N. V., Hong, S.-W., Bhela, S., Gangadhara, S., Weyu, E., Matchett, W. E., Thiede, J., Krishna, V., Cheeran, M. C.-J., Bold, T. D., Amara, R., . . . Masopust, D. (2021). Cutting Edge: Mouse SARS-CoV-2 Epitope Reveals Infection and Vaccine-Elicited CD8 T Cell Responses. Journal of Immunology, 206(5), 931-935.
Jungreis, I., Sealfon, R., & Kellis, M. (2021). SARS-CoV-2 gene content and COVID-19 mutation impact by comparing 44 Sarbecovirus genomes. Nature Communications, 12(1), 2642.
Kared, H., Redd, A. D., Bloch, E. M., Bonny, T. S., Sumatoh, H. R., Kairi, F., Carbajo, D., Abel, B., Newell, E. W., Bettinotti, M., Benner, S. E., Patel, E. U., Littlefield, K., Laeyendecker, O., Shoham, S., Sullivan, D., Casadevall, A., Pekosz, A., Nardin, A., . . . Quinn, T. C. (2021). SARS-CoV-2-specific CD8+ T cell responses in convalescent COVID-19 individuals. The Journal of Clinical Investigation. https://doi.org/10.1172/JCI145476
Keller, M. D., Harris, K. M., Jensen-Wachspress, M. A., Kankate, V. V., Lang, H., Lazarski, C. A., Durkee-Shock, J., Lee, P.-H., Chaudhry, K., Webber, K., Datar, A., Terpilowski, M., Reynolds, E. K., Stevenson, E. M., Val, S., Shancer, Z., Zhang, N., Ulrey, R., Ekanem, U., Sanojevic, M., Geiger, A., Liang, H., Hoq, F., Abraham. A. A., Hanley, P. J., Cruz, C. R., Ferrer, K., Dropulic, L., Gangler, K., Burbelo, P. D., Jones, R. B., Cohen, J. I, & Bollard, C. M. (2020). SARS-CoV-2-specific T cells are rapidly expanded for therapeutic use and target conserved regions of the membrane protein. Blood, 136(25), 2905-2917.
Knierman, M. D., Lannan, M. B., Spindler, L. J., McMillian, C. L., Konrad, R. J., & Siegel, R. W. (2020). The Human Leukocyte Antigen Class II Immunopeptidome of the SARS-CoV-2 Spike Glycoprotein. Cell Reports, 33(9), 108454.
Krammer, F. (2020). SARS-CoV-2 vaccines in development. Nature, 586(7830), 516-527.
Kyriakidis, N. C., López-Cortés, A., González, E. V., Grimaldos, A. B., & Prado, E. O. (2021). SARS-CoV-2 vaccines strategies: a comprehensive review of phase 3 candidates. NPJ Vaccines, 6(1), 28.
Le Bert, N., Clapham, H. E., Tan, A. T., Chia, W. N., Tham, C. Y. L., Lim, J. M., Kunasegaran, K., Tan, L. W. L., Dutertre, C.-A., Shankar, N., Lim, J. M. E., Sun, L. J., Zahari, M., Tun, Z. M., Kumar, V., Lim, B. L., Lim, S. H., Chia, A., Tan, Y.-J., Tambyah, P. A., Kalimuddin, S., Lye, D., Low, J. G. H., Wang, L-F., Wan, W.-Y., Hsu, L. Y., Bertoletti, A., Tam, C. C. (2021). Highly functional virus-specific cellular immune response in asymptomatic SARS-CoV-2 infection. The Journal of Experimental Medicine, 218(5). doi.org/10.1084/jem.20202617.
Le Bert, N., Tan, A. T., Kunasegaran, K., Tham, C. Y. L., Hafezi, M., Chia, A., Chng, M. H. Y., Lin, M., Tan, N., Linster, M., Chia, W. N., Chen, M. I.-C., Wang, L.-F., Ooi, E. E., Kalimuddin, S., Tambyah, P. A., Low, J. G.-H., Tan, Y.-J., & Bertoletti, A. (2020). SARS-CoV-2-specific T cell immunity in cases of COVID-19 and SARS, and uninfected controls. Nature. doi.org/10.1038/s41586-020-2550-z.
Lee, E., Sandgren, K., Duette, G., Stylianou, V. V., Khanna, R., Eden, J.-S., Blyth, E., Gottlieb, D., Cunningham, A. L., & Palmer, S. (2021). Identification of SARS-CoV-2 Nucleocapsid and Spike T-Cell Epitopes for Assessing T-Cell Immunity. Journal of Virology, 95(6). https://doi.org/10.1128/JVI.02002-20
Lippolis, J. D., White, F. M., Marto, J. A., Luckey, C. J., Bullock, T. N. J., Shabanowitz, J., Hunt, D. F., & Engelhard, V. H. (2002). Analysis of MHC class II antigen processing by quantitation of peptides that constitute nested sets. Journal of Immunology, 169(9), 5089-5097.
Mahajan, S., Kode, V., Bhojak, K., Karunakaran, C., Lee, K., Manoharan, M., Ramesh, A., Hv, S., Srivastava, A., Sathian, R., Khan, T., Kumar, P., Gupta, R., Chakraborty, P., & Chaudhuri, A. (2021). Immunodominant T-cell epitopes from the SARS-CoV-2 spike antigen reveal robust pre-existing T-cell immunity in unexposed individuals. Scientific Reports, 11(1), 13164.
Martínez-Flores, D., Zepeda-Cervantes, J., Cruz-Reséndiz, A., Aguirre-Sampieri, S., Sampieri, A., & Vaca, L. (2021). SARS-CoV-2 Vaccines Based on the Spike Glycoprotein and Implications of New Viral Variants. Frontiers in Immunology, 12. https://doi.org/10.3389/fimmu.2021.701501
Matchett, W. E., Joag, V., Stolley, J. M., Shepherd, F. K., Quarnstrom, C. F., Mickelson, C. K., Wijeyesinghe, S., Soerens, A. G., Becker, S., Thiede, J. M., Weyu, E., O'Flanagan, S. D., Walter, J. A., Vu, M. N., Menachery, V. D., Bold, T. D., Vezys, V., Jenkins, M. K., Langlois, R. A., & Masopust, D. (2021). Cutting Edge: Nucleocapsid Vaccine Elicits Spike-Independent SARS-CoV-2 Protective Immunity. Journal of Immunology, 207(2), 376-379.
Mateus, J., Grifoni, A., Tarke, A., Sidney, J., Ramirez, S. I., Dan, J. M., Burger, Z. C., Rawlings, S. A., Smith, D. M., Phillips, E., Mallal, S., Lammers, M., Rubiro, P., Quiambao, L., Sutherland, A., Yu, E. D., da Silva Antunes, R., Greenbaum, J., Frazier, A., Markmann, A. J., Premkumar, L., de Silva, A., Peters, B., Crotty, S. Sette, A., Weiskopf, D. (2020). Selective and cross-reactive SARS-CoV-2 T cell epitopes in unexposed humans. Science, 370(6512), 89-94.
McMurtrey, C. P., Lelic, A., Piazza, P., Chakrabarti, A. K., Yablonsky, E. J., Wahl, A., Bardet, W., Eckerd, A., Cook, R. L., Hess, R., Buchli, R., Loeb, M., Rinaldo, C. R., Bramson, J., & Hildebrand, W. H. (2008). Epitope discovery in West Nile virus infection: Identification and immune recognition of viral epitopes. Proceedings of the National Academy of Sciences of the United States of America, 105(8), 2981-2986.
Nagler, A., Kalaora, S., Barbolin, C., Gangaev, A., Ketelaars, S. L. C., Alon, M., Pai, J., Benedek, G., Yahalom-Ronen, Y., Erez, N., Greenberg, P., Yagel, G., Peri, A., Levin, Y., Satpathy, A. T., Bar-Haim, E., Paran, N., Kvistborg, P., & Samuels, Y. (2021). Identification of presented SARS-CoV-2 HLA class I and HLA class II peptides using HLA peptidomics. Cell Reports, 35(13), 109305.
Neale, I., Ali, M., Kronsteiner, B., Longet, S., Abraham, P., Deeks, A. S., Brown, A., Moore, S. C., Stafford, L., Dobson, S. L., Plowright, M., Newman, T. A. H., Wu, M. Y., Carr, E. J., Beale, R., Otter, A. D., Hopkins, S., Hall, V., Tomic, A., . . . Crick COVID Immunity Pipeline. (2023). CD4+ and CD8+ T cell and antibody correlates of protection against Delta vaccine breakthrough infection: A nested case-control study within the PITCH study. In medRxiv. https://doi.org/10.1101/2023.02.16.23285748
Nelde, A., Bilich, T., Heitmann, J. S., Maringer, Y., Salih, H. R., Roerden, M., Lübke, M., Bauer, J., Rieth, J., Wacker, M., Peter, A., Hörber, S., Traenkle, B., Kaiser, P. D., Rothbauer, U., Becker, M., Junker, D., Krause, G., Strengert, M., Schneiderhan-Marra, N., Templin, M. F., Joos, T. O., Kowalewski, D. J., Stos-Zweifel, V., Fehr, M., Rabsteyn, A., Mirakaj, V., Karbach, J., Jäger, E., Graf, M., Gruber, L.-C., Rachfalski, D., Preuß, B., Hagelstein, I., Märklin, M., Bakchoul, T., Gouttefangeas, C., Kohlbacher, O., Klein, R., Stevanović, S., Rammensee, H-G., Walz, J. S. (2021). SARS-CoV-2-derived peptides define heterologous and COVID-19-induced T cell recognition. Nature Immunology, 22(1), 74-85.
Neuwelt, A. J., Kimball, A. K., Johnson, A. M., Arnold, B. W., Bullock, B. L., Kaspar, R. E., Kleczko, E. K., Kwak, J. W., Wu, M.-H., Heasley, L. E., Doebele, R. C., Li, H. Y., Nemenoff, R. A., & Clambey, E. T. (2020). Cancer cell-intrinsic expression of MHC II in lung cancer cell lines is actively restricted by MEK/ERK signaling and epigenetic mechanisms. Journal for Immunotherapy of Cancer, 8(1). doi.org/10.1136/jitc-2019-000441.
Nielsen, S. S., Vibholm, L. K., Monrad, I., Olesen, R., Frattari, G. S., Pahus, M. H., Højen, J. F., Gunst, J. D., Erikstrup, C., Holleufer, A., Hartmann, R., Østergaard, L., Søgaard, O. S., Schleimann, M. H., & Tolstrup, M. (2021). SARS-CoV-2 elicits robust adaptive immune responses regardless of disease severity. EBioMedicine, 68, 103410.
Oronsky, B., Larson, C., Caroen, S., Hedjran, F., Sanchez, A., Prokopenko, E., & Reid, T. (2022). Nucleocapsid as a next-generation COVID-19 vaccine candidate. International Journal of Infectious Diseases: IJID: Official Publication of the International Society for Infectious Diseases, 122, 529-530.
Ovsyannikova, I. G., Johnson, K. L., Naylor, S., Muddiman, D. C., & Poland, G. A. (2003). Naturally processed measles virus peptide eluted from class II HLA-DRB1*03 recognized by T lymphocytes from human blood. Virology, 312(2), 495-506.
Pardi, N., Hogan, M. J., Porter, F. W., & Weissman, D. (2018). mRNA vaccines—a new era in vaccinology. In Nature Reviews Drug Discovery (Vol. 17, Issue 4, pp. 261-279). doi.org/10.1038/nrd.2017.243.
Parker, R., Partridge, T., Wormald, C., Kawahara, R., Stalls, V., Aggelakopoulou, M., Parker, J., Powell Doherty, R., Ariosa Morejon, Y., Lee, E., Saunders, K., Haynes, B. F., Acharya, P., Thaysen-Andersen, M., Borrow, P., & Ternette, N. (2021). Mapping the SARS-CoV-2 spike glycoprotein-derived peptidome presented by HLA class II on dendritic cells. Cell Reports, 35(8), 109179.
Peng, Y., Mentzer, A. J., Liu, G., Yao, X., Yin, Z., Dong, D., Dejnirattisai, W., Rostron, T., Supasa, P., Liu, C., López-Camacho, C., Slon-Campos, J., Zhao, Y., Stuart, D. I., Paesen, G. C., Grimes, J. M., Antson, A. A., Bayfield, O. W., Hawkins, D. E. D. P., Ker, D.-S., Wang, B., Turtle, L., Subramaniam, K., Thomson, P., Zhang, P., Dold, C., Ratcliff, J., Simmonds, P., de Silva, T., Sopp, P., Wellington, D., Rajapaksa, U., Chen, Y.-L., Salio, M., Napolitani, G., Paes, W., Borrow, P., Kessler, B. M., Fry, J. W., Schwabe, N. F., Semple, M. G., Baillie, J. K., Moore, S. C., Openshaw, P. J. M., Ansari, M. A., Dunachie, S., Barnes, E., Frater, J., Kerr, G., Goulder, P., Lockett, T., Levin, R., Zhang, Y., Jing, R., Ho, L.-P., Oxford Immunology Network Covid-19 Response T cell Consortium, ISARIC4C Investigators, Cornall, R. J., Conlon, C. P., Klenerman, P., Screaton, G. R., Mongkolsapaya, J., McMichael, A., Knight, J. C., Ogg, G., Dong, T. (2020). Broad and strong memory CD4+ and CD8+ T cells induced by SARS-CoV-2 in UK convalescent individuals following COVID-19. Nature Immunology, 21(11), 1336-1345.
Prakash, S., Srivastava, R., Coulon, P.-G., Dhanushkodi, N. R., Chentoufi, A. A., Tifrea, D. F., Edwards, R. A., Figueroa, C. J., Schubl, S. D., Hsieh, L., Buchmeier, M. J., Bouziane, M., Nesburn, A. B., Kuppermann, B. D., & BenMohamed, L. (2021). Genome-Wide B Cell, CD4+, and CD8+ T Cell Epitopes That Are Highly Conserved between Human and Animal Coronaviruses, Identified from SARS-CoV-2 as Targets for Preemptive Pan-Coronavirus Vaccines. Journal of Immunology, 206(11), 2566-2582.
Prakash, S., Srivastava, R., Coulon, P.-G., Dhanushkodi, N. R., Chentoufi, A. A., Tifrea, D. F., Edwards, R. A., Figueroa, C., Schubl, S. D., Hsieh, L., Buchmeier, M. J., Bouziane, M., Nesburn, A. B., Kuppermann, B. D., & Benmohamed, L. (n.d.). Genome-Wide Asymptomatic B-Cell, CD4 and CD8 T-Cell Epitopes, that are Highly Conserved between Human and Animal Coronaviruses, Identified from SARS-CoV-2 as Immune Targets for Pre-Emptive Pan-Coronavirus Vaccines. In SSRN Electronic Journal. doi.org/10.2139/ssrn.3712675.
Rha, M.-S., Jeong, H. W., Ko, J.-H., Choi, S. J., Seo, I.-H., Lee, J. S., Sa, M., Kim, A. R., Joo, E.-J., Ahn, J. Y., Kim, J. H., Song, K.-H., Kim, E. S., Oh, D. H., Ahn, M. Y., Choi, H. K., Jeon, J. H., Choi, J.-P., Kim, H. B., . . . Shin, E.-C. (2021). PD-1-Expressing SARS-CoV-2-Specific CD8+ T Cells Are Not Exhausted, but Functional in Patients with COVID-19. Immunity, 54(1), 44-52.e3.
Rucevic, M., Kourjian, G., Boucau, J., Blatnik, R., Garcia Bertran, W., Berberich, M. J., Walker, B. D., Riemer, A. B., & Le Gall, S. (2016). Analysis of Major Histocompatibility Complex-Bound HIV Peptides Identified from Various Cell Types Reveals Common Nested Peptides and Novel T Cell Responses. Journal of Virology, 90(19), 8605-8620.
Ruiz Cuevas, M. V., Hardy, M.-P., Hollý, J., Bonneil, É., Durette, C., Courcelles, M., Lanoix, J., Côté, C., Staudt, L. M., Lemieux, S., Thibault, P., Perreault, C., & Yewdell, J. W. (2021). Most non-canonical proteins uniquely populate the proteome or immunopeptidome. Cell Reports, 34(10), 108815.
Sahin, U., Muik, A., Vogler, I., Derhovanessian, E., Kranz, L. M., Vormehr, M., Quandt, J., Bidmon, N., Ulges, A., Baum, A., Pascal, K. E., Maurus, D., Brachtendorf, S., Lörks, V., Sikorski, J., Koch, P., Hilker, R., Becker, D., Eller, A.-K., . . . Türeci, Ö. (2021). BNT162b2 vaccine induces neutralizing antibodies and poly-specific T cells in humans. Nature, 595(7868), 572-577.
Sarkizova, S., Klaeger, S., Le, P. M., Li, L. W., Oliveira, G., Keshishian, H., Hartigan, C. R., Zhang, W., Braun, D. A., Ligon, K. L., Bachireddy, P., Zervantonakis, I. K., Rosenbluth, J. M., Ouspenskaia, T., Law, T., Justesen, S., Stevens, J., Lane, W. J., Eisenhaure, T., . . . Keskin, D. B. (2020). A large peptidome dataset improves HLA class I epitope prediction across most of the human population. Nature Biotechnology, 38(2), 199-209.
Schellens, I. M., Meiring, H. D., Hoof, I., Spijkers, S. N., Poelen, M. C. M., van Gaans-van den Brink, J. A. M., Costa, A. I., Vennema, H., Keşmir, C., van Baarle, D., & van Els, C. A. C. M. (2015). Measles Virus Epitope Presentation by HLA: Novel Insights into Epitope Selection, Dominance, and Microvariation. Frontiers in Immunology, 6, 546.
Sette, A., & Crotty, S. (2021). Adaptive immunity to SARS-CoV-2 and COVID-19. Cell, 184(4), 861-880.
Shomuradova, A. S., Vagida, M. S., Sheetikov, S. A., Zornikova, K. V., Kiryukhin, D., Titov, A., Peshkova, I. O., Khmelevskaya, A., Dianov, D. V., Malasheva, M., Shmelev, A., Serdyuk, Y., Bagaev, D. V., Pivnyuk, A., Shcherbinin, D. S., Maleeva, A. V., Shakirova, N. T., Pilunov, A., Malko, D. B., . . . Efimov, G. A. (2020). SARS-CoV-2 Epitopes Are Recognized by a Public and Diverse Repertoire of Human T Cell Receptors. Immunity, 53(6), 1245-1257.e5.
Silva, E. K. V. B., Bomfim, C. G., Barbosa, A. P., Noda, P., Noronha, I. L., Fernandes, B. H. V., Machado, R. R. G., Durigon, E. L., Catanozi, S., Rodrigues, L. G., Pieroni, F., Lima, S. G., Teodoro, W. R., Queiroz, Z. A. J., Silveira, L. K. R., Charlie-Silva, I., Capelozzi, V. L., Guzzo, C. R., & Fanelli, C. (2022). Immunization with SARS-CoV-2 Nucleocapsid protein triggers a pulmonary immune response in rats. PloS One, 17(5), e0268434.
Stewart, H., Lu, Y., O'Keefe, S., Valpadashi, A., Cruz-Zaragoza, L. D., Michel, H. A., Nguyen, S. K., Carnell, G. W., Lukhovitskaya, N., Milligan, R., Jungreis, I., Lulla, V., Davidson, A. D., Matthews, D. A., High, S., Rehling, P., Emmott, E., Heeney, J. L., Edgar, J. R., Smith, G. L., & Firth, A. E. (2022). The SARS-CoV-2 protein ORF3c is a mitochondrial modulator of innate immunity. In bioRxiv (p. 2022.11.15.516323). doi.org/10.1101/2022.11.15.516323.
Tarke, A., Sidney, J., Kidd, C. K., Dan, J. M., Ramirez, S. I., Yu, E. D., Mateus, J., da Silva Antunes, R., Moore, E., Rubiro, P., Methot, N., Phillips, E., Mallal, S., Frazier, A., Rawlings, S. A., Greenbaum, J. A., Peters, B., Smith, D. M., Crotty, S., Weiskopf, D, Grifoni, A., Sette, A. (2021). Comprehensive analysis of T cell immunodominance and immunoprevalence of SARS-CoV-2 epitopes in COVID-19 cases. Cell Reports. Medicine, 2(2), 100204.
Taylor, H. B., Klaeger, S., Clauser, K. R., Sarkizova, S., Weingarten-Gabbay, S., Graham, D. B., Carr, S. A., & Abelin, J. G. (2021). MS-Based HLA-II Peptidomics Combined With Multiomics Will Aid the Development of Future Immunotherapies. Molecular & Cellular Proteomics: MCP, 20, 100116.
Ternette, N., Yang, H., Partridge, T., Llano, A., Cedeño, S., Fischer, R., Charles, P. D., Dudek, N. L., Mothe, B., Crespo, M., Fischer, W. M., Korber, B. T. M., Nielsen, M., Borrow, P., Purcell, A. W., Brander, C., Dorrell, L., Kessler, B. M., & Hanke, T. (2016). Defining the HLA class I-associated viral antigen repertoire from HIV-1-infected human cells. European Journal of Immunology, 46(1), 60-69.
Weingarten-Gabbay, S., Klaeger, S., Sarkizova, S., Pearlman, L. R., Chen, D.-Y., Gallagher, K. M. E., Bauer, M. R., Taylor, H. B., Dunn, W. A., Tarr, C., Sidney, J., Rachimi, S., Conway, H. L., Katsis, K., Wang, Y., Leistritz-Edwards, D., Durkin, M. R., Tomkins-Tinch, C. H., Finkel, Y., Nachshon, A., Gentili, M., Rivera, K. D., Carulli, I. P., Chea, V. A., Chandrashekar, A., Bozkus, C. C., Carrington, M., MGH COVID-19 Collection & Processing Team, Bhardwaj, N., Barouch, D. H., Sette, A., Maus, M. V., Rice, C. M., Clauser, K. R., Keskin, D. B., Pregibon, D. C., Hacohen, N., Carr, S. A., Abelin, J. G., Saeed, M., & Sabeti, P. C. (2021). Profiling SARS-CoV-2 HLA-I peptidome reveals T cell epitopes from out-of-frame ORFs. Cell, 184(15), 3962-3980.e17.
Weingarten-Gabbay, S., Pearlman, L. R., Chen, D.-Y., Klaeger, S., Taylor, H. B., Welch, N. L., Keskin, D. B., Carr, S. A., Abelin, J. G., Saeed, M., & Sabeti, P. C. (2022). HLA-I immunopeptidome profiling of human cells infected with high-containment enveloped viruses. STAR Protocols, 3(4), 101910.
Wherry, E. J., & Barouch, D. H. (2022). T cell immunity to COVID-19 vaccines. Science, 377(6608), 821-822.
Wosen, J. E., Mukhopadhyay, D., Macaubas, C., & Mellins, E. D. (2018). Epithelial MHC Class II Expression and Its Role in Antigen Presentation in the Gastrointestinal and Respiratory Tracts. Frontiers in Immunology, 9, 2144.
Wu, F., Zhao, S., Yu, B., Chen, Y.-M., Wang, W., Song, Z.-G., Hu, Y., Tao, Z.-W., Tian, J.-H., Pei, Y.-Y., Yuan, M.-L., Zhang, Y.-L., Dai, F.-H., Liu, Y., Wang, Q.-M., Zheng, J.-J., Xu, L., Holmes, E. C., & Zhang, Y.-Z. (2020). A new coronavirus associated with human respiratory disease in China. Nature, 579(7798), 265-269.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. An immunogenic composition comprising one or more peptides, wherein the one or more peptides are:
a. capable of binding to Major Histocompatibility Complex (MHC) class II, wherein the MHC class II is Human Leukocyte Antigen class II (HLA-II), and
b. derived from translation products of SARS-CoV-2.

2. (canceled)

3. The immunogenic composition according to claim 2, wherein the one or more peptides have a peptide-HLA-II binding affinity of less than 500 nMa.

4. The immunogenic composition according to claim 1, wherein the one or more peptides have a peptide-HLA-II binding affinity of less than 500 nMa, and wherein the HLA-II is encoded by an HLA allele selected from the group consisting of:
$$\begin{matrix}HLA - DRB 1 * 07 : 01 , \\ HLA - DRB 1 * 11 : 04 , \\ HLA - DRB 1 * 15 : 01 , \\ HLA - DRB 3 * 02 : 02 , \\ HLA - DRB 4 * 01 : 01 , \\ HLA - DRB 5 * 01 : 01 , \\ HLA - DPB 1 * 03 : 01 / HLA - DPA 1 * 01 : 03 , \\ HLA - DPB 1 * 04 : 02 / HLA - DPA 1 * 01 : 03 , \\ HLA - DPB 1 * 06 : 01 / HLA - DPA 1 * 01 : 03 , \\ HLA - DQB 1 * 02 : 02 / HLA - DQA 1 * 02 : 01 , \\ HLA - DQB 1 * 02 : 02 / HLA - DQA 1 * 05 : 05 , \\ HLA - DQB 1 * 03 : 01 / HLA - DQA 1 * 02 : 01 , \\ HLA - DQB 1 * 03 : 01 / HLA - DQA 1 * 05 : 05 , and \\ HLA - DQB 1 * 06 : 02 / HLA - DQA 1 * 01 : 02 .\end{matrix}$$

5. (canceled)

6. The immunogenic composition according to claim 1, wherein at least one of the peptides is derived from translation of one or more internal out-of-frame open reading frames (ORFs) of SARS-CoV-2, wherein at least one of the internal out-of-frame ORFs is selected from the group consisting of ORF3c (ORF3a.iORF1) and/or ORF9b (NiORF1): one or more canonical ORFs of SARS-CoV-2, wherein at least one of the canonical ORFs is selected from the group consisting of non-structural protein 3 (nsp3) ORF, non-structural protein 4 (nsp4) ORF, ORF3a, ORF6, S protein ORF, M protein ORF, N protein ORF, and any combination thereof; or any combination thereof.

7. (canceled)

8. (canceled)

9. The immunogenic composition according to claim 1, wherein at least one of the peptides comprises a peptide sequence selected from the group consisting of internal ORF protein peptide sequences, canonical ORF protein peptide sequences, any subsequence thereof, and any combination thereof, and wherein at least one of the peptide sequences is selected from the group consisting of peptide sequences of Table 1, Table 2, and Table 4, any subsequence thereof, and any combination thereof, and wherein
a. the ORF3c overlaps with the ORF3a;
b. ORF9b overlaps with the N protein ORF;
c. at least one of the peptides derived from translation of ORF3c comprises a peptide sequence of LLFFRALPK (SEQ ID NO: 546), ALHFLLFFRALPKS (SEQ ID NO: 374), or any subsequence thereof; and
d. at least one of the peptides derived from translation of ORF9b comprises a peptide sequence selected from the group consisting of PKVYPIILR (SEQ ID NO: 547), ISEMHPALR (SEQ ID NO: 548), VGPKVYPIILRLGSPLS (SEQ ID NO: 384), MDPKISEMHPALRLVDPQIQLAVTRMENA (SEQ ID NO: 382), any subsequence thereof, and any combination thereof.

10. (canceled)

11. (canceled)

12. (canceled)

13. (canceled)

14. (canceled)

15. (canceled)

16. The immunogenic composition according to claim 6, wherein at least one of the peptides derived from translation of the nsp3 ORF comprises a peptide sequence of
(SEQ ID NO: 549)
VTAYNGYLT,
(SEQ ID NO: 363)
DGSEDNQTTTIQTIVE,
(SEQ ID NO: 364)
SPDAVTAYNGYLTSSSK,
or any subsequence thereof.

17. (canceled)

18. The immunogenic composition according to claim 6, wherein at least one of the peptides derived from translation of the nsp4 ORF comprises a peptide sequence of
(SEQ ID NO: 550)
IIQFPNTYL,
(SEQ ID NO: 365)
MDGSIIQFPNTYLEGSVR,
or any subsequence thereof.

19. (canceled)

20. The immunogenic composition according to claim 6, wherein at least one of the peptides derived from translation of ORF3a comprises a peptide sequence selected from the group consisting of
(SEQ ID NO: 551)
IKDATPSDF,
(SEQ ID NO: 552)
FTIGTVTLK,
(SEQ ID NO: 99)
MDLFMRIFTIGTVTLKQGEIKDATPSDF,
any subsequence thereof, and any combination thereof.

21. (canceled)

22. The immunogenic composition according to claim 6, wherein at least one of the peptides derived from translation of ORF6 comprises a peptide sequence (Original) of
(SEQ ID NO: 533)
INLIIKNLS,
(SEQ ID NO: 100)
YIINLIIKNLSKS,
or any subsequence thereof.

23. (canceled)

24. The immunogenic composition according to claim 6, wherein at least one of the peptides derived from translation of the S protein ORF comprises a peptide sequence selected from the group consisting of
(SEQ ID NO: 554)
YTNSFTRGV,
(SEQ ID NO: 555)
FKNIDGYFK,
(SEQ ID NO: 556)
FQTLLALHR,
(SEQ ID NO: 557)
IYQTSNFRV,
(SEQ ID NO: 558)
FASVYAWNR,
(SEQ ID NO: 559)
FVIRGDEVR,
(SEQ ID NO: 560)
VIAWNSNNL,
(SEQ ID NO: 561)
IAWNSNNLD,
(SEQ ID NO: 562)
YQAGSTPCN,
(SEQ ID NO: 563)
FLPFQQFGR,
(SEQ ID NO: 564)
VYSTGSNVE,
(SEQ ID NO: 565)
YQTQTNSPR,
(SEQ ID NO: 566)
YTMSLGAEN,
(SEQ ID NO: 567)
LLQYGSFCT,
(SEQ ID NO: 568)
IAQYTSALL,
(SEQ ID NO: 569)
LQIPFAMQM,
(SEQ ID NO: 570)
FAMQMAYRF,
(SEQ ID NO: 571)
LIRAAEIRA,
(SEQ ID NO: 572)
IITTDNTFV,
(SEQ ID NO: 529)
TQLPPAYTNSFTRGVYYPDKVFRSSVLHSTQDLFLPFFS,
(SEQ ID NO: 530)
FKNLREFVFKNIDGYFKIYSKHTPINLVRDL,
(SEQ ID NO: 418)
INITRFQTLLALHRSYL,
(SEQ ID NO: 532)
TVEKGIYQTSNERVQPTES,
(SEQ ID NO: 424)
ATRFASVYAWNRKRISN,
(SEQ ID NO: 425)
DSFVIRGDEVRQIAPG,
(SEQ ID NO: 535)
NYKLPDDFTGCVIAWNSNNLDSKVG,
(SEQ ID NO: 440)
TEIYQAGSTPCNGVEG,
(SEQ ID NO: 442)
ESNKKFLPFQQFGRDIADTTDAVRDPQT,
(SEQ ID NO: 538)
TPTWRVYSTGSNVFQTRAG,
(SEQ ID NO: 445)
ICASYQTQTNSPRRA,
(SEQ ID NO: 446)
SVASQSIIAYTMSLGAEN,
(SEQ ID NO: 541)
CSNLLLQYGSFCTQLNRALTGIAVEQDKNTQE,
(SEQ ID NO: 119)
EPQIITTDNTFVSGN,
(SEQ ID NO: 460)
VLPPLLTDEMIAQYTSALLAGTIT,
(SEQ ID NO: 543)
AALQIPFAMQMAYRFNGIG,
(SEQ ID NO: 498)
TQQLIRAAEIRASANLA,
any subsequence thereof, and any combination thereof.

25. (canceled)

26. The immunogenic composition according to claim 6, wherein at least one of the peptides derived from translation of the M protein ORF comprises a peptide sequence selected from the group consisting of
(SEQ ID NO: 573)
LHGTILTRP,
(SEQ ID NO: 574)
YYKLGASQR,
(SEQ ID NO: 509)
NVPLHGTILTRPLLESELVIGAVILRGHLR
IAGHHLGRCDIKDLPKEITVA,
(SEQ ID NO: 139)
TSRTLSYYKLGASQRVAGDSG,
(SEQ ID NO: 22)
TDHSSSSDNIALLVQ,
any subsequence thereof, and any combination thereof.

27. (canceled)

28. The immunogenic composition according to claim 6, wherein at least one of the peptides derived from translation of the N protein ORF comprises a peptide sequence selected from the group consisting of
(SEQ ID NO: 575)
FTALTQHGK,
(SEQ ID NO: 576)
TGPEAGLPY,
(SEQ ID NO: 577)
LPQGTTLPK,
(SEQ ID NO: 578)
LLLLDRLNQ,
(SEQ ID NO: 579)
VTQAFGRRG,
(SEQ ID NO: 580)
FAPSASAFF,
(SEQ ID NO: 581)
VTPSGTWLT,
(SEQ ID NO: 582)
TQALPQRQK,
(SEQ ID NO: 513)
GLPNNTASWFTALTQHGKEDLKFPRGQGVPI
NTNSSPDDQIGYYRRATRRIR,
(SEQ ID NO: 32)
TGPEAGLPYGANKDG,
(SEQ ID NO: 164)
VATEGALNTPKDHIGTRNPANNAAIVLQLPQGTTLPKG,
(SEQ ID NO: 34)
MAGNGGDAALALLLLDRLNQLESKMSGKGQQQQGQTVT,
(SEQ ID NO: 517)
AAEASKKPRQKRTATKAYNVTQAFGRRGPEQTQGNFGDQELIRQGTD,
(SEQ ID NO: 55)
IAQFAPSASAFFG,
(SEQ ID NO: 24)
SDNGPQNQRNAPRITF,
(SEQ ID NO: 520)
KKKADETQALPQRQKKQQTVTLLPAADLDDFSKQLQQSMSSADSTQA,
(SEQ ID NO: 519)
RIGMEVTPSGTWLTYTGAIKLDDKDPNFKDQVILLNKHIDAYKTFPP,
any subsequence thereof, and any combination thereof.

29. (canceled)

30. The immunogenic composition according to claim 1, wherein at least one of the peptides comprises a peptide sequence selected from the group consisting of N₁XXN₂XN₃XXN₄, any subsequence thereof, and any combination thereof, and wherein: N₁ is F, I, L, M, V, W, or Y;
N₂ is A, I, F, L, M, N, T, Q, S, V, W, or Y;
N₃ is A, D, E, G, H, K, N, P, R, S, or T;
N₄ is A, E, F, G, K, I, L, N, M, R, S, V, or Q; and wherein:
N₁ is F, I, L, M, V, W, or Y; N₂ is N, T, S, or V: or Y; N₃ is A, G, N, P, S, or T; and N₄ is F, I, L, N, M, or V;
N₁ is I, L, M, or V; N₂ is I, L, M, or V; N₃ is H, K, or R; and N₄ is A, G, S, or Q;
N₁ is F, I, L, V, W, or Y; N₂ is F, I, N, M, W, or Y; N₃ is D, G, N, or S; and N₄ is K, L, N, S, or V; or
N₁ is F, I, L, M, V, W, or Y; N₂ is A, E, I, L, M, Q, or V; N₃ is A, E, G, or S; and N₄ is E, K, or R.

31. (canceled)

32. (canceled)

33. A vector comprising a polynucleotide encoding one or more peptides of claim 1, wherein the vector is a synthetic mRNA vaccine.

34. (canceled)

35. An immunogenic composition comprising:
a. one or more peptides of claim 1; and
b. one or more antigenic components capable of stimulating production of an antibody targeting SARS-CoV-2, wherein the one or more antigenic components comprises one or more antigenic peptides from a nucleocapsid phosphoprotein of SARS-CoV-2, a spike glycoprotein of SARS-CoV-2, or any combination thereof, one or more polynucleotides encoding the one or more antigenic peptides, or any combination thereof.

36. (canceled)

37. (canceled)

38. (canceled)

39. (canceled)

40. (canceled)

41. (canceled)

42. A method of identifying immunogenic peptides comprising:
a. lysing cells having a potential to express the immunogenic peptides of interest with a lysis buffer comprising a cell membrane disrupting detergent, wherein the cell membrane disrupting agent is a nonylphenol ethoxylate surfactant;
b. enzymatic shearing of nucleic acids in the lysed cells, wherein the nucleic acids in the lysed cells are enzymatically sheared using an endonuclease from Serratia marcescens and MgCl₂;
c. isolating HLA-II from the lysed cells, wherein the HLA-II is in complex with one or more peptides from the lysed cells, wherein isolating HLA-II comprises immunoprecipitation of the HLA-II complex with an anti-HLA-II antibody; and
d. determining sequences of the one or more peptides in complex with the HLA-II from (c), wherein (d) is performed by liquid chromatography tandem mass spectrometry analysis.

43. The method according to claim 42, further comprising
(e) identifying HLA alleles that bind the peptides identified in using a HLA-II epitope binding predictor;
(f) selecting a subset of peptides that bind a defined percentage of HLA-II alleles;
(g) selecting immunogenic peptides demonstrating a relative abundance above a defined threshold as determined by analysis of the complete cellular transcriptome and or proteome; and
(h) performing ribosome sequencing to identify actively translated peptides and selecting immunogenic peptides that are being actively translated at one or more time points.

44. (canceled)

45. (canceled)

46. (canceled)

47. (canceled)

48. (canceled)

49. (canceled)

50. The method according to claim 42, wherein the immunogenic peptides of interest are expressed by a pathogen and wherein the cells have been infected with the pathogen, wherein
(a) the infected cells are engineered to express one or more cell surface receptors used by the pathogen to infect the cells;
(b) the cells are treated with one or more cell signaling molecules related to infection by the pathogen;
(c) the pathogen is a virus, optionally SARS-CoV-2;
(d) the cells are engineered to express CIITA, ACE2, and TMPRSS2;
(e) the cells are engineered to increase or decrease HLA presentation;
(f) the cells are engineered to increase or decrease expression of one or more of CIITA, proteasome subunits, tPA, POMP, or ubiquitin-proteasome genes.

51. (canceled)

52. (canceled)

53. (canceled)

54. (canceled)

55. (canceled)

56. (canceled)

57. (canceled)
